(19) United States
(12) Patent Application Publication
(10) Pub. No.: US 2024/0167058 A1
(43) Pub. Date: May 23, 2024

(54) NUCLEIC ACID CONSTRUCTS AND USES THEREOF

(71) Applicant: Kanglin Biotech (Hangzhou) Co., Ltd., Hangzhou (CN)

(72) Inventors: Haoquan Wu, Hangzhou (CN); Ying Dang, Hangzhou (CN); Lingling Su, Hangzhou (CN); Qing Ye, Hangzhou (CN); Jianjun Tao, Hangzhou (CN); Xiaodong Hu, Hangzhou (CN); Chao Jiang, Hangzhou (CN)

(21) Appl. No.: 18/383,596

(22) Filed: Oct. 25, 2023

Related U.S. Application Data
(60) Provisional application No. 63/419,143, filed on Oct. 25, 2022.

(51) Int. Cl. C12N 15/86; A61K 31/10; A61K 31/675; A61K 35/28; A61K 48/00; A61P 7/00; C12N 5/0789

(52) U.S. Cl. CPC C12N 15/86; A61K 31/10; A61K 31/675; A61K 35/28; A61K 48/0066; A61P 7/00; C12N 5/0647; C12N 2510/00

(57) ABSTRACT

Disclosed herein are gene therapy vectors used for efficiently transducing cells to express a human β-globin gene. Specifically disclosed is an expression vector which comprises: an expression cassette for a β-globin gene, which comprises exons and introns of human β-globin gene, as well as cis-acting elements including one or more of WPRE, SV40 polyadenylation signal and/or SV40 ori. The disclosed expression vectors have significantly enhanced viral vector packaging efficiency in viral vector packaging cell lines, which leads to effective integration of lentiviral vectors and high expression level of β-globin gene in target cells. Also disclosed are pharmaceutical compositions and therapeutic methods utilizing such expression vectors.

### CROSS REFERENCE TO RELATED APPLICATIONS

This application claims the benefit of U.S. Provisional Application No. 63/419,143, filed Oct. 25, 2022. The contents of the aforesaid application are hereby incorporated by reference in their entirety.

### SEQUENCE LISTING

The instant application contains a Sequence Listing which has been submitted electronically in XML file format and is hereby incorporated by reference in its entirety. Said XML copy, created on Jan. 5, 2024, is named K2046-700610_SL.xml and is 177,672 bytes in size.

### FIELD OF DISCLOSURE

The present disclosure relates to the field of medical technology, in particular to gene therapy and nucleic acid constructs.

### BACKGROUND

Inherited anemias such as thalassemia and sickle cell anemia are rare inherited blood diseases, most commonly in patients of Mediterranean, Middle Eastern, Indian and South Asian descent. Thalassemia is typically derived from the imbalance between the single chains of globin hemoglobin tetramer. The imbalance of red blood cell (RBC) α-globin and β-globin often produces various clinical symptoms, for example, 1) lack of sufficient red blood cells and hemoglobin, resulting in inadequacy of oxygen delivered to the whole body; 2) an increase in the hemolysis rate of red blood cells, leading to an increase in the mortality rate of chronic vascular system damage; and 3) spleen and liver damages caused by extreme load of ferine.

Current treatment methods for inherited anemias include, for example, blood transfusion therapy, iron chelation therapy, and splenectomy or splenic artery embolization. Allogeneic hematopoietic stem cell transplantation (e.g., allogeneic bone marrow transplantation, peripheral blood hematopoietic stem cell transplantation, or cord blood transplantation) could be a potential cure for thalassemia. However, the lack of transplant donors and the risk associated with transplantation limit the widespread use of allogeneic hematopoietic cell transplantation in patients with thalassemia.

Thus, there still exists a need for developing new therapies for thalassemia.

### SUMMARY

In one aspect, the disclosure provides a vector comprising: a) a left (5′) retroviral LTR; b) human β-globin gene; c) human β-globin gene upstream locus control region (LCR); d) a cis-acting posttranscriptional regulatory element; e) a right (3′) retroviral LTR; and f) a SV40 polyadenylation signal and/or SV40 origin.

In some embodiments, the sequence of the human β-globin gene comprises human β-globin gene exon 1, intron 1, exon 2, intron 2, and exon 3. In certain embodiments, the sequence of the human β-globin gene is according to Ensembl Database Gene: HBB (ENSG00000244734) Transcript: HBB-201 (ENST00000335295.4). In some embodiment, the human β-globin gene comprises a human β-globin promoter. In some embodiments, human β-globin promoter is about 250 to about 275 bp (e.g., 268 bp) upstream of exon 1 of the human β-globin promoter. In some embodiments, the human β-globin gene comprises a human β-globin 3′-enhancer. In certain embodiments, the human β-globin 3′-enhancer is about 850 bp to about 900 bp (e.g., 878 bp) downstream of exon 3 of the human 3-globin gene. In some embodiments, the human β-globin gene comprises one or more (e.g., 2 or 3) wild-type exons. In some embodiments, the human β-globin gene comprises one or more (e.g., 2 or 3) codon-optimized exons. In some embodiments, the human β-globin gene comprises one or more wild-type introns. In certain embodiments, the human β-globin gene comprises a wild-type intron 2. In some embodiments, the human β-globin gene comprises one or more truncated introns. In certain embodiments, the human β-globin gene comprises a truncated intron 2. In some embodiments, the human β-globin gene comprises a wild-type exon 2. In some embodiments, the human β-globin gene comprises an exon 2 that encodes a threonine to glutamine mutation at codon 87 (T87Q). In some embodiments, the human β-globin gene comprises the nucleotide sequence of SEQ ID NO: 23, SEQ ID NO: 24, SEQ ID NO: 25, or a nucleotide sequence having at least 85%, 90%, 95%, 98%, or 99% sequence identity thereto, or any combination thereof.

In some embodiments, the upstream locus control region (LCR) comprises one or more (e.g., 2 or 3) truncated DNase I hypersensitive sites, HS2, HS3 and HS4 of the LCR. In some embodiments, the posttranscriptional regulatory element is a woodchuck hepatitis virus post-transcriptional regulatory element (WPRE). In some embodiments, the SV40 polyadenylation signal and/or SV40 origin is located 3′ of the right (3′) retroviral LTR.

In some embodiments, the WPRE is wildtype WPRE or a mutated WPRE, e.g., a mutated WPRE described herein. In some embodiments, the wildtype WPRE comprises the nucleotide sequence of SEQ ID NO: 32, or a nucleotide sequence having at least 85%, 90%, 95%, 98%, or 99% sequence identity thereto. In some embodiments, the mutated WPRE comprises the nucleotide sequence of SEQ ID NO: 33, or a nucleotide sequence having at least 85%, 90%, 95%, 98%, or 99% sequence identity thereto.

In some embodiments, the vector is a lentivirus vector. In some embodiments, the left (5′) retroviral LTR is a lentiviral LTR. In some embodiments, the right (3′) LTR is a lentivirus LTR. In some embodiments, the left (5′) and right (3′) retroviral LTRs are lentivirus LTRs. In some embodiments, the promoter of the left (5′) retroviral LTR is replaced with a heterologous promoter. In some embodiments, the right (3′) LTR is a self-inactivating (SIN) LTR.

In some embodiments, the vector further comprises one or more (e.g., 2 or 3) of a Psi packaging sequence (Ψ+), a central polypurine tract/DNA flap (cPPT/FLAP), or a retroviral export element-rev response element (RRE).

In some embodiments, the vector comprises the nucleotide sequence of SEQ ID NO: 5, SEQ ID NO: 7, SEQ ID NO: 8, SEQ ID NO: 13, SEQ ID NO: 15, SEQ ID NO: 17, SEQ ID NO: 18, SEQ ID NO: 19, and SEQ ID NO: 20, or a nucleotide sequence having at least 85%, 90%, 95%, 98%, or 99% sequence identity thereto.

In another aspect, the disclosure features a composition comprising a vector described herein and a pharmaceutically acceptable carrier.

In yet another aspect, the disclosure provides a cell comprising a vector described herein.

In some embodiments, the cell is a human cell. In some embodiments, the cell is selected from the group consisting of an embryonic stem cell, an adult stem cell, an adult progenitor cell, and a differentiated adult cell. In some embodiments, the cell is a hematopoietic stem cell or a hematopoietic progenitor cell. In some embodiments, the cell is a hematopoietic stem cell or a hematopoietic progenitor cell. In certain embodiments, the source of the stem or progenitor cell is bone marrow, cord blood, placental blood, or peripheral blood. In some embodiments, the cell is transduced with the vector.

In still another aspect, the disclosure provides a composition comprising a cell described herein and a pharmaceutically acceptable carrier.

In another aspect, the disclosure provides a method of treating β-thalassemia, comprising administering to a subject in need thereof an effective amount of a cell described herein, or a cell transduced with a vector described herein, thereby treating β-thalassemia.

In some embodiments, the method further comprises obtaining a cell from the subject. In some embodiments, the method further comprises transducing the cell with the vector. In some embodiments, the cell is a hematopoietic stem cell or a hematopoietic progenitor cell.

In some embodiments, the method further comprises administering to the subject an effective amount of busulfan and cyclophosphamide prior to administering the cell transduced with the vector to the subject. In some embodiments, busulfan is administered at a dose of 2 to 5 mg/kg/day, e.g., 2.4 to 4.8 mg/kg/day, intravenously. In some embodiments, busulfan is administered once every 6 hours. In some embodiments, cyclophosphamide is administered at a dose of 30-80 mg/kg/day, e.g., 45-65 mg/kg/day, intravenously. In some embodiments, cyclophosphamide is administered 18 to 30 hours, e.g., 24 hours, after busulfan is administered. In some embodiments, busulfan is administered for 2-4 days and cyclophosphamide is administered for 1-5 days. In some embodiments, the administration of the cell transduced the vector is initiated 24-72 hours after the administration of cyclophoshamide is completed.

In yet another aspect, the disclosure provides a method of pretreating a subject, comprising administering to the subject an effective amount of busulfan and cyclophosphamide prior to administering to the subject a therapy for β-thalassemia. In some embodiments, the therapy for β-thalassemia comprises a cell transduced with a vector comprising a human β-globin gene, e.g., a cell described herein, or a cell transduced with a vector described herein, to the subject. In some embodiments, busulfan is administered at a dose of 2 to 5 mg/kg/day, e.g., 2.4 to 4.8 mg/kg/day, intravenously. In some embodiments, busulfan is administered once every 6 hours. In some embodiments, cyclophosphamide is administered at a dose of 30-80 mg/kg/day, e.g., 45-65 mg/kg/day, intravenously. In some embodiments, cyclophosphamide is administered 18 to 30 hours, e.g., 24 hours, after busulfan is administered. In some embodiments, busulfan is administered for 2-4 days and cyclophosphamide is administered for 1-5 days. In some embodiments, the administration of the cell transduced the vector is initiated 24-72 hours after the administration of cyclophshamide is completed. In some embodiments, the subject is pretreated in accordance with a method described in Examples 9-12.

In still another aspect, the disclosure provides a formulation comprising the vector described herein, a buffer, a stabilizer, and sodium chloride.

In some embodiments, the vector is present at a concentration of 1×10⁸ TU/mL to 1×10¹⁰ TU/mL. In some embodiments, the vector is present at a concentration of 5×10⁸ TU/mL to 5×10⁹ TU/mL. In some embodiments, the vector is present at a concentration of 5×10⁸ TU/mL to 1×10⁹ TU/mL, e.g., 6×10⁸ TU/mL or 6.2*10⁸ TU/mL. In some embodiments, the vector is present at a concentration of 1×10⁹ TU/mL to 5×10⁹ TU/mL. In some embodiments, the vector is present at a concentration of 2*10⁹ TU/mL to 3*10⁹ TU/mL, e.g., 2.5*10⁹ TU/mL or 2.8×10⁹ TU/mL.

In some embodiments, the buffer is phosphate buffer, sodium citrate, or PIPES. In some embodiments, the buffer is present at a concentration of 10 mM to 50 mM, e.g., 10 mM to 30 mM, 20 mM to 40 mM, 30 mM to 50 mM, or 10 mM to 40 mM. In some embodiments, the buffer is present at a concentration of 10 mM, 20 mM, or 40 mM.

In some embodiments, the stabilizer comprises a sugar or a polyhydric alcohol, e.g., sucrose, trehalose, sorbitol, inositol, glucose, or dextran. In some embodiments, the stabilizer is present at a concentration of 1% to 5%, e.g., 1% to 3%, 2% to 3%, or 1% to 2.5%. In some embodiments, the stabilizer is present at a concentration of 1%, 2%, or 2.5%.

In some embodiments, sodium chloride is present at a concentration of 50 mM to 200 mM, e.g., 50 mM to 70 mM, 70 mM to 90 mM, 80 mM to 100 mM, 100 mM to 120 mM, 140 mM to 160 mM, 100 mM to 150 mM, or 50 mM to 150 mM. In some embodiments, sodium chloride is present at a concentration of 50 mM, 60 mM, 75 mM, 80 mM, 90 mM, 110 mM, 140 mM, or 150 mM.

In some embodiments, the formulation comprises a vector described herein, sodium citrate, sucrose, and sodium chloride. In some embodiments, the vector is present at a concentration of 5×10⁸ TU/mL to 5×10⁹ TU/mL, sodium citrate is present at a concentration of 20 mM to 40 mM, sucrose is present at a concentration of 1% to 2%, and sodium chloride is present at a concentration of 100 mM to 150 mM.

In some embodiments, the formulation is any of the formulations described in Examples 13-14.

### BRIEF DESCRIPTION OF THE DRAWINGS

FIG. 1 depicts the schematic design of exemplary viral vector constructs.

FIG. 2 depicts a diagram of an exemplary lentiviral vector packaging backbone plasmid without insertion of gene of interest.

FIG. 3 depicts viral vector packaging efficiency for exemplary viral vector constructs shown in FIG. 1.

FIG. 4 depicts the schematic design of additional exemplary viral vector constructs.

FIG. 5 is a graph depicting VCN values in PBMCs of recipient mice in the mock (without transduction), LV-TH04 (transduction), and positive (wild-type C57BL/6 cells) groups. Blood samples were collected 4, 6 and 8 weeks after bone marrow transplantation of the recipient mice.

FIG. 6 is a graph depicting the chimeric rate (%) of PBMCs of recipient mice in the mock (without transduction), LV-TH04 (transduction), and positive (wild-type C57BL/6 cells) groups. Blood samples were collected 4, 6 and 8 weeks after bone marrow transplantation of the recipient mice.

FIG. 7 is a series of graphs depicting the hemoglobin content (HGB (g/L); top left graph), percentage of reticulocytes (RET %; top right graph), hematocrit levels (HCT (%); bottom left graph), and average red blood cell volume (MCV(fL); bottom right graph) of recipient mice in the mock (without transduction), LV-TH04 (transduction), and positive (wild-type C57BL/6 cells) groups. Blood samples were collected 4, 6 and 8 weeks after bone marrow transplantation of the recipient animals.

FIG. 8 is a series of chromatograms from HPLC assays using supernatants collected from cells transduced LV-TH04 (top chromatogram) or cells without LV-TH04 (bottom chromatogram). The β-globin and α-globin peaks are indicated.

FIG. 9 is a graph depicting the absolute neutrophil count (10⁹/L) after infusion of LV-TH04-transduced hematopoietic stem cells. Subjects PJYU and ZRHA were treated with myeloablative conditioning based on BU/CY; subject FAZH was treated with myeloablative conditioning based on BU.

FIG. 10 is a graph depicting platelet count (10⁹/L) after infusion of LV-TH04-transduced hematopoietic stem cells. Subjects PJYU and ZRHA were treated with myeloablative conditioning based on BU/CY; subject FAZH was treated with myeloablative conditioning based on BU.

FIG. 11 is a graph depicting the loss (%) of lentiviral particles in the formulations described in Table 15 after being placed at room temperature for 1 day, placed at 4° C. for 3 days, or placed under conditions of freeze-thaw for 3 times.

FIG. 12 is a graph depicting the lentiviral titers (TU/mL) of the formulations described in Table 16 after being placed under conditions of freeze-thaw for 3 times, or placed under conditions of freeze-thaw for 9 times.

FIG. 13 is a graph depicting the lentiviral titers (TU/mL) of the formulations described in Table 17 after being placed under conditions of freeze-thaw for 3 times.

FIG. 14 is a graph depicting the loss (%) of lentiviral particles in the formulations described in Table 18 after being placed at room temperature for 1 day, placed at 4° C. for 3 days, or placed under conditions of freeze-thaw for 3 times.

FIG. 15 is a graph depicting the lentiviral titers (TU/mL) of a formulation comprising sodium citrate (20 mM), sodium chloride (110 mM), and sucrose (1%), or PBS under control conditions, or after being placed at 4° C. for 1 day, placed at 4° C. for 3 days, or placed at room temperature for 1 day.

### DETAILED DESCRIPTION

Lentiviral vectors have the characteristic of host genome integration and therefor are widely considered desirable gene deliver vectors for various genetic diseases, such as hereditary anemia, caused by the loss expression of a single gene. Autologous hematopoietic stem cell therapy can be a promising curative therapy for severe hereditary anemia. Basically, a functional gene encoding a human β-globin peptide chain is introduced into a patient's hematopoietic stem cells ex vivo by lentiviral vector transduction and the transduced cells are infused back to the patient. Without being limited by the availability of donors and associated risk of rejection of transplanted cells and/or graft-versus-host disease, the methods described herein may achieve complete cure of thalassemia by a one-time treatment.

The human β-globin gene comprise a promoter region, 3 exons and 2 introns, a downstream enhancer region, and an endogenous upstream gene expression control region sequence DNase I hypersensitive sites (HSs). The total length of the gene exceeds 60,000 base pairs (bp), which is difficult to be included in any kind of gene therapy vector. For decades, scientists have worked to develop a relatively small β-globin gene expression framework so that it can be used in gene therapy.

However, the commercial production of existing vectors for treating disorders associated with a defective human β-globin gene face a number of difficulties. For example, the vector production rate tends to be low, which greatly increases the production cost and eventually the drug cost. In addition, the gene expression efficiency is oftentimes not optimized, and the viral copy number (VCN) in hematopietic stem cells, one of the most critical quality attributes, still needs to be significantly improved.

The nucleic acid constructs, vectors, compositions, cells, and methods described herein can have various beneficial effects, for example, 1) significantly enhanced virus packaging efficiency; 2) stronger and more efficient vector integration into target cell genome; 3) better clinical efficacy with a lower dose, reducing immunogenicity; 4) higher vector production efficiency and lower production cost; and 5) a wide range of applications, including generation of various forms of vectors for the gene therapy of hereditary anemia.

### Definitions

Unless defined otherwise, all technical and scientific terms used herein have the same meaning as commonly understood by those of ordinary skill in the art to which the disclosure pertains.

As used herein, the term “a” and “an” refers to one or to more than one (i.e., to at least one) of the grammatical object of the article.

As used herein, the term “about” or “approximately” when referring to a measurable value such as an amount, a temporal duration, and the like, are meant to encompass variations of 20% or in some instances ±10%, or in some instances ±5%, or in some instances ±1%, or in some instances ±0.10% from the specified value, as such variations are appropriate in the context of the disclosure.

As used herein, the term “allogeneic” refers to a cell of the same species that differs genetically to the cell in comparison.

As used herein, the term “associated with” or “linked,” when used with respect to two or more moieties, means that the moieties are associated or connected, e.g., physically or chemically, with one another, either directly or via one or more additional moieties that serves as a linking agent, to form a structure that is sufficiently stable so that the moieties remain physically associated under the conditions in which the structure is used, e.g., physiological conditions. In some embodiments, the two or more moieties are covalently or non-covalently attached, coupled, linked, or tethered. In some embodiments, an association is through direct covalent chemical bonding. In other embodiments, the association is through ionic or hydrogen bonding or a hybridization based connectivity sufficiently stable such that the associated or linked entities remain physically associated.

As used herein the term “autologous” refers to a cell from the same subject.

As used herein, the term “carrier” includes any and all solvents, dispersion media, vehicles, coatings, diluents, antibacterial and antifungal agents, isotonic and absorption delaying agents, buffers, carrier solutions, suspensions, colloids, and the like.

As used herein, the term “complementary” when used to describe a first nucleotide sequence in relation to a second nucleotide sequence, refers to the ability of an oligonucleotide or polynucleotide comprising the first nucleotide sequence to hybridize and form base pairs, e.g., a duplex, with an oligonucleotide or polynucleotide comprising the second nucleotide sequence. In some embodiments, base pairs are formed by hydrogen bonds between nucleotide units in antiparallel polynucleotide strands. In some embodiments, complementary polynucleotide or oligonucleotide strands can form base pairs in the Watson-Crick manner or in any other manner that allows for the formation of duplexes. The term “complementary” as used herein can encompass fully complementary, partially complementary, or substantially complementary. “Fully complementary” refers to the situation in which each nucleotide unit of one polynucleotide or oligonucleotide strand can base-pair with a nucleotide unit of a second polynucleotide or oligonucleotide strand. “Substantially complementary” refers to the situation in which two polynucleotides or oligonucleotide strands can be fully complementary or they may form one or more, but generally not more than 1, 2, 3, 4, or 5 mismatched or non-complimentary base pairs upon hybridization for a duplex, while still retaining the ability to hybridize under the conditions most relevant to their ultimate application.

As used herein, the term “control element,” “regulatory control element,” or “regulatory sequence” refers to an element used for expression of a gene or gene product. Exemplary “control elements,” “regulatory control elements,” or “regulatory sequences” include, but are not limited to, promoter regions, polyadenylation signals, transcription termination sequences, upstream regulatory domains, origins of replication, internal ribosome entry sites (“IRES”), enhancers, and the like, which provide for the replication, transcription and translation of a coding sequence in a recipient cell.

As used herein, the term “effective amount” refers to an amount of a compound, formulation, material, or composition, to achieve a particular biological result. In some embodiments, the effective amount is a therapeutically effective amount. In some embodiments, the effective amount of an agent is that amount sufficient to effect a beneficial or desired result, for example, a clinical result. For example, in the context of administering an agent that treats a disorder, an effective amount of an agent is, for example, an amount sufficient to achieve treatment of the disorder, as compared to the response obtained without administration of the agent.

As used herein, the term “enhancer” refers to a segment of DNA which contains a sequence capable of providing enhanced transcription and in some instances can function independent of their orientation relative to another control sequence. An enhancer can function cooperatively or additively with promoters and/or other enhancer elements.

As used herein, the term “export element” refers to a cis-acting post-transcriptional regulatory element that regulates the transport of an RNA transcript from the nucleus to the cytoplasm of a cell. Examples of RNA export elements include, but are not limited to, the human immunodeficiency virus (HIV) rev response element (RRE) and the hepatitis B virus post-transcriptional regulatory element (HPRE). In some embodiments, the RNA export element is located within the 3′ UTR of a gene and is inserted as one or multiple copies.

As used herein, the term “expression” refers to transcription and/or translation of a particular nucleotide sequence. Expression can generally include one or more of the following: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

As used herein, the term “expression control sequence” refers to a polynucleotide sequence that comprises one or more promoters, enhancers, or other transcriptional control elements or combinations thereof that are capable of directing, increasing, regulating, or controlling the transcription or expression of an operatively linked polynucleotide.

As used herein, the term “FLAP element” refers to a nucleic acid whose sequence includes the central polypurine tract and the central termination sequence (cPPT and CTS) of a retrovirus (e.g., HIV-1 or HIV-2). Without wishing to be bound by theory, it is believed that during retrovirus reverse transcription, central initiation of the plus-strand DNA at the central polypurine tract (cPPT) and central termination at the central termination sequence (CTS) lead to the formation of a three-stranded DNA structure: the central DNA flap. The central DNA flap may act as a cis-active determinant of retroviral genome nuclear import and/or may increase the titer of the virus. Exemplary FLAP elements are described, e.g., in Zennou, et al., 2000, Cell, 101:173 and U.S. Pat. No. 6,682,907, the contents of which are incorporated by reference in their entirety. In an embodiment, a retroviral or lentiviral vector backbone described herein comprises one or more FLAP elements upstream or downstream of the heterologous genes of interest in the vector. For example, a transfer plasmid can include a FLAP element. In an embodiment, a viral vector described herein comprises a FLAP element isolated from HIV-1.

As used herein, the term “hematopoietic stem cell” or “HSC” refers to multipotent stem cells that give rise to the all the blood cell types of an organism, including myeloid (e.g., monocytes and macrophages, neutrophils, basophils, eosinophils, erythrocytes, megakaryocytes/platelets, dendritic cells), and lymphoid lineages (e.g., T-cells, B-cells, NK-cells), and others known in the art.

As used herein, the term “host cell” refers to a cell transfected, infected, or transduced in vivo, ex vivo, or in vitro with a vector or a polynucleotide. Host cells may include packaging cells, producer cells, and cells infected with viral vectors. In certain embodiments, the term “target cell” is used interchangeably with host cell and refers to transfected, infected, or transduced cells of a desired cell type.

As used herein, the term “identity” refers to the subunit sequence identity between two polymeric molecules, e.g., between two nucleic acid molecules (e.g. two DNA molecules and/or two RNA molecules) and/or between two polypeptide molecules. When a subunit position in both of the two molecules is occupied by the same monomeric subunit, they are considered identical at that position. The percent identity between the two sequences is a function of the number of identical positions shared by the sequences, taking into account the number of gaps, and the length of each gap, which can to be introduced for optimal alignment of the two sequences. For example, calculation of the percent identity of two sequences can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, at least 99%, or 100% of the length of the reference sequence. When a position in the first sequence is occupied by the same nucleotide or amino acid as the corresponding position in the second sequence, then the sequences are identical at that position. The comparison of sequences and determination of percent identity between two sequences can be accomplished using a mathematical algorithm known in the art.

As used herein, the term “isolated” means a material (e.g., a polynucleotide, a polypeptide, a cell) that is substantially or essentially free from components that normally accompany it in its native state. In some embodiments, the term “obtained” or “derived” is used interchangeably with the term “isolated.” For example, an “isolated polynucleotide,” as used herein, refers to a polynucleotide that has been purified from the sequences that flank it in a naturally-occurring state, e.g., a DNA fragment that has been removed from the sequences that are normally adjacent to the fragment.

As used herein, the term “lentiviral vector” refers to a viral vector containing a structural or functional element, or a portion thereof, that is primarily derived from a lentivirus.

As used herein, the term “lentivirus” refers to a genus of retroviruses. Exemplary lentiviruses include, but are not limited to, HIV (human immunodeficiency virus, e.g., HIV type 1 and HIV type 2), bovine immune deficiency virus (BIV), caprine arthritis-encephalitis virus (CAEV), equine infectious anemia virus (EIAV), feline immunodeficiency virus (FIV), simian immunodeficiency virus (SIV), and visna-maedi virus (VMV) virus. In an embodiment, the lentivirus is an HIV.

As used herein, the term “long terminal repeat” or “LTR” refers to domains of base pairs located at the ends of retroviral DNAs that, in their natural sequence context, are direct repeats and contain U3, R and U5 regions. Without wishing to be bound by theory, it is believed that in some embodiments, LTRs provide functions fundamental to the expression of retroviral genes (e.g., promotion, initiation and polyadenylation of gene transcripts) and to viral replication. The LTR contains a number of regulatory signals, for example, transcriptional control elements, polyadenylation signals and sequences needed for replication and integration of the viral genome. The LTR typically includes U3, R and U5 regions and appears at both the 5′ and 3′ ends of the viral genome. The U3 region contains the enhancer and promoter elements. The U5 region is the sequence between the primer binding site and the R region and contains the polyadenylation sequence. The R (repeat) region is flanked by the U3 and U5 regions. Adjacent to the 5′ LTR are sequences necessary for reverse transcription of the genome (the tRNA primer binding site) and for efficient packaging of viral RNA into particles (the Psi site).

As used herein, the term “nucleic acid cassette” refers to a sequence within the vector which can express an RNA, and subsequently a polypeptide. Foe example, the nucleic acid cassette may contains a gene-of-interest and/or one or more expression control sequences. Vectors may comprise one, two, three, four, five or more nucleic acid cassettes. The nucleic acid cassette can be positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. In some embodiments, the nucleic acid cassette has its 5′ and 3′ ends adapted for ready insertion into a vector, e.g., it has restriction endonuclease sites at each end. In some embodiments, the nucleic acid cassette contains a polynucleotide sequence that can be used to treat or prevent a disorder. The cassette can typically be removed and inserted into a plasmid or viral vector as a single unit.

As used herein, the term “operably linked” refers to a functional connection between two or more molecules, constructs, transcripts, entities, moieties or the like. In some embodiments, “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

As used herein, the term “or” means either one, both, or any combination of the alternatives, and is used interchangeably with the term “and/or”, unless context clearly indicates otherwise.

As used herein, the term “packaging cell line” refers to a cell line that does not contain a packaging signal, but does stably or transiently express viral structural proteins and replication enzymes (e.g., gag, pol, and env) which are necessary for the correct packaging of viral particles.

As used herein, the term “packaging signal” or “packaging sequence” refers to a sequence located within a retroviral genome that is required for insertion of the viral RNA into the viral capsid or particle. Several retroviral vectors use the minimal packaging signal (also referred to as the psi [Ψ] sequence) needed for encapsidation of the viral genome. Thus, in some embodiments, the terms “packaging sequence,” “packaging signal,” “psi” and the symbol “Ψ” are used interchangeably to describe the non-coding sequence required for encapsidation of retroviral RNA strands during viral particle formation.

As used herein, the term “pharmaceutically acceptable” refers to molecular entities and compositions that do not produce an allergic or similar untoward reaction when administered to a human.

As used herein, the term “pharmaceutically acceptable carrier” includes any and all solvents, dispersion media, coatings, antibacterial and antifungal agents, isotonic and absorption delaying agents, and the like that are physiologically compatible, including pharmaceutically acceptable cell culture media.

As used herein, the term “prevent” or “prevention” means that a subject (e.g., a human) is less likely to have the disorder, e.g., a myeloma, if the subject receives the antibody molecule.

As used herein, the term “promoter” refers to a recognition site of a polynucleotide (DNA or RNA) to which an RNA polymerase binds.

As used herein, the term “promoter/enhancer” refers to a segment of DNA which contains a sequence capable of providing both promoter and enhancer functions.

As used herein, the term “prophylactically effective amount” refers to an amount effective to achieve the desired prophylactic effect or result.

As used herein the term “retroviral vector” refers to a viral vector containing a structural or functional element, or a portion thereof, that is primarily derived from a retrovirus.

As used herein, the term “R region” refers to a region within an LTR beginning at the start of the capping group (i.e., the start of transcription) and ending immediately prior to the start of the poly A tract. The R region is also defined as being flanked by the U3 and U5 regions. Without wishing to be bound by theory, it is believed that in some embodiments, the R region plays a role during reverse transcription in permitting the transfer of nascent DNA from one end of the genome to the other.

As used herein, the term “retrovirus” refers to an RNA virus that reverse transcribes its genomic RNA into DNA and subsequently integrates the DNA into a host genome. Exemplary retroviruses include, but are not limited to, lentiviruses, oncoretroviruses, and spumaviruses. Exemplary oncoretroviruses include, but are not limited to, feline leukemia virus (FLV), Friend murine leukemia virus, gibbon ape leukemia virus (GaLV), Harvey murine sarcoma virus (HaMuSV), Moloney murine leukemia virus (M-MuLV), Moloney murine sarcoma virus (MoMSV), murine mammary tumor virus (MuMTV), murine stem cell virus (MSCV), and Rous sarcoma virus (RSV). In an embodiment, the retrovirus is a lentivirus.

As used herein, the term “thalassemia” refers to a hereditary disorder characterized by defective production of hemoglobin. The term “thalassemia” encompasses hereditary anemias that occur due to mutations affecting the synthesis of hemoglobin. Thus, the term includes any symptomatic anemia resulting from thalassemic conditions such as severe or β-thalassemia, thalassemia major, thalassemia intermedia, α-thalassemias such as hemoglobin H disease. Examples of thalassemias include α-thalassemia and β-thalassemia. α-thalassemia is caused by deletion of a gene or genes from the globin chain. β-thalassemia is caused by a mutation in the β-globin chain, and can occur in a major or minor form. In the major form of β-thalassemia, children typically are normal at birth, but develop anemia during the first year of life. The mild form of β-thalassemia produces small red blood cells.

As used herein, the term “therapeutically effective amount” refers to an amount effective to achieve the desired therapeutic effect or result.

As used herein, the term “self-inactivating vector” or “SIN vector” refers to a replication-defective vector (e.g., a retroviral or lentiviral vector) in which the right 3′ LTR enhancer-promoter region, known as the U3 region, has been modified (e.g., by deletion or substitution) to prevent viral transcription beyond the first round of viral replication. Without wishing to be bound by theory, it is believed that the right 3′ LTR U3 region is used as a template for the left 5′ LTR U3 region during viral replication and, thus, the viral transcript cannot be made without a functional U3 enhancer-promoter. In some embodiments, the 3′ LTR is modified such that the U5 region is replaced, for example, with a poly(A) sequence.

As used herein, the term “stem cell” refers to a cell which is an undifferentiated cell capable of (1) long term self-renewal, or the ability to generate at least one identical copy of the original cell, (2) differentiation at the single cell level into multiple, and in some instance only one, specialized cell type and (3) of in vivo functional regeneration of tissues.

As used herein, the term “subject” is intended to include human and non-human animals. In some embodiments, the subject is a human subject, e.g., a human patient having a disorder described herein, or at risk of having a disorder described herein. The term “non-human animals” includes mammals and non-mammals, such as non-human primates. The vectors, cells, and compositions described herein are suitable for treating human patients a disorder described herein. Patients having a disorder described herein include, e.g., those who have developed a disorder described herein but are (at least temporarily) asymptomatic, patients who have exhibited a symptom of a disorder described herein, and patients having a disorder related to or associated with a disorder described herein.

As used herein, the term “trans-activation response” or “TAR” refers to a genetic element located in the R region of retroviral or lentiviral LTRs. Without wishing to be bound by theory, it is believed that in some embodiments, this element interacts with the retroviral or lentiviral trans-activator (tat) genetic element to enhance viral replication. In some embodiments, this element is not required wherein the U3 region of the 5′ LTR is replaced by a heterologous promoter. In an embodiment, a viral vector described herein comprises a TAR element.

As used herein, the term “treat” or “treatment” means that a subject (e.g., a human) who has a disorder and/or experiences a symptom of a disorder will, in some embodiments, suffer less a severe symptom and/or recover faster when a therapy is administered than if the therapy were never administered. Treatment can, partially or completely, alleviate, ameliorate, relieve, inhibit, or reduce the severity of, and/or reduce incidence, and optionally, delay onset of, one or more manifestations of the effects or symptoms, features, and/or causes of a disorder. In some embodiments, treatment is of a subject who does not exhibit certain signs of a disorder, and/or of a subject who exhibits only early signs of a disorder. In some embodiments, treatment is of a subject who exhibits one or more established signs of a disorder. In some embodiments, treatment is of a subject diagnosed as suffering from a disorder.

As used herein, the term “variant” refers to a polypeptide that is distinguished from a reference polypeptide by the addition, deletion, truncations, and/or substitution of at least one amino acid residue, and that retain a biological activity. In certain embodiments, a polypeptide variant is distinguished from a reference polypeptide by one or more substitutions, which may be conservative or non-conservative, as known in the art.

As used herein, the term “vector” refers to a nucleic acid molecule that is used as a vehicle to transfer another nucleic acid molecule into a host cell. In an embodiment, the transferred nucleic acid molecule is inserted to the vector nucleic acid molecule. A vector may include a sequence that directs autonomous replication in a cell, or may include a sequence sufficient to allow integration into the host cell genome. Exemplary vectors include, but are not limited to, viral vectors, plasmids (e.g., DNA plasmids or RNA plasmids), transposons, cosmids, and bacterial artificial chromosomes.

Exemplary viral vectors include, but are not limited to, retroviral vectors (e.g., replication defective retroviral vectors) and lentiviral vectors.

As used herein, the term “viral vector” refers to a nucleic acid molecule (e.g., a plasmid) that includes one or more virus-derived nucleic acid elements that facilitate transfer of a nucleic acid molecule into a cell, integration of a nucleic acid molecule into the genome of cell, or delivery of a nucleic acid molecule to a viral particle.

### Nucleic Acid Constructs and Vectors

The disclosure provides a nucleic acid construct or vector (e.g., a viral vector) comprising a human β-globin gene or a functional fragment thereof.

In some embodiments, the sequence of the human β-globin gene comprises human β-globin gene exon 1, intron 1, exon 2, intron 2, and exon 3. In certain embodiments, the sequence of the human β-globin gene is according to Ensemble Database Gene: HBB (ENSG00000244734) Transcript: HBB-201 (ENST00000335295.4).

In some embodiments, nucleic acid construct or vector comprises a regulatory control element. In some embodiments, nucleic acid construct or vector comprises an expression control sequence. In some embodiments, the nucleic acid construct or vector comprises a promoter or a promoter/enhancer.

In some embodiment, the human β-globin gene comprises a human β-globin promoter. In some embodiment, the human β-globin gene does not comprise a human β-globin promoter. In some embodiments, human β-globin promoter is about 250 to about 275 bp (e.g., 268 bp) upstream of exon 1 of the human β-globin promoter. In some embodiments, the human β-globin gene comprises a human β-globin 3′-enhancer. In certain embodiments, the human β-globin 3′-enhancer is about 850 bp to about 900 bp (e.g., 878 bp) downstream of exon 3 of the human β-globin gene. In some embodiments, the human β-globin gene comprises one or more (e.g., 2 or 3) wild-type exons. In some embodiments, the human β-globin gene comprises one or more (e.g., 2 or 3) codon-optimized exons. In some embodiments, the human β-globin gene comprises one or more wild-type introns. In certain embodiments, the human β-globin gene comprises a wild-type intron 2. In some embodiments, the human β-globin gene comprises one or more truncated introns. In certain embodiments, the human (β-globin gene comprises a truncated intron 2. In some embodiments, the human β-globin gene comprises a wild-type exon 2. In some embodiments, the human β-globin gene comprises an exon 2 that encodes a threonine to glutamine mutation at codon 87 (T87Q). In some embodiments, the human β-globin gene comprises the nucleotide sequence of SEQ ID NO: 23, SEQ ID NO: 24, SEQ ID NO: 25, or a nucleotide sequence having at least 75%, 80%, 85%, 90%, 91%, 92%, 93%, 94%, 95%, 96%, 97%, 98%, or 99% identity thereto, or any combination thereof. In some embodiments, the nucleic acid construct or vector comprises a nucleotide sequence that is complementary to the nucleotide sequence of SEQ ID NO: 23, SEQ ID NO: 24, SEQ ID NO: 25, or a nucleotide sequence having at least 75%, 80%, 85%, 90%, 91%, 92%, 93%, 94%, 95%, 96%, 97%, 98%, or 99% identity thereto, or any combination thereof.

In some embodiments, the human β-globin gene encodes a human β-globin variant. For example, the variant may include an amino acid sequence having at least about 50%, 55%, 60%, 65%, 70%, 75%, 80%, 85%, 90%, 91%, 92%, 93%, 94%, 95%, 96%, 97%, 98%, 99% or more sequence identity to the wild-type human β-globin amino acid sequence.

In some embodiments, the nucleic acid construct or vector comprises a retroviral (e.g., lentiviral) LTR. In some embodiments, the nucleic acid construct or vector comprises a left (5′) retroviral LTR and a right (3′) retroviral LTR. In some embodiments, the right (3′) LTR is a self-inactivating (SIN) LTR. In some embodiments, the retroviral LTR is unmodified, e.g., a wild-type retroviral LTR. In some embodiments, the retroviral LTR is modified, e.g., comprising one or more substitutions, insertions, and/or deletions. In some embodiments, the left (5′) retroviral LTR is replaced with a heterologous promoter, e.g., cytomegalovirus (CMV) promoter, a Rous Sarcoma Virus (RSV) promoter, a thymidine kinase promoter, or an Simian Virus 40 (SV40) promoter. In some embodiments, the right (3′) retroviral LTR is absent. In some embodiments, the retroviral LTR is a lentiviral LTR.

In some embodiments, the nucleic acid construct or vector comprises a human β-globin gene upstream locus control region (LCR). In some embodiments, the upstream locus control region (LCR) comprises one or more (e.g., 2 or 3) truncated DNase I hypersensitive sites, HS2, HS3 and HS4 of the LCR.

In some embodiments, the nucleic acid construct or vector comprises a cis-acting posttranscriptional regulatory element. In some embodiments, the posttranscriptional regulatory element is a woodchuck hepatitis virus post-transcriptional regulatory element (WPRE).

In some embodiments, the nucleic acid construct or vector comprises a polyadenylation signal and/or origin. In some embodiments, the polyadenylation signal is an SV40 polyadenylation signal. In some embodiments, the original is an SV40 origin. The polyadenylation signal and/or origin can be located 3′ of the right (3′) retroviral LTR.

In some embodiments, the nucleic acid construct or vector comprises one or more (e.g., two, three, four, or all) of a) a left (5′) retroviral LTR; b) human β-globin gene; c) human β-globin gene upstream locus control region (LCR); d) a cis-acting posttranscriptional regulatory element; e) a right (3′) retroviral LTR; and f) a SV40 polyadenylation signal and/or SV40 origin.

In some embodiments, the nucleic acid construct or vector comprises a nucleic acid cassette comprising one or more (e.g., two, three, four, or all) of a) a left (5′) retroviral LTR; b) human (β-globin gene; c) human β-globin gene upstream locus control region (LCR); d) a cis-acting posttranscriptional regulatory element; e) a right (3′) retroviral LTR; and f) a SV40 polyadenylation signal and/or SV40 origin.

In some embodiments, the nucleic acid construct or vector further comprises one or more (e.g., 2 or 3) of a Psi packaging sequence (Ψ+), a central polypurine tract/DNA flap (cPPT/FLAP), or a retroviral export element-rev response element (RRE).

In some embodiments, the nucleic acid construct or vector comprises the nucleotide sequence SEQ ID NO: 5, SEQ ID NO: 7, SEQ ID NO: 8, SEQ ID NO: 13, SEQ ID NO: 15, SEQ ID NO: 17, SEQ ID NO: 18, SEQ ID NO: 19, SEQ ID NO: 20, or a nucleotide sequence having at least 75%, 80%, 85%, 90%, 91%, 92%, 93%, 94%, 95%, 96%, 97%, 98%, or 99% identity thereto. In some embodiments, the nucleic acid construct or vector comprises a nucleotide sequence that is complementary to the nucleotide sequence SEQ ID NO: 5, SEQ ID NO: 7, SEQ ID NO: 8, SEQ ID NO: 13, SEQ ID NO: 15, SEQ ID NO: 17, SEQ ID NO: 18, SEQ ID NO: 19, SEQ ID NO: 20, or a nucleotide sequence having at least 75%, 80%, 85%, 90%, 91%, 92%, 93%, 94%, 95%, 96%, 97%, 98%, or 99% identity thereto.

In some embodiments, the nucleic acid construcxt or vector further comprises a truncated erythroid cell expression control sequence.

In some embodiments, the lentiviral nucleic acid construct or vector is an HIV nucleic acid construct or vector. For example, the lentiviral nucleic acid construct or vector may be derived from human immunodeficiency-1 (HIV-1), human immunodeficiency-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), caprine arthritis encephalitis virus (CAEV), and the like.

The nucleic acid construct or vector components described herein can be operably linked to allow for expression of human β-globin. The vectors described herein can be self-inactivating vector.

Large scale viral particle production is typically necessary to achieve a reasonable viral titer. Viral particles can be produced by transfecting a transfer nucleic acid construct or vector into a packaging cell line that comprises viral structural and/or accessory genes, e.g., gag, pol, env, tat, rev, vif, vpr, vpu, vpx, or nef genes or other retroviral genes.

### Pharmaceutical Compositions and Formulations

The disclosure provides pharmaceutical compositions comprising a nucleic acid construct or vector described herein or a cell described herein, and a pharmaceutically acceptable carrier.

In some embodiments, the pharmaceutically acceptable carrier is suitable for parenteral administration, e.g., intravascular (intravenous or intraarterial), intraperitoneal, or intramuscular administration. Exemplary pharmaceutically acceptable carriers include sterile aqueous solutions or dispersions and sterile powders for the preparation of sterile injectable solutions or dispersion. The use of such media and agents for pharmaceutically active substances is well known in the art.

The compositions of the disclosure can comprise, for example, one or more polypeptides, polynucleotides, vectors comprising same, or transduced cells, formulated in pharmaceutically-acceptable or physiologically-acceptable solutions for administration to a cell or an animal, either alone, or in combination with one or more other therapeutic agents or modalities. The compositions of the disclosure may also be administered in combination with other agents, including, but not limited to, cytokines, growth factors, hormones, small molecules, or other pharmaceutically-active agents.

In the pharmaceutical compositions of the disclosure, formulation of pharmaceutically-acceptable excipients and carrier solutions is well-known to those of skill in the art, as is the development of suitable dosing and treatment regimens for using the particular compositions described herein in a variety of treatment regimens, including, but not limited to, parenteral, intravenous, and intramuscular administration and formulation.

In all cases the form should be sterile and should be fluid to the extent that easy syringeability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. Some variation in dosage will necessarily occur depending on the condition of the subject being treated. The person responsible for administration will, in any event, determine the appropriate dose for the individual subject.

Sterile injectable solutions can be prepared by incorporating the active compounds in the required amount in the appropriate solvent with the various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective.

The formulations are easily administered in a variety of dosage forms such as injectable solutions, drug-release capsules, and the like.

The compositions or formulations described herein can comprises a cell contacted with a combination of any number of polypeptides, polynucleotides, and small molecules, as described herein.

In another aspect, the disclosure provides compositions that comprise a therapeutically-effective amount of one or more polynucleotides or polypeptides, as described herein, formulated together with one or more pharmaceutically acceptable carriers (additives) and/or diluents (e.g., pharmaceutically acceptable cell culture medium).

In yet another aspect, the disclosure provides formulations or compositions suitable for the delivery of viral vector systems (e.g., viral-mediated transduction), including, but not limited to, retroviral (e.g., lentiviral) vectors.

Exemplary formulations for ex vivo delivery include, but are not limited to, the use of various transfection agents known in the art, such as calcium phosphate, electroporation, heat shock, and various liposome formulations (e.g., lipid-mediated transfection).

### Therapeutic Use

The nucleic acid constructs, vectors, compositions, and cells described herein can be used in methods of treating or preventing thalassemia, e.g., β-thalassemia.

In some embodiments, the vector is administered by direct injection to a cell, tissue, or organ of a subject in need of gene therapy, e.g., in vivo. In some embodiments, the cell is transduced in vitro or ex vivo with a nucleic acid construct or vector described herein, and optionally expanded ex vivo. The transduced cell is then administered to a subject in need of gene therapy. Cells suitable for transduction or administration in the methods described herein include, but are not limited to, stem cells, progenitor cells, and differentiated cells. In some embodiments, the transduced cell is a hematopoietic stem cell.

In some embodiments, the transduced cells are hematopoietic stem and/or progenitor cells, e.g., isolated from bone marrow, umbilical cord blood, or peripheral circulation. In certain embodiments, the transduced cells are hematopoietic stem cells, e.g., isolated from bone marrow, umbilical cord blood, or peripheral circulation.

Hemapoietic stem or pluripotent cells may be identified according to certain phenotypic or genotypic markers, which are known in the art.

In another aspect, the disclosure provides a method of treating a disorder. The method comprises administering to a subject (e.g., a human subject) in need thereof an effective amount of a nucleic acid construct or vector described herein, or a cell (e.g., a hematopoietic stem or progenitor cell) transduced with a nucleic acid construct or vector described herein, thereby treating the disorder. In some embodiments, the effective amount is a therapeutically effective amount. In some embodiments, the effective amount is a prophylactically effective amount.

In some embodiments, the disorder is a disorder is associated with a defective β-globin gene. In some embodiments, the disorder is thalassemia (e.g., β-thalassemia). In some embodiments, the method further comprises obtaining a cell (e.g., a hematopoietic stem or pluripotent cell) from the subject. In some embodiments, the method further comprises transducing a cell (e.g., a hematopoietic stem or pluripotent cell) from the subject with a nucleic acid construct or vector described herein. In some embodiments, the method further comprises isolating the transduced cell. In some embodiments, the method further comprises administering to the subject a second therapeutic agent or modality.

In another aspect, the disclosure provides a method of providing a transduced cell. The method comprises administering to a subject (e.g., a human subject) in need thereof a cell (e.g., a hematopoietic stem or progenitor cell) transduced with a nucleic acid construct or vector described herein.

In another aspect, the disclosure provides a method of treating a hemoglobinopathy. The method comprises administering to a subject (e.g., a human subject) in need thereof a nucleic acid construct or vector described herein, or a cell (e.g., a hematopoietic stem or progenitor cell) transduced with a vector described herein.

In another aspect, the disclosure provides a method of selectively expanding the number erythroid cells. The method comprises administering to a subject (e.g., a human subject) in need thereof a nucleic acid construct or vector described herein, or a cell (e.g., a hematopoietic stem or progenitor cell) transduced with a nucleic construct or vector described herein.

In another aspect, the disclosure provides a method of increasing the proportion of red blood cells or erythrocytes compared to white blood cells or leukocytes in a subject. The method comprises administering a nucleic acid construct or vector described herein, or a cell (e.g., a hematopoietic stem or progenitor cell) transduced with a nucleic acid construct or vector described herein.

In some embodiments, the transduced cell is administered to the subject intravenously. In some embodiments, the transduced cells are administered to the subject at a dose of about 1×10⁵ to about 1×10⁸ cells, e.g., about 1×10⁶ to about 1×10⁷ cells, about 1×10⁶ to about 1×10⁸ cells, about 1×10⁷ to about 1×10⁸ cells, about 1×10⁵ to about 1×10⁷ cells, or about 1×10⁵ to about 1×10⁶ cells. In some embodiments, the transduced cells are administered as a single dose.

### Enumerated Embodiments

1. A vector comprising:
a) a left (5′) retroviral LTR;
b) a human β-globin gene;
c) a human β-globin gene upstream locus control region (LCR);
d) a cis-acting posttranscriptional regulatory element;
e) a right (3′) retroviral LTR; and
f) a cis-acting element SV40 polyadenylation signal and/or SV40 origin.
   2. The vector of embodiment 1, wherein the human β-globin gene comprises exon 1, intron 1, exon 2, intron 2, and exon 3.
   3. The vector of embodiment 1 or 2, wherein the human β-globin gene comprises a human β-globin promoter.
   4. The vector of embodiment 3, wherein the human β-globin gene comprises exon 1 and the human β-globin promoter is located 268 bp upstream of exon 1.
   5. The vector of any of embodiments 1-3, wherein the β-globin gene comprises a human β-globin 3′-enhancer.
   6. The vector of embodiment 5, wherein the human β-globin gene comprises exon 3 and the human β-globin 3′-enhancer is located 878 bp downstream of exon 3.
   7. The vector of any of embodiments 1-6, wherein the human β-globin gene comprises wild-type exons or codon-optimized exons.
   8. The vector of any of embodiments 1-7, wherein the human β-globin gene comprises a wild-type intron 2 or an truncated intron 2.
   9. The vector of any of embodiments 1-8, wherein the human β-globin gene comprises a wild-type exon 2 or an exon 2 encoding a threonine to glutamine mutation at codon 87 (T87Q).
   10. The vector of any of embodiments 1-9, wherein the upstream locus control region (LCR) comprises truncated DNase I hypersensitive sites, HS2, HS3 and HS4.
   11. The vector of any of embodiments 1-10, wherein the posttranscriptional regulatory element is a woodchuck hepatitis virus post-transcriptional regulatory element (WPRE).
   12. The vector of embodiment 11, wherein the WPRE is wildtype WPRE or a mutated WPRE, e.g., a mutated WPRE comprising one or more, or all, of the mutations described herein.
   13. The vector of embodiment 12, wherein the wildtype WPRE comprises the nucleotide sequence of SEQ ID NO: 32, or a nucleotide sequence having at least 80%, 85%, 90%, 95%, or 99% identity thereto.
   14. The vector of embodiment 12, wherein the mutated WPRE comprises the nucleotide sequence of SEQ ID NO: 33, or a nucleotide sequence having at least 80%, 85%, 90%, 95%, or 99% identity thereto.
   15. The vector of any of embodiments 1-14, wherein the SV40 polyadenylation signal and/or SV40 origin is located 3′ downstream of the right (3′) retroviral LTR.
   16. The vector of any of embodiments 1-15, wherein the human β-globin gene comprises one, two, or all of the nucleotide sequences of SEQ ID NO: 23, SEQ ID NO: 24, or SEQ ID NO: 25, or a nucleotide sequence having at least 80%, 85%, 90%, 95%, or 99% identical thereto.
   17. The vector of any of embodiments 1-16, which is a lentivirus vector.
   18. The vector of any of embodiments 1-17, wherein the left (5′) retroviral LTR, the right (3′) retroviral LTR, or both, is a lentiviral LTR.
   19. The vector of any of embodiments 1-18, wherein the right (5′) LTR comprises a promoter that is replaced with a heterologous promoter.
   20. The vector of any of embodiments 1-19, wherein the right (3′) LTR is a self-inactivating (SIN) LTR.
   21. The vector of any of embodiments 1-20, further comprising one or more of a Psi packaging sequence (Ψ+), a central polypurine tract/DNA flap (cPPT/FLAP), or a retroviral export element-rev response element (RRE).
   22. The vector of any of embodiments 1-21, which comprises the nucleotide sequence of SEQ ID NO: 5, SEQ ID NO: 7, SEQ ID NO: 8, SEQ ID NO: 13, SEQ ID NO: 15, SEQ ID NO: 17, SEQ ID NO: 18, SEQ ID NO: 19, or SEQ ID NO: 20, or a nucleotide sequence having at least 80%, 85%, 90%, 95%, or 99% identity thereto.
   23. A composition comprising the vector of embodiment 1 and a pharmaceutically acceptable carrier.
   24. A human cell transduced with the vector of any of embodiments 1-22.
   25. The cell of embodiment 24, which is an embryonic stem cell, an adult stem cell, an adult progenitor cell, or a differentiated adult cell.
   26. The cell of embodiment 24, which is a hematopoietic stem cell or a hematopoietic progenitor cell.
   27. The cell of embodiment 26, wherein the hematopoietic stem cell or hematopoietic progenitor cell is obtained from bone marrow, cord blood, placental blood, or peripheral blood.
   28. A composition comprising a cell transduced with the vector of any of embodiments 1-22 and a pharmaceutically acceptable carrier.
   29. A method of treating β-thalassemia, comprising administering to a subject in need thereof an effective amount of a cell transduced with the vector of any of embodiments 1-22, thereby treating β-thalassemia.
   30. The method of embodiment 29, further comprising obtaining a cell from the subject.
   31. The method of embodiment 29 or 30, further comprising transducing the cell with the vector.
   32. The method of any of embodiments 29-31, wherein the cell is a hematopoietic stem cell or a hematopoietic progenitor cell.
   33. The method of any of embodiments 29-32, further comprising administering to the subject an effective amount of busulfan and cyclophosphamide prior to administering the cell transduced with the vector to the subject.
   34. The method of embodiment 33, wherein busulfan is administered at a dose of 2 to 5 mg/kg/day, e.g., 2.4 to 4.8 mg/kg/day, intravenously.
   35. The method of embodiment 33 or 34, wherein cyclophosphamide is administered at a dose of 30-80 mg/kg/day, e.g., 45-65 mg/kg/day, intravenously.
   36. The method of any of embodiments 33-35, wherein cyclophosphamide is administered 18 to 30 hours, e.g., 24 hours, after busulfan is administered.
   37. The method of any of embodiments 33-36, wherein busulfan is administered for 2-4 days and cyclophosphamide is administered for 1-5 days.
   38. The method of any of embodiments 33-37, wherein the administration of the cell transduced the vector is initiated 24-72 hours after the administration of cyclophshamide is completed.
   39. A formulation comprising the vector of any of embodiments 1-22, a buffer, a stabilizer, and sodium chloride.
   40. The formulation of embodiment 39, wherein the vector is present at a concentration of 1×10⁸ TU/mL to 1×10¹⁰ TU/mL.
   41. The formulation of embodiment 40, wherein the vector is present at a concentration of 5×10⁸ TU/mL to 5×10⁹ TU/mL.
   42. The formulation of any of embodiments 39-41, wherein the vector is present at a concentration of 5×10⁸ TU/mL to 1×10⁹ TU/mL, e.g., 6×10⁸ TU/mL.
   43. The formulation of any of embodiments 39-42, wherein the buffer is phosphate buffer, sodium citrate, or PIPES.
   44. The formulation of any of embodiments 39-43, wherein the buffer is present at a concentration of 10 mM to 50 mM, e.g., 10 mM to 30 mM, 20 mM to 40 mM, 30 mM to 50 mM, or 10 mM to 40 mM.
   45. The formulation of any of embodiments 39-44, wherein the buffer is present at a concentration of 10 mM, 20 mM, or 40 mM.
   46. The formulation of any of embodiments 39-45 wherein the stabilizer comprises a sugar or a polyhydric alcohol, e.g., sucrose, trehalose, sorbitol, inositol, glucose, or dextran.
   47. The formulation of any of embodiments 39-46, wherein the stabilizer is present at a concentration of 1% to 5%, e.g., 1% to 3%, 2% to 3%, or 1% to 2.5%.
   48. The formulation of any of embodiments 39-47, wherein the stabilizer is present at a concentration of 1%, 2%, or 2.5%.
   49. The formulation of any of embodiments 39-48, wherein sodium chloride is present at a concentration of 50 mM to 200 mM, e.g., 50 mM to 70 mM, 70 mM to 90 mM, 80 mM to 100 mM, 100 mM to 120 mM, 140 mM to 160 mM, or 50 mM to 150 mM.
   50. The formulation of any of embodiments 39-49, wherein sodium chloride is present at a concentration of 50 mM, 60 mM, 80 mM, 90 mM, 110 mM, or 150 mM.
   51. The formulation of any of embodiments 39-50, comprising the vector of embodiment 1, sodium citrate, sucrose, and sodium chloride.
   52. The formulation of embodiment 51, wherein the vector is present at a concentration of 5×10⁸ TU/mL to 5×10⁹ TU/mL, sodium citrate is present at a concentration of 20 mM to 40 mM, sucrose is present at a concentration of 1% to 2%, and sodium chloride is present at a concentration of 100 mM to 150 mM.

### EXAMPLES

### Example 1. Design of Lentiviral Vectors of Human β-Globin Gene

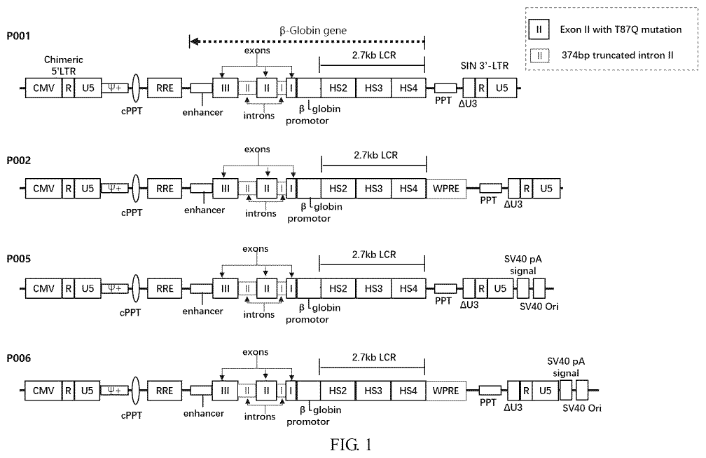

As shown in FIG. 1, three vectors, P002, P005, and P006, were designed with woodchuck hepatitis virus post-transcriptional regulatory element (WPRE), SV40 virus polyadenylation signal (SV40 pA signal), and/or SV40 virus replication origin (SV40 ori) incorporated. In FIG. 1 and FIG. 4, the upstream artificial gene expression control regions represent the truncated locus control regions. A human β-globin gene expression frame that is similar to the gene therapy drug, betibeglogene autotemcel, was designed and used as a control, named as P001 (May C. et al. Nature. 2000; 406(6791):82-6).

### Example 2. Construction of Lentiviral Vectors of Human β-Globin Gene

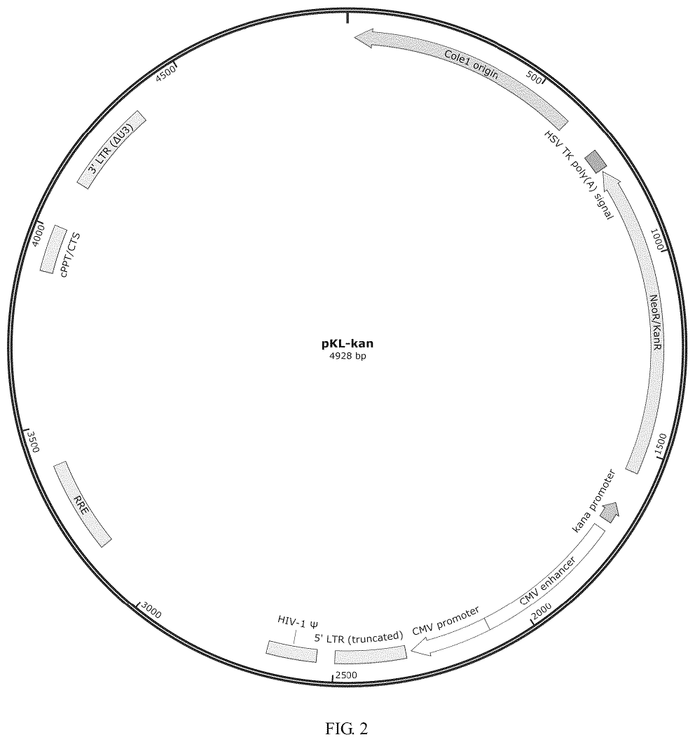

The gene expression frames designed in Example 1 were cloned into a lentiviral vector backbone, which was from the third generation lentiviral vector backbone made in-house by Kanglin Biotech (Hangzhou) Co., Ltd., pKL-Kan (SEQ ID NO: 1) (FIG. 2).

The β-globin gene expression frame P001 (SEQ ID NO: 2) designed in Example 1 was synthesized by Nanjing Genscript Biotechnology Co., Ltd. and cloned in between the multi-cloning sites XhoI/KpnI of the lentiviral vector backbone pKL-Kan by the homologous recombination method well-known to the field. The sequence of the resultant construct was confirmed by sequencing and named as pKL-Kan-TH-P001 (SEQ ID NO: 3).

The β-globin gene expression frame P002 (SEQ ID NO: 4) with an incorporated WPRE was synthesized by Nanjing Genscript Biotechnology Co., Ltd. and cloned in between LCR and 3′ LTR of the lentiviral vector pKL-Kan-TH-P001 by the homologous recombination method. The sequence of the resultant construct was confirmed by sequencing and named as pKL-Kan-TH-P002 (SEQ ID NO: 5).

The β-globin gene expression frame P005 (SEQ ID NO: 6) with an incorporated SV40 pA signal plus SV40 on was synthesized by Nanjing Genscript Biotechnology Co., Ltd. and cloned in between 3′ LTR and kan ori of the lentiviral vector pKL-Kan-TH-P001 by the homologous recombination method. The sequence of the resultant construct was confirmed by sequencing and named as pKL-Kan-TH-P005 (SEQ ID NO: 7).

The WPRE fragment was amplified by PCR and using pKL-Kan-TH-P002 as a template and cloned in between LCR and SV40 pA signal of the lentiviral vector pKL-Kan-TH-P005 by the homologous recombination method. The sequence of the resultant construct was confirmed by sequencing and named as pKL-Kan-TH-P006 (SEQ ID NO: 8).

### Example 3. Packaging of Lentiviruses Containing Human β-Globin Gene

For packaging of β-globin gene therapy lentiviruses, the lentiviral vectors of β-globin gene (pKL-Kan-TH-P001, pKL-Kan-TH-P002, pKL-Kan-TH-P005, or pKL-Kan-TH-P006) constructed in Example 2, envelope plasmid (pKL-Kan-Vsvg; SEQ ID NO: 9), and packaging plasmids (pKL-Kan-Rev (SEQ ID NO: 10) and pKL-Kan-GagPol (SEQ ID NO: 11) were used to co-transfect 293T cells (purchased from ATCC; stock number: CRL-3216) on a 10-cm² cell culture dish, by PEI (a cationic polymer)-mediated transient transfection of eukaryotic cells according to manufacturer's instructions.

PEI-Max transfection reagent was from Polysciences (Catalog Number: 24765-1). 48 hours after transfection, lentiviruses (supernatant of the transfected cells) were harvested, and aliquots were stored at −80° C. Variable volumes of lentivirus was inoculated into human CD4+ T cell line-MT4 cell line (purchased from Shanghai Suoer Biotechnology Co., Ltd.) pre-plated in 96-well cell culture plates. Culture supernatant from the cells transfected with a lentiviral vector containing an EGFP reporter gene (lentivirus packaged with pCCL-sin-EF1α-WPRE-EGFP using the above-described method) was used as positive control, and initial transfection titers of lentivirus in the harvested supernatant were calculated by quantitative PCR (qPCR) and flow cytometry data based on GFP signal using the well-known method in the field. The sequences of primers and probe used in qPCR were as follows.

LV Forward primer:
(SEQ ID NO: 26)
5′-AGTAAGACCACCGCACAGCA-3′
LV Reverse primer:
(SEQ ID NO: 27)
5′-CCTTGGTGGGTGCTACTCCT-3′
LV probe:
(SEQ ID NO: 28)
5′-CCTCCAGGTCTGAAGATCAGCGGCCGC-3′
HK Forward primer:
(SEQ ID NO: 29)
5′-GCTGTCATCTCTTGTGGGCTGT-3′
HK probe:
(SEQ ID NO: 30)
5′-CCTGTCATGCCCACACAAATCTCTCC-3′
HK Reverse primer:
(SEQ ID NO: 31)
5′-ACTCATGGGAGCTGCTGGTTC -3′

The LV probe carries 6-FAM fluorescent dye at the 5′-end and TAMRA fluorescent dye at the 3′-end. The HK probe carries CY5 fluorescent dye at the 5′-end and BHQ2 fluorescent dye at the 3′-end.

The qPCR program: 94° C. 5 min; 95° C. 10 sec, 60° C. 30 sec, 40 cycles.

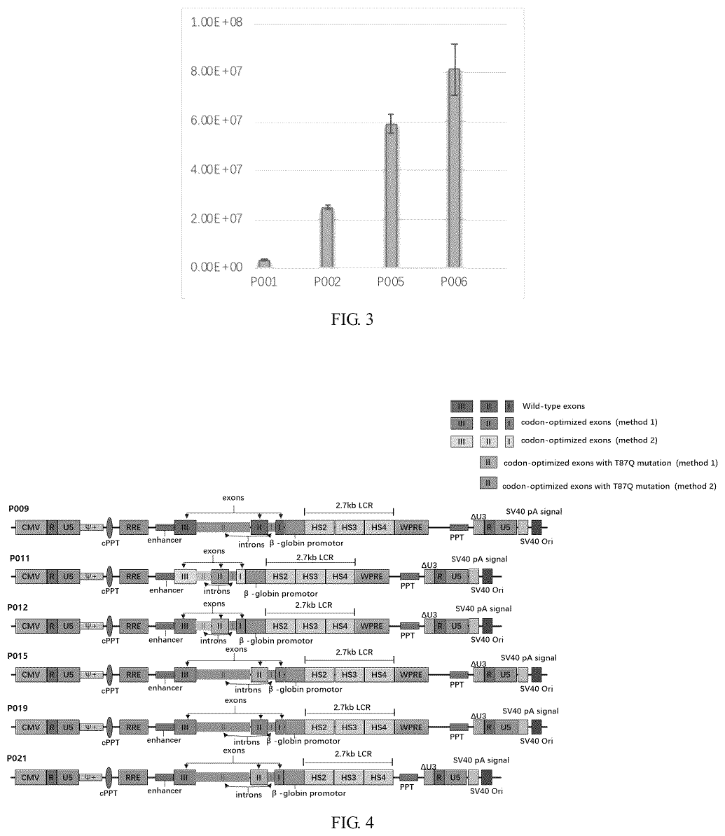

The initial transfection titers of the lentiviruses in the harvested supernatants of the four different β-globin gene lentiviral vectors (pKL-Kan-TH-P001, pKL-Kan-TH-P002, pKL-Kan-TH-P005, pKL-Kan-TH-P006) are shown in FIG. 3. The data show that the cis-acting WPRE and the sequence of SV40 pA signal combined with SV40 on all significantly increased the initial transfection titers of the lentiviruses in the harvested supernatants, and these two enhancements can be additive.

### Example 4. Evaluation of Expression Efficiency of Human β-Globin Gene in Lentivirus

The β-globin gene lentiviral vectors (pKL-Kan-TH-P005, pKL-Kan-TH-P006) were used to transfect 293T cells cultured on 2 of the 15-cm² cell culture dishes using the same protocol as in Example 3, to package lentiviruses. 48 hours after transfection, lentiviruses (supernatant of the transfected cells) were harvested and centrifuged in a table-top bucket centrifuge for 5 min at 4000 rpm and room temperature to remove cell debris, followed by centrifuging at 10000 g, 4° C. for 4 hours. After removing the clear supernatant, 1 mL of RPMI complete culture medium was added to the virus pellet to resuspend the virus particles using a micro sample injector. The virus resuspension was aliquoted and stored at −80° C. for future use.

Variable volumes of lentivirus resuspension was inoculated into MT4 cell line, and transfection titers of the lentivirus resuspension were calculated by qPCR and flow cytometry data based on GFP signal following the protocol described in Example 3.

The expression of β-globin gene mediated by lentivirus was tested in cultured cells. K562 cells are of the erythroleukemia type, derived from a patient of chronic granulocytic leukemia (acute phase), which can produce a small amount of hemoglobin during the fetal development and bear some potential to differentiate into erythrocytes. K562 cells were purchased from ATCC (stock number CCL-243). Based on the above calculated transfection titers of the lentiviruses containing β-globin gene of the resuspension, the lentiviruses were inoculated into K562 cells pre-plated in 96-well plates at various multiplicity of infection (MOI). Some cells were harvested at day 5, 10, and 13 after transfection and used for the following experiments.

1. K562 cells transduced by lentivirus were harvested and washed with PBS. Then the cells were collected after centrifuged at 4200 rpm for 5 min and resuspended in 50 μL QuickExtract™ DNA Extraction Solution (purchased from Lucigen; Catalog Number QE09050). The resuspended cells were lysed in a PCR machine running at the following conditions (Table 1) and total DNAs were isolated.

TABLE 1
PCR conditions

| Temperature | Time |
|---|---|
| 65° C. | 15 min |
| 68° C. | 15 min |
| 95° C. | 10 min |

The vector copy number (VCN) of the lentivirus of the transduced K562 cells were calculated by qPCR and flow cytometry data based on GFP signal using the well-known method in the field. Data show that the two lentiviral vectors P005 and P006 resulted in very similar VCNs in K562 cells when transduced at the same MOI (Table 2).

TABLE 2
VCN in transduced K562 cells

| | |
|---|---|
| Day 5 | Day 10 |

| MOI | P0005 | P0006 | P0005 | P0006 |
|---|---|---|---|---|
| 0.1 | 0.096 | 0.100 | 0.111 | 0.108 |
| 0.2 | 0.179 | 0.170 | 0.230 | 0.213 |
| 0.4 | 0.502 | 0.453 | 0.487 | 0.443 |
| 0.8 | 0.779 | 0.760 | 0.816 | 0.788 |

2. The transduced K562 cells were fixed in 4% paraformaldehyde in PBS and permeabilized with 0.1% Triton-X100 in PBS, and stained with FITC-labeled mouse anti-human β-globin mAb. Flow cytometry based on FITC signal was used to determine the percentage of K562 cells expressing human β-globin protein as well as the relative signal intensity of the expressed human β-globin protein.

Data in Table 3 showed that when K562 cells were transduced at the same MOI and very similar VCNs were obtained, the lentiviral vector P005 resulted in significantly higher expression of β-globin than the lentiviral vector P006.

TABLE 3
Expression levels of β-globin in transduced K562 cells

| | Day 5 | | | | Day 10 | | | | Day 13 | | | |
|---|---|---|---|---|---|---|---|---|---|---|---|---|
| | P0005 | | P0006 | | P0005 | | P0006 | | P0005 | | P0006 | |
| | | P4 | | P4 | | P4 | | P4 | | P4 | | P4 |
| | P4 % | Mean | P4 % | Mean | P4 % | Mean | P4 % | Mean | P4 % | Mean | P4 % | Mean |
| MOI | Parent | FITC-H | Parent | FITC-H | Parent | FITC-H | Parent | FITC-H | Parent | FITC-H | Parent | FITC-H |
| 0.1 | 2.01% | 5685 | 2.02% | 5889 | 4.24% | 7752 | 2.84% | 6532 | 5.06% | 13885 | 4.49% | 10616 |
| | 1.63% | 5345 | 1.59% | 5942 | 4.67% | 8981 | 4.03% | 6646 | 5.99% | 15754 | 5.11% | 12667 |
| 0.2 | 2.83% | 5627 | 2.79% | 5611 | 8.51% | 10549 | 8.59% | 8440 | 9.78% | 14967 | 8.40% | 11332 |
| | 2.60% | 4985 | 2.67% | 5686 | 10.20% | 11107 | 8.21% | 8914 | 11.22% | 15988 | 9.71% | 13522 |
| 0.4 | 6.32% | 5783 | 6.04% | 5510 | 21.25% | 11354 | 17.88% | 9269 | 22.62% | 15672 | 19.68% | 12913 |
| | 5.95% | 5827 | 5.70% | 5295 | 22.01% | 12149 | 15.87% | 8407 | 24.01% | 18199 | 20.81% | 14450 |
| 0.8 | 12.30% | 6463 | 11.82% | 5846 | 33.51% | 11759 | 30.74% | 10305 | 38.82% | 17943 | 36.52% | 15494 |
| | 12.19% | 6044 | 11.05% | 5645 | 33.49% | 12888 | 36.15% | 12914 | 40.56% | 20148 | 39.03% | 16725 |

### Example 5. Additional Design of Lentiviral Vectors of Human β-Globin Gene

Besides the optimization of the cis-acting elements in the lentiviral vectors of β-globin gene, this invention also optimized the expression frame of β-globin gene, including intron sequences and coding sequences.

Based on P006, we designed 6 other vectors (FIG. 4), comprising of, wild-type human β-globin gene sequence (P009), codon-optimized exons with T87Q mutation using method 3 (P011), codon-optimized exons with T87Q mutation using method 4 (P012), coding sequence optimized human β-globin gene with full-length intron 2 and T87Q mutation using method 4 (P015), coding sequence optimized human β-globin gene with full-length intron 2 but no T87Q mutation using method 2 (P019), or coding sequence optimized human β-globin gene with full-length intron 2 and T87Q mutation using method 4 but with WPRE removed (P021).

### Example 6. Construction of Lentiviral Vectors of Human β-Globin Gene

The wild-type human β-globin gene sequence (SEQ ID NO: 12) was amplified by the well-known PCR method in the field, using genomic DNAs isolated from 293T cells, which was derived from human (purchased from ATCC; stock number CRL-3216), as template. The amplified PCR fragment was cloned in between cPPT/CTS and LCR of pKL-Kan-TH-P006 by the homologous recombination method well-known to the field. The sequence of the resultant vector was confirmed by sequencing and named as pKL-Kan-TH-P009 (SEQ ID NO: 13).

The sequence of human β-globin gene with the codon-optimized exons and T87Q mutation using method 3, as designed in Example 5 (SEQ ID NO: 14), was synthesized by Nanjing Genscript Biotechnology Co., Ltd. and cloned in between β-globin-enhancer and β-globin-promoter of pKL-Kan-TH-P006 by homologous recombination. The sequence of the resultant vector was confirmed by sequencing and named as pKL-Kan-TH-PO 11 (SEQ ID NO: 15).

The sequence of human β-globin gene with the codon-optimized exons with T87Q mutation using method 4, as designed in Example 5 (SEQ ID NO: 16), was synthesized by Nanjing Genscript Biotechnology Co., Ltd. and cloned in between β-globin-enhancer and β-globin-promoter of pKL-Kan-TH-P006 by homologous recombination. The sequence of the resultant vector was confirmed by sequencing and named as pKL-Kan-TH-P012 (SEQ ID NO: 17).

The sequence of the full-length intron 2 of human β-globin gene was amplified by PCR using the plasmid DNA of pKL-Kan-TH-P009 as a template and cloned in between exon 2 and exon 3 of pKL-Kan-TH-P012 by homologous recombination. The sequence of the resultant vector was confirmed by sequencing and named as pKL-Kan-TH-P015 (SEQ ID NO: 18).

The T87Q mutation of pKL-Kan-TH-P015 was changed back to T87 by a site-directed mutagenesis kit (purchased from Vazyme Biotech Co., Ltd.; catalog number C214) and confirmed by sequencing. The new vector was named as pKL-Kan-TH-P019 (SEQ ID NO: 19).

Using pKL-Kan-TH-P015 plasmid DNA as a template, two fragments not including the WPRE sequence were amplified and recombined by homologous recombination. The sequence of the resultant new vector was confirmed by sequencing and named as pKL-Kan-TH-P021 (SEQ ID NO: 20).

### Example 7. Evaluation of Expression Efficiency of β-Globin Gene in Lentivirus

The expression efficiency of human β-globin gene using the lentiviral vectors constructed in Example 6 was evaluated using the method described in Example 4.

First, lentiviruses were packaged in 293T cells for the lentiviral vectors pKL-Kan-TH-P006, pKL-Kan-TH-PO 11, and pKL-Kan-TH-P012 constructed in Example 6, and the lentivirus resuspension was aliquoted and stored at −80° C. for future use.

The expression of β-globin gene mediated by lentivirus was tested in cultured cells. Based on the calculated transfection titers of the lentiviruses containing β-globin gene of the resuspension, the lentiviruses were inoculated into K562 cells pre-plated in 96-well plates at the multiplicity of infection (MOI) shown in Table 4. Some cells were harvested at day 5, 10, and 15 after transduction and used for the following experiments.

1. K562 cells transduced by lentiviruses were harvested and lysed, and total DNAs were isolated. The vector copy number (VCN) of the lentivirus of the transduced K562 cells were calculated by qPCR and flow cytometry data based on GFP signal (Table 4).

TABLE 4
VCN in transduced K562 cells

| | MOI | P006 | | P011 | | P012 | |
|---|---|---|---|---|---|---|---|
| Day 5 | 0.5 | 0.553 | 0.568 | 0.462 | 0.440 | 0.365 | 0.367 |
| | 1 | 0.911 | 0.996 | 0.755 | 0.695 | 0.631 | 0.635 |
| | 2 | 1.449 | 1.500 | 1.343 | 1.343 | 1.129 | 1.098 |
| Day 10 | 0.5 | 0.578 | 0.641 | 0.558 | 0.574 | 0.429 | 0.528 |
| | 1 | 1.042 | 1.109 | 0.882 | 0.841 | 0.806 | 0.697 |
| | 2 | 1.602 | 1.777 | 1.505 | 1.484 | 1.257 | 1.205 |
| Day 15 | 0.5 | 0.584 | 0.710 | 0.459 | 0.485 | 0.333 | 0.375 |
| | 1 | 0.990 | 1.185 | 0.850 | 0.874 | 0.622 | 0.648 |
| | 2 | 1.619 | 1.912 | 1.459 | 1.449 | 1.010 | 1.177 |

2. K562 cells transduced by lentiviruses were harvested. Flow cytometry based on PE signal was used to determine the percentage of K562 cells expressing human β-globin protein as well as the relative signal intensity of the expressed human β-globin protein.

TABLE 5
Expression levels of β-globin in transduced K562 cells

| | | P006 | | P011 | | P012 | |
|---|---|---|---|---|---|---|---|
| | | | P4 | | P4 | | P4 |
| | | P4 % | Mean | P4 % | Mean | P4 % | Mean |
| | MOI | Parent | FITC-H | Parent | FITC-H | Parent | FITC-H |
| Day 5 | 0.5 | 10.60% | 7846 | 10.27% | 9344 | 12.23% | 9683 |
| | | 12.21% | 8143 | 9.72% | 9432 | 11.76% | 9020 |
| | 1 | 18.60% | 7928 | 18.61% | 9130 | 19.73% | 9640 |
| | | 21.30% | 8614 | 17.44% | 8885 | 19.72% | 9992 |
| | 2 | 29.35% | 8880 | 31.83% | 10071 | 33.57% | 10141 |
| | | 32.65% | 8999 | 28.81% | 9860 | 33.07% | 10451 |
| Day 10 | 0.5 | 23.19% | 9052 | 14.34% | 7429 | 17.99% | 9465 |
| | | 24.26% | 9507 | 18.74% | 8826 | 15.94% | 7934 |
| | 1 | 39.62% | 11100 | 30.27% | 9436 | 29.86% | 9735 |
| | | 43.41% | 10362 | 28.77% | 9030 | 27.85% | 9051 |
| | 2 | 61.95% | 11382 | 54.04% | 10962 | 57.48% | 11824 |
| | | 63.45% | 11160 | 50.42% | 11347 | 53.49% | 11411 |
| Day 15 | 0.5 | 24.81% | 11122 | 16.16% | 9911 | 16.71% | 10635 |
| | | 26.90% | 11326 | 17.46% | 10046 | 16.88% | 10816 |
| | 1 | 42.48% | 11475 | 27.24% | 10284 | 26.15% | 11664 |
| | | 45.36% | 12691 | 28.99% | 10883 | 26.63% | 11015 |
| | 2 | 62.50% | 13820 | 47.19% | 11653 | 48.18% | 14046 |
| | | 65.41% | 14093 | 45.74% | 11749 | 50.04% | 14211 |

Data showed that when K562 cells were transduced at the same MOI and very similar VCNs were obtained, the lentiviral vectors P011 and P012 resulted in significantly higher expression of β-globin than the lentiviral vector P006 (Table 5). Among them, P012 is more advantageous.

### Example 8. Evaluation of Expression Efficiency of β-Globin Gene in Lentivirus

The expression efficiency of human β-globin gene using the lentiviral vectors constructed in Example 6 was evaluated using the method described in Example 4.

First, lentiviruses were packaged in 293T cells for the lentiviral vectors pKL-Kan-TH-P006, pKL-Kan-TH-P009, pKL-Kan-TH-P012, pKL-Kan-TH-P015, and pKL-Kan-TH-P019 constructed in Example 6, and the lentivirus resuspension was aliquoted and stored at −80° C. for future use.

The expression of β-globin gene mediated by lentivirus was then tested in cultured cells. Based on the calculated transfection titers of the lentiviruses containing β-globin gene of the resuspension, the lentiviruses were inoculated into K562 cells pre-plated in 96-well plates at the multiplicity of infection (MOI) shown in Table 6. Some cells were harvested at day 5 and day 10 after transduction and used for the following experiments.

1. K562 cells transfected by lentiviruses were harvested and lysed, and total DNAs were isolated. The vector copy number (VCN) of the lentivirus of the transfected K562 cells were calculated by qPCR and flow cytometry data based on GFP signal (Table 6).

TABLE 6
VCN in transduced K562 cells

| | Day 5 | | | | | Day 10 | | | | |
|---|---|---|---|---|---|---|---|---|---|---|
| MOI | P006 | P009 | P012 | P015 | P019 | P006 | P009 | P012 | P015 | P019 |
| 0.2 | 0.141 | 0.125 | 0.094 | 0.168 | 0.148 | 0.120 | 0.119 | 0.073 | 0.114 | 0.127 |
| 0.4 | 0.247 | 0.232 | 0.158 | 0.275 | 0.290 | 0.291 | 0.227 | 0.164 | 0.233 | 0.282 |
| 0.8 | 0.471 | 0.429 | 0.294 | 0.455 | 0.524 | 0.498 | 0.365 | 0.296 | 0.398 | 0.480 |
| 1.6 | 0.824 | 0.774 | 0.522 | 0.768 | 0.895 | 0.777 | 0.693 | 0.527 | 0.730 | 0.886 |

2. K562 cells transfected by lentiviruses were harvested. Flow cytometry based on PE signal was used to determine the percentage of K562 cells expressing human β-globin protein as well as the relative signal intensity of the expressed human β-globin protein.

Data in Table 7 showed that when K562 cells were transfected at the same MOI and very similar VCNs were obtained, the lentiviral vectors P012 and P015 resulted in significantly higher expression of β-globin than the other lentiviral vectors.

TABLE 7
Expression levels of β-globin in transduced K562 cells

| | | P006 | | P009 | | P012 | | P015 | | P019 | |
|---|---|---|---|---|---|---|---|---|---|---|---|
| | | | P4 | | P4 | | P4 | | P4 | | P4 |
| | | P4 % | Mean | P4 % | Mean | P4 % | Mean | P4 % | Mean | P4 % | Mean |
| | MOI | Parent | FITC-H | Parent | FITC-H | Parent | FITC-H | Parent | FITC-H | Parent | FITC-H |
| Day 5 | 0.2 | 3.30% | 5127 | 4.53% | 6317 | 2.81% | 6859 | 5.77% | 7693 | 5.64% | 6554 |
| | | 3.20% | 4832 | 3.62% | 5836 | 2.96% | 6309 | 5.40% | 6965 | 5.92% | 5761 |
| | 0.4 | 6.45% | 5270 | 7.82% | 6256 | 5.93% | 6580 | 9.81% | 7224 | 11.80% | 6386 |
| | | 6.50% | 6205 | 7.52% | 6154 | 5.64% | 7033 | 10.59% | 7380 | 12.34% | 6597 |
| | 0.8 | 11.66% | 5622 | 12.75% | 6833 | 9.83% | 6278 | 17.58% | 7604 | 19.15% | 6700 |
| | | 11.01% | 5768 | 11.94% | 5818 | 10.22% | 6570 | 15.54% | 7300 | 20.94% | 6702 |
| | 1.6 | 18.36% | 5574 | 20.96% | 6437 | 17.21% | 6298 | 27.51% | 7838 | 33.57% | 7412 |
| | | 18.94% | 6288 | 20.90% | 6364 | 17.50% | 6822 | 28.40% | 7899 | 34.58% | 7861 |
| Day 10 | 0.2 | 6.09% | 12935 | 6.21% | 12437 | 4.53% | 14780 | 7.62% | 16326 | 8.54% | 12406 |
| | | 6.72% | 12693 | 5.99% | 11447 | 4.72% | 13604 | 7.86% | 17718 | 7.73% | 11368 |
| | 0.4 | 12.27% | 12227 | 11.65% | 12305 | 9.08% | 15661 | 13.43% | 16084 | 17.30% | 15995 |
| | | 12.90% | 13692 | 11.54% | 14547 | 8.89% | 14650 | 12.60% | 14972 | 15.17% | 13145 |
| | 0.8 | 23.85% | 15172 | 19.12% | 13626 | 16.14% | 16451 | 19.80% | 13285 | 29.96% | 17692 |
| | | 23.19% | 14288 | 19.23% | 14099 | 14.54% | 15323 | 19.55% | 14549 | 28.54% | 16324 |
| | 1.6 | 39.30% | 14501 | 34.66% | 15021 | 28.15% | 16029 | 34.14% | 16404 | 60.57% | 18577 |
| | | 35.85% | 14588 | 34.21% | 15909 | 27.17% | 15576 | 33.74% | 15719 | 49.74% | 18207 |

### Example 9. Construction of TH04 Vector

Considering the safety for clinical trials, the wild-type WPRE (SEQ ID NO: 32) in the lentiviral vector P0012 for beta-thalassemia gene therapy, was replaced with a mutated WPRE (SEQ ID NO: 33). WPRE (Woodchuck hepatitis virus Post-transcriptional Regulatory Element) is a commonly used regulatory element in lentiviral vectors. The WPRE, when placed in the 3′ UTR of the gene of interest, can enhance the expression of the transgene in the early stages of RNA transcription by increasing mRNA levels in the nucleus and cytoplasm. When placed in the upstream of 3′ LTR, transcription termination is improved, and therefore, transcript read-through can be significantly reduced. In the P0012 plasmid, in order to prevent the intron in the β-globin gene from being sliced out before reverse transcription of the lentiviral genome, its reverse complementary sequence was cloned into the lentiviral vector (according to the transcription direction of the lentivirus). The WPRE, just as an element placed in the 3′ LTR, theoretically cannot increase the expression efficiency of β-globin gene. In the P0012 plasmid, the inclusion of WPRE surprisingly significantly increased lentiviral packaging yield by at least about 50%. However, it was discovered that the use of WPRE has risks because of its sequence overlap with the woodchuck hepatitis virus protein X (WHX). Thus, the WPRE sequence was mutated at 6 bases (mut6), including 5 bases in the predicted WHX promoter region and 1 base of the starting codon, so that WHX could not initiate expression of the WPRE sequence, thereby enhancing safety. The mutated version of WPRE was named mWPRE (SEQ ID NO:33), and P0012, after modification, was named TH04.

The mWPRE gene was synthesized and inserted between MluI and KpnI of P0012 by restriction enzyme cleavage and ligation. The new construct was confirmed by sequencing and named TH04.

SEQ ID NO: 32
AATCAACCTCTGGATTACAAAATTTGTGAAAGATTGACTGGTATT
CTTAACTATGTTGCTCCTTTTACGCTATGTGGATACGCTGCTTTA
ATGCCTTTGTATCATGCTATTGCTTCCCGTATGGCTTTCATTTTC
TCCTCCTTGTATAAATCCTGGTTGCTGTCTCTTTATGAGGAGTTG
TGGCCCGTTGTCAGGCAACGTGGCGTGGTGTGCACTGTGTTTGCT
GACGCAACCCCCACTGGTTGGGGCATTGCCACCACCTGTCAGCTC
CTTTCCGGGACTTTCGCTTTCCCCCTCCCTATTGCCACGGCGGAA
CTCATCGCCGCCTGCCTTGCCCGCTGCTGGACAGGGGCTCGGCTG
TTGGGCACTGACAATTCCGTGGTGTTGTCGGGGAAGCTGACGTCC
TTTCCATGGCTGCTCGCCTGTGTTGCCACCTGGATTCTGCGCGGG
ACGTCCTTCTGCTACGTCCCTTCGGCCCTCAATCCAGCGGACCTT
CCTTCCCGCGGCCTGCTGCCGGCTCTGCGGCCTCTTCCGCGTCTT
CGCCTTCGCCCTCAGACGAGTCGGATCTCCCTTTGGGCCGCCTCC
CCGC
SEQ ID NO: 33
AATCAACCTCTGGATTACAAAATTTGTGAAAGATTGACTGGTATT
CTTAACTATGTTGCTCCTTTTACGCTATGTGGATACGCTGCTTTA
ATGCCTTTGTATCATGCTATTGCTTCCCGTATGGCTTTCATTTTC
TCCTCCTTGTATAAATCCTGGTTGCTGTCTCTTTATGAGGAGTTG
TGGCCCGTTGTCAGGCAACGTGGCGTGGTGTGCACTGTGTTTGCT
GACGCAACCCCCACTGGTTGGGGCATTGCCACCACCTGTCAGCTC
CTTTCCGGGACTTTCGCTTTCCCCCTCCCTATTGCCACGGCGGAA
CTCATCGCCGCCTGCCTTGCCCGCTGCTGGACAGGGGCTCGGCTG
TTGGGCACTGACAATTCCGTGGTGTTGTCGGGGAAATCATCGTCC
TTTCCTTGGCTGCTCGCCTGTGTTGCCACCTGGATTCTGCGCGGG
ACGTCCTTCTGCTACGTCCCTTCGGCCCTCAATCCAGCGGACCTT
CCTTCCCGCGGCCTGCTGCCGGCTCTGCGGCCTCTTCCGCGTCTT
CGCCTTCGCCCTCAGACGAGTCGGATCTCCCTTTGGGCCGCCTCC
CCGC

### Example 10. Efficacy Test in Mouse Model of Thalassemia

This Example describes evaluation of the efficacy of the TH04 vector in a mouse model of thalassemia.

The thalassemia model Hbbth-4/Hbb+ mice (purchased from Southern Model Organisms) were used to test efficacy of the TH04 vector. The mice were 11 weeks old, with females as donors and males as recipients. The bone marrow hematopoietic stem/progenitor cells were collected and purified from donor mice, transduced with TH04 lentivirus ex vivo, and infused into the recipient mice via the tail vein injection. The therapeutic effect of TH04 lentivirus was assessed by determining the vector copy number, chimeric rate, and changes in thalassaemia-related blood indicators.

Experimental protocol: Three groups were tested: the treatment group, the negative control group, and the positive control group. For the negative control group (G1), purified bone marrow stem cells from female Hbbth4 mice without LV-TH04 transduction were transplanted into male Hbbth4 mice. For the treatment group (G2), purified bone marrow stem cells from female Hbbth4 mice after LV-TH04 transduction were transplanted into male Hbbth4 mice. For the positive control group (G3), purified bone marrow stem cells from female C57BL/6 mice were transplanted into male Hbbth4 mice. Table 8 includes details of the experimental groups.

TABLE 8
Overview of experimental groups

| | | Irradiation dose | | | |
|---|---|---|---|---|---|
| | | (X-ray), Irradiated | | Number of | |
| | | twice, | Recipient | transfused cells | |
| Group | Name | Time interval 3 h | Animal | (cells/recipient) | Donor Animal |
| G1 | Mock | 4.5Gy*2 | 8♂, Hbbth-4 | 1E+06 | 8♀, Hbbth-4 |
| | (without | | | | |
| | transduction) | | | | |
| G2 | LV-TH04 | 4.5Gy*2 | 8♂, Hbbth-4 | 1E+06 | 8♀, Hbbth-4 |
| | (transduction) | | | | |
| G3 | Positive (Wild-type | 4.5Gy*2 | 8♂, Hbbth-4 | 1E+06 | 8♀, C57BL/6 |
| | C57BL/6 cells) | | | | mice |

Isolation of mouse bone marrow stem cells: After euthanizing the donor animals with carbon dioxide, the femur, tibia, and iliac were quickly separated in a biosafety cabinet and immediately transferred to a sterile Petri dish containing DPBS to prevent the bones from drying out. Bone marrows were rinsed with 5 mL DPBS (2% fetal bovine serum)/mouse into a new 50-mL tube with a 70 m cell strainer, and bone marrow cells suspensions were centrifuged at 500×g for 5 min at room temperature. Bone marrow cells were resuspended with 5 mL of DPBS and slowly added to 4 mL of Ficoll-Paque (p=1.084 g/mL) pre-warmed at room temperature. Mature red blood cells were removed after centrifugation for 15 minutes at room temperature with ramp down to 0/0. After density gradient centrifugation, the liquid except the bottom red blood cell layer was slowly aspirated, the samples were centrifuged at 500×g for 5 min at 4° C., and the pellets were resuspended with 5 mL of DPBS after centrifugation.

Purification of mouse bone marrow stem cells: Stem cells were purified with Lineage Cell Depletion Kit and c-Kit positive sorting kit from MACS according to manufacturer's instructions.

Transduction of bone marrow stem cells and culture: Cells sorted by Lineage Cell Depletion Kit and c-Kit-positive sorting kit were cultured overnight in a 37° C., 5% CO₂ cell culture incubator, and then transduced with LV-TH04 lentivirus with a MOI=200 for the treatment group. The transduced cells for the treatment group, as well as the cells of the negative control group and the positive control group, were continued to be cultured in a 5% CO₂ cell culture incubator for two days.

Irradiation of recipient animals irradiation and cell transfusion: On the day when culturing of bone marrow cells was finished, recipient animals were subjected to myeloablative conditioning with X-ray irradiation (4.5 Gy), twice, 3 h apart. The cultured and collected cell suspensions were infused individually by tail vein injection to groups of animals within two hours after the myeloablative conditioning was completed, and the cells were infused at 1E+06 cells/animal.

Determination of VCN and chimeric rate in PBMCs of mice: 100 μL of whole blood samples were collected by retro-orbital bleeding at 4, 6 and 8 weeks after bone marrow transplantation of recipient animals, and about 5E+05 PBMCs were isolated after density gradient centrifugation and used for chimeric rate and VCN analysis.

The PBMC genomic DNAs were extracted by magnetic bead genome extraction kit, and the concentration of each extracted template DNA was determined by microspectrophotometer. The genomic DNA samples were uniformly diluted with ultrapure water to about 50 ng/μL. Determination of chimeric rate and VCN was accomplished with real-time PCR.

Determination of VCN: Mouse MKL3 gene was used as the reference gene, and LTR as the gene detection primer probe for integration of lentiviral vectors into cells. The sequences for primers and probes are shown in Table 9, the reaction system is shown in Table 10, and the reaction procedure is shown in Table 11.

TABLE 9
Primer and probe sequences
used for VCN qPCR

| | | Fluorescent |
|---|---|---|
| Name | Sequence | labeling |
| Mouse MKL3- | AGACCTCATT | N/A |
| For | GAACGCCTGA | |
| Mouse MKL3- | ATGGTTGCTG | N/A |
| Rev | ATGACACTGC | |
| Mouse MKL3- | ACCAGTAGTAA | 5′-Cy5 3′-BHQ-2 |
| probe | CCTTGCCACTG | |
| | GCA | |
| LTR-For | CTGTTGTGTGA | N/A |
| | CTCTGGTAACT | |
| LTR-Rev | TTCGCTTTCAA | N/A |
| | GTCCCTGTT | |
| LTR-probe | AAATCTCTAGCA | 5′-FAM 3′-MGB |
| | GTGGCGCCCG | |

TABLE 10
Preparation of qPCR reaction system
for VCN determination (20 μL)

| Formulation | Volume (μL) | Remark |
|---|---|---|
| 2 x Probe Premix | 10 | 1x |
| Sterile water | 0.4 | N/A |
| Mouse MKL3-F (10 μM) | 0.8 | N/A |
| Mouse MKL3-R (10 μM) | 0.8 | N/A |
| Mouse MKL3-CY5 (2.5 μM) | 0.7 | N/A |
| LTR-F (10 μM) | 0.8 | N/A |
| LTR-R (10 μM) | 0.8 | N/A |
| LTR-FAM (2.5 μM) | 0.7 | N/A |
| Template DNA | 5.0 | N/A |

TABLE 11
Program of the qPCR

| | | |
|---|---|---|
| Reaction temperature (° C.) | Time | Number of cycles |

| | | | |
|---|---|---|---|
| 95 | 30 | sec | 1 |
| 95 | 5 | sec | 40 |
| 60 (Acquire signals) | 30 | sec | |

The VCN values in the samples were calculated as follows:

2\hat{ }MKL3/2\hat{ }LTR=relative VCN

Relative VCN/single-copy relative VCN average=absolute VCN

Each assay included RAW264.7 cell DNA template containing a single-copy vector per genome (with the integration of only one lentiviral vector).

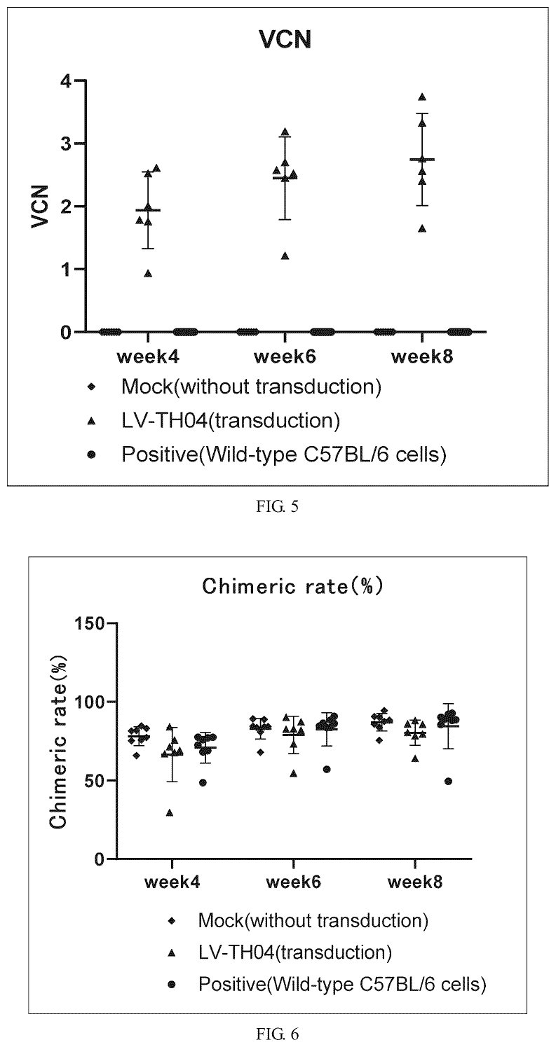

Results of the VCN determination are shown in FIG. 5. The VCN determination in PBMC showed that the VCN of PBMC in the LV-TH04 transduction group was between 1-4, which was as expected.

Determination of chimeric rate: The sample preparation and method of chimeric rate determination were the same as those of VCN determination, except that the LTR primers and probe were replaced with those for the SRY gene, which is a gene unique to the mouse Y chromosome. The SRY sequences are shown in Table 12. The reaction system is the same as Table 10, except that the LTR primers and probe were replaced with those for SRY. The reaction procedure is the same as Table 11.

TABLE 12
Sequences of primers and probe used in
qPCR for chimeric rate determination

| | | Fluorescent |
|---|---|---|
| Name | Sequence | labeling |
| Mouse-SRY-For | TATGGTGTGG | N/A |
| | TCCTGTGGTG | |
| Mouse-SRY-Rev | TTGGGTATTT | N/A |
| | CTCTCTGTGT | |
| | AGG | |
| Mouse-SRY-probe | AGTTGGCCCA | 5′-FAM 3′-MGB |
| | GCAGAATCCC | |
| | AGC | |

Chimeric rate was analyzed as follows:
(1) Calculation of the VCN value in the samples: 2\hat{ }MKL3/2\hat{ }SRY=relative VCN
(2) Relative VCN of the samples/mean relative VCN of male mouse bone marrow cells of the thalassemia model=absolute VCN
(3) Calculation of chimeric rate after female mouse cells reinfused to male mice: 1−VCN then converted to percentage

Results of the chimeric rate analyses are shown in FIG. 6. The chimeric rate assay of PBMCs showed that the implantations of bone marrow stem cells in the three groups were consistent, indicating that LV-TH04 transduction did not affect the implantation of stem cells.

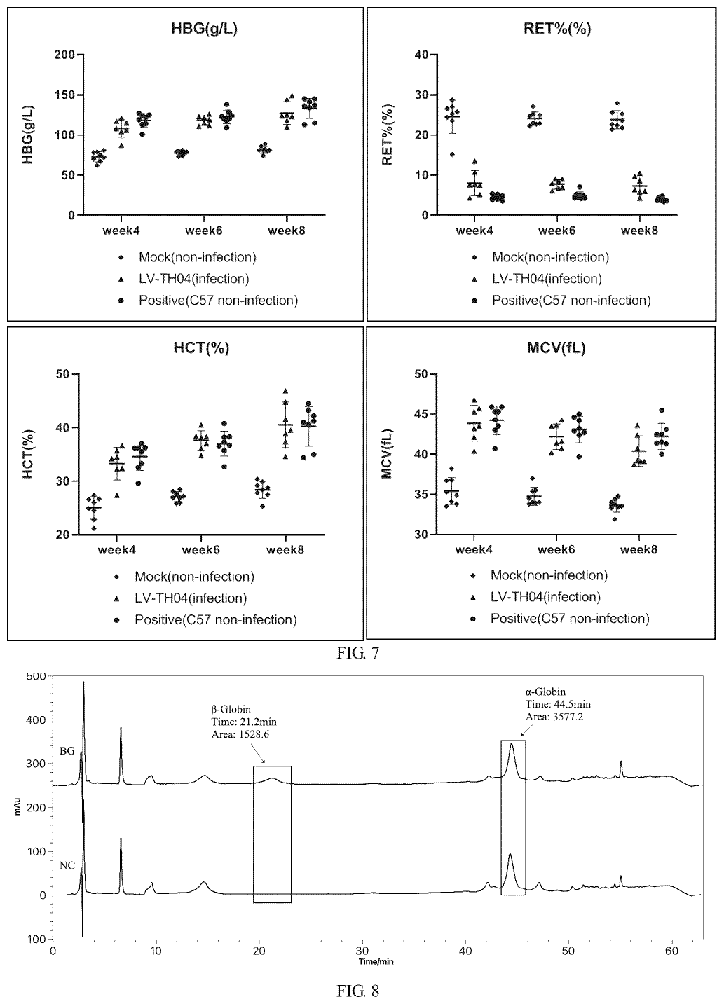

Routine blood tests: Whole blood samples of 50 μL were collected by retro-orbital bleeding from recipient animals for complete blood count (CBC) at 4, 6, and 8 weeks after bone marrow transplantation. The results of main indicators related to thalassaemia are shown in FIG. 7. After transduction and infusion, the LV-TH04 transduction group had significantly improved the hemoglobin content, hematocrit, and average red blood cell volume compared with the Mock group, while the levels of reticulocytes was significantly decreased in the TH04 transduction group compared with the Mock group (FIG. 7). The levels of hemoglobin content, reticulocytes, hematocrit, and average red blood cell volume were similar between the LV-TH04 transduction group and the positive control group (FIG. 7). These data demonstrates the high therapeutic efficacy of TH04 in animal models of the thalassaemia, with curative effects achieved.

### Example 11. Clinical Data 1—Expression Assay in Red Blood Cells Directionally Differentiated from CD34+ Stem Cells Isolated from Thalassemia Major Patients after Ex Vivo Transduction with LV-TH04

This Example describes the effects of TH04 on red blood cells directionally differentiated from CD34+ stem cells isolated from thalassemia major patients.

CD34+ stem cells were isolated from thalassemia major patients by apheresis and transduced with TH04 at MOI=100. After transduction, medium containing erythropoietin was used to promote the directional differentiation of stem cells to red blood cells. The cells were collected after 15 days of culture. 200 μL of ultrapure water was added to and fully mixed with 1E7 cells to lyse the cells, and the supernatants were collected after centrifuging at 12000 rpm for 5 min and used for HPLC assays.

The conditions for the HPLC assays are shown in Table 13. HPLC assay conditions include: column C4 4.6×250 nm; UV 220 nm detection; Sample loading, 20 uL.

TABLE 13
HPLC assay conditions
Mobile phase A: 20% Acetonitrile, 80% Water, 0.1% TFA
Mobile phase B: 60% Acetonitrile, 40% Water, 0.1% TFA

| Time (min) | Flow rate (ml/min) | A % | B % |
|---|---|---|---|
| 0 | 0.8 | 60 | 40 |
| 5 | 0.8 | 50 | 50 |
| 40 | 0.8 | 30 | 70 |
| 50 | 0.8 | 0 | 100 |
| 52 | 0.8 | 60 | 40 |
| 67 | 0.8 | 60 | 40 |

As shown in the chromatograms of FIG. 8 and the peak area calculations in Table 14, the proportion of β/α increased from 0 to 43%. Clinical trial results indicate that patients who have this β/α ratio no longer need blood transfusions and achieve complete cure.

TABLE 14
Peak area of HPLC assays

| | β-globin^{T87Q} | | | | |
|---|---|---|---|---|---|
| | (transduction) | β-globin | α-globin | proportion of | |
| Sample number | Peak Area | Peak Area | Peak Area | β-globin | β/α |
| With LV-TH04 | 1528.5 | 0 | 3577.2 | 29.9 | 0.43 |
| transduction | | | | | |
| Without LV-TH04 | 0 | 0 | 3429.7 | 0 | 0 |
| transduction | | | | | |

### Example 12. Clinical Data 2—Myeloablative Conditioning Protocols Generally Shortened Neutrophil Engraftment Time and Platelet Recovery Time

This Example describes the effects of different myeloablative condition protocols on neutrophil engraftment time and platelet recovery time in patients infused with lentivirally transduced hematopoietic stem cells.

In order to make LV-TH04-transduced CD34+ hematopoietic stem cells (compositions) implant better and faster after infusion into patients, it may be necessary to perform myeloablative conditioning on patients before the composition is reinfused into the patient. In previous clinical trials of gene therapy for thalassemia, including those that have used lentiviral vectors to transduce CD34+ hematopoietic stem cells or gene-editing technology to modify CD34+ hematopoietic stem cells, myeloablative conditioning based on busulfan (BU) was performed. In this Example, myeloablative conditioning based on busulfan combined with cyclophosphamide (BU/CY) was performed, and the protocol was optimized for individual patients. As described below, the results of clinical trials showed that myeloablative conditioning based on BU/CY generally shortened neutrophil engraftment time and platelet recovery time compared with myeloablative conditioning based on BU alone.

The specific steps of myeloablative conditioning are as follows:

### Myeloablative Conditioning Based on BU CY:

Busulfan was intravenously administered for 2 hours and every 6 hours at a dosage of 2.4-4.8 mg/kg/day. Cyclophosphamide was administered 24 hours after busulfan administration. The dosage of cyclophosphamide was 45 to 65 mg/kg/day intravenously. The duration of administration of busulfan was 2 to 4 days, and the duration of administration of cyclophosphamide was 1 to 5 days. Infusion of the composition began 24-72 hours after cyclophosphamide administration.

### Myeloablative Conditioning Based on BU:

Busulfan was intravenously infused for 2 hours and every 6 hours at a dosage of 3.2 mg/kg/day. The duration of administration of busulfan was 4 days. Reinfusion of the composition began 72 hours after busulfan administration.

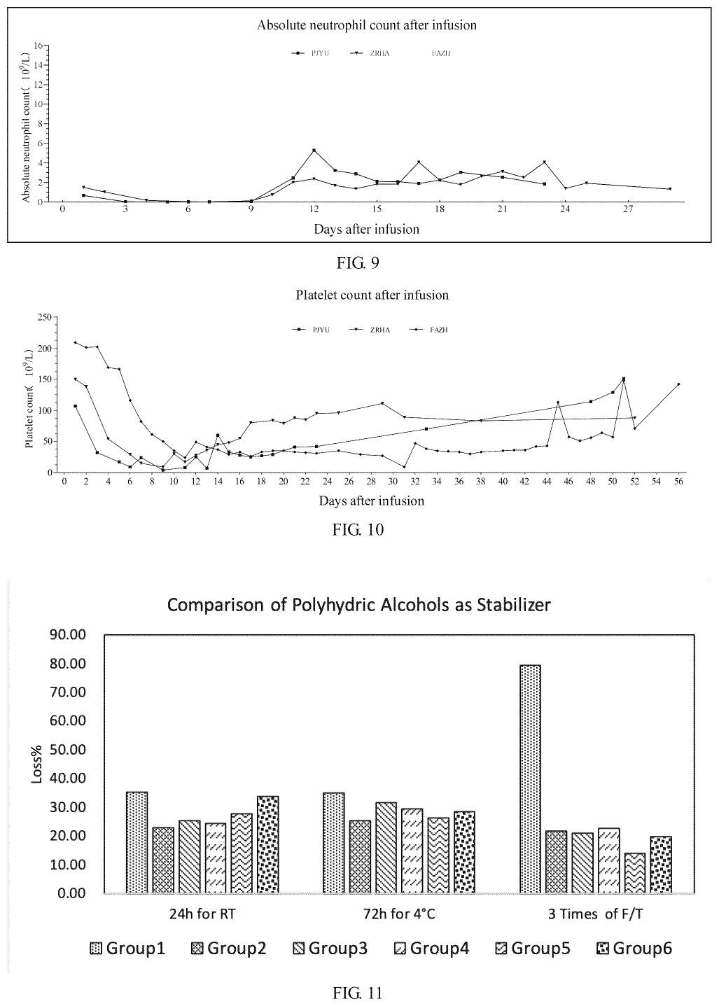

The results of the clinical trial showed that neutrophils engrafted on the 10th day after the reinfusion (ANC≥0.5×10⁹/L for 3 consecutive days) in subjects treated with myeloablative conditioning based on BU/CY (PJYU, ZRHA), while neutrophils engrafted on the 14th day after the reinfusion in subjects treated with myeloablative conditioning based on BU (FAZH) (FIG. 9). Similarly, subjects treated with myeloablative conditioning based on BU/CY (PJYU, ZRHA) had faster platelet recovery than subjects treated with myeloablative conditioning based on BU (FAZH) (FIG. 10). Platelet levels in PJYU and ZRHA showed a continuous upward trend from days 17 and 11, respectively, and recovered to normal levels (≥100×10⁹/L) on days 48 and 29, respectively. Platelet levels in FAZH, on the other hand, fluctuated between 20-40×10⁹/L for up to 28 days, and only began to rise on day 43 and recovered to normal levels for the first time by day 56 (FIG. 10).

### Example 13. Determination of Stabilizers for Pharmaceutical Compositions Comprising Lentiviral Vectors

This Example describes screening and evaluation of stabilizers for pharmaceutical compositions comprising, e.g., lentiviral vectors for thalassemia gene therapy.

### Initial Screening of Stabilizers:

Initial screening of stabilizers was performed by dispersing purified lentiviral vectors for thalassemia gene therapy into the systems shown in Table 15 to form a formulation. Each formulation was tested as follows:
Placing at room temperature for 1 day,
Placing at 4° C. for 3 days, or
Placing under conditions of freeze-thaw for 3 times (freezing at −80° C., with the freezing duration not less than 6 hours, thawing at 23° C., with the thawing duration not less than 30 minutes).

Then, a certain amount of sample was analyzed for loss rate of the viral particles to characterize the stability of the formulations.

TABLE 15
Components of formulations tested during
initial screening of stabilizers.

| | Titer of | | | |
|---|---|---|---|---|
| | Lentiviral | Concentration | Concentration | Concentration |
| No. | vectors | of Buffer | of Stabilizer | of NaCl (mM) |
| 1 | 6*10⁸TU/mL | 20 mM PB | N/A | 150 |
| 2 | 6*10⁸TU/mL | 20 mM PB | 2.5% Trehalose | 75 |
| 3 | 6*10⁸TU/mL | 20 mM PB | 2.5% Sorbitol | 75 |
| 4 | 6*10⁸TU/mL | 20 mM PB | 2.5% Inositol | 75 |
| 5 | 6*10⁸TU/mL | 20 mM PB | 2.5% Sucrose | 75 |
| 6 | 6*10⁸TU/mL | 20 mM PB | 2.5% Glucose | 75 |

As shown in FIG. 11, the addition of sugars or polyhydric alcohols to the formulation inhibited the inactivation of lentiviral particles caused by microtherm and repeated freeze-thaw processes. In particular, the formulation with sucrose exhibited the best performance, demonstrating significant protection compared with the formulation without sugars or polyhydric alcohols.

### Effect of Stabilizers:

To evaluate the effect of stabilizers, purified lentiviral vectors for thalassemia gene therapy were dispersed into the systems shown in Table 16 to form a formulation. Each formulation was tested as follows:

Placing under conditions of freeze-thaw for 3 times (freezing at −80° C., with the freezing duration not less than 6 hours, thawing at 23° C., with the thawing duration not less than 30 minutes), or

Placing under conditions of freeze-thaw for 9 times (freezing at −80° C., with the freezing duration not less than 6 hours, thawing at 23° C., with the thawing duration not less than 30 minutes).

Then, a certain amount of sample was analyzed using biometric titer determination (PCR method).

TABLE 16
Components of formulations tested for
comparing the effect of stabilizers.

| | Titer of | Concen- | Concen- | Concen- |
|---|---|---|---|---|
| | Lentiviral | tration of | tration of | tration of |
| No. | Vectors | Buffer | NaCl (mM) | Sucrose |
| 1 | 2.5*10⁹TU/mL | 20 mM PB | 150 | N/A |
| 2 | 2.5*10⁹TU/mL | 20 mM | 110 | 1% |
| | | Sodium Citrate | | |
| 3 | 2.5*10⁹TU/mL | 20 mM | 150 | N/A |
| | | Sodium Citrate | | |

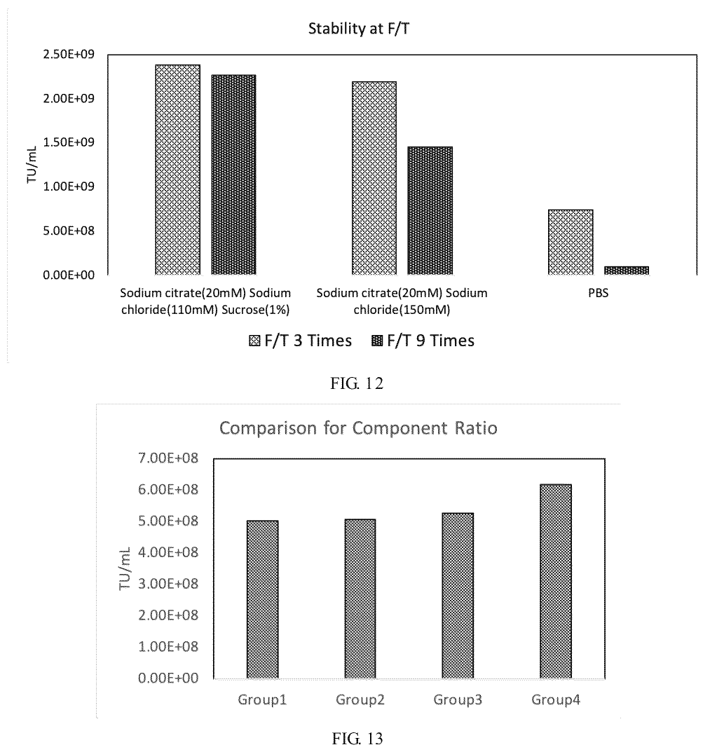

As shown in FIG. 12, the combination of sodium citrate and sucrose in the formulation resulted in good bioactivity of lentiviral particles after repeated freeze-thaw, regardless of the ratio of sodium citrate and sucrose.

### Concentrations of Stabilizers:

To determine how the concentrations of stabilizers affect the stability of lentiviral particles, purified lentiviral vectors for thalassemia gene therapy were dispersed into the systems shown in Table 17 to form a formulation. Each formulation was tested as follows:

Placing under conditions of freeze-thaw for 3 times (freezing at −80° C., with the freezing duration not less than 6 hours, melting at 23° C., with the thawing duration not less than 30 minutes).

Then, a certain amount of sample was analyzed using biometric titer determination (PCR method).

TABLE 17
Components of formulations tested for comparing
concentrations of stabilizers.

| | Titer of | Concen- | Concen- | Concen- |
|---|---|---|---|---|
| | Lentiviral | tration of | tration of | tration of |
| No. | vectors | Buffer | stabilizers | NaCl (mM) |
| 1 | 6.2*10⁸TU/mL | 40 mM | 2% Sucrose | 60 |
| | | Sodium Citrate | | |
| 2 | 6.2*10⁸TU/mL | 40 mM | 1% Sucrose | 80 |
| | | Sodium Citrate | | |
| 3 | 6.2*10⁸TU/mL | 20 mM | 2% Sucrose | 90 |
| | | Sodium Citrate | | |
| 4 | 6.2*10⁸TU/mL | 20 mM | 1% Sucrose | 110 |
| | | Sodium Citrate | | |

As shown in FIG. 13, formulations comprising varying ratios of sodium citrate and sucrose, under isotonic conditions, effectively inhibited loss of the titers and bioactivity of the lentiviral vector particles.

### Example 14. Determination of Buffers for Pharmaceutical Compositions Comprising Lentiviral Vectors

This Example describes screening and evaluation of buffers for pharmaceutical compositions comprising, e.g., lentiviral vectors for thalassemia gene therapy.

### Initial Screening of Buffers:

Initial screening of buffers was performed by dispersing purified lentiviral vectors for thalassemia gene therapy into the systems shown in Table 18 to form a formulation. Each formulation was tested as follows:
Placing at room temperature for 1 day,
Placing at 4° C. for 3 days, or
Placing under conditions of freeze-thaw for 3 times (freezing at −80° C., with the freezing duration not less than 6 hours, thawing at 23° C., with the thawing duration not less than 30 minutes).

Then, a certain amount of sample was analyzed for loss rate of the viral particles to characterize the stability of the formulations.

TABLE 18
Components of formulations tested during
initial screening of buffers.

| | Titer of | | | |
|---|---|---|---|---|
| | Lentiviral | Concentration | Concentration | Concentration |
| No. | vectors | of Buffer | of Stabilizer | of NaCl (mM) |
| 1 | 6*10⁸TU/mL | 20 mM | N/A | 150 |
| | | Sodium Citrate | | |
| 2 | 6*10⁸TU/mL | 20 mM | N/A | 150 |
| | | PE | | |
| 3 | 6*10⁸TU/mL | 20 mM PIPES | N/A | 150 |

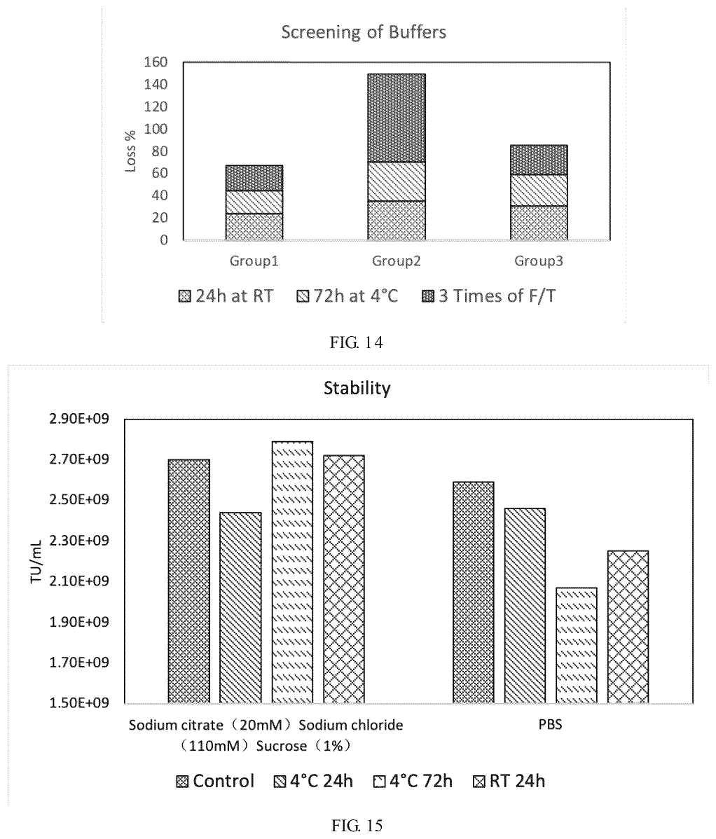

As shown in FIG. 14, the sodium citrate buffer exhibited a better effect on stabilizing the biological activity of lentiviral particles when compared with other buffers. No obvious inactivation of viral particles was observed when using the sodium citrate buffer, indicating that viral particles are more stable in formulations with this buffer than in traditional formulations.

### Effect of Buffers:

To evaluate the effects of buffers, purified lentiviral vectors for thalassemia gene therapy were dispersed into the systems shown in Table 19 to form a formulation. Each group of formulation was tested as follows:
Placing at 4° C. for 1 day,
Placing at room temperature for 1 day, or
Placing at 4° C. for 3 days.

Then, a certain amount of sample was analyzed using biometric titer determination (PCR method).

TABLE 19
Components of formulations tested for
comparing the effects of buffers.

| | Titer of | | | |
|---|---|---|---|---|
| | Lentiviral | Concentration | Concentration | Concentration |
| No. | Vectors | of Buffer | of NaCl (mM) | of Sucrose |
| 1 | 2.8*10⁹TU/mL | 10 mM PB | 140 mM | N/A |
| 2 | 2.8*10⁹TU/mL | 20 mM | 110 mM | 1% |
| | | Sodium Citrate | | |
| 3 | 2.8*10⁹TU/mL | 20 mM | 150 mM | N/A |
| | | Sodium Citrate | | |

As shown in FIG. 15, when compared with traditional PBS, the combination of sodium citrate and sucrose exhibited a better effect on stabilizing the lentiviral bioactivity under isotonic conditions, and there was no significant change in the biological titer of lentiviral particles under various test conditions.

### Summary

The formulations tested, e.g., comprising the stabilizers and/or buffers described in Examples 13 and 14, provide a number of benefits, including good freeze-thaw stability and storage stability for lentiviral particles. The formulations can effectively prevent loss of the biological activity of viral particles caused by repeated freeze-thaw and storage at low temperature, and the viral particles still exhibit good bioactivity after repeated freeze-thaw and long-term storage. Moreover, the formulations tested can disperse more active lentiviral particles in solution, with no obvious precipitation and turbidity observed even with 2-3*10⁹ TU/mL viral particles.

The specific examples described above are to explain the implementation plan of this disclosure and should not be regarded as the limit of the scope of this disclosure. In addition, any modifications and/or variations described in this disclosure are obvious to those skilled in the art to which the present disclosure pertains, as long as they do not depart from the concepts of the present disclosure or go beyond the scope defined by the claims. Although various preferred examples were used to describe the details of the present disclosure, it should be noted that the present disclosure is not limited to these specific examples. In fact, any modifications to the described specific examples, which are obvious to those skilled in the art to which the present disclosure pertains, should all belong to the scope of the present patent protection.

### INCORPORATION BY REFERENCE

All publications, patents, and Accession numbers mentioned herein are hereby incorporated by reference in their entirety as if each individual publication or patent was specifically and individually indicated to be incorporated by reference.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

LISTING OF SEQUENCES
SEQ ID NO: 1
ggccgcgttg ctggcgtttt tccataggct ccgcccccct gacgagcatc acaaaaatcg 60
acgctcaagt cagaggtggc gaaacccgac aggactataa agataccagg cgtttccccc 120
tggaagctcc ctcgtgcgct ctcctgttcc gaccctgccg cttaccggat acctgtccgc 180
ctttctccct tcgggaagcg tggcgctttc tcatagctca cgctgtaggt atctcagttc 240
ggtgtaggtc gttcgctcca agctgggctg tgtgcacgaa ccccccgttc agcccgaccg 300
ctgcgcctta tccggtaact atcgtcttga gtccaacccg gtaagacacg acttatcgcc 360
actggcagca gccactggta acaggattag cagagcgagg tatgtaggcg gtgctacaga 420
gttcttgaag tggtggccta actacggcta cactagaaga acagtatttg gtatctgcgc 480
tctgctgaag ccagttacct tcggaaaaag agttggtagc tcttgatccg gcaaacaaac 540
caccgctggt agcggtggtt tttttgtttg caagcagcag attacgcgca gaaaaaaagg 600
atctcaagaa gatcctttga tcttttctac ggggtctgac gctcagtgga acgaaaactc 660
acgttaaggg attttggtca tgaagcgctt ttgaagctcg gatccgaaca aacgacccaa 720
cacccgtgcg ttttattctg tctttttatt gccgatcccc tcagaagaac tcgtcaagaa 780
ggcgatagaa ggcgatgcgc tgcgaatcgg gagcggcgat accgtaaagc acgaggaagc 840
ggtcagccca ttcgccgcca agctcttcag caatatcacg ggtagccaac gctatgtcct 900
gatagcggtc cgccacaccc agccggccac agtcgatgaa tccagaaaag cggccatttt 960
ccaccatgat attcggcaag caggcatcgc catgggtcac gacgagatcc tcgccgtcgg 1020
gcatgctcgc cttgagcctg gcgaacagtt cggctggcgc gagcccctga tgctcttcgt 1080
ccagatcatc ctgatcgaca agaccggctt ccatccgagt acgtgctcgc tcgatgcgat 1140
gtttcgcttg gtggtcgaat gggcaggtag ccggatcaag cgtatgcagc cgccgcattg 1200
catcagccat gatggatact ttctcggcag gagcaaggtg agatgacagg agatcctgcc 1260
ccggcacttc gcccaatagc agccagtccc ttcccgcttc agtgacaacg tcgagcacag 1320
ctgcgcaagg aacgcccgtc gtggccagcc acgatagccg cgctgcctcg tcttgcagtt 1380
cattcagggc accggacagg tcggtcttga caaaaagaac cgggcgcccc tgcgctgaca 1440
gccggaacac ggcggcatca gagcagccga ttgtctgttg tgcccagtca tagccgaata 1500
gcctctccac ccaagcggcc ggagaacctg cgtgcaatcc atcttgttca atcatgcgaa 1560
acgatcctca tcctgtctct tgatcagagc ttgatcccct gcgccatcag atccttggcg 1620
gcaagaaagc catccagttt actttgcagg gcttcccaac cttaccagag gcctgcgccg 1680
cggccagctg gctagcaatt cccgggttaa ctctagagac attgattatt gactagttat 1740
taatagtaat caattacggg gtcattagtt catagcccat atatggagtt ccgcgttaca 1800
taacttacgg taaatggccc gcctggctga ccgcccaacg acccccgccc attgacgtca 1860
ataatgacgt atgttcccat agtaacgcca atagggactt tccattgacg tcaatgggtg 1920
gagtatttac ggtaaactgc ccacttggca gtacatcaag tgtatcatat gccaagtacg 1980
ccccctattg acgtcaatga cggtaaatgg cccgcctggc attatgccca gtacatgacc 2040
ttatgggact ttcctacttg gcagtacatc tacgtattag tcatcgctat taccatggtg 2100
atgcggtttt ggcagtacat caatgggcgt ggatagcggt ttgactcacg gggatttcca 2160
agtctccacc ccattgacgt caatgggagt ttgttttggc accaaaatca acgggacttt 2220
ccaaaatgtc gtaacaactc cgccccattg acgcaaatgg gcggtaggcg tgtacggtgg 2280
gaggtctata taagcagagc tcgtttagtg aaccggggtc tctctggtta gaccagatct 2340
gagcctggga gctctctggc taactaggga acccactgct taagcctcaa taaagcttgc 2400
cttgagtgct tcaagtagtg tgtgcccgtc tgttgtgtga ctctggtaac tagagatccc 2460
tcagaccctt ttagtcagtg tggaaaatct ctagcagtgg cgcccgaaca gggacttgaa 2520
agcgaaaggg aaaccagagg agctctctcg acgcaggact cggcttgctg aagcgcgcac 2580
ggcaagaggc gaggggcggc gactggtgag tacgccaaaa attttgacta gcggaggcta 2640
gaaggagaga gatgggtgcg agagcgtcag tattaagcgg gggagaatta gatcgcgatg 2700
ggaaaaaatt cggttaaggc cagggggaaa gaaaaaatat aaattaaaac atatagtatg 2760
ggcaagcagg gagctagaac gattcgcagt taatcctggc ctgttagaaa catcagaagg 2820
ctgtagacaa atactgggac agctacaacc atcccttcag acaggatcag aagaacttag 2880
atcattatat aatacagtag caaccctcta ttgtgtgcat caaaggatag agataaaaga 2940
caccaaggaa gctttagaca agatagagga agagcaaaac aaaagtaaga ccaccgcaca 3000
gcaagcggcc gctgatcttc agacctggag gaggagatat gagggacaat tggagaagtg 3060
aattatataa atataaagta gtaaaaattg aaccattagg agtagcaccc accaaggcaa 3120
agagaagagt ggtgcagaga gaaaaaagag cagtgggaat aggagctttg ttccttgggt 3180
tcttgggagc agcaggaagc actatgggcg cagcgtcaat gacgctgacg gtacaggcca 3240
gacaattatt gtctggtata gtgcagcagc agaacaattt gctgagggct attgaggcgc 3300
aacagcatct gttgcaactc acagtctggg gcatcaagca gctccaggca agaatcctgg 3360
ctgtggaaag atacctaaag gatcaacagc tcctggggat ttggggttgc tctggaaaac 3420
tcatttgcac cactgctgtg ccttggaatg ctagttggag taataaatct ctggaacaga 3480
tttggaatca cacgacctgg atggagtggg acagagaaat taacaattac acaagcttaa 3540
tacactcctt aattgaagaa tcgcaaaacc agcaagaaaa gaatgaacaa gaattattgg 3600
aattagataa atgggcaagt ttgtggaatt ggtttaacat aacaaattgg ctgtggtata 3660
taaaattatt cataatgata gtaggaggct tggtaggttt aagaatagtt tttgctgtac 3720
tttctatagt gaatagagtt aggcagggat attcaccatt atcgtttcag acccacctcc 3780
caaccccgag gggacccgac aggcccgaag gaatagaaga agaaggtgga gagagagaca 3840
gagacagatc cattcgatta gtgaacggat ctcgacggta tcggttaact tttaaaagaa 3900
aaggggggat tggggggtac agtgcagggg aaagaatagt agacataata gcaacagaca 3960
tacaaactaa agaattacaa aaacaaatta caaaaattca aaattttatc gatcacgaga 4020
ctagcctcga ggcatgcctg caggaattcg agctcggtac ctttaagacc aatgacttac 4080
aaggcagctg tagatcttag ccacttttta aaagaaaagg ggggactgga agggctaatt 4140
cactcccaac gaagacaaga tctgcttttt gcttgtactg ggtctctctg gttagaccag 4200
atctgagcct gggagctctc tggctaacta gggaacccac tgcttaagcc tcaataaagc 4260
ttgccttgag tgcttcaagt agtgtgtgcc cgtctgttgt gtgactctgg taactagaga 4320
tccctcagac ccttttagtc agtgtggaaa atctctagca gtagtagttc atgtcatctt 4380
attattcagt atttataact tgcaaagaaa tgaatatcag agagtgagag ggtcgaccat 4440
tacttattgt tttagctgtc ctcatgaatg tcttttcact acccatttgc ttatcctgca 4500
tctctcagcc ttgactccac tcagttctct tgcttagaga taccaccttt cccctgaagt 4560
gttccttcca tgttttacgg cgagatggtt tctcctcgcc tggccactca gccttagttg 4620
tctctgttgt cttatagagg tctacttgaa gaaggaaaaa cagggggcat ggtttgactg 4680
tcctgtgagc ccttcttccc tgcctccccc actcacagtg acccggaatc cctcgacatg 4740
gcagtctagc actagtgcgg ccgcagatct gcttcctcgc tcactgactc gctgcgctcg 4800
gtcgttcggc tgcggcgagc ggtatcagct cactcaaagg cggtaatacg gttatccaca 4860
gaatcagggg ataacgcagg aaagaacatg tgagcaaaag gccagcaaaa ggccaggaac 4920
cgtaaaaa 4928
SEQ ID NO: 2
cctcaagatg ataactttta ttttctggac ttgtaatagc tttctcttgt attcaccatg 60
ttgtaacttt cttagagtag taacaatata aagttattgt gagtttttgc aaacacagca 120
aacacaacga cccatataga cattgatgtg aaattgtcta ttgtcaattt atgggaaaac 180
aagtatgtac tttttctact aagccattga aacaggaata acagaacaag attgaaagaa 240
tacattttcc gaaattactt gagtattata caaagacaag cacgtggacc tgggaggagg 300
gttattgtcc atgactggtg tgtggagaca aatgcaggtt tataatagat gggatggcat 360
ctagcgcaat gactttgcca tcacttttag agagctcttg gggaccccag tacacaagag 420
gggacgcagg gtatatgtag acatctcatt ctttttctta gtgtgagaat aagaatagcc 480
atgacctgag tttatagaca atgagccctt ttctctctcc cactcagcag ctatgagatg 540
gcttgccctg cctctctact aggctgactc actccaaggc ccagcaatgg gcagggctct 600
gtcagggctt tgatagcact atctgcagag ccagggccga gaaggggtgg actccagaga 660
ctctccctcc cattcccgag cagggtttgc ttatttatgc atttaaatga tatatttatt 720
ttaaaagaaa taacaggaga ctgcccagcc ctggctgtga catggaaact atgtagaata 780
ttttgggttc catttttttt tccttctttc agttagagga aaaggggctc actgcacata 840
cactagacag aaagtcagga gctttgaatc caagcctgat catttccatg tcatactgag 900
aaagtcccca cccttctctg agcctcagtt tctcttttta taagtaggag tctggagtaa 960
atgatttcca atggctctca tttcaataca aaatttccgt ttattaaatg catgagcttc 1020
tgttactcca agactgagaa ggaaattgaa cctgagactc attgactggc aagatgtccc 1080
cagaggctct cattcagcaa taaaattctc accttcaccc aggcccactg agtgtcagat 1140
ttgcatgcac tagagaagag tcaagcattt gcctaaggtc ggacatgtca gaggcagtgc 1200
cagacctatg tgagactctg cagctactgc tcatgggccc tgtgctgcac tgatgaggag 1260
gatcagatgg atggggcaat gaagcaaagg aatcattctg tggataaagg agacagccat 1320
gaagaagtct atgactgtaa atttgggagc aggagtctct aaggacttgg atttcaagga 1380
attttgactc agcaaacaca agaccctcac ggtgactttg cgagctggtg tgccagatgt 1440
gtctatcaga ggttccaggg agggtggggt ggggtcaggg ctggccacca gctatcaggg 1500
cccagatggg ttataggctg gcaggctcag ataggtggtt aggtcaggtt ggtggtgctg 1560
ggtggagtcc atgactccca ggagccagga gagatagacc atgagtagag ggcagacatg 1620
ggaaaggtgg gggaggcaca gcatagcagc atttttcatt ctactactac atgggactgc 1680
tcccctatac ccccagctag gggcaagtgc cttgactcct atgttttcag gatcatcatc 1740
tataaagtaa gagtaataat tgtgtctatc tcatagggtt attatgagga tcaaaggaga 1800
tgcacactct ctggaccagt ggcctaacag ttcaggacag agctatgggc ttcctatgta 1860
tgggtcagtg gtctcaatgt agcaggcaag ttccagaaga tagcatcaac cactgttaga 1920
gatatactgc cagtctcaga gcctgatgtt aatttagcaa tgggctggga ccctcctcca 1980
gtagaacctt ctaaccagcc cgggaggcgg aggttgcagt gagctgagat cgtgccactg 2040
cactccagcc tgggggacag agcacattat aattaactgt tattttttac ttggactctt 2100
gtggggaata agatacatgt tttattctta tttatgattc aagcactgaa aatagtgttt 2160
agcatccagc aggtgcttca aaaccatttg ctgaatgatt actatacttt ttacaagctc 2220
agctccctct atcccttcca gcatcctcat ctctgattaa ataagcttca gtttttcctt 2280
agttcctgtt acatttctgt gtgtctccat tagtgacctc ccatagtcca agcatgagca 2340
gttctggcca ggcccctgtc ggggtcagtg ccccaccccc gccttctggt tctgtgtaac 2400
cttctaagca aaccttctgg ctcaagcaca gcaatgctga gtcatgatga gtcatgctga 2460
ggcttagggt gtgtgcccag atgttctcag cctagagtga tgactcctat ctgggtcccc 2520
agcaggatgc ttacagggca gatggcaaaa aaaaggagaa gctgaccacc tgactaaaac 2580
tccacctcaa acggcatcat aaagaaaatg gatgcctgag acagaatgtg acatattcta 2640
gatacgtaaa tacacttgca aaggaggatg tttttagtag caatttgtac tgatggtatg 2700
gggccaagag atatatctta gagggagggc tgagggtttg aagtccaact cctaagccag 2760
tgccagaaga gccaaggaca ggtacggctg tcatcactta gacctcaccc tgtggagcca 2820
caccctaggg ttggccaatc tactcccagg agcagggagg gcaggagcca gggctgggca 2880
taaaagtcag ggcagagcca tctattgctt acatttgctt ctgacacaac tgtgttcact 2940
agcaacctca aacagacacc atggtgcatc tgactcctga ggagaagtct gccgttactg 3000
ccctgtgggg caaggtgaac gtggatgaag ttggtggtga ggccctgggc aggttggtat 3060
caaggttaca agacaggttt aaggagacca atagaaactg ggcatgtgga gacagagaag 3120
actcttgggt ttctgatagg cactgactct ctctgcctat tggtctattt tcccaccctt 3180
aggctgctgg tggtctaccc ttggacccag aggttctttg agtcctttgg ggatctgtcc 3240
actcctgatg ctgttatggg caaccctaag gtgaaggctc atggcaagaa agtgctcggt 3300
gcctttagtg atggcctggc tcacctggac aacctcaagg gcacctttgc ccagctgagt 3360
gagctgcact gtgacaagct gcacgtggat cctgagaact tcagggtgag tctatgggac 3420
gcttgatgtt ttctttcccc ttcttttcta tggttaagtt catgtcatag gaaggggata 3480
agtaacaggg tacacatatt gaccaaatca gggtaatttt gcatttgtaa ttttaaaaaa 3540
tgctttcttc ttttaatata cttttttgtt tatcttattt ctaatacttt ccctaatctc 3600
tttctttcag ggcaataatg atacaatgta tcatgcctct ttgcaccatt ctaaagaata 3660
acagtgataa tttctgggtt aaggcaatag caatatctct gcatataaat atttctgcat 3720
ataaattgta actgatgtaa gaggtttcat attgctaata gcagctacaa tccagctacc 3780
attctgcttt tattttatgg ttgggataag gctggattat tctgagtcca agctaggccc 3840
ttttgctaat catgttcata cctcttatct tcctcccaca gctcctgggc aacgtgctgg 3900
tctgtgtgct ggcccatcac tttggcaaag aattcacccc accagtgcag gctgcctatc 3960
agaaagtggt ggctggtgtg gctaatgccc tggcccacaa gtatcactaa gctcgctttc 4020
ttgctgtcca atttctatta aaggttcctt tgttccctaa gtccaactac taaactgggg 4080
gatattatga agggccttga gcatctggat tctgcctaat aaaaaacatt tattttcatt 4140
gcaatgatgt atttaaatta tttctgaata ttttactaaa aagggaatgt gggaggtcag 4200
tgcatttaaa acataaagaa atgaagagct agttcaaacc ttgggaaaat acactatatc 4260
ttaaactcca tgaaagaagg tgaggctgca aacagctaat gcacattggc aacagcccct 4320
gatgcatatg ccttattcat ccctcagaaa aggattcaag tagaggcttg atttggaggt 4380
taaagttttg ctatgctgta ttttacatta cttattgttt tagctgtcct catgaatgtc 4440
ttttcactac ccatttgctt atcctgcatc tctcagcctt gactccactc agttctcttg 4500
cttagagata ccacctttcc cctgaagtgt tccttccatg ttttacggcg agatggtttc 4560
tcctcgcctg gccactcagc cttagttgtc tctgttgtct tatagaggtc tacttgaaga 4620
aggaaaaaca ggggtcatgg tttgactgtc ctgtgagccc ttcttccctg cctcccccac 4680
tcacagtgac ccggaatctg cagtgctagt ctcccggaac tatcactctt tcacagtctg 4740
ctttggaagg actgggctta gtatgaaaag ttaggactga gaagaatttg aaaggcggct 4800
ttttgtagct tgatattcac tactgtctta ttaccctgtc ataggcccac cccaaatgga 4860
agtcccattc ttcctcagga tgtttaagat tagcattcag gaagagatca gaggtctgct 4920
ggctccctta tcatgtccct tatggtgctt ctggctctgc agttattagc atagtgttac 4980
catcaaccac cttaacttca tttttcttat tcaataccta gg 5022
SEQ ID NO: 3
ggccgcgttg ctggcgtttt tccataggct ccgcccccct gacgagcatc acaaaaatcg 60
acgctcaagt cagaggtggc gaaacccgac aggactataa agataccagg cgtttccccc 120
tggaagctcc ctcgtgcgct ctcctgttcc gaccctgccg cttaccggat acctgtccgc 180
ctttctccct tcgggaagcg tggcgctttc tcatagctca cgctgtaggt atctcagttc 240
ggtgtaggtc gttcgctcca agctgggctg tgtgcacgaa ccccccgttc agcccgaccg 300
ctgcgcctta tccggtaact atcgtcttga gtccaacccg gtaagacacg acttatcgcc 360
actggcagca gccactggta acaggattag cagagcgagg tatgtaggcg gtgctacaga 420
gttcttgaag tggtggccta actacggcta cactagaaga acagtatttg gtatctgcgc 480
tctgctgaag ccagttacct tcggaaaaag agttggtagc tcttgatccg gcaaacaaac 540
caccgctggt agcggtggtt tttttgtttg caagcagcag attacgcgca gaaaaaaagg 600
atctcaagaa gatcctttga tcttttctac ggggtctgac gctcagtgga acgaaaactc 660
acgttaaggg attttggtca tgaagcgctt ttgaagctcg gatccgaaca aacgacccaa 720
cacccgtgcg ttttattctg tctttttatt gccgatcccc tcagaagaac tcgtcaagaa 780
ggcgatagaa ggcgatgcgc tgcgaatcgg gagcggcgat accgtaaagc acgaggaagc 840
ggtcagccca ttcgccgcca agctcttcag caatatcacg ggtagccaac gctatgtcct 900
gatagcggtc cgccacaccc agccggccac agtcgatgaa tccagaaaag cggccatttt 960
ccaccatgat attcggcaag caggcatcgc catgggtcac gacgagatcc tcgccgtcgg 1020
gcatgctcgc cttgagcctg gcgaacagtt cggctggcgc gagcccctga tgctcttcgt 1080
ccagatcatc ctgatcgaca agaccggctt ccatccgagt acgtgctcgc tcgatgcgat 1140
gtttcgcttg gtggtcgaat gggcaggtag ccggatcaag cgtatgcagc cgccgcattg 1200
catcagccat gatggatact ttctcggcag gagcaaggtg agatgacagg agatcctgcc 1260
ccggcacttc gcccaatagc agccagtccc ttcccgcttc agtgacaacg tcgagcacag 1320
ctgcgcaagg aacgcccgtc gtggccagcc acgatagccg cgctgcctcg tcttgcagtt 1380
cattcagggc accggacagg tcggtcttga caaaaagaac cgggcgcccc tgcgctgaca 1440
gccggaacac ggcggcatca gagcagccga ttgtctgttg tgcccagtca tagccgaata 1500
gcctctccac ccaagcggcc ggagaacctg cgtgcaatcc atcttgttca atcatgcgaa 1560
acgatcctca tcctgtctct tgatcagagc ttgatcccct gcgccatcag atccttggcg 1620
gcaagaaagc catccagttt actttgcagg gcttcccaac cttaccagag gcctgcgccg 1680
cggccagctg gctagcaatt cccgggttaa ctctagagac attgattatt gactagttat 1740
taatagtaat caattacggg gtcattagtt catagcccat atatggagtt ccgcgttaca 1800
taacttacgg taaatggccc gcctggctga ccgcccaacg acccccgccc attgacgtca 1860
ataatgacgt atgttcccat agtaacgcca atagggactt tccattgacg tcaatgggtg 1920
gagtatttac ggtaaactgc ccacttggca gtacatcaag tgtatcatat gccaagtacg 1980
ccccctattg acgtcaatga cggtaaatgg cccgcctggc attatgccca gtacatgacc 2040
ttatgggact ttcctacttg gcagtacatc tacgtattag tcatcgctat taccatggtg 2100
atgcggtttt ggcagtacat caatgggcgt ggatagcggt ttgactcacg gggatttcca 2160
agtctccacc ccattgacgt caatgggagt ttgttttggc accaaaatca acgggacttt 2220
ccaaaatgtc gtaacaactc cgccccattg acgcaaatgg gcggtaggcg tgtacggtgg 2280
gaggtctata taagcagagc tcgtttagtg aaccggggtc tctctggtta gaccagatct 2340
gagcctggga gctctctggc taactaggga acccactgct taagcctcaa taaagcttgc 2400
cttgagtgct tcaagtagtg tgtgcccgtc tgttgtgtga ctctggtaac tagagatccc 2460
tcagaccctt ttagtcagtg tggaaaatct ctagcagtgg cgcccgaaca gggacttgaa 2520
agcgaaaggg aaaccagagg agctctctcg acgcaggact cggcttgctg aagcgcgcac 2580
ggcaagaggc gaggggcggc gactggtgag tacgccaaaa attttgacta gcggaggcta 2640
gaaggagaga gatgggtgcg agagcgtcag tattaagcgg gggagaatta gatcgcgatg 2700
ggaaaaaatt cggttaaggc cagggggaaa gaaaaaatat aaattaaaac atatagtatg 2760
ggcaagcagg gagctagaac gattcgcagt taatcctggc ctgttagaaa catcagaagg 2820
ctgtagacaa atactgggac agctacaacc atcccttcag acaggatcag aagaacttag 2880
atcattatat aatacagtag caaccctcta ttgtgtgcat caaaggatag agataaaaga 2940
caccaaggaa gctttagaca agatagagga agagcaaaac aaaagtaaga ccaccgcaca 3000
gcaagcggcc gctgatcttc agacctggag gaggagatat gagggacaat tggagaagtg 3060
aattatataa atataaagta gtaaaaattg aaccattagg agtagcaccc accaaggcaa 3120
agagaagagt ggtgcagaga gaaaaaagag cagtgggaat aggagctttg ttccttgggt 3180
tcttgggagc agcaggaagc actatgggcg cagcgtcaat gacgctgacg gtacaggcca 3240
gacaattatt gtctggtata gtgcagcagc agaacaattt gctgagggct attgaggcgc 3300
aacagcatct gttgcaactc acagtctggg gcatcaagca gctccaggca agaatcctgg 3360
ctgtggaaag atacctaaag gatcaacagc tcctggggat ttggggttgc tctggaaaac 3420
tcatttgcac cactgctgtg ccttggaatg ctagttggag taataaatct ctggaacaga 3480
tttggaatca cacgacctgg atggagtggg acagagaaat taacaattac acaagcttaa 3540
tacactcctt aattgaagaa tcgcaaaacc agcaagaaaa gaatgaacaa gaattattgg 3600
aattagataa atgggcaagt ttgtggaatt ggtttaacat aacaaattgg ctgtggtata 3660
taaaattatt cataatgata gtaggaggct tggtaggttt aagaatagtt tttgctgtac 3720
tttctatagt gaatagagtt aggcagggat attcaccatt atcgtttcag acccacctcc 3780
caaccccgag gggacccgac aggcccgaag gaatagaaga agaaggtgga gagagagaca 3840
gagacagatc cattcgatta gtgaacggat ctcgacggta tcggttaact tttaaaagaa 3900
aaggggggat tggggggtac agtgcagggg aaagaatagt agacataata gcaacagaca 3960
tacaaactaa agaattacaa aaacaaatta caaaaattca aaattttatc gatcacgaga 4020
ctagcctcga gaagcttgat atccctaggt attgaataag aaaaatgaag ttaaggtggt 4080
tgatggtaac actatgctaa taactgcaga gccagaagca ccataaggga catgataagg 4140
gagccagcag acctctgatc tcttcctgaa tgctaatctt aaacatcctg aggaagaatg 4200
ggacttccat ttggggtggg cctatgacag ggtaataaga cagtagtgaa tatcaagcta 4260
caaaaagccg cctttcaaat tcttctcagt cctaactttt catactaagc ccagtccttc 4320
caaagcagac tgtgaaagag tgatagttcc gggagactag cactgcagat tccgggtcac 4380
tgtgagtggg ggaggcaggg aagaagggct cacaggacag tcaaaccatg acccctgttt 4440
ttccttcttc aagtagacct ctataagaca acagagacaa ctaaggctga gtggccaggc 4500
gaggagaaac catctcgccg taaaacatgg aaggaacact tcaggggaaa ggtggtatct 4560
ctaagcaaga gaactgagtg gagtcaaggc tgagagatgc aggataagca aatgggtagt 4620
gaaaagacat tcatgaggac agctaaaaca ataagtaatg taaaatacag catagcaaaa 4680
ctttaacctc caaatcaagc ctctacttga atccttttct gagggatgaa taaggcatat 4740
gcatcagggg ctgttgccaa tgtgcattag ctgtttgcag cctcaccttc tttcatggag 4800
tttaagatat agtgtatttt cccaaggttt gaactagctc ttcatttctt tatgttttaa 4860
atgcactgac ctcccacatt ccctttttag taaaatattc agaaataatt taaatacatc 4920
attgcaatga aaataaatgt tttttattag gcagaatcca gatgctcaag gcccttcata 4980
atatccccca gtttagtagt tggacttagg gaacaaagga acctttaata gaaattggac 5040
agcaagaaag cgagcttagt gatacttgtg ggccagggca ttagccacac cagccaccac 5100
tttctgatag gcagcctgca ctggtggggt gaattctttg ccaaagtgat gggccagcac 5160
acagaccagc acgttgccca ggagctgtgg gaggaagata agaggtatga acatgattag 5220
caaaagggcc tagcttggac tcagaataat ccagccttat cccaaccata aaataaaagc 5280
agaatggtag ctggattgta gctgctatta gcaatatgaa acctcttaca tcagttacaa 5340
tttatatgca gaaatattta tatgcagaga tattgctatt gccttaaccc agaaattatc 5400
actgttattc tttagaatgg tgcaaagagg catgatacat tgtatcatta ttgccctgaa 5460
agaaagagat tagggaaagt attagaaata agataaacaa aaaagtatat taaaagaaga 5520
aagcattttt taaaattaca aatgcaaaat taccctgatt tggtcaatat gtgtaccctg 5580
ttacttatcc ccttcctatg acatgaactt aaccatagaa aagaagggga aagaaaacat 5640
caagcgtccc atagactcac cctgaagttc tcaggatcca cgtgcagctt gtcacagtgc 5700
agctcactca gctgggcaaa ggtgcccttg aggttgtcca ggtgagccag gccatcacta 5760
aaggcaccga gcactttctt gccatgagcc ttcaccttag ggttgcccat aacagcatca 5820
ggagtggaca gatccccaaa ggactcaaag aacctctggg tccaagggta gaccaccagc 5880
agcctaaggg tgggaaaata gaccaatagg cagagagagt cagtgcctat cagaaaccca 5940
agagtcttct ctgtctccac atgcccagtt tctattggtc tccttaaacc tgtcttgtaa 6000
ccttgatacc aacctgccca gggcctcacc accaacttca tccacgttca ccttgcccca 6060
cagggcagta acggcagact tctcctcagg agtcagatgc accatggtgt ctgtttgagg 6120
ttgctagtga acacagttgt gtcagaagca aatgtaagca atagatggct ctgccctgac 6180
ttttatgccc agccctggct cctgccctcc ctgctcctgg gagtagattg gccaacccta 6240
gggtgtggct ccacagggtg aggtctaagt gatgacagcc gtacctgtcc ttggctcttc 6300
tggcactggc ttaggagttg gacttcaaac cctcagccct ccctctaaga tatatctctt 6360
ggccccatac catcagtaca aattgctact aaaaacatcc tcctttgcaa gtgtatttac 6420
gtatctagaa tatgtcacat tctgtctcag gcatccattt tctttatgat gccgtttgag 6480
gtggagtttt agtcaggtgg tcagcttctc cttttttttg ccatctgccc tgtaagcatc 6540
ctgctgggga cccagatagg agtcatcact ctaggctgag aacatctggg cacacaccct 6600
aagcctcagc atgactcatc atgactcagc attgctgtgc ttgagccaga aggtttgctt 6660
agaaggttac acagaaccag aaggcggggg tggggcactg accccgacag gggcctggcc 6720
agaactgctc atgcttggac tatgggaggt cactaatgga gacacacaga aatgtaacag 6780
gaactaagga aaaactgaag cttatttaat cagagatgag gatgctggaa gggatagagg 6840
gagctgagct tgtaaaaagt atagtaatca ttcagcaaat ggttttgaag cacctgctgg 6900
atgctaaaca ctattttcag tgcttgaatc ataaataaga ataaaacatg tatcttattc 6960
cccacaagag tccaagtaaa aaataacagt taattataat gtgctctgtc ccccaggctg 7020
gagtgcagtg gcacgatctc agctcactgc aacctccgcc tcccgggctg gttagaaggt 7080
tctactggag gagggtccca gcccattgct aaattaacat caggctctga gactggcagt 7140
atatctctaa cagtggttga tgctatcttc tggaacttgc ctgctacatt gagaccactg 7200
acccatacat aggaagccca tagctctgtc ctgaactgtt aggccactgg tccagagagt 7260
gtgcatctcc tttgatcctc ataataaccc tatgagatag acacaattat tactcttact 7320
ttatagatga tgatcctgaa aacataggag tcaaggcact tgcccctagc tgggggtata 7380
ggggagcagt cccatgtagt agtagaatga aaaatgctgc tatgctgtgc ctcccccacc 7440
tttcccatgt ctgccctcta ctcatggtct atctctcctg gctcctggga gtcatggact 7500
ccacccagca ccaccaacct gacctaacca cctatctgag cctgccagcc tataacccat 7560
ctgggccctg atagctggtg gccagccctg accccacccc accctccctg gaacctctga 7620
tagacacatc tggcacacca gctcgcaaag tcaccgtgag ggtcttgtgt ttgctgagtc 7680
aaaattcctt gaaatccaag tccttagaga ctcctgctcc caaatttaca gtcatagact 7740
tcttcatggc tgtctccttt atccacagaa tgattccttt gcttcattgc cccatccatc 7800
tgatcctcct catcagtgca gcacagggcc catgagcagt agctgcagag tctcacatag 7860
gtctggcact gcctctgaca tgtccgacct taggcaaatg cttgactctt ctctagtgca 7920
tgcaaatctg acactcagtg ggcctgggtg aaggtgagaa ttttattgct gaatgagagc 7980
ctctggggac atcttgccag tcaatgagtc tcaggttcaa tttccttctc agtcttggag 8040
taacagaagc tcatgcattt aataaacgga aattttgtat tgaaatgaga gccattggaa 8100
atcatttact ccagactcct acttataaaa agagaaactg aggctcagag aagggtgggg 8160
actttctcag tatgacatgg aaatgatcag gcttggattc aaagctcctg actttctgtc 8220
tagtgtatgt gcagtgagcc ccttttcctc taactgaaag aaggaaaaaa aaatggaacc 8280
caaaatattc tacatagttt ccatgtcaca gccagggctg ggcagtctcc tgttatttct 8340
tttaaaataa atatatcatt taaatgcata aataagcaaa ccctgctcgg gaatgggagg 8400
gagagtctct ggagtccacc ccttctcggc cctggctctg cagatagtgc tatcaaagcc 8460
ctgacagagc cctgcccatt gctgggcctt ggagtgagtc agcctagtag agaggcaggg 8520
caagccatct catagctgct gagtgggaga gagaaaaggg ctcattgtct ataaactcag 8580
gtcatggcta ttcttattct cacactaaga aaaagaatga gatgtctaca tataccctgc 8640
gtcccctctt gtgtactggg gtccccaaga gctctctaaa agtgatggca aagtcattgc 8700
gctagatgcc atcccatcta ttataaacct gcatttgtct ccacacacca gtcatggaca 8760
ataaccctcc tcccaggtcc acgtgcttgt ctttgtataa tactcaagta atttcggaaa 8820
atgtattctt tcaatcttgt tctgttattc ctgtttcaat ggcttagtag aaaaagtaca 8880
tacttgtttt cccataaatt gacaatagac aatttcacat caatgtctat atgggtcgtt 8940
gtgtttgctg tgtttgcaaa aactcacaat aactttatat tgttactact ctaagaaagt 9000
tacaacatgg tgaatacaag agaaagctat tacaagtcca gaaaataaaa gttatcatct 9060
tgagggtcga cctggaattc gagctcggta cctttaagac caatgactta caaggcagct 9120
gtagatctta gccacttttt aaaagaaaag gggggactgg aagggctaat tcactcccaa 9180
cgaagacaag atctgctttt tgcttgtact gggtctctct ggttagacca gatctgagcc 9240
tgggagctct ctggctaact agggaaccca ctgcttaagc ctcaataaag cttgccttga 9300
gtgcttcaag tagtgtgtgc ccgtctgttg tgtgactctg gtaactagag atccctcaga 9360
cccttttagt cagtgtggaa aatctctagc agtagtagtt catgtcatct tattattcag 9420
tatttataac ttgcaaagaa atgaatatca gagagtgaga gggtcgacca ttacttattg 9480
ttttagctgt cctcatgaat gtcttttcac tacccatttg cttatcctgc atctctcagc 9540
cttgactcca ctcagttctc ttgcttagag ataccacctt tcccctgaag tgttccttcc 9600
atgttttacg gcgagatggt ttctcctcgc ctggccactc agccttagtt gtctctgttg 9660
tcttatagag gtctacttga agaaggaaaa acagggggca tggtttgact gtcctgtgag 9720
cccttcttcc ctgcctcccc cactcacagt gacccggaat ccctcgacat ggcagtctag 9780
cactagtgcg gccgcagatc tgcttcctcg ctcactgact cgctgcgctc ggtcgttcgg 9840
ctgcggcgag cggtatcagc tcactcaaag gcggtaatac ggttatccac agaatcaggg 9900
gataacgcag gaaagaacat gtgagcaaaa ggccagcaaa aggccaggaa ccgtaaaaa 9959
SEQ ID NO: 4
aatcaacctc tggattacaa aatttgtgaa agattgactg gtattcttaa ctatgttgct 60
ccttttacgc tatgtggata cgctgcttta atgcctttgt atcatgctat tgcttcccgt 120
atggctttca ttttctcctc cttgtataaa tcctggttgc tgtctcttta tgaggagttg 180
tggcccgttg tcaggcaacg tggcgtggtg tgcactgtgt ttgctgacgc aacccccact 240
ggttggggca ttgccaccac ctgtcagctc ctttccggga ctttcgcttt ccccctccct 300
attgccacgg cggaactcat cgccgcctgc cttgcccgct gctggacagg ggctcggctg 360
ttgggcactg acaattccgt ggtgttgtcg gggaagctga cgtcctttcc atggctgctc 420
gcctgtgttg ccacctggat tctgcgcggg acgtccttct gctacgtccc ttcggccctc 480
aatccagcgg accttccttc ccgcggcctg ctgccggctc tgcggcctct tccgcgtctt 540
cgccttcgcc ctcagacgag tcggatctcc ctttgggccg cctccccgc 589
SEQ ID NO: 5
ggccgcgttg ctggcgtttt tccataggct ccgcccccct gacgagcatc acaaaaatcg 60
acgctcaagt cagaggtggc gaaacccgac aggactataa agataccagg cgtttccccc 120
tggaagctcc ctcgtgcgct ctcctgttcc gaccctgccg cttaccggat acctgtccgc 180
ctttctccct tcgggaagcg tggcgctttc tcatagctca cgctgtaggt atctcagttc 240
ggtgtaggtc gttcgctcca agctgggctg tgtgcacgaa ccccccgttc agcccgaccg 300
ctgcgcctta tccggtaact atcgtcttga gtccaacccg gtaagacacg acttatcgcc 360
actggcagca gccactggta acaggattag cagagcgagg tatgtaggcg gtgctacaga 420
gttcttgaag tggtggccta actacggcta cactagaaga acagtatttg gtatctgcgc 480
tctgctgaag ccagttacct tcggaaaaag agttggtagc tcttgatccg gcaaacaaac 540
caccgctggt agcggtggtt tttttgtttg caagcagcag attacgcgca gaaaaaaagg 600
atctcaagaa gatcctttga tcttttctac ggggtctgac gctcagtgga acgaaaactc 660
acgttaaggg attttggtca tgaagcgctt ttgaagctcg gatccgaaca aacgacccaa 720
cacccgtgcg ttttattctg tctttttatt gccgatcccc tcagaagaac tcgtcaagaa 780
ggcgatagaa ggcgatgcgc tgcgaatcgg gagcggcgat accgtaaagc acgaggaagc 840
ggtcagccca ttcgccgcca agctcttcag caatatcacg ggtagccaac gctatgtcct 900
gatagcggtc cgccacaccc agccggccac agtcgatgaa tccagaaaag cggccatttt 960
ccaccatgat attcggcaag caggcatcgc catgggtcac gacgagatcc tcgccgtcgg 1020
gcatgctcgc cttgagcctg gcgaacagtt cggctggcgc gagcccctga tgctcttcgt 1080
ccagatcatc ctgatcgaca agaccggctt ccatccgagt acgtgctcgc tcgatgcgat 1140
gtttcgcttg gtggtcgaat gggcaggtag ccggatcaag cgtatgcagc cgccgcattg 1200
catcagccat gatggatact ttctcggcag gagcaaggtg agatgacagg agatcctgcc 1260
ccggcacttc gcccaatagc agccagtccc ttcccgcttc agtgacaacg tcgagcacag 1320
ctgcgcaagg aacgcccgtc gtggccagcc acgatagccg cgctgcctcg tcttgcagtt 1380
cattcagggc accggacagg tcggtcttga caaaaagaac cgggcgcccc tgcgctgaca 1440
gccggaacac ggcggcatca gagcagccga ttgtctgttg tgcccagtca tagccgaata 1500
gcctctccac ccaagcggcc ggagaacctg cgtgcaatcc atcttgttca atcatgcgaa 1560
acgatcctca tcctgtctct tgatcagagc ttgatcccct gcgccatcag atccttggcg 1620
gcaagaaagc catccagttt actttgcagg gcttcccaac cttaccagag gcctgcgccg 1680
cggccagctg gctagcaatt cccgggttaa ctctagagac attgattatt gactagttat 1740
taatagtaat caattacggg gtcattagtt catagcccat atatggagtt ccgcgttaca 1800
taacttacgg taaatggccc gcctggctga ccgcccaacg acccccgccc attgacgtca 1860
ataatgacgt atgttcccat agtaacgcca atagggactt tccattgacg tcaatgggtg 1920
gagtatttac ggtaaactgc ccacttggca gtacatcaag tgtatcatat gccaagtacg 1980
ccccctattg acgtcaatga cggtaaatgg cccgcctggc attatgccca gtacatgacc 2040
ttatgggact ttcctacttg gcagtacatc tacgtattag tcatcgctat taccatggtg 2100
atgcggtttt ggcagtacat caatgggcgt ggatagcggt ttgactcacg gggatttcca 2160
agtctccacc ccattgacgt caatgggagt ttgttttggc accaaaatca acgggacttt 2220
ccaaaatgtc gtaacaactc cgccccattg acgcaaatgg gcggtaggcg tgtacggtgg 2280
gaggtctata taagcagagc tcgtttagtg aaccggggtc tctctggtta gaccagatct 2340
gagcctggga gctctctggc taactaggga acccactgct taagcctcaa taaagcttgc 2400
cttgagtgct tcaagtagtg tgtgcccgtc tgttgtgtga ctctggtaac tagagatccc 2460
tcagaccctt ttagtcagtg tggaaaatct ctagcagtgg cgcccgaaca gggacttgaa 2520
agcgaaaggg aaaccagagg agctctctcg acgcaggact cggcttgctg aagcgcgcac 2580
ggcaagaggc gaggggcggc gactggtgag tacgccaaaa attttgacta gcggaggcta 2640
gaaggagaga gatgggtgcg agagcgtcag tattaagcgg gggagaatta gatcgcgatg 2700
ggaaaaaatt cggttaaggc cagggggaaa gaaaaaatat aaattaaaac atatagtatg 2760
ggcaagcagg gagctagaac gattcgcagt taatcctggc ctgttagaaa catcagaagg 2820
ctgtagacaa atactgggac agctacaacc atcccttcag acaggatcag aagaacttag 2880
atcattatat aatacagtag caaccctcta ttgtgtgcat caaaggatag agataaaaga 2940
caccaaggaa gctttagaca agatagagga agagcaaaac aaaagtaaga ccaccgcaca 3000
gcaagcggcc gctgatcttc agacctggag gaggagatat gagggacaat tggagaagtg 3060
aattatataa atataaagta gtaaaaattg aaccattagg agtagcaccc accaaggcaa 3120
agagaagagt ggtgcagaga gaaaaaagag cagtgggaat aggagctttg ttccttgggt 3180
tcttgggagc agcaggaagc actatgggcg cagcgtcaat gacgctgacg gtacaggcca 3240
gacaattatt gtctggtata gtgcagcagc agaacaattt gctgagggct attgaggcgc 3300
aacagcatct gttgcaactc acagtctggg gcatcaagca gctccaggca agaatcctgg 3360
ctgtggaaag atacctaaag gatcaacagc tcctggggat ttggggttgc tctggaaaac 3420
tcatttgcac cactgctgtg ccttggaatg ctagttggag taataaatct ctggaacaga 3480
tttggaatca cacgacctgg atggagtggg acagagaaat taacaattac acaagcttaa 3540
tacactcctt aattgaagaa tcgcaaaacc agcaagaaaa gaatgaacaa gaattattgg 3600
aattagataa atgggcaagt ttgtggaatt ggtttaacat aacaaattgg ctgtggtata 3660
taaaattatt cataatgata gtaggaggct tggtaggttt aagaatagtt tttgctgtac 3720
tttctatagt gaatagagtt aggcagggat attcaccatt atcgtttcag acccacctcc 3780
caaccccgag gggacccgac aggcccgaag gaatagaaga agaaggtgga gagagagaca 3840
gagacagatc cattcgatta gtgaacggat ctcgacggta tcggttaact tttaaaagaa 3900
aaggggggat tggggggtac agtgcagggg aaagaatagt agacataata gcaacagaca 3960
tacaaactaa agaattacaa aaacaaatta caaaaattca aaattttatc gatcacgaga 4020
ctagcctcga gaagcttgat atccctaggt attgaataag aaaaatgaag ttaaggtggt 4080
tgatggtaac actatgctaa taactgcaga gccagaagca ccataaggga catgataagg 4140
gagccagcag acctctgatc tcttcctgaa tgctaatctt aaacatcctg aggaagaatg 4200
ggacttccat ttggggtggg cctatgacag ggtaataaga cagtagtgaa tatcaagcta 4260
caaaaagccg cctttcaaat tcttctcagt cctaactttt catactaagc ccagtccttc 4320
caaagcagac tgtgaaagag tgatagttcc gggagactag cactgcagat tccgggtcac 4380
tgtgagtggg ggaggcaggg aagaagggct cacaggacag tcaaaccatg acccctgttt 4440
ttccttcttc aagtagacct ctataagaca acagagacaa ctaaggctga gtggccaggc 4500
gaggagaaac catctcgccg taaaacatgg aaggaacact tcaggggaaa ggtggtatct 4560
ctaagcaaga gaactgagtg gagtcaaggc tgagagatgc aggataagca aatgggtagt 4620
gaaaagacat tcatgaggac agctaaaaca ataagtaatg taaaatacag catagcaaaa 4680
ctttaacctc caaatcaagc ctctacttga atccttttct gagggatgaa taaggcatat 4740
gcatcagggg ctgttgccaa tgtgcattag ctgtttgcag cctcaccttc tttcatggag 4800
tttaagatat agtgtatttt cccaaggttt gaactagctc ttcatttctt tatgttttaa 4860
atgcactgac ctcccacatt ccctttttag taaaatattc agaaataatt taaatacatc 4920
attgcaatga aaataaatgt tttttattag gcagaatcca gatgctcaag gcccttcata 4980
atatccccca gtttagtagt tggacttagg gaacaaagga acctttaata gaaattggac 5040
agcaagaaag cgagcttagt gatacttgtg ggccagggca ttagccacac cagccaccac 5100
tttctgatag gcagcctgca ctggtggggt gaattctttg ccaaagtgat gggccagcac 5160
acagaccagc acgttgccca ggagctgtgg gaggaagata agaggtatga acatgattag 5220
caaaagggcc tagcttggac tcagaataat ccagccttat cccaaccata aaataaaagc 5280
agaatggtag ctggattgta gctgctatta gcaatatgaa acctcttaca tcagttacaa 5340
tttatatgca gaaatattta tatgcagaga tattgctatt gccttaaccc agaaattatc 5400
actgttattc tttagaatgg tgcaaagagg catgatacat tgtatcatta ttgccctgaa 5460
agaaagagat tagggaaagt attagaaata agataaacaa aaaagtatat taaaagaaga 5520
aagcattttt taaaattaca aatgcaaaat taccctgatt tggtcaatat gtgtaccctg 5580
ttacttatcc ccttcctatg acatgaactt aaccatagaa aagaagggga aagaaaacat 5640
caagcgtccc atagactcac cctgaagttc tcaggatcca cgtgcagctt gtcacagtgc 5700
agctcactca gctgggcaaa ggtgcccttg aggttgtcca ggtgagccag gccatcacta 5760
aaggcaccga gcactttctt gccatgagcc ttcaccttag ggttgcccat aacagcatca 5820
ggagtggaca gatccccaaa ggactcaaag aacctctggg tccaagggta gaccaccagc 5880
agcctaaggg tgggaaaata gaccaatagg cagagagagt cagtgcctat cagaaaccca 5940
agagtcttct ctgtctccac atgcccagtt tctattggtc tccttaaacc tgtcttgtaa 6000
ccttgatacc aacctgccca gggcctcacc accaacttca tccacgttca ccttgcccca 6060
cagggcagta acggcagact tctcctcagg agtcagatgc accatggtgt ctgtttgagg 6120
ttgctagtga acacagttgt gtcagaagca aatgtaagca atagatggct ctgccctgac 6180
ttttatgccc agccctggct cctgccctcc ctgctcctgg gagtagattg gccaacccta 6240
gggtgtggct ccacagggtg aggtctaagt gatgacagcc gtacctgtcc ttggctcttc 6300
tggcactggc ttaggagttg gacttcaaac cctcagccct ccctctaaga tatatctctt 6360
ggccccatac catcagtaca aattgctact aaaaacatcc tcctttgcaa gtgtatttac 6420
gtatctagaa tatgtcacat tctgtctcag gcatccattt tctttatgat gccgtttgag 6480
gtggagtttt agtcaggtgg tcagcttctc cttttttttg ccatctgccc tgtaagcatc 6540
ctgctgggga cccagatagg agtcatcact ctaggctgag aacatctggg cacacaccct 6600
aagcctcagc atgactcatc atgactcagc attgctgtgc ttgagccaga aggtttgctt 6660
agaaggttac acagaaccag aaggcggggg tggggcactg accccgacag gggcctggcc 6720
agaactgctc atgcttggac tatgggaggt cactaatgga gacacacaga aatgtaacag 6780
gaactaagga aaaactgaag cttatttaat cagagatgag gatgctggaa gggatagagg 6840
gagctgagct tgtaaaaagt atagtaatca ttcagcaaat ggttttgaag cacctgctgg 6900
atgctaaaca ctattttcag tgcttgaatc ataaataaga ataaaacatg tatcttattc 6960
cccacaagag tccaagtaaa aaataacagt taattataat gtgctctgtc ccccaggctg 7020
gagtgcagtg gcacgatctc agctcactgc aacctccgcc tcccgggctg gttagaaggt 7080
tctactggag gagggtccca gcccattgct aaattaacat caggctctga gactggcagt 7140
atatctctaa cagtggttga tgctatcttc tggaacttgc ctgctacatt gagaccactg 7200
acccatacat aggaagccca tagctctgtc ctgaactgtt aggccactgg tccagagagt 7260
gtgcatctcc tttgatcctc ataataaccc tatgagatag acacaattat tactcttact 7320
ttatagatga tgatcctgaa aacataggag tcaaggcact tgcccctagc tgggggtata 7380
ggggagcagt cccatgtagt agtagaatga aaaatgctgc tatgctgtgc ctcccccacc 7440
tttcccatgt ctgccctcta ctcatggtct atctctcctg gctcctggga gtcatggact 7500
ccacccagca ccaccaacct gacctaacca cctatctgag cctgccagcc tataacccat 7560
ctgggccctg atagctggtg gccagccctg accccacccc accctccctg gaacctctga 7620
tagacacatc tggcacacca gctcgcaaag tcaccgtgag ggtcttgtgt ttgctgagtc 7680
aaaattcctt gaaatccaag tccttagaga ctcctgctcc caaatttaca gtcatagact 7740
tcttcatggc tgtctccttt atccacagaa tgattccttt gcttcattgc cccatccatc 7800
tgatcctcct catcagtgca gcacagggcc catgagcagt agctgcagag tctcacatag 7860
gtctggcact gcctctgaca tgtccgacct taggcaaatg cttgactctt ctctagtgca 7920
tgcaaatctg acactcagtg ggcctgggtg aaggtgagaa ttttattgct gaatgagagc 7980
ctctggggac atcttgccag tcaatgagtc tcaggttcaa tttccttctc agtcttggag 8040
taacagaagc tcatgcattt aataaacgga aattttgtat tgaaatgaga gccattggaa 8100
atcatttact ccagactcct acttataaaa agagaaactg aggctcagag aagggtgggg 8160
actttctcag tatgacatgg aaatgatcag gcttggattc aaagctcctg actttctgtc 8220
tagtgtatgt gcagtgagcc ccttttcctc taactgaaag aaggaaaaaa aaatggaacc 8280
caaaatattc tacatagttt ccatgtcaca gccagggctg ggcagtctcc tgttatttct 8340
tttaaaataa atatatcatt taaatgcata aataagcaaa ccctgctcgg gaatgggagg 8400
gagagtctct ggagtccacc ccttctcggc cctggctctg cagatagtgc tatcaaagcc 8460
ctgacagagc cctgcccatt gctgggcctt ggagtgagtc agcctagtag agaggcaggg 8520
caagccatct catagctgct gagtgggaga gagaaaaggg ctcattgtct ataaactcag 8580
gtcatggcta ttcttattct cacactaaga aaaagaatga gatgtctaca tataccctgc 8640
gtcccctctt gtgtactggg gtccccaaga gctctctaaa agtgatggca aagtcattgc 8700
gctagatgcc atcccatcta ttataaacct gcatttgtct ccacacacca gtcatggaca 8760
ataaccctcc tcccaggtcc acgtgcttgt ctttgtataa tactcaagta atttcggaaa 8820
atgtattctt tcaatcttgt tctgttattc ctgtttcaat ggcttagtag aaaaagtaca 8880
tacttgtttt cccataaatt gacaatagac aatttcacat caatgtctat atgggtcgtt 8940
gtgtttgctg tgtttgcaaa aactcacaat aactttatat tgttactact ctaagaaagt 9000
tacaacatgg tgaatacaag agaaagctat tacaagtcca gaaaataaaa gttatcatct 9060
tgagggtcga caatcaacct ctggattaca aaatttgtga aagattgact ggtattctta 9120
actatgttgc tccttttacg ctatgtggat acgctgcttt aatgcctttg tatcatgcta 9180
ttgcttcccg tatggctttc attttctcct ccttgtataa atcctggttg ctgtctcttt 9240
atgaggagtt gtggcccgtt gtcaggcaac gtggcgtggt gtgcactgtg tttgctgacg 9300
caacccccac tggttggggc attgccacca cctgtcagct cctttccggg actttcgctt 9360
tccccctccc tattgccacg gcggaactca tcgccgcctg ccttgcccgc tgctggacag 9420
gggctcggct gttgggcact gacaattccg tggtgttgtc ggggaagctg acgtcctttc 9480
catggctgct cgcctgtgtt gccacctgga ttctgcgcgg gacgtccttc tgctacgtcc 9540
cttcggccct caatccagcg gaccttcctt cccgcggcct gctgccggct ctgcggcctc 9600
ttccgcgtct tcgccttcgc cctcagacga gtcggatctc cctttgggcc gcctccccgc 9660
ctggaattcg agctcggtac ctttaagacc aatgacttac aaggcagctg tagatcttag 9720
ccacttttta aaagaaaagg ggggactgga agggctaatt cactcccaac gaagacaaga 9780
tctgcttttt gcttgtactg ggtctctctg gttagaccag atctgagcct gggagctctc 9840
tggctaacta gggaacccac tgcttaagcc tcaataaagc ttgccttgag tgcttcaagt 9900
agtgtgtgcc cgtctgttgt gtgactctgg taactagaga tccctcagac ccttttagtc 9960
agtgtggaaa atctctagca gtagtagttc atgtcatctt attattcagt atttataact 10020
tgcaaagaaa tgaatatcag agagtgagag ggtcgaccat tacttattgt tttagctgtc 10080
ctcatgaatg tcttttcact acccatttgc ttatcctgca tctctcagcc ttgactccac 10140
tcagttctct tgcttagaga taccaccttt cccctgaagt gttccttcca tgttttacgg 10200
cgagatggtt tctcctcgcc tggccactca gccttagttg tctctgttgt cttatagagg 10260
tctacttgaa gaaggaaaaa cagggggcat ggtttgactg tcctgtgagc ccttcttccc 10320
tgcctccccc actcacagtg acccggaatc cctcgacatg gcagtctagc actagtgcgg 10380
ccgcagatct gcttcctcgc tcactgactc gctgcgctcg gtcgttcggc tgcggcgagc 10440
ggtatcagct cactcaaagg cggtaatacg gttatccaca gaatcagggg ataacgcagg 10500
aaagaacatg tgagcaaaag gccagcaaaa ggccaggaac cgtaaaaa 10548
SEQ ID NO: 6
aacttgttta ttgcagctta taatggttac aaataaagca atagcatcac aaatttcaca 60
aataaagcat ttttttcact gcattctagt tgtggtttgt ccaaactcat caatgtatct 120
tatcatgtct ggctctagct atcccgcccc taactccgcc catcccgccc ctaactccgc 180
ccagttccgc ccattctccg ccccatggct gactaatttt ttttatttat gcagaggccg 240
aggccgcctc ggcctctgag ctattccaga agtagtgagg aggctttttt ggaggcc 297
SEQ ID NO: 7
ggccgcgttg ctggcgtttt tccataggct ccgcccccct gacgagcatc acaaaaatcg 60
acgctcaagt cagaggtggc gaaacccgac aggactataa agataccagg cgtttccccc 120
tggaagctcc ctcgtgcgct ctcctgttcc gaccctgccg cttaccggat acctgtccgc 180
ctttctccct tcgggaagcg tggcgctttc tcatagctca cgctgtaggt atctcagttc 240
ggtgtaggtc gttcgctcca agctgggctg tgtgcacgaa ccccccgttc agcccgaccg 300
ctgcgcctta tccggtaact atcgtcttga gtccaacccg gtaagacacg acttatcgcc 360
actggcagca gccactggta acaggattag cagagcgagg tatgtaggcg gtgctacaga 420
gttcttgaag tggtggccta actacggcta cactagaaga acagtatttg gtatctgcgc 480
tctgctgaag ccagttacct tcggaaaaag agttggtagc tcttgatccg gcaaacaaac 540
caccgctggt agcggtggtt tttttgtttg caagcagcag attacgcgca gaaaaaaagg 600
atctcaagaa gatcctttga tcttttctac ggggtctgac gctcagtgga acgaaaactc 660
acgttaaggg attttggtca tgaagcgctt ttgaagctcg gatccgaaca aacgacccaa 720
cacccgtgcg ttttattctg tctttttatt gccgatcccc tcagaagaac tcgtcaagaa 780
ggcgatagaa ggcgatgcgc tgcgaatcgg gagcggcgat accgtaaagc acgaggaagc 840
ggtcagccca ttcgccgcca agctcttcag caatatcacg ggtagccaac gctatgtcct 900
gatagcggtc cgccacaccc agccggccac agtcgatgaa tccagaaaag cggccatttt 960
ccaccatgat attcggcaag caggcatcgc catgggtcac gacgagatcc tcgccgtcgg 1020
gcatgctcgc cttgagcctg gcgaacagtt cggctggcgc gagcccctga tgctcttcgt 1080
ccagatcatc ctgatcgaca agaccggctt ccatccgagt acgtgctcgc tcgatgcgat 1140
gtttcgcttg gtggtcgaat gggcaggtag ccggatcaag cgtatgcagc cgccgcattg 1200
catcagccat gatggatact ttctcggcag gagcaaggtg agatgacagg agatcctgcc 1260
ccggcacttc gcccaatagc agccagtccc ttcccgcttc agtgacaacg tcgagcacag 1320
ctgcgcaagg aacgcccgtc gtggccagcc acgatagccg cgctgcctcg tcttgcagtt 1380
cattcagggc accggacagg tcggtcttga caaaaagaac cgggcgcccc tgcgctgaca 1440
gccggaacac ggcggcatca gagcagccga ttgtctgttg tgcccagtca tagccgaata 1500
gcctctccac ccaagcggcc ggagaacctg cgtgcaatcc atcttgttca atcatgcgaa 1560
acgatcctca tcctgtctct tgatcagagc ttgatcccct gcgccatcag atccttggcg 1620
gcaagaaagc catccagttt actttgcagg gcttcccaac cttaccagag gcctgcgccg 1680
cggccagctg gctagcaatt cccgggttaa ctctagagac attgattatt gactagttat 1740
taatagtaat caattacggg gtcattagtt catagcccat atatggagtt ccgcgttaca 1800
taacttacgg taaatggccc gcctggctga ccgcccaacg acccccgccc attgacgtca 1860
ataatgacgt atgttcccat agtaacgcca atagggactt tccattgacg tcaatgggtg 1920
gagtatttac ggtaaactgc ccacttggca gtacatcaag tgtatcatat gccaagtacg 1980
ccccctattg acgtcaatga cggtaaatgg cccgcctggc attatgccca gtacatgacc 2040
ttatgggact ttcctacttg gcagtacatc tacgtattag tcatcgctat taccatggtg 2100
atgcggtttt ggcagtacat caatgggcgt ggatagcggt ttgactcacg gggatttcca 2160
agtctccacc ccattgacgt caatgggagt ttgttttggc accaaaatca acgggacttt 2220
ccaaaatgtc gtaacaactc cgccccattg acgcaaatgg gcggtaggcg tgtacggtgg 2280
gaggtctata taagcagagc tcgtttagtg aaccggggtc tctctggtta gaccagatct 2340
gagcctggga gctctctggc taactaggga acccactgct taagcctcaa taaagcttgc 2400
cttgagtgct tcaagtagtg tgtgcccgtc tgttgtgtga ctctggtaac tagagatccc 2460
tcagaccctt ttagtcagtg tggaaaatct ctagcagtgg cgcccgaaca gggacttgaa 2520
agcgaaaggg aaaccagagg agctctctcg acgcaggact cggcttgctg aagcgcgcac 2580
ggcaagaggc gaggggcggc gactggtgag tacgccaaaa attttgacta gcggaggcta 2640
gaaggagaga gatgggtgcg agagcgtcag tattaagcgg gggagaatta gatcgcgatg 2700
ggaaaaaatt cggttaaggc cagggggaaa gaaaaaatat aaattaaaac atatagtatg 2760
ggcaagcagg gagctagaac gattcgcagt taatcctggc ctgttagaaa catcagaagg 2820
ctgtagacaa atactgggac agctacaacc atcccttcag acaggatcag aagaacttag 2880
atcattatat aatacagtag caaccctcta ttgtgtgcat caaaggatag agataaaaga 2940
caccaaggaa gctttagaca agatagagga agagcaaaac aaaagtaaga ccaccgcaca 3000
gcaagcggcc gctgatcttc agacctggag gaggagatat gagggacaat tggagaagtg 3060
aattatataa atataaagta gtaaaaattg aaccattagg agtagcaccc accaaggcaa 3120
agagaagagt ggtgcagaga gaaaaaagag cagtgggaat aggagctttg ttccttgggt 3180
tcttgggagc agcaggaagc actatgggcg cagcgtcaat gacgctgacg gtacaggcca 3240
gacaattatt gtctggtata gtgcagcagc agaacaattt gctgagggct attgaggcgc 3300
aacagcatct gttgcaactc acagtctggg gcatcaagca gctccaggca agaatcctgg 3360
ctgtggaaag atacctaaag gatcaacagc tcctggggat ttggggttgc tctggaaaac 3420
tcatttgcac cactgctgtg ccttggaatg ctagttggag taataaatct ctggaacaga 3480
tttggaatca cacgacctgg atggagtggg acagagaaat taacaattac acaagcttaa 3540
tacactcctt aattgaagaa tcgcaaaacc agcaagaaaa gaatgaacaa gaattattgg 3600
aattagataa atgggcaagt ttgtggaatt ggtttaacat aacaaattgg ctgtggtata 3660
taaaattatt cataatgata gtaggaggct tggtaggttt aagaatagtt tttgctgtac 3720
tttctatagt gaatagagtt aggcagggat attcaccatt atcgtttcag acccacctcc 3780
caaccccgag gggacccgac aggcccgaag gaatagaaga agaaggtgga gagagagaca 3840
gagacagatc cattcgatta gtgaacggat ctcgacggta tcggttaact tttaaaagaa 3900
aaggggggat tggggggtac agtgcagggg aaagaatagt agacataata gcaacagaca 3960
tacaaactaa agaattacaa aaacaaatta caaaaattca aaattttatc gatcacgaga 4020
ctagcctcga gaagcttgat atccctaggt attgaataag aaaaatgaag ttaaggtggt 4080
tgatggtaac actatgctaa taactgcaga gccagaagca ccataaggga catgataagg 4140
gagccagcag acctctgatc tcttcctgaa tgctaatctt aaacatcctg aggaagaatg 4200
ggacttccat ttggggtggg cctatgacag ggtaataaga cagtagtgaa tatcaagcta 4260
caaaaagccg cctttcaaat tcttctcagt cctaactttt catactaagc ccagtccttc 4320
caaagcagac tgtgaaagag tgatagttcc gggagactag cactgcagat tccgggtcac 4380
tgtgagtggg ggaggcaggg aagaagggct cacaggacag tcaaaccatg acccctgttt 4440
ttccttcttc aagtagacct ctataagaca acagagacaa ctaaggctga gtggccaggc 4500
gaggagaaac catctcgccg taaaacatgg aaggaacact tcaggggaaa ggtggtatct 4560
ctaagcaaga gaactgagtg gagtcaaggc tgagagatgc aggataagca aatgggtagt 4620
gaaaagacat tcatgaggac agctaaaaca ataagtaatg taaaatacag catagcaaaa 4680
ctttaacctc caaatcaagc ctctacttga atccttttct gagggatgaa taaggcatat 4740
gcatcagggg ctgttgccaa tgtgcattag ctgtttgcag cctcaccttc tttcatggag 4800
tttaagatat agtgtatttt cccaaggttt gaactagctc ttcatttctt tatgttttaa 4860
atgcactgac ctcccacatt ccctttttag taaaatattc agaaataatt taaatacatc 4920
attgcaatga aaataaatgt tttttattag gcagaatcca gatgctcaag gcccttcata 4980
atatccccca gtttagtagt tggacttagg gaacaaagga acctttaata gaaattggac 5040
agcaagaaag cgagcttagt gatacttgtg ggccagggca ttagccacac cagccaccac 5100
tttctgatag gcagcctgca ctggtggggt gaattctttg ccaaagtgat gggccagcac 5160
acagaccagc acgttgccca ggagctgtgg gaggaagata agaggtatga acatgattag 5220
caaaagggcc tagcttggac tcagaataat ccagccttat cccaaccata aaataaaagc 5280
agaatggtag ctggattgta gctgctatta gcaatatgaa acctcttaca tcagttacaa 5340
tttatatgca gaaatattta tatgcagaga tattgctatt gccttaaccc agaaattatc 5400
actgttattc tttagaatgg tgcaaagagg catgatacat tgtatcatta ttgccctgaa 5460
agaaagagat tagggaaagt attagaaata agataaacaa aaaagtatat taaaagaaga 5520
aagcattttt taaaattaca aatgcaaaat taccctgatt tggtcaatat gtgtaccctg 5580
ttacttatcc ccttcctatg acatgaactt aaccatagaa aagaagggga aagaaaacat 5640
caagcgtccc atagactcac cctgaagttc tcaggatcca cgtgcagctt gtcacagtgc 5700
agctcactca gctgggcaaa ggtgcccttg aggttgtcca ggtgagccag gccatcacta 5760
aaggcaccga gcactttctt gccatgagcc ttcaccttag ggttgcccat aacagcatca 5820
ggagtggaca gatccccaaa ggactcaaag aacctctggg tccaagggta gaccaccagc 5880
agcctaaggg tgggaaaata gaccaatagg cagagagagt cagtgcctat cagaaaccca 5940
agagtcttct ctgtctccac atgcccagtt tctattggtc tccttaaacc tgtcttgtaa 6000
ccttgatacc aacctgccca gggcctcacc accaacttca tccacgttca ccttgcccca 6060
cagggcagta acggcagact tctcctcagg agtcagatgc accatggtgt ctgtttgagg 6120
ttgctagtga acacagttgt gtcagaagca aatgtaagca atagatggct ctgccctgac 6180
ttttatgccc agccctggct cctgccctcc ctgctcctgg gagtagattg gccaacccta 6240
gggtgtggct ccacagggtg aggtctaagt gatgacagcc gtacctgtcc ttggctcttc 6300
tggcactggc ttaggagttg gacttcaaac cctcagccct ccctctaaga tatatctctt 6360
ggccccatac catcagtaca aattgctact aaaaacatcc tcctttgcaa gtgtatttac 6420
gtatctagaa tatgtcacat tctgtctcag gcatccattt tctttatgat gccgtttgag 6480
gtggagtttt agtcaggtgg tcagcttctc cttttttttg ccatctgccc tgtaagcatc 6540
ctgctgggga cccagatagg agtcatcact ctaggctgag aacatctggg cacacaccct 6600
aagcctcagc atgactcatc atgactcagc attgctgtgc ttgagccaga aggtttgctt 6660
agaaggttac acagaaccag aaggcggggg tggggcactg accccgacag gggcctggcc 6720
agaactgctc atgcttggac tatgggaggt cactaatgga gacacacaga aatgtaacag 6780
gaactaagga aaaactgaag cttatttaat cagagatgag gatgctggaa gggatagagg 6840
gagctgagct tgtaaaaagt atagtaatca ttcagcaaat ggttttgaag cacctgctgg 6900
atgctaaaca ctattttcag tgcttgaatc ataaataaga ataaaacatg tatcttattc 6960
cccacaagag tccaagtaaa aaataacagt taattataat gtgctctgtc ccccaggctg 7020
gagtgcagtg gcacgatctc agctcactgc aacctccgcc tcccgggctg gttagaaggt 7080
tctactggag gagggtccca gcccattgct aaattaacat caggctctga gactggcagt 7140
atatctctaa cagtggttga tgctatcttc tggaacttgc ctgctacatt gagaccactg 7200
acccatacat aggaagccca tagctctgtc ctgaactgtt aggccactgg tccagagagt 7260
gtgcatctcc tttgatcctc ataataaccc tatgagatag acacaattat tactcttact 7320
ttatagatga tgatcctgaa aacataggag tcaaggcact tgcccctagc tgggggtata 7380
ggggagcagt cccatgtagt agtagaatga aaaatgctgc tatgctgtgc ctcccccacc 7440
tttcccatgt ctgccctcta ctcatggtct atctctcctg gctcctggga gtcatggact 7500
ccacccagca ccaccaacct gacctaacca cctatctgag cctgccagcc tataacccat 7560
ctgggccctg atagctggtg gccagccctg accccacccc accctccctg gaacctctga 7620
tagacacatc tggcacacca gctcgcaaag tcaccgtgag ggtcttgtgt ttgctgagtc 7680
aaaattcctt gaaatccaag tccttagaga ctcctgctcc caaatttaca gtcatagact 7740
tcttcatggc tgtctccttt atccacagaa tgattccttt gcttcattgc cccatccatc 7800
tgatcctcct catcagtgca gcacagggcc catgagcagt agctgcagag tctcacatag 7860
gtctggcact gcctctgaca tgtccgacct taggcaaatg cttgactctt ctctagtgca 7920
tgcaaatctg acactcagtg ggcctgggtg aaggtgagaa ttttattgct gaatgagagc 7980
ctctggggac atcttgccag tcaatgagtc tcaggttcaa tttccttctc agtcttggag 8040
taacagaagc tcatgcattt aataaacgga aattttgtat tgaaatgaga gccattggaa 8100
atcatttact ccagactcct acttataaaa agagaaactg aggctcagag aagggtgggg 8160
actttctcag tatgacatgg aaatgatcag gcttggattc aaagctcctg actttctgtc 8220
tagtgtatgt gcagtgagcc ccttttcctc taactgaaag aaggaaaaaa aaatggaacc 8280
caaaatattc tacatagttt ccatgtcaca gccagggctg ggcagtctcc tgttatttct 8340
tttaaaataa atatatcatt taaatgcata aataagcaaa ccctgctcgg gaatgggagg 8400
gagagtctct ggagtccacc ccttctcggc cctggctctg cagatagtgc tatcaaagcc 8460
ctgacagagc cctgcccatt gctgggcctt ggagtgagtc agcctagtag agaggcaggg 8520
caagccatct catagctgct gagtgggaga gagaaaaggg ctcattgtct ataaactcag 8580
gtcatggcta ttcttattct cacactaaga aaaagaatga gatgtctaca tataccctgc 8640
gtcccctctt gtgtactggg gtccccaaga gctctctaaa agtgatggca aagtcattgc 8700
gctagatgcc atcccatcta ttataaacct gcatttgtct ccacacacca gtcatggaca 8760
ataaccctcc tcccaggtcc acgtgcttgt ctttgtataa tactcaagta atttcggaaa 8820
atgtattctt tcaatcttgt tctgttattc ctgtttcaat ggcttagtag aaaaagtaca 8880
tacttgtttt cccataaatt gacaatagac aatttcacat caatgtctat atgggtcgtt 8940
gtgtttgctg tgtttgcaaa aactcacaat aactttatat tgttactact ctaagaaagt 9000
tacaacatgg tgaatacaag agaaagctat tacaagtcca gaaaataaaa gttatcatct 9060
tgagggtcga cctggaattc gagctcggta cctttaagac caatgactta caaggcagct 9120
gtagatctta gccacttttt aaaagaaaag gggggactgg aagggctaat tcactcccaa 9180
cgaagacaag atctgctttt tgcttgtact gggtctctct ggttagacca gatctgagcc 9240
tgggagctct ctggctaact agggaaccca ctgcttaagc ctcaataaag cttgccttga 9300
gtgcttcaag tagtgtgtgc ccgtctgttg tgtgactctg gtaactagag atccctcaga 9360
cccttttagt cagtgtggaa aatctctagc agtagtagtt catgtcatct tattattcag 9420
tatttataac ttgcaaagaa atgaatatca gagagtgaga ggaacttgtt tattgcagct 9480
tataatggtt acaaataaag caatagcatc acaaatttca caaataaagc atttttttca 9540
ctgcattcta gttgtggttt gtccaaactc atcaatgtat cttatcatgt ctggctctag 9600
ctatcccgcc cctaactccg cccatcccgc ccctaactcc gcccagttcc gcccattctc 9660
cgccccatgg ctgactaatt ttttttattt atgcagaggc cgaggccgcc tcggcctctg 9720
agctattcca gaagtagtga ggaggctttt ttggaggccg ctagcgtcga ccattactta 9780
ttgttttagc tgtcctcatg aatgtctttt cactacccat ttgcttatcc tgcatctctc 9840
agccttgact ccactcagtt ctcttgctta gagataccac ctttcccctg aagtgttcct 9900
tccatgtttt acggcgagat ggtttctcct cgcctggcca ctcagcctta gttgtctctg 9960
ttgtcttata gaggtctact tgaagaagga aaaacagggg gcatggtttg actgtcctgt 10020
gagcccttct tccctgcctc ccccactcac agtgacccgg aatccctcga catggcagtc 10080
tagcactagt gcggccgcag atctgcttcc tcgctcactg actcgctgcg ctcggtcgtt 10140
cggctgcggc gagcggtatc agctcactca aaggcggtaa tacggttatc cacagaatca 10200
ggggataacg caggaaagaa catgtgagca aaaggccagc aaaaggccag gaaccgtaaa 10260
aa 10262
SEQ ID NO: 8
ggccgcgttg ctggcgtttt tccataggct ccgcccccct gacgagcatc acaaaaatcg 60
acgctcaagt cagaggtggc gaaacccgac aggactataa agataccagg cgtttccccc 120
tggaagctcc ctcgtgcgct ctcctgttcc gaccctgccg cttaccggat acctgtccgc 180
ctttctccct tcgggaagcg tggcgctttc tcatagctca cgctgtaggt atctcagttc 240
ggtgtaggtc gttcgctcca agctgggctg tgtgcacgaa ccccccgttc agcccgaccg 300
ctgcgcctta tccggtaact atcgtcttga gtccaacccg gtaagacacg acttatcgcc 360
actggcagca gccactggta acaggattag cagagcgagg tatgtaggcg gtgctacaga 420
gttcttgaag tggtggccta actacggcta cactagaaga acagtatttg gtatctgcgc 480
tctgctgaag ccagttacct tcggaaaaag agttggtagc tcttgatccg gcaaacaaac 540
caccgctggt agcggtggtt tttttgtttg caagcagcag attacgcgca gaaaaaaagg 600
atctcaagaa gatcctttga tcttttctac ggggtctgac gctcagtgga acgaaaactc 660
acgttaaggg attttggtca tgaagcgctt ttgaagctcg gatccgaaca aacgacccaa 720
cacccgtgcg ttttattctg tctttttatt gccgatcccc tcagaagaac tcgtcaagaa 780
ggcgatagaa ggcgatgcgc tgcgaatcgg gagcggcgat accgtaaagc acgaggaagc 840
ggtcagccca ttcgccgcca agctcttcag caatatcacg ggtagccaac gctatgtcct 900
gatagcggtc cgccacaccc agccggccac agtcgatgaa tccagaaaag cggccatttt 960
ccaccatgat attcggcaag caggcatcgc catgggtcac gacgagatcc tcgccgtcgg 1020
gcatgctcgc cttgagcctg gcgaacagtt cggctggcgc gagcccctga tgctcttcgt 1080
ccagatcatc ctgatcgaca agaccggctt ccatccgagt acgtgctcgc tcgatgcgat 1140
gtttcgcttg gtggtcgaat gggcaggtag ccggatcaag cgtatgcagc cgccgcattg 1200
catcagccat gatggatact ttctcggcag gagcaaggtg agatgacagg agatcctgcc 1260
ccggcacttc gcccaatagc agccagtccc ttcccgcttc agtgacaacg tcgagcacag 1320
ctgcgcaagg aacgcccgtc gtggccagcc acgatagccg cgctgcctcg tcttgcagtt 1380
cattcagggc accggacagg tcggtcttga caaaaagaac cgggcgcccc tgcgctgaca 1440
gccggaacac ggcggcatca gagcagccga ttgtctgttg tgcccagtca tagccgaata 1500
gcctctccac ccaagcggcc ggagaacctg cgtgcaatcc atcttgttca atcatgcgaa 1560
acgatcctca tcctgtctct tgatcagagc ttgatcccct gcgccatcag atccttggcg 1620
gcaagaaagc catccagttt actttgcagg gcttcccaac cttaccagag gcctgcgccg 1680
cggccagctg gctagcaatt cccgggttaa ctctagagac attgattatt gactagttat 1740
taatagtaat caattacggg gtcattagtt catagcccat atatggagtt ccgcgttaca 1800
taacttacgg taaatggccc gcctggctga ccgcccaacg acccccgccc attgacgtca 1860
ataatgacgt atgttcccat agtaacgcca atagggactt tccattgacg tcaatgggtg 1920
gagtatttac ggtaaactgc ccacttggca gtacatcaag tgtatcatat gccaagtacg 1980
ccccctattg acgtcaatga cggtaaatgg cccgcctggc attatgccca gtacatgacc 2040
ttatgggact ttcctacttg gcagtacatc tacgtattag tcatcgctat taccatggtg 2100
atgcggtttt ggcagtacat caatgggcgt ggatagcggt ttgactcacg gggatttcca 2160
agtctccacc ccattgacgt caatgggagt ttgttttggc accaaaatca acgggacttt 2220
ccaaaatgtc gtaacaactc cgccccattg acgcaaatgg gcggtaggcg tgtacggtgg 2280
gaggtctata taagcagagc tcgtttagtg aaccggggtc tctctggtta gaccagatct 2340
gagcctggga gctctctggc taactaggga acccactgct taagcctcaa taaagcttgc 2400
cttgagtgct tcaagtagtg tgtgcccgtc tgttgtgtga ctctggtaac tagagatccc 2460
tcagaccctt ttagtcagtg tggaaaatct ctagcagtgg cgcccgaaca gggacttgaa 2520
agcgaaaggg aaaccagagg agctctctcg acgcaggact cggcttgctg aagcgcgcac 2580
ggcaagaggc gaggggcggc gactggtgag tacgccaaaa attttgacta gcggaggcta 2640
gaaggagaga gatgggtgcg agagcgtcag tattaagcgg gggagaatta gatcgcgatg 2700
ggaaaaaatt cggttaaggc cagggggaaa gaaaaaatat aaattaaaac atatagtatg 2760
ggcaagcagg gagctagaac gattcgcagt taatcctggc ctgttagaaa catcagaagg 2820
ctgtagacaa atactgggac agctacaacc atcccttcag acaggatcag aagaacttag 2880
atcattatat aatacagtag caaccctcta ttgtgtgcat caaaggatag agataaaaga 2940
caccaaggaa gctttagaca agatagagga agagcaaaac aaaagtaaga ccaccgcaca 3000
gcaagcggcc gctgatcttc agacctggag gaggagatat gagggacaat tggagaagtg 3060
aattatataa atataaagta gtaaaaattg aaccattagg agtagcaccc accaaggcaa 3120
agagaagagt ggtgcagaga gaaaaaagag cagtgggaat aggagctttg ttccttgggt 3180
tcttgggagc agcaggaagc actatgggcg cagcgtcaat gacgctgacg gtacaggcca 3240
gacaattatt gtctggtata gtgcagcagc agaacaattt gctgagggct attgaggcgc 3300
aacagcatct gttgcaactc acagtctggg gcatcaagca gctccaggca agaatcctgg 3360
ctgtggaaag atacctaaag gatcaacagc tcctggggat ttggggttgc tctggaaaac 3420
tcatttgcac cactgctgtg ccttggaatg ctagttggag taataaatct ctggaacaga 3480
tttggaatca cacgacctgg atggagtggg acagagaaat taacaattac acaagcttaa 3540
tacactcctt aattgaagaa tcgcaaaacc agcaagaaaa gaatgaacaa gaattattgg 3600
aattagataa atgggcaagt ttgtggaatt ggtttaacat aacaaattgg ctgtggtata 3660
taaaattatt cataatgata gtaggaggct tggtaggttt aagaatagtt tttgctgtac 3720
tttctatagt gaatagagtt aggcagggat attcaccatt atcgtttcag acccacctcc 3780
caaccccgag gggacccgac aggcccgaag gaatagaaga agaaggtgga gagagagaca 3840
gagacagatc cattcgatta gtgaacggat ctcgacggta tcggttaact tttaaaagaa 3900
aaggggggat tggggggtac agtgcagggg aaagaatagt agacataata gcaacagaca 3960
tacaaactaa agaattacaa aaacaaatta caaaaattca aaattttatc gatcacgaga 4020
ctagcctcga gaagcttgat atccctaggt attgaataag aaaaatgaag ttaaggtggt 4080
tgatggtaac actatgctaa taactgcaga gccagaagca ccataaggga catgataagg 4140
gagccagcag acctctgatc tcttcctgaa tgctaatctt aaacatcctg aggaagaatg 4200
ggacttccat ttggggtggg cctatgacag ggtaataaga cagtagtgaa tatcaagcta 4260
caaaaagccg cctttcaaat tcttctcagt cctaactttt catactaagc ccagtccttc 4320
caaagcagac tgtgaaagag tgatagttcc gggagactag cactgcagat tccgggtcac 4380
tgtgagtggg ggaggcaggg aagaagggct cacaggacag tcaaaccatg acccctgttt 4440
ttccttcttc aagtagacct ctataagaca acagagacaa ctaaggctga gtggccaggc 4500
gaggagaaac catctcgccg taaaacatgg aaggaacact tcaggggaaa ggtggtatct 4560
ctaagcaaga gaactgagtg gagtcaaggc tgagagatgc aggataagca aatgggtagt 4620
gaaaagacat tcatgaggac agctaaaaca ataagtaatg taaaatacag catagcaaaa 4680
ctttaacctc caaatcaagc ctctacttga atccttttct gagggatgaa taaggcatat 4740
gcatcagggg ctgttgccaa tgtgcattag ctgtttgcag cctcaccttc tttcatggag 4800
tttaagatat agtgtatttt cccaaggttt gaactagctc ttcatttctt tatgttttaa 4860
atgcactgac ctcccacatt ccctttttag taaaatattc agaaataatt taaatacatc 4920
attgcaatga aaataaatgt tttttattag gcagaatcca gatgctcaag gcccttcata 4980
atatccccca gtttagtagt tggacttagg gaacaaagga acctttaata gaaattggac 5040
agcaagaaag cgagcttagt gatacttgtg ggccagggca ttagccacac cagccaccac 5100
tttctgatag gcagcctgca ctggtggggt gaattctttg ccaaagtgat gggccagcac 5160
acagaccagc acgttgccca ggagctgtgg gaggaagata agaggtatga acatgattag 5220
caaaagggcc tagcttggac tcagaataat ccagccttat cccaaccata aaataaaagc 5280
agaatggtag ctggattgta gctgctatta gcaatatgaa acctcttaca tcagttacaa 5340
tttatatgca gaaatattta tatgcagaga tattgctatt gccttaaccc agaaattatc 5400
actgttattc tttagaatgg tgcaaagagg catgatacat tgtatcatta ttgccctgaa 5460
agaaagagat tagggaaagt attagaaata agataaacaa aaaagtatat taaaagaaga 5520
aagcattttt taaaattaca aatgcaaaat taccctgatt tggtcaatat gtgtaccctg 5580
ttacttatcc ccttcctatg acatgaactt aaccatagaa aagaagggga aagaaaacat 5640
caagcgtccc atagactcac cctgaagttc tcaggatcca cgtgcagctt gtcacagtgc 5700
agctcactca gctgggcaaa ggtgcccttg aggttgtcca ggtgagccag gccatcacta 5760
aaggcaccga gcactttctt gccatgagcc ttcaccttag ggttgcccat aacagcatca 5820
ggagtggaca gatccccaaa ggactcaaag aacctctggg tccaagggta gaccaccagc 5880
agcctaaggg tgggaaaata gaccaatagg cagagagagt cagtgcctat cagaaaccca 5940
agagtcttct ctgtctccac atgcccagtt tctattggtc tccttaaacc tgtcttgtaa 6000
ccttgatacc aacctgccca gggcctcacc accaacttca tccacgttca ccttgcccca 6060
cagggcagta acggcagact tctcctcagg agtcagatgc accatggtgt ctgtttgagg 6120
ttgctagtga acacagttgt gtcagaagca aatgtaagca atagatggct ctgccctgac 6180
ttttatgccc agccctggct cctgccctcc ctgctcctgg gagtagattg gccaacccta 6240
gggtgtggct ccacagggtg aggtctaagt gatgacagcc gtacctgtcc ttggctcttc 6300
tggcactggc ttaggagttg gacttcaaac cctcagccct ccctctaaga tatatctctt 6360
ggccccatac catcagtaca aattgctact aaaaacatcc tcctttgcaa gtgtatttac 6420
gtatctagaa tatgtcacat tctgtctcag gcatccattt tctttatgat gccgtttgag 6480
gtggagtttt agtcaggtgg tcagcttctc cttttttttg ccatctgccc tgtaagcatc 6540
ctgctgggga cccagatagg agtcatcact ctaggctgag aacatctggg cacacaccct 6600
aagcctcagc atgactcatc atgactcagc attgctgtgc ttgagccaga aggtttgctt 6660
agaaggttac acagaaccag aaggcggggg tggggcactg accccgacag gggcctggcc 6720
agaactgctc atgcttggac tatgggaggt cactaatgga gacacacaga aatgtaacag 6780
gaactaagga aaaactgaag cttatttaat cagagatgag gatgctggaa gggatagagg 6840
gagctgagct tgtaaaaagt atagtaatca ttcagcaaat ggttttgaag cacctgctgg 6900
atgctaaaca ctattttcag tgcttgaatc ataaataaga ataaaacatg tatcttattc 6960
cccacaagag tccaagtaaa aaataacagt taattataat gtgctctgtc ccccaggctg 7020
gagtgcagtg gcacgatctc agctcactgc aacctccgcc tcccgggctg gttagaaggt 7080
tctactggag gagggtccca gcccattgct aaattaacat caggctctga gactggcagt 7140
atatctctaa cagtggttga tgctatcttc tggaacttgc ctgctacatt gagaccactg 7200
acccatacat aggaagccca tagctctgtc ctgaactgtt aggccactgg tccagagagt 7260
gtgcatctcc tttgatcctc ataataaccc tatgagatag acacaattat tactcttact 7320
ttatagatga tgatcctgaa aacataggag tcaaggcact tgcccctagc tgggggtata 7380
ggggagcagt cccatgtagt agtagaatga aaaatgctgc tatgctgtgc ctcccccacc 7440
tttcccatgt ctgccctcta ctcatggtct atctctcctg gctcctggga gtcatggact 7500
ccacccagca ccaccaacct gacctaacca cctatctgag cctgccagcc tataacccat 7560
ctgggccctg atagctggtg gccagccctg accccacccc accctccctg gaacctctga 7620
tagacacatc tggcacacca gctcgcaaag tcaccgtgag ggtcttgtgt ttgctgagtc 7680
aaaattcctt gaaatccaag tccttagaga ctcctgctcc caaatttaca gtcatagact 7740
tcttcatggc tgtctccttt atccacagaa tgattccttt gcttcattgc cccatccatc 7800
tgatcctcct catcagtgca gcacagggcc catgagcagt agctgcagag tctcacatag 7860
gtctggcact gcctctgaca tgtccgacct taggcaaatg cttgactctt ctctagtgca 7920
tgcaaatctg acactcagtg ggcctgggtg aaggtgagaa ttttattgct gaatgagagc 7980
ctctggggac atcttgccag tcaatgagtc tcaggttcaa tttccttctc agtcttggag 8040
taacagaagc tcatgcattt aataaacgga aattttgtat tgaaatgaga gccattggaa 8100
atcatttact ccagactcct acttataaaa agagaaactg aggctcagag aagggtgggg 8160
actttctcag tatgacatgg aaatgatcag gcttggattc aaagctcctg actttctgtc 8220
tagtgtatgt gcagtgagcc ccttttcctc taactgaaag aaggaaaaaa aaatggaacc 8280
caaaatattc tacatagttt ccatgtcaca gccagggctg ggcagtctcc tgttatttct 8340
tttaaaataa atatatcatt taaatgcata aataagcaaa ccctgctcgg gaatgggagg 8400
gagagtctct ggagtccacc ccttctcggc cctggctctg cagatagtgc tatcaaagcc 8460
ctgacagagc cctgcccatt gctgggcctt ggagtgagtc agcctagtag agaggcaggg 8520
caagccatct catagctgct gagtgggaga gagaaaaggg ctcattgtct ataaactcag 8580
gtcatggcta ttcttattct cacactaaga aaaagaatga gatgtctaca tataccctgc 8640
gtcccctctt gtgtactggg gtccccaaga gctctctaaa agtgatggca aagtcattgc 8700
gctagatgcc atcccatcta ttataaacct gcatttgtct ccacacacca gtcatggaca 8760
ataaccctcc tcccaggtcc acgtgcttgt ctttgtataa tactcaagta atttcggaaa 8820
atgtattctt tcaatcttgt tctgttattc ctgtttcaat ggcttagtag aaaaagtaca 8880
tacttgtttt cccataaatt gacaatagac aatttcacat caatgtctat atgggtcgtt 8940
gtgtttgctg tgtttgcaaa aactcacaat aactttatat tgttactact ctaagaaagt 9000
tacaacatgg tgaatacaag agaaagctat tacaagtcca gaaaataaaa gttatcatct 9060
tgagggtcga caatcaacct ctggattaca aaatttgtga aagattgact ggtattctta 9120
actatgttgc tccttttacg ctatgtggat acgctgcttt aatgcctttg tatcatgcta 9180
ttgcttcccg tatggctttc attttctcct ccttgtataa atcctggttg ctgtctcttt 9240
atgaggagtt gtggcccgtt gtcaggcaac gtggcgtggt gtgcactgtg tttgctgacg 9300
caacccccac tggttggggc attgccacca cctgtcagct cctttccggg actttcgctt 9360
tccccctccc tattgccacg gcggaactca tcgccgcctg ccttgcccgc tgctggacag 9420
gggctcggct gttgggcact gacaattccg tggtgttgtc ggggaagctg acgtcctttc 9480
catggctgct cgcctgtgtt gccacctgga ttctgcgcgg gacgtccttc tgctacgtcc 9540
cttcggccct caatccagcg gaccttcctt cccgcggcct gctgccggct ctgcggcctc 9600
ttccgcgtct tcgccttcgc cctcagacga gtcggatctc cctttgggcc gcctccccgc 9660
ctggaattcg agctcggtac ctttaagacc aatgacttac aaggcagctg tagatcttag 9720
ccacttttta aaagaaaagg ggggactgga agggctaatt cactcccaac gaagacaaga 9780
tctgcttttt gcttgtactg ggtctctctg gttagaccag atctgagcct gggagctctc 9840
tggctaacta gggaacccac tgcttaagcc tcaataaagc ttgccttgag tgcttcaagt 9900
agtgtgtgcc cgtctgttgt gtgactctgg taactagaga tccctcagac ccttttagtc 9960
agtgtggaaa atctctagca gtagtagttc atgtcatctt attattcagt atttataact 10020
tgcaaagaaa tgaatatcag agagtgagag gaacttgttt attgcagctt ataatggtta 10080
caaataaagc aatagcatca caaatttcac aaataaagca tttttttcac tgcattctag 10140
ttgtggtttg tccaaactca tcaatgtatc ttatcatgtc tggctctagc tatcccgccc 10200
ctaactccgc ccatcccgcc cctaactccg cccagttccg cccattctcc gccccatggc 10260
tgactaattt tttttattta tgcagaggcc gaggccgcct cggcctctga gctattccag 10320
aagtagtgag gaggcttttt tggaggccgc tagcgtcgac cattacttat tgttttagct 10380
gtcctcatga atgtcttttc actacccatt tgcttatcct gcatctctca gccttgactc 10440
cactcagttc tcttgcttag agataccacc tttcccctga agtgttcctt ccatgtttta 10500
cggcgagatg gtttctcctc gcctggccac tcagccttag ttgtctctgt tgtcttatag 10560
aggtctactt gaagaaggaa aaacaggggg catggtttga ctgtcctgtg agcccttctt 10620
ccctgcctcc cccactcaca gtgacccgga atccctcgac atggcagtct agcactagtg 10680
cggccgcaga tctgcttcct cgctcactga ctcgctgcgc tcggtcgttc ggctgcggcg 10740
agcggtatca gctcactcaa aggcggtaat acggttatcc acagaatcag gggataacgc 10800
aggaaagaac atgtgagcaa aaggccagca aaaggccagg aaccgtaaaa a 10851
SEQ ID NO: 9
ggccgcgttg ctggcgtttt tccataggct ccgcccccct gacgagcatc acaaaaatcg 60
acgctcaagt cagaggtggc gaaacccgac aggactataa agataccagg cgtttccccc 120
tggaagctcc ctcgtgcgct ctcctgttcc gaccctgccg cttaccggat acctgtccgc 180
ctttctccct tcgggaagcg tggcgctttc tcatagctca cgctgtaggt atctcagttc 240
ggtgtaggtc gttcgctcca agctgggctg tgtgcacgaa ccccccgttc agcccgaccg 300
ctgcgcctta tccggtaact atcgtcttga gtccaacccg gtaagacacg acttatcgcc 360
actggcagca gccactggta acaggattag cagagcgagg tatgtaggcg gtgctacaga 420
gttcttgaag tggtggccta actacggcta cactagaaga acagtatttg gtatctgcgc 480
tctgctgaag ccagttacct tcggaaaaag agttggtagc tcttgatccg gcaaacaaac 540
caccgctggt agcggtggtt tttttgtttg caagcagcag attacgcgca gaaaaaaagg 600
atctcaagaa gatcctttga tcttttctac ggggtctgac gctcagtgga acgaaaactc 660
acgttaaggg attttggtca tgaagcgctt ttgaagctcg gatccgaaca aacgacccaa 720
cacccgtgcg ttttattctg tctttttatt gccgatcccc tcagaagaac tcgtcaagaa 780
ggcgatagaa ggcgatgcgc tgcgaatcgg gagcggcgat accgtaaagc acgaggaagc 840
ggtcagccca ttcgccgcca agctcttcag caatatcacg ggtagccaac gctatgtcct 900
gatagcggtc cgccacaccc agccggccac agtcgatgaa tccagaaaag cggccatttt 960
ccaccatgat attcggcaag caggcatcgc catgggtcac gacgagatcc tcgccgtcgg 1020
gcatgctcgc cttgagcctg gcgaacagtt cggctggcgc gagcccctga tgctcttcgt 1080
ccagatcatc ctgatcgaca agaccggctt ccatccgagt acgtgctcgc tcgatgcgat 1140
gtttcgcttg gtggtcgaat gggcaggtag ccggatcaag cgtatgcagc cgccgcattg 1200
catcagccat gatggatact ttctcggcag gagcaaggtg agatgacagg agatcctgcc 1260
ccggcacttc gcccaatagc agccagtccc ttcccgcttc agtgacaacg tcgagcacag 1320
ctgcgcaagg aacgcccgtc gtggccagcc acgatagccg cgctgcctcg tcttgcagtt 1380
cattcagggc accggacagg tcggtcttga caaaaagaac cgggcgcccc tgcgctgaca 1440
gccggaacac ggcggcatca gagcagccga ttgtctgttg tgcccagtca tagccgaata 1500
gcctctccac ccaagcggcc ggagaacctg cgtgcaatcc atcttgttca atcatgcgaa 1560
acgatcctca tcctgtctct tgatcagagc ttgatcccct gcgccatcag atccttggcg 1620
gcaagaaagc catccagttt actttgcagg gcttcccaac cttaccagag gcctgcgccg 1680
cggccagctg gctagcaatt cccgggttaa ctctagagac attgattatt gactagttat 1740
taatagtaat caattacggg gtcattagtt catagcccat atatggagtt ccgcgttaca 1800
taacttacgg taaatggccc gcctggctga ccgcccaacg acccccgccc attgacgtca 1860
ataatgacgt atgttcccat agtaacgcca atagggactt tccattgacg tcaatgggtg 1920
gagtatttac ggtaaactgc ccacttggca gtacatcaag tgtatcatat gccaagtacg 1980
ccccctattg acgtcaatga cggtaaatgg cccgcctggc attatgccca gtacatgacc 2040
ttatgggact ttcctacttg gcagtacatc tacgtattag tcatcgctat taccatggtg 2100
atgcggtttt ggcagtacat caatgggcgt ggatagcggt ttgactcacg gggatttcca 2160
agtctccacc ccattgacgt caatgggagt ttgttttggc accaaaatca acgggacttt 2220
ccaaaatgtc gtaacaactc cgccccattg acgcaaatgg gcggtaggcg tgtacggtgg 2280
gaggtctata taagcagagc tcgtttagtg aaccgtcaga tcgcctggag acgccatcca 2340
cgctgttttg acctccatag aagacaccgg gaccgatcca gcctcccctc gaagcttaca 2400
tgtggtaccg agctcggatc ctgagaactt cagggtgagt ctatgggacc cttgatgttt 2460
tctttcccct tcttttctat ggttaagttc atgtcatagg aaggggagaa gtaacagggt 2520
acacatattg accaaatcag ggtaattttg catttgtaat tttaaaaaat gctttcttct 2580
tttaatatac ttttttgttt atcttatttc taatactttc cctaatctct ttctttcagg 2640
gcaataatga tacaatgtat catgcctctt tgcaccattc taaagaataa cagtgataat 2700
ttctgggtta aggcaatagc aatatttctg catataaata tttctgcata taaattgtaa 2760
ctgatgtaag aggtttcata ttgctaatag cagctacaat ccagctacca ttctgctttt 2820
attttatggt tgggataagg ctggattatt ctgagtccaa gctaggccct tttgctaatc 2880
atgttcatac ctcttatctt cctcccacag ctcctgggca acgtgctggt ctgtgtgctg 2940
gcccatcact ttggcaaagc acgtgagatc tgaattctga cactatgaag tgccttttgt 3000
acttagcctt tttattcatt ggggtgaatt gcaagttcac catagttttt ccacacaacc 3060
aaaaaggaaa ctggaaaaat gttccttcta attaccatta ttgcccgtca agctcagatt 3120
taaattggca taatgactta ataggcacag ccttacaagt caaaatgccc aagagtcaca 3180
aggctattca agcagacggt tggatgtgtc atgcttccaa atgggtcact acttgtgatt 3240
tccgctggta tggaccgaag tatataacac attccatccg atccttcact ccatctgtag 3300
aacaatgcaa ggaaagcatt gaacaaacga aacaaggaac ttggctgaat ccaggcttcc 3360
ctcctcaaag ttgtggatat gcaactgtga cggatgccga agcagtgatt gtccaggtga 3420
ctcctcacca tgtgctggtt gatgaataca caggagaatg ggttgattca cagttcatca 3480
acggaaaatg cagcaattac atatgcccca ctgtccataa ctctacaacc tggcattctg 3540
actataaggt caaagggcta tgtgattcta acctcatttc catggacatc accttcttct 3600
cagaggacgg agagctatca tccctgggaa aggagggcac agggttcaga agtaactact 3660
ttgcttatga aactggaggc aaggcctgca aaatgcaata ctgcaagcat tggggagtca 3720
gactcccatc aggtgtctgg ttcgagatgg ctgataagga tctctttgct gcagccagat 3780
tccctgaatg cccagaaggg tcaagtatct ctgctccatc tcagacctca gtggatgtaa 3840
gtctaattca ggacgttgag aggatcttgg attattccct ctgccaagaa acctggagca 3900
aaatcagagc gggtcttcca atctctccag tggatctcag ctatcttgct cctaaaaacc 3960
caggaaccgg tcctgctttc accataatca atggtaccct aaaatacttt gagaccagat 4020
acatcagagt cgatattgct gctccaatcc tctcaagaat ggtcggaatg atcagtggaa 4080
ctaccacaga aagggaactg tgggatgact gggcaccata tgaagacgtg gaaattggac 4140
ccaatggagt tctgaggacc agttcaggat ataagtttcc tttatacatg attggacatg 4200
gtatgttgga ctccgatctt catcttagct caaaggctca ggtgttcgaa catcctcaca 4260
ttcaagacgc tgcttcgcaa cttcctgatg atgagagttt attttttggt gatactgggc 4320
tatccaaaaa tccaatcgag cttgtagaag gttggttcag tagttggaaa agctctattg 4380
cctctttttt ctttatcata gggttaatca ttggactatt cttggttctc cgagttggta 4440
tccatctttg cattaaatta aagcacacca agaaaagaca gatttataca gacatagaga 4500
tgaaccgact tggaaagtaa ctcaaatcct gcacaacaga ttcttcatgt ttggaccaaa 4560
tcaacttgtg ataccatgct caaagaggcc tcaattatat ttgagttttt aatttttatg 4620
aaaaaaaaaa aaaaaaacgg aattcacccc accagtgcag gctgcctatc agaaagtggt 4680
ggctggtgtg gctaatgccc tggcccacaa gtatcactaa gctcgctttc ttgctgtcca 4740
atttctatta aaggttcctt tgttccctaa gtccaactac taaactgggg gatattatga 4800
agggccttga gcatctggat tctgcctaat aaaaaacatt tattttcatt gcaatgatgt 4860
atttaaatta tttctgaata ttttactaaa aagggaatgt gggaggtcag tgcatttaaa 4920
acataaagaa atgaagagct agttcaaacc ttgggaaaat acactatatc ttaaactcca 4980
tgaaagaagg tgaggctgca aacagctaat gcacattggc aacagcccct gatgcctatg 5040
ccttattcat ccctcagaaa aggattcaag tagaggcttg atttggaggt taaagttttg 5100
ctatgctgta ttttagtcga ccattactta ttgttttagc tgtcctcatg aatgtctttt 5160
cactacccat ttgcttatcc tgcatctctc agccttgact ccactcagtt ctcttgctta 5220
gagataccac ctttcccctg aagtgttcct tccatgtttt acggcgagat ggtttctcct 5280
cgcctggcca ctcagcctta gttgtctctg ttgtcttata gaggtctact tgaagaagga 5340
aaaacagggg gcatggtttg actgtcctgt gagcccttct tccctgcctc ccccactcac 5400
agtgacccgg aatccctcga catggcagtc tagcactagt gcggccgcag atctgcttcc 5460
tcgctcactg actcgctgcg ctcggtcgtt cggctgcggc gagcggtatc agctcactca 5520
aaggcggtaa tacggttatc cacagaatca ggggataacg caggaaagaa catgtgagca 5580
aaaggccagc aaaaggccag gaaccgtaaa aa 5612
SEQ ID NO: 10
ggccgcgttg ctggcgtttt tccataggct ccgcccccct gacgagcatc acaaaaatcg 60
acgctcaagt cagaggtggc gaaacccgac aggactataa agataccagg cgtttccccc 120
tggaagctcc ctcgtgcgct ctcctgttcc gaccctgccg cttaccggat acctgtccgc 180
ctttctccct tcgggaagcg tggcgctttc tcatagctca cgctgtaggt atctcagttc 240
ggtgtaggtc gttcgctcca agctgggctg tgtgcacgaa ccccccgttc agcccgaccg 300
ctgcgcctta tccggtaact atcgtcttga gtccaacccg gtaagacacg acttatcgcc 360
actggcagca gccactggta acaggattag cagagcgagg tatgtaggcg gtgctacaga 420
gttcttgaag tggtggccta actacggcta cactagaaga acagtatttg gtatctgcgc 480
tctgctgaag ccagttacct tcggaaaaag agttggtagc tcttgatccg gcaaacaaac 540
caccgctggt agcggtggtt tttttgtttg caagcagcag attacgcgca gaaaaaaagg 600
atctcaagaa gatcctttga tcttttctac ggggtctgac gctcagtgga acgaaaactc 660
acgttaaggg attttggtca tgaagcgctt ttgaagctcg gatccgaaca aacgacccaa 720
cacccgtgcg ttttattctg tctttttatt gccgatcccc tcagaagaac tcgtcaagaa 780
ggcgatagaa ggcgatgcgc tgcgaatcgg gagcggcgat accgtaaagc acgaggaagc 840
ggtcagccca ttcgccgcca agctcttcag caatatcacg ggtagccaac gctatgtcct 900
gatagcggtc cgccacaccc agccggccac agtcgatgaa tccagaaaag cggccatttt 960
ccaccatgat attcggcaag caggcatcgc catgggtcac gacgagatcc tcgccgtcgg 1020
gcatgctcgc cttgagcctg gcgaacagtt cggctggcgc gagcccctga tgctcttcgt 1080
ccagatcatc ctgatcgaca agaccggctt ccatccgagt acgtgctcgc tcgatgcgat 1140
gtttcgcttg gtggtcgaat gggcaggtag ccggatcaag cgtatgcagc cgccgcattg 1200
catcagccat gatggatact ttctcggcag gagcaaggtg agatgacagg agatcctgcc 1260
ccggcacttc gcccaatagc agccagtccc ttcccgcttc agtgacaacg tcgagcacag 1320
ctgcgcaagg aacgcccgtc gtggccagcc acgatagccg cgctgcctcg tcttgcagtt 1380
cattcagggc accggacagg tcggtcttga caaaaagaac cgggcgcccc tgcgctgaca 1440
gccggaacac ggcggcatca gagcagccga ttgtctgttg tgcccagtca tagccgaata 1500
gcctctccac ccaagcggcc ggagaacctg cgtgcaatcc atcttgttca atcatgcgaa 1560
acgatcctca tcctgtctct tgatcagagc ttgatcccct gcgccatcag atccttggcg 1620
gcaagaaagc catccagttt actttgcagg gcttcccaac cttaccagag gcctgcgccg 1680
cggccagctg gctagcaatt cccgggttaa ctctagagaa tgtagtctta tgcaatactc 1740
ttgtagtctt gcaacatggt aacgatgagt tagcaacatg ccttacaagg agagaaaaag 1800
caccgtgcat gccgattggt ggaagtaagg tggtacgatc gtgccttatt aggaaggcaa 1860
cagacgggtc tgacatggat tggacgaacc actgaattcc gcattgcaga gatattgtat 1920
ttaagtgcct agctcgatac aataaacgcc atttgaccat tcaccacatt ggtgtgcacc 1980
tccaagctcg agctcgttta gtgaaccgtc agatcgcctg gagacgccat ccacgctgtt 2040
ttgacctcca tagaagacac cgggaccgat ccagcctccc ctcgaagcta gtcgattagg 2100
catctcctat ggcaggaaga agcggagaca gcgacgaaga cctcctcaag gcagtcagac 2160
tcatcaagtt tctctatcaa agcaacccac ctcccaatcc cgaggggacc cgacaggccc 2220
gaaggaatag aagaagaagg tggagagaga gacagagaca gatccattcg attagtgaac 2280
ggatccttag cacttatctg ggacgatctg cggagcctgt gcctcttcag ctaccaccgc 2340
ttgagagact tactcttgat tgtaacgagg attgtggaac ttctgggacg cagggggtgg 2400
gaagccctca aatattggtg gaatctccta caatattgga gtcaggagct aaagaatagt 2460
gctgttagct tgctcaatgc cacagctata gcagtagctg aggggacaga tagggttata 2520
gaagtagtac aagaagcttg gcactggccg tcgttttaca acgtcgtgat ctgagcctgg 2580
gagatctctg gctaactagg gaacccactg cttaagcctc aataaagctt gccttgagtg 2640
cttcaagtag tgtgtgcccg tctgttgtgt gactctggta actagagatc aggaaaaccc 2700
tggcgttacc caacttaatc gccttgcagc acatccccct ttcgccagct ggcgtaatag 2760
cgaagaggcc cgcaccgatc gcccttccca acagttgcgc agcctgaatg gcgaatggcg 2820
cctgatgcgg tattttctcc ttacgcatct gtgcggtatt tcacaccgca tacgtcaaag 2880
caaccatagt gtcgaccatt acttattgtt ttagctgtcc tcatgaatgt cttttcacta 2940
cccatttgct tatcctgcat ctctcagcct tgactccact cagttctctt gcttagagat 3000
accacctttc ccctgaagtg ttccttccat gttttacggc gagatggttt ctcctcgcct 3060
ggccactcag ccttagttgt ctctgttgtc ttatagaggt ctacttgaag aaggaaaaac 3120
agggggcatg gtttgactgt cctgtgagcc cttcttccct gcctccccca ctcacagtga 3180
cccggaatcc ctcgacatgg cagtctagca ctagtgcggc cgcagatctg cttcctcgct 3240
cactgactcg ctgcgctcgg tcgttcggct gcggcgagcg gtatcagctc actcaaaggc 3300
ggtaatacgg ttatccacag aatcagggga taacgcagga aagaacatgt gagcaaaagg 3360
ccagcaaaag gccaggaacc gtaaaaa 3387
SEQ ID NO: 11
ggccgcgttg ctggcgtttt tccataggct ccgcccccct gacgagcatc acaaaaatcg 60
acgctcaagt cagaggtggc gaaacccgac aggactataa agataccagg cgtttccccc 120
tggaagctcc ctcgtgcgct ctcctgttcc gaccctgccg cttaccggat acctgtccgc 180
ctttctccct tcgggaagcg tggcgctttc tcatagctca cgctgtaggt atctcagttc 240
ggtgtaggtc gttcgctcca agctgggctg tgtgcacgaa ccccccgttc agcccgaccg 300
ctgcgcctta tccggtaact atcgtcttga gtccaacccg gtaagacacg acttatcgcc 360
actggcagca gccactggta acaggattag cagagcgagg tatgtaggcg gtgctacaga 420
gttcttgaag tggtggccta actacggcta cactagaaga acagtatttg gtatctgcgc 480
tctgctgaag ccagttacct tcggaaaaag agttggtagc tcttgatccg gcaaacaaac 540
caccgctggt agcggtggtt tttttgtttg caagcagcag attacgcgca gaaaaaaagg 600
atctcaagaa gatcctttga tcttttctac ggggtctgac gctcagtgga acgaaaactc 660
acgttaaggg attttggtca tgaagcgctt ttgaagctcg gatccgaaca aacgacccaa 720
cacccgtgcg ttttattctg tctttttatt gccgatcccc tcagaagaac tcgtcaagaa 780
ggcgatagaa ggcgatgcgc tgcgaatcgg gagcggcgat accgtaaagc acgaggaagc 840
ggtcagccca ttcgccgcca agctcttcag caatatcacg ggtagccaac gctatgtcct 900
gatagcggtc cgccacaccc agccggccac agtcgatgaa tccagaaaag cggccatttt 960
ccaccatgat attcggcaag caggcatcgc catgggtcac gacgagatcc tcgccgtcgg 1020
gcatgctcgc cttgagcctg gcgaacagtt cggctggcgc gagcccctga tgctcttcgt 1080
ccagatcatc ctgatcgaca agaccggctt ccatccgagt acgtgctcgc tcgatgcgat 1140
gtttcgcttg gtggtcgaat gggcaggtag ccggatcaag cgtatgcagc cgccgcattg 1200
catcagccat gatggatact ttctcggcag gagcaaggtg agatgacagg agatcctgcc 1260
ccggcacttc gcccaatagc agccagtccc ttcccgcttc agtgacaacg tcgagcacag 1320
ctgcgcaagg aacgcccgtc gtggccagcc acgatagccg cgctgcctcg tcttgcagtt 1380
cattcagggc accggacagg tcggtcttga caaaaagaac cgggcgcccc tgcgctgaca 1440
gccggaacac ggcggcatca gagcagccga ttgtctgttg tgcccagtca tagccgaata 1500
gcctctccac ccaagcggcc ggagaacctg cgtgcaatcc atcttgttca atcatgcgaa 1560
acgatcctca tcctgtctct tgatcagagc ttgatcccct gcgccatcag atccttggcg 1620
gcaagaaagc catccagttt actttgcagg gcttcccaac cttaccagag gcctgcgccg 1680
cggccagctg gctagcaatt cccgggttaa ctctagagac attgattatt gactagttat 1740
taatagtaat caattacggg gtcattagtt catagcccat atatggagtt ccgcgttaca 1800
taacttacgg taaatggccc gcctggctga ccgcccaacg acccccgccc attgacgtca 1860
ataatgacgt atgttcccat agtaacgcca atagggactt tccattgacg tcaatgggtg 1920
gagtatttac ggtaaactgc ccacttggca gtacatcaag tgtatcatat gccaagtacg 1980
ccccctattg acgtcaatga cggtaaatgg cccgcctggc attatgccca gtacatgacc 2040
ttatgggact ttcctacttg gcagtacatc tacgtattag tcatcgctat taccatggtg 2100
atgcggtttt ggcagtacat caatgggcgt ggatagcggt ttgactcacg gggatttcca 2160
agtctccacc ccattgacgt caatgggagt ttgttttggc accaaaatca acgggacttt 2220
ccaaaatgtc gtaacaactc cgccccattg acgcaaatgg gcggtaggcg tgtacggtgg 2280
gaggtctata taagcagagc tcgtttagtg aaccgtcaga tcgcctggag acgccatcca 2340
cgctgttttg acctccatag aagacaccgg gaccgatcca gcctcccctc gaagcttaca 2400
tgtggtaccg agctcggatc ctgagaactt cagggtgagt ctatgggacc cttgatgttt 2460
tctttcccct tcttttctat ggttaagttc atgtcatagg aaggggagaa gtaacagggt 2520
acacatattg accaaatcag ggtaattttg catttgtaat tttaaaaaat gctttcttct 2580
tttaatatac ttttttgttt atcttatttc taatactttc cctaatctct ttctttcagg 2640
gcaataatga tacaatgtat catgcctctt tgcaccattc taaagaataa cagtgataat 2700
ttctgggtta aggcaatagc aatatttctg catataaata tttctgcata taaattgtaa 2760
ctgatgtaag aggtttcata ttgctaatag cagctacaat ccagctacca ttctgctttt 2820
attttatggt tgggataagg ctggattatt ctgagtccaa gctaggccct tttgctaatc 2880
atgttcatac ctcttatctt cctcccacag ctcctgggca acgtgctggt ctgtgtgctg 2940
gcccatcact ttggcaaagc acgtgagatc tgaattcgag atctgccgcc gccatgggtg 3000
cgagagcgtc agtattaagc gggggagaat tagatcgatg ggaaaaaatt cggttaaggc 3060
cagggggaaa gaaaaaatat aaattaaaac atatagtatg ggcaagcagg gagctagaac 3120
gattcgcagt taatcctggc ctgttagaaa catcagaagg ctgtagacaa atactgggac 3180
agctacaacc atcccttcag acaggatcag aagaacttag atcattatat aatacagtag 3240
caaccctcta ttgtgtgcat caaaggatag agataaaaga caccaaggaa gctttagaca 3300
agatagagga agagcaaaac aaaagtaaga aaaaagcaca gcaagcagca gctgacacag 3360
gacacagcaa tcaggtcagc caaaattacc ctatagtgca gaacatccag gggcaaatgg 3420
tacatcaggc catatcacct agaactttaa atgcatgggt aaaagtagta gaagagaagg 3480
ctttcagccc agaagtgata cccatgtttt cagcattatc agaaggagcc accccacaag 3540
atttaaacac catgctaaac acagtggggg gacatcaagc agccatgcaa atgttaaaag 3600
agaccatcaa tgaggaagct gcagaatggg atagagtgca tccagtgcat gcagggccta 3660
ttgcaccagg ccagatgaga gaaccaaggg gatcagacat cgctggaact actagtaccc 3720
ttcaggaaca aataggatgg atgacacata atccacctat cccagtagga gaaatctata 3780
aaagatggat aatcctggga ttaaataaaa tagtaagaat gtatagccct accagcattc 3840
tggacataag acaaggacca aaggaaccct ttagagacta tgtagaccga ttctataaaa 3900
ctctaagagc cgagcaagct tcacaagagg taaaaaattg gatgacagaa accttgttgg 3960
tccaaaatgc gaacccagat tgtaagacta ttttaaaagc attgggacca ggagcgacac 4020
tagaagaaat gatgacagca tgtcagggag tggggggacc cggccataaa gcaagagttt 4080
tggctgaagc aatgagccaa gtaacaaatc cagctaccat aatgatacag aaaggcaatt 4140
ttaggaacca aagaaagact gttaagtgtt tcaattgtgg caaagaaggg cacatagcca 4200
aaaattgcag ggcccctagg aaaaagggct gttggaaatg tggaaaggaa ggacaccaaa 4260
tgaaagattg tactgagaga caggctaatt ttttagggaa gatctggcct tcccacaagg 4320
gaaggccagg gaattttctt cagagcagac cagagccaac agccccacca gaagagagct 4380
tcaggtttgg ggaagagaca acaactccct ctcagaagca ggagccgata gacaaggaac 4440
tgtatccttt agcttccctc agatcactct ttggcagcga cccctcgtca caataaagat 4500
aggggggcaa ttaaaggaag ctctattaga tactggtgct gacgacacag tattagaaga 4560
aatgaatttg ccaggaagat ggaaaccaaa aatgataggg ggaattggag gttttatcaa 4620
agtaagacag tatgatcaga tactcataga aatctgcgga cataaagcta taggtacagt 4680
attagtagga cctacacctg tcaacataat tggaagaaat ctgttgactc agattggctg 4740
cactttaaat tttcccatta gtcctattga gactgtacca gtaaaattaa agccaggaat 4800
ggatggccca aaagttaaac aatggccatt gacagaagaa aaaataaaag cattagtaga 4860
aatttgtaca gaaatggaaa aggaaggaaa aatttcaaaa attgggcctg aaaatccata 4920
caatactcca gtatttgcca taaagaaaaa agacagtact aaatggagaa aattagtaga 4980
tttcagagaa cttaataaga gaactcaaga tttctgggaa gttcaattag gaataccaca 5040
tcctgcaggg ttaaaacaga aaaaatcagt aacagtactg gatgtgggcg atgcatattt 5100
ttcagttccc ttagataaag acttcaggaa gtatactgca tttaccatac ctagtataaa 5160
caatgagaca ccagggatta gatatcagta caatgtgctt ccacagggat ggaaaggatc 5220
accagcaata ttccagtgta gcatgacaaa aatcttagag ccttttagaa aacaaaatcc 5280
agacatagtc atctatcaat acatggatga tttgtatgta ggatctgact tagaaatagg 5340
gcagcataga acaaaaatag aggaactgag acaacatctg ttgaggtggg gatttaccac 5400
accagacaaa aaacatcaga aagaacctcc attcctttgg atgggttatg aactccatcc 5460
tgataaatgg acagtacagc ctatagtgct gccagaaaag gacagctgga ctgtcaatga 5520
catacagaaa ttagtgggaa aattgaattg ggcaagtcag atttatgcag ggattaaagt 5580
aaggcaatta tgtaaacttc ttaggggaac caaagcacta acagaagtag taccactaac 5640
agaagaagca gagctagaac tggcagaaaa cagggagatt ctaaaagaac cggtacatgg 5700
agtgtattat gacccatcaa aagacttaat agcagaaata cagaagcagg ggcaaggcca 5760
atggacatat caaatttatc aagagccatt taaaaatctg aaaacaggaa agtatgcaag 5820
aatgaagggt gcccacacta atgatgtgaa acaattaaca gaggcagtac aaaaaatagc 5880
cacagaaagc atagtaatat ggggaaagac tcctaaattt aaattaccca tacaaaagga 5940
aacatgggaa gcatggtgga cagagtattg gcaagccacc tggattcctg agtgggagtt 6000
tgtcaatacc cctcccttag tgaagttatg gtaccagtta gagaaagaac ccataatagg 6060
agcagaaact ttctatgtag atggggcagc caatagggaa actaaattag gaaaagcagg 6120
atatgtaact gacagaggaa gacaaaaagt tgtcccccta acggacacaa caaatcagaa 6180
gactgagtta caagcaattc atctagcttt gcaggattcg ggattagaag taaacatagt 6240
gacagactca caatatgcat tgggaatcat tcaagcacaa ccagataaga gtgaatcaga 6300
gttagtcagt caaataatag agcagttaat aaaaaaggaa aaagtctacc tggcatgggt 6360
accagcacac aaaggaattg gaggaaatga acaagtagat aaattggtca gtgctggaat 6420
caggaaagta ctatttttag atggaataga taaggcccaa gaagaacatg agaaatatca 6480
cagtaattgg agagcaatgg ctagtgattt taacctacca cctgtagtag caaaagaaat 6540
agtagccagc tgtgataaat gtcagctaaa aggggaagcc atgcatggac aagtagactg 6600
tagcccagga atatggcagc tagattgtac acatttagaa ggaaaagtta tcttggtagc 6660
agttcatgta gccagtggat atatagaagc agaagtaatt ccagcagaga cagggcaaga 6720
aacagcatac ttcctcttaa aattagcagg aagatggcca gtaaaaacag tacatacaga 6780
caatggcagc aatttcacca gtactacagt taaggccgcc tgttggtggg cggggatcaa 6840
gcaggaattt ggcattccct acaatccgca gtcacaagga gtaatagaat ctatgaataa 6900
agaattaaag aaaattatag gacaggtaag agatcaggct gaacatctta aaacagcagt 6960
acaaatggca gtattcatcc acaattttaa aagaaaaggg gggattgggg ggtacagtgc 7020
aggggaaaga atagtagaca taatagcaac agacatacaa actaaagaat tacaaaaaca 7080
aattacaaaa attcaaaatt ttcgggttta ttacagggac agcagagatc cagtttggaa 7140
aggaccagca aagctcctct ggaaaggtga aggggcagta gtaatacaag ataatagtga 7200
cataaaagta gtgccaagaa gaaaagcaaa gatcatcagg gattatggaa aacagatggc 7260
aggtgatgat tgtgtggcaa gtagacagga tgaggattaa cacatggaat tccggagcgg 7320
ccgcaggagc tttgttcctt gggttcttgg gagcagcagg aagcactatg ggcgcagcgt 7380
caatgacgct gacggtacag gccagacaat tattgtctgg tatagtgcag cagcagaaca 7440
atttgctgag ggctattgag gcgcaacagc atctgttgca actcacagtc tggggcatca 7500
agcagctcca ggcaagaatc ctggctgtgg aaagatacct aaaggatcaa cagctcctgg 7560
ggatttgggg ttgctctgga aaactcattt gcaccactgc tgtgccttgg aatgctagtt 7620
ggagtaataa atctctggaa cagatttgga atcacacgac ctggatggag tgggacagag 7680
aaattaacaa ttacacaagc ttccgcggaa ttcaccccac cagtgcaggc tgcctatcag 7740
aaagtggtgg ctggtgtggc taatgccctg gcccacaagt atcactaagc tcgctttctt 7800
gctgtccaat ttctattaaa ggttcctttg ttccctaagt ccaactacta aactggggga 7860
tattatgaag ggccttgagc atctggattc tgcctaataa aaaacattta ttttcattgc 7920
aatgatgtat ttaaattatt tctgaatatt ttactaaaaa gggaatgtgg gaggtcagtg 7980
catttaaaac ataaagaaat gaagagctag ttcaaacctt gggaaaatac actatatctt 8040
aaactccatg aaagaaggtg aggctgcaaa cagctaatgc acattggcaa cagcccctga 8100
tgcctatgcc ttattcatcc ctcagaaaag gattcaagta gaggcttgat ttggaggtta 8160
aagttttgct atgctgtatt ttacattact tattgtttta gctgtcctca tgaatgtctt 8220
ttcactaccc atttgcttat cctgcatctc tcagccttga ctccactcag ttctcttgct 8280
tagagatacc acctttcccc tgaagtgttc cttccatgtt ttacggcgag atggtttctc 8340
ctcgcctggc cactcagcct tagttgtctc tgttgtctta tagaggtcta cttgaagaag 8400
gaaaaacagg gggcatggtt tgactgtcct gtgagccctt cttccctgcc tcccccactc 8460
acagtgaccc ggaatccctc gacatggcag tctagcacta gtgcggccgc agatctgctt 8520
cctcgctcac tgactcgctg cgctcggtcg ttcggctgcg gcgagcggta tcagctcact 8580
caaaggcggt aatacggtta tccacagaat caggggataa cgcaggaaag aacatgtgag 8640
caaaaggcca gcaaaaggcc aggaaccgta aaaagtcgac cattacttat tgttttagct 8700
gtcctcatga atgtcttttc actacccatt tgcttatcct gcatctctca gccttgactc 8760
cactcagttc tcttgcttag agataccacc tttcccctga agtgttcctt ccatgtttta 8820
cggcgagatg gtttctcctc gcctggccac tcagccttag ttgtctctgt tgtcttatag 8880
aggtctactt gaagaaggaa aaacaggggg catggtttga ctgtcctgtg agcccttctt 8940
ccctgcctcc cccactcaca gtgacccgga atccctcgac atggcagtct agcactagtg 9000
cggccgcaga tctgcttcct cgctcactga ctcgctgcgc tcggtcgttc ggctgcggcg 9060
agcggtatca gctcactcaa aggcggtaat acggttatcc acagaatcag gggataacgc 9120
aggaaagaac atgtgagcaa aaggccagca aaaggccagg aaccgtaaaa a 9171
SEQ ID NO: 12
tacgtaaata cacttgcaaa ggaggatgtt tttagtagca atttgtactg atggtatggg 60
gccaagagat atatcttaga gggagggctg agggtttgaa gtccaactcc taagccagtg 120
ccagaagagc caaggacagg tacggctgtc atcacttaga cctcaccctg tggagccaca 180
ccctagggtt ggccaatcta ctcccaggag cagggagggc aggagccagg gctgggcata 240
aaagtcaggg cagagccatc tattgcttac atttgcttct gacacaactg tgttcactag 300
caacctcaaa cagacaccat ggtgcatctg actcctgagg agaagtctgc cgttactgcc 360
ctgtggggca aggtgaacgt ggatgaagtt ggtggtgagg ccctgggcag gttggtatca 420
aggttacaag acaggtttaa ggagaccaat agaaactggg catgtggaga cagagaagac 480
tcttgggttt ctgataggca ctgactctct ctgcctattg gtctattttc ccacccttag 540
gctgctggtg gtctaccctt ggacccagag gttctttgag tcctttgggg atctgtccac 600
tcctgatgct gttatgggca accctaaggt gaaggctcat ggcaagaaag tgctcggtgc 660
ctttagtgat ggcctggctc acctggacaa cctcaagggc acctttgcca cactgagtga 720
gctgcactgt gacaagctgc acgtggatcc tgagaacttc agggtgagtc tatgggacgc 780
ttgatgtttt ctttcccctt cttttctatg gttaagttca tgtcatagga aggggataag 840
taacagggta cagtttagaa tgggaaacag acgaatgatt gcatcagtgt ggaagtctca 900
ggatcgtttt agtttctttt atttgctgtt cataacaatt gttttctttt gtttaattct 960
tgctttcttt ttttttcttc tccgcaattt ttactattat acttaatgcc ttaacattgt 1020
gtataacaaa aggaaatatc tctgagatac attaagtaac ttaaaaaaaa actttacaca 1080
gtctgcctag tacattacta tttggaatat atgtgtgctt atttgcatat tcataatctc 1140
cctactttat tttcttttat ttttaattga tacataatca ttatacatat ttatgggtta 1200
aagtgtaatg ttttaatatg tgtacacata ttgaccaaat cagggtaatt ttgcatttgt 1260
aattttaaaa aatgctttct tcttttaata tacttttttg tttatcttat ttctaatact 1320
ttccctaatc tctttctttc agggcaataa tgatacaatg tatcatgcct ctttgcacca 1380
ttctaaagaa taacagtgat aatttctggg ttaaggcaat agcaatatct ctgcatataa 1440
atatttctgc atataaattg taactgatgt aagaggtttc atattgctaa tagcagctac 1500
aatccagcta ccattctgct tttattttat ggttgggata aggctggatt attctgagtc 1560
caagctaggc ccttttgcta atcatgttca tacctcttat cttcctccca cagctcctgg 1620
gcaacgtgct ggtctgtgtg ctggcccatc actttggcaa agaattcacc ccaccagtgc 1680
aggctgccta tcagaaagtg gtggctggtg tggctaatgc cctggcccac aagtatcact 1740
aagctcgctt tcttgctgtc caatttctat taaaggttcc tttgttccct aagtccaact 1800
actaaactgg gggatattat gaagggcctt gagcatctgg attctgccta ataaaaaaca 1860
tttattttca ttgcaatgat gtatttaaat tatttctgaa tattttacta aaaagggaat 1920
gtgggaggtc agtgcattta aaacataaag aaatgaagag ctagttcaaa ccttgggaaa 1980
atacactata tcttaaactc catgaaagaa ggtgaggctg caaacagcta atgcacattg 2040
gcaacagccc ctgatgcata tgccttattc atccctcaga aaaggattca agtagaggct 2100
tgatttggag gttaaagttt tgctatgctg tattttacat tacttattgt tttagctgtc 2160
ctcatgaatg tcttttcact acccatttgc ttatcctgca tctctcagcc ttgactccac 2220
tcagttctct tgcttagaga taccaccttt cccctgaagt gttccttcca tgttttacgg 2280
cgagatggtt tctcctcgcc tggccactca gccttagttg tctctgttgt cttatagagg 2340
tctacttgaa gaaggaaaaa caggggtcat ggtttgactg tcctgtgagc ccttcttccc 2400
tgcctccccc actcacagtg acccggaatc tgcagtgcta gtctcccgga actatcactc 2460
tttcacagtc tgctttggaa ggactgggct tagtatgaaa agttaggact gagaagaatt 2520
tgaaaggcgg ctttttgtag cttgatattc actactgtct tattaccctg tcataggccc 2580
accccaaatg gaagtcccat tcttcctcag gatgtttaag attagcattc aggaagagat 2640
cagaggtctg ctggctccct tatcatgtcc cttatggtgc ttctggctct gcagttatta 2700
gcatagtgtt accatcaacc accttaactt catttttctt attcaatacc tagg 2754
SEQ ID NO: 13
ggccgcgttg ctggcgtttt tccataggct ccgcccccct gacgagcatc acaaaaatcg 60
acgctcaagt cagaggtggc gaaacccgac aggactataa agataccagg cgtttccccc 120
tggaagctcc ctcgtgcgct ctcctgttcc gaccctgccg cttaccggat acctgtccgc 180
ctttctccct tcgggaagcg tggcgctttc tcatagctca cgctgtaggt atctcagttc 240
ggtgtaggtc gttcgctcca agctgggctg tgtgcacgaa ccccccgttc agcccgaccg 300
ctgcgcctta tccggtaact atcgtcttga gtccaacccg gtaagacacg acttatcgcc 360
actggcagca gccactggta acaggattag cagagcgagg tatgtaggcg gtgctacaga 420
gttcttgaag tggtggccta actacggcta cactagaaga acagtatttg gtatctgcgc 480
tctgctgaag ccagttacct tcggaaaaag agttggtagc tcttgatccg gcaaacaaac 540
caccgctggt agcggtggtt tttttgtttg caagcagcag attacgcgca gaaaaaaagg 600
atctcaagaa gatcctttga tcttttctac ggggtctgac gctcagtgga acgaaaactc 660
acgttaaggg attttggtca tgaagcgctt ttgaagctcg gatccgaaca aacgacccaa 720
cacccgtgcg ttttattctg tctttttatt gccgatcccc tcagaagaac tcgtcaagaa 780
ggcgatagaa ggcgatgcgc tgcgaatcgg gagcggcgat accgtaaagc acgaggaagc 840
ggtcagccca ttcgccgcca agctcttcag caatatcacg ggtagccaac gctatgtcct 900
gatagcggtc cgccacaccc agccggccac agtcgatgaa tccagaaaag cggccatttt 960
ccaccatgat attcggcaag caggcatcgc catgggtcac gacgagatcc tcgccgtcgg 1020
gcatgctcgc cttgagcctg gcgaacagtt cggctggcgc gagcccctga tgctcttcgt 1080
ccagatcatc ctgatcgaca agaccggctt ccatccgagt acgtgctcgc tcgatgcgat 1140
gtttcgcttg gtggtcgaat gggcaggtag ccggatcaag cgtatgcagc cgccgcattg 1200
catcagccat gatggatact ttctcggcag gagcaaggtg agatgacagg agatcctgcc 1260
ccggcacttc gcccaatagc agccagtccc ttcccgcttc agtgacaacg tcgagcacag 1320
ctgcgcaagg aacgcccgtc gtggccagcc acgatagccg cgctgcctcg tcttgcagtt 1380
cattcagggc accggacagg tcggtcttga caaaaagaac cgggcgcccc tgcgctgaca 1440
gccggaacac ggcggcatca gagcagccga ttgtctgttg tgcccagtca tagccgaata 1500
gcctctccac ccaagcggcc ggagaacctg cgtgcaatcc atcttgttca atcatgcgaa 1560
acgatcctca tcctgtctct tgatcagagc ttgatcccct gcgccatcag atccttggcg 1620
gcaagaaagc catccagttt actttgcagg gcttcccaac cttaccagag gcctgcgccg 1680
cggccagctg gctagcaatt cccgggttaa ctctagagac attgattatt gactagttat 1740
taatagtaat caattacggg gtcattagtt catagcccat atatggagtt ccgcgttaca 1800
taacttacgg taaatggccc gcctggctga ccgcccaacg acccccgccc attgacgtca 1860
ataatgacgt atgttcccat agtaacgcca atagggactt tccattgacg tcaatgggtg 1920
gagtatttac ggtaaactgc ccacttggca gtacatcaag tgtatcatat gccaagtacg 1980
ccccctattg acgtcaatga cggtaaatgg cccgcctggc attatgccca gtacatgacc 2040
ttatgggact ttcctacttg gcagtacatc tacgtattag tcatcgctat taccatggtg 2100
atgcggtttt ggcagtacat caatgggcgt ggatagcggt ttgactcacg gggatttcca 2160
agtctccacc ccattgacgt caatgggagt ttgttttggc accaaaatca acgggacttt 2220
ccaaaatgtc gtaacaactc cgccccattg acgcaaatgg gcggtaggcg tgtacggtgg 2280
gaggtctata taagcagagc tcgtttagtg aaccggggtc tctctggtta gaccagatct 2340
gagcctggga gctctctggc taactaggga acccactgct taagcctcaa taaagcttgc 2400
cttgagtgct tcaagtagtg tgtgcccgtc tgttgtgtga ctctggtaac tagagatccc 2460
tcagaccctt ttagtcagtg tggaaaatct ctagcagtgg cgcccgaaca gggacttgaa 2520
agcgaaaggg aaaccagagg agctctctcg acgcaggact cggcttgctg aagcgcgcac 2580
ggcaagaggc gaggggcggc gactggtgag tacgccaaaa attttgacta gcggaggcta 2640
gaaggagaga gatgggtgcg agagcgtcag tattaagcgg gggagaatta gatcgcgatg 2700
ggaaaaaatt cggttaaggc cagggggaaa gaaaaaatat aaattaaaac atatagtatg 2760
ggcaagcagg gagctagaac gattcgcagt taatcctggc ctgttagaaa catcagaagg 2820
ctgtagacaa atactgggac agctacaacc atcccttcag acaggatcag aagaacttag 2880
atcattatat aatacagtag caaccctcta ttgtgtgcat caaaggatag agataaaaga 2940
caccaaggaa gctttagaca agatagagga agagcaaaac aaaagtaaga ccaccgcaca 3000
gcaagcggcc gctgatcttc agacctggag gaggagatat gagggacaat tggagaagtg 3060
aattatataa atataaagta gtaaaaattg aaccattagg agtagcaccc accaaggcaa 3120
agagaagagt ggtgcagaga gaaaaaagag cagtgggaat aggagctttg ttccttgggt 3180
tcttgggagc agcaggaagc actatgggcg cagcgtcaat gacgctgacg gtacaggcca 3240
gacaattatt gtctggtata gtgcagcagc agaacaattt gctgagggct attgaggcgc 3300
aacagcatct gttgcaactc acagtctggg gcatcaagca gctccaggca agaatcctgg 3360
ctgtggaaag atacctaaag gatcaacagc tcctggggat ttggggttgc tctggaaaac 3420
tcatttgcac cactgctgtg ccttggaatg ctagttggag taataaatct ctggaacaga 3480
tttggaatca cacgacctgg atggagtggg acagagaaat taacaattac acaagcttaa 3540
tacactcctt aattgaagaa tcgcaaaacc agcaagaaaa gaatgaacaa gaattattgg 3600
aattagataa atgggcaagt ttgtggaatt ggtttaacat aacaaattgg ctgtggtata 3660
taaaattatt cataatgata gtaggaggct tggtaggttt aagaatagtt tttgctgtac 3720
tttctatagt gaatagagtt aggcagggat attcaccatt atcgtttcag acccacctcc 3780
caaccccgag gggacccgac aggcccgaag gaatagaaga agaaggtgga gagagagaca 3840
gagacagatc cattcgatta gtgaacggat ctcgacggta tcggttaact tttaaaagaa 3900
aaggggggat tggggggtac agtgcagggg aaagaatagt agacataata gcaacagaca 3960
tacaaactaa agaattacaa aaacaaatta caaaaattca aaattttatc gatcacgaga 4020
ctagcctcga gaagcttgat atccctaggt attgaataag aaaaatgaag ttaaggtggt 4080
tgatggtaac actatgctaa taactgcaga gccagaagca ccataaggga catgataagg 4140
gagccagcag acctctgatc tcttcctgaa tgctaatctt aaacatcctg aggaagaatg 4200
ggacttccat ttggggtggg cctatgacag ggtaataaga cagtagtgaa tatcaagcta 4260
caaaaagccg cctttcaaat tcttctcagt cctaactttt catactaagc ccagtccttc 4320
caaagcagac tgtgaaagag tgatagttcc gggagactag cactgcagat tccgggtcac 4380
tgtgagtggg ggaggcaggg aagaagggct cacaggacag tcaaaccatg acccctgttt 4440
ttccttcttc aagtagacct ctataagaca acagagacaa ctaaggctga gtggccaggc 4500
gaggagaaac catctcgccg taaaacatgg aaggaacact tcaggggaaa ggtggtatct 4560
ctaagcaaga gaactgagtg gagtcaaggc tgagagatgc aggataagca aatgggtagt 4620
gaaaagacat tcatgaggac agctaaaaca ataagtaatg taaaatacag catagcaaaa 4680
ctttaacctc caaatcaagc ctctacttga atccttttct gagggatgaa taaggcatat 4740
gcatcagggg ctgttgccaa tgtgcattag ctgtttgcag cctcaccttc tttcatggag 4800
tttaagatat agtgtatttt cccaaggttt gaactagctc ttcatttctt tatgttttaa 4860
atgcactgac ctcccacatt ccctttttag taaaatattc agaaataatt taaatacatc 4920
attgcaatga aaataaatgt tttttattag gcagaatcca gatgctcaag gcccttcata 4980
atatccccca gtttagtagt tggacttagg gaacaaagga acctttaata gaaattggac 5040
agcaagaaag cgagcttagt gatacttgtg ggccagggca ttagccacac cagccaccac 5100
tttctgatag gcagcctgca ctggtggggt gaattctttg ccaaagtgat gggccagcac 5160
acagaccagc acgttgccca ggagctgtgg gaggaagata agaggtatga acatgattag 5220
caaaagggcc tagcttggac tcagaataat ccagccttat cccaaccata aaataaaagc 5280
agaatggtag ctggattgta gctgctatta gcaatatgaa acctcttaca tcagttacaa 5340
tttatatgca gaaatattta tatgcagaga tattgctatt gccttaaccc agaaattatc 5400
actgttattc tttagaatgg tgcaaagagg catgatacat tgtatcatta ttgccctgaa 5460
agaaagagat tagggaaagt attagaaata agataaacaa aaaagtatat taaaagaaga 5520
aagcattttt taaaattaca aatgcaaaat taccctgatt tggtcaatat gtgtacacat 5580
attaaaacat tacactttaa cccataaata tgtataatga ttatgtatca attaaaaata 5640
aaagaaaata aagtagggag attatgaata tgcaaataag cacacatata ttccaaatag 5700
taatgtacta ggcagactgt gtaaagtttt tttttaagtt acttaatgta tctcagagat 5760
atttcctttt gttatacaca atgttaaggc attaagtata atagtaaaaa ttgcggagaa 5820
gaaaaaaaaa gaaagcaaga attaaacaaa agaaaacaat tgttatgaac agcaaataaa 5880
agaaactaaa acgatcctga gacttccaca ctgatgcaat cattcgtctg tttcccattc 5940
taaactgtac cctgttactt atccccttcc tatgacatga acttaaccat agaaaagaag 6000
gggaaagaaa acatcaagcg tcccatagac tcaccctgaa gttctcagga tccacgtgca 6060
gcttgtcaca gtgcagctca ctcagtgtgg caaaggtgcc cttgaggttg tccaggtgag 6120
ccaggccatc actaaaggca ccgagcactt tcttgccatg agccttcacc ttagggttgc 6180
ccataacagc atcaggagtg gacagatccc caaaggactc aaagaacctc tgggtccaag 6240
ggtagaccac cagcagccta agggtgggaa aatagaccaa taggcagaga gagtcagtgc 6300
ctatcagaaa cccaagagtc ttctctgtct ccacatgccc agtttctatt ggtctcctta 6360
aacctgtctt gtaaccttga taccaacctg cccagggcct caccaccaac ttcatccacg 6420
ttcaccttgc cccacagggc agtaacggca gacttctcct caggagtcag atgcaccatg 6480
gtgtctgttt gaggttgcta gtgaacacag ttgtgtcaga agcaaatgta agcaatagat 6540
ggctctgccc tgacttttat gcccagccct ggctcctgcc ctccctgctc ctgggagtag 6600
attggccaac cctagggtgt ggctccacag ggtgaggtct aagtgatgac agccgtacct 6660
gtccttggct cttctggcac tggcttagga gttggacttc aaaccctcag ccctccctct 6720
aagatatatc tcttggcccc ataccatcag tacaaattgc tactaaaaac atcctccttt 6780
gcaagtgtat ttacgtatct agaatatgtc acattctgtc tcaggcatcc attttcttta 6840
tgatgccgtt tgaggtggag ttttagtcag gtggtcagct tctccttttt tttgccatct 6900
gccctgtaag catcctgctg gggacccaga taggagtcat cactctaggc tgagaacatc 6960
tgggcacaca ccctaagcct cagcatgact catcatgact cagcattgct gtgcttgagc 7020
cagaaggttt gcttagaagg ttacacagaa ccagaaggcg ggggtggggc actgaccccg 7080
acaggggcct ggccagaact gctcatgctt ggactatggg aggtcactaa tggagacaca 7140
cagaaatgta acaggaacta aggaaaaact gaagcttatt taatcagaga tgaggatgct 7200
ggaagggata gagggagctg agcttgtaaa aagtatagta atcattcagc aaatggtttt 7260
gaagcacctg ctggatgcta aacactattt tcagtgcttg aatcataaat aagaataaaa 7320
catgtatctt attccccaca agagtccaag taaaaaataa cagttaatta taatgtgctc 7380
tgtcccccag gctggagtgc agtggcacga tctcagctca ctgcaacctc cgcctcccgg 7440
gctggttaga aggttctact ggaggagggt cccagcccat tgctaaatta acatcaggct 7500
ctgagactgg cagtatatct ctaacagtgg ttgatgctat cttctggaac ttgcctgcta 7560
cattgagacc actgacccat acataggaag cccatagctc tgtcctgaac tgttaggcca 7620
ctggtccaga gagtgtgcat ctcctttgat cctcataata accctatgag atagacacaa 7680
ttattactct tactttatag atgatgatcc tgaaaacata ggagtcaagg cacttgcccc 7740
tagctggggg tataggggag cagtcccatg tagtagtaga atgaaaaatg ctgctatgct 7800
gtgcctcccc cacctttccc atgtctgccc tctactcatg gtctatctct cctggctcct 7860
gggagtcatg gactccaccc agcaccacca acctgaccta accacctatc tgagcctgcc 7920
agcctataac ccatctgggc cctgatagct ggtggccagc cctgacccca ccccaccctc 7980
cctggaacct ctgatagaca catctggcac accagctcgc aaagtcaccg tgagggtctt 8040
gtgtttgctg agtcaaaatt ccttgaaatc caagtcctta gagactcctg ctcccaaatt 8100
tacagtcata gacttcttca tggctgtctc ctttatccac agaatgattc ctttgcttca 8160
ttgccccatc catctgatcc tcctcatcag tgcagcacag ggcccatgag cagtagctgc 8220
agagtctcac ataggtctgg cactgcctct gacatgtccg accttaggca aatgcttgac 8280
tcttctctag tgcatgcaaa tctgacactc agtgggcctg ggtgaaggtg agaattttat 8340
tgctgaatga gagcctctgg ggacatcttg ccagtcaatg agtctcaggt tcaatttcct 8400
tctcagtctt ggagtaacag aagctcatgc atttaataaa cggaaatttt gtattgaaat 8460
gagagccatt ggaaatcatt tactccagac tcctacttat aaaaagagaa actgaggctc 8520
agagaagggt ggggactttc tcagtatgac atggaaatga tcaggcttgg attcaaagct 8580
cctgactttc tgtctagtgt atgtgcagtg agcccctttt cctctaactg aaagaaggaa 8640
aaaaaaatgg aacccaaaat attctacata gtttccatgt cacagccagg gctgggcagt 8700
ctcctgttat ttcttttaaa ataaatatat catttaaatg cataaataag caaaccctgc 8760
tcgggaatgg gagggagagt ctctggagtc caccccttct cggccctggc tctgcagata 8820
gtgctatcaa agccctgaca gagccctgcc cattgctggg ccttggagtg agtcagccta 8880
gtagagaggc agggcaagcc atctcatagc tgctgagtgg gagagagaaa agggctcatt 8940
gtctataaac tcaggtcatg gctattctta ttctcacact aagaaaaaga atgagatgtc 9000
tacatatacc ctgcgtcccc tcttgtgtac tggggtcccc aagagctctc taaaagtgat 9060
ggcaaagtca ttgcgctaga tgccatccca tctattataa acctgcattt gtctccacac 9120
accagtcatg gacaataacc ctcctcccag gtccacgtgc ttgtctttgt ataatactca 9180
agtaatttcg gaaaatgtat tctttcaatc ttgttctgtt attcctgttt caatggctta 9240
gtagaaaaag tacatacttg ttttcccata aattgacaat agacaatttc acatcaatgt 9300
ctatatgggt cgttgtgttt gctgtgtttg caaaaactca caataacttt atattgttac 9360
tactctaaga aagttacaac atggtgaata caagagaaag ctattacaag tccagaaaat 9420
aaaagttatc atcttgaggg tcgacaatca acctctggat tacaaaattt gtgaaagatt 9480
gactggtatt cttaactatg ttgctccttt tacgctatgt ggatacgctg ctttaatgcc 9540
tttgtatcat gctattgctt cccgtatggc tttcattttc tcctccttgt ataaatcctg 9600
gttgctgtct ctttatgagg agttgtggcc cgttgtcagg caacgtggcg tggtgtgcac 9660
tgtgtttgct gacgcaaccc ccactggttg gggcattgcc accacctgtc agctcctttc 9720
cgggactttc gctttccccc tccctattgc cacggcggaa ctcatcgccg cctgccttgc 9780
ccgctgctgg acaggggctc ggctgttggg cactgacaat tccgtggtgt tgtcggggaa 9840
gctgacgtcc tttccatggc tgctcgcctg tgttgccacc tggattctgc gcgggacgtc 9900
cttctgctac gtcccttcgg ccctcaatcc agcggacctt ccttcccgcg gcctgctgcc 9960
ggctctgcgg cctcttccgc gtcttcgcct tcgccctcag acgagtcgga tctccctttg 10020
ggccgcctcc ccgcctggaa ttcgagctcg gtacctttaa gaccaatgac ttacaaggca 10080
gctgtagatc ttagccactt tttaaaagaa aaggggggac tggaagggct aattcactcc 10140
caacgaagac aagatctgct ttttgcttgt actgggtctc tctggttaga ccagatctga 10200
gcctgggagc tctctggcta actagggaac ccactgctta agcctcaata aagcttgcct 10260
tgagtgcttc aagtagtgtg tgcccgtctg ttgtgtgact ctggtaacta gagatccctc 10320
agaccctttt agtcagtgtg gaaaatctct agcagtagta gttcatgtca tcttattatt 10380
cagtatttat aacttgcaaa gaaatgaata tcagagagtg agaggaactt gtttattgca 10440
gcttataatg gttacaaata aagcaatagc atcacaaatt tcacaaataa agcatttttt 10500
tcactgcatt ctagttgtgg tttgtccaaa ctcatcaatg tatcttatca tgtctggctc 10560
tagctatccc gcccctaact ccgcccatcc cgcccctaac tccgcccagt tccgcccatt 10620
ctccgcccca tggctgacta atttttttta tttatgcaga ggccgaggcc gcctcggcct 10680
ctgagctatt ccagaagtag tgaggaggct tttttggagg ccgctagcgt cgaccattac 10740
ttattgtttt agctgtcctc atgaatgtct tttcactacc catttgctta tcctgcatct 10800
ctcagccttg actccactca gttctcttgc ttagagatac cacctttccc ctgaagtgtt 10860
ccttccatgt tttacggcga gatggtttct cctcgcctgg ccactcagcc ttagttgtct 10920
ctgttgtctt atagaggtct acttgaagaa ggaaaaacag ggggcatggt ttgactgtcc 10980
tgtgagccct tcttccctgc ctcccccact cacagtgacc cggaatccct cgacatggca 11040
gtctagcact agtgcggccg cagatctgct tcctcgctca ctgactcgct gcgctcggtc 11100
gttcggctgc ggcgagcggt atcagctcac tcaaaggcgg taatacggtt atccacagaa 11160
tcaggggata acgcaggaaa gaacatgtga gcaaaaggcc agcaaaaggc caggaaccgt 11220
aaaaa 11225
SEQ ID NO: 14
acatttgctt ctgacacaac tgtgttcact agcaacctca aacagacacc atggtgcacc 60
tgacccctga ggagaagagc gccgtgacag ccctgtgggg caaggtgaac gtggacgaag 120
tgggaggaga ggccctgggc aggttggtat caaggttaca agacaggttt aaggagacca 180
atagaaactg ggcatgtgga gacagagaag actcttgggt ttctgatagg cactgactct 240
ctctgcctat tggtctattt tcccaccctt aggctgctgg tggtgtaccc atggacccag 300
agattctttg agagcttcgg cgacctgtcc acaccagatg ccgtgatggg caaccccaag 360
gtgaaggccc acggcaagaa ggtgctggga gccttctctg acggactggc ccacctggat 420
aatctgaagg gcacctttgc ccagctgagc gagctgcact gcgacaagct gcacgtggat 480
cccgagaact tcagggtgag tctatgggac gcttgatgtt ttctttcccc ttcttttcta 540
tggttaagtt catgtcatag gaaggggata agtaacaggg tacacatatt gaccaaatca 600
gggtaatttt gcatttgtaa ttttaaaaaa tgctttcttc ttttaatata cttttttgtt 660
tatcttattt ctaatacttt ccctaatctc tttctttcag ggcaataatg atacaatgta 720
tcatgcctct ttgcaccatt ctaaagaata acagtgataa tttctgggtt aaggcaatag 780
caatatctct gcatataaat atttctgcat ataaattgta actgatgtaa gaggtttcat 840
attgctaata gcagctacaa tccagctacc attctgcttt tattttatgg ttgggataag 900
gctggattat tctgagtcca agctaggccc ttttgctaat catgttcata cctcttatct 960
tcctcccaca gctcctgggc aacgtgctgg tgtgcgtgct ggcccaccac ttcggcaagg 1020
agtttacacc ccctgtgcag gcagcatacc agaaggtggt ggcaggagtg gcaaatgcac 1080
tggcccacaa gtatcactga gctcgctttc ttgctgtcca atttctatta aaggttcctt 1140
tgttccctaa gtccaactac taaactgggg gatattatga agggccttga gcatctggat 1200
tctgcctaat aaaaaacatt tattttcatt gcaa 1234
SEQ ID NO: 15
ggccgcgttg ctggcgtttt tccataggct ccgcccccct gacgagcatc acaaaaatcg 60
acgctcaagt cagaggtggc gaaacccgac aggactataa agataccagg cgtttccccc 120
tggaagctcc ctcgtgcgct ctcctgttcc gaccctgccg cttaccggat acctgtccgc 180
ctttctccct tcgggaagcg tggcgctttc tcatagctca cgctgtaggt atctcagttc 240
ggtgtaggtc gttcgctcca agctgggctg tgtgcacgaa ccccccgttc agcccgaccg 300
ctgcgcctta tccggtaact atcgtcttga gtccaacccg gtaagacacg acttatcgcc 360
actggcagca gccactggta acaggattag cagagcgagg tatgtaggcg gtgctacaga 420
gttcttgaag tggtggccta actacggcta cactagaaga acagtatttg gtatctgcgc 480
tctgctgaag ccagttacct tcggaaaaag agttggtagc tcttgatccg gcaaacaaac 540
caccgctggt agcggtggtt tttttgtttg caagcagcag attacgcgca gaaaaaaagg 600
atctcaagaa gatcctttga tcttttctac ggggtctgac gctcagtgga acgaaaactc 660
acgttaaggg attttggtca tgaagcgctt ttgaagctcg gatccgaaca aacgacccaa 720
cacccgtgcg ttttattctg tctttttatt gccgatcccc tcagaagaac tcgtcaagaa 780
ggcgatagaa ggcgatgcgc tgcgaatcgg gagcggcgat accgtaaagc acgaggaagc 840
ggtcagccca ttcgccgcca agctcttcag caatatcacg ggtagccaac gctatgtcct 900
gatagcggtc cgccacaccc agccggccac agtcgatgaa tccagaaaag cggccatttt 960
ccaccatgat attcggcaag caggcatcgc catgggtcac gacgagatcc tcgccgtcgg 1020
gcatgctcgc cttgagcctg gcgaacagtt cggctggcgc gagcccctga tgctcttcgt 1080
ccagatcatc ctgatcgaca agaccggctt ccatccgagt acgtgctcgc tcgatgcgat 1140
gtttcgcttg gtggtcgaat gggcaggtag ccggatcaag cgtatgcagc cgccgcattg 1200
catcagccat gatggatact ttctcggcag gagcaaggtg agatgacagg agatcctgcc 1260
ccggcacttc gcccaatagc agccagtccc ttcccgcttc agtgacaacg tcgagcacag 1320
ctgcgcaagg aacgcccgtc gtggccagcc acgatagccg cgctgcctcg tcttgcagtt 1380
cattcagggc accggacagg tcggtcttga caaaaagaac cgggcgcccc tgcgctgaca 1440
gccggaacac ggcggcatca gagcagccga ttgtctgttg tgcccagtca tagccgaata 1500
gcctctccac ccaagcggcc ggagaacctg cgtgcaatcc atcttgttca atcatgcgaa 1560
acgatcctca tcctgtctct tgatcagagc ttgatcccct gcgccatcag atccttggcg 1620
gcaagaaagc catccagttt actttgcagg gcttcccaac cttaccagag gcctgcgccg 1680
cggccagctg gctagcaatt cccgggttaa ctctagagac attgattatt gactagttat 1740
taatagtaat caattacggg gtcattagtt catagcccat atatggagtt ccgcgttaca 1800
taacttacgg taaatggccc gcctggctga ccgcccaacg acccccgccc attgacgtca 1860
ataatgacgt atgttcccat agtaacgcca atagggactt tccattgacg tcaatgggtg 1920
gagtatttac ggtaaactgc ccacttggca gtacatcaag tgtatcatat gccaagtacg 1980
ccccctattg acgtcaatga cggtaaatgg cccgcctggc attatgccca gtacatgacc 2040
ttatgggact ttcctacttg gcagtacatc tacgtattag tcatcgctat taccatggtg 2100
atgcggtttt ggcagtacat caatgggcgt ggatagcggt ttgactcacg gggatttcca 2160
agtctccacc ccattgacgt caatgggagt ttgttttggc accaaaatca acgggacttt 2220
ccaaaatgtc gtaacaactc cgccccattg acgcaaatgg gcggtaggcg tgtacggtgg 2280
gaggtctata taagcagagc tcgtttagtg aaccggggtc tctctggtta gaccagatct 2340
gagcctggga gctctctggc taactaggga acccactgct taagcctcaa taaagcttgc 2400
cttgagtgct tcaagtagtg tgtgcccgtc tgttgtgtga ctctggtaac tagagatccc 2460
tcagaccctt ttagtcagtg tggaaaatct ctagcagtgg cgcccgaaca gggacttgaa 2520
agcgaaaggg aaaccagagg agctctctcg acgcaggact cggcttgctg aagcgcgcac 2580
ggcaagaggc gaggggcggc gactggtgag tacgccaaaa attttgacta gcggaggcta 2640
gaaggagaga gatgggtgcg agagcgtcag tattaagcgg gggagaatta gatcgcgatg 2700
ggaaaaaatt cggttaaggc cagggggaaa gaaaaaatat aaattaaaac atatagtatg 2760
ggcaagcagg gagctagaac gattcgcagt taatcctggc ctgttagaaa catcagaagg 2820
ctgtagacaa atactgggac agctacaacc atcccttcag acaggatcag aagaacttag 2880
atcattatat aatacagtag caaccctcta ttgtgtgcat caaaggatag agataaaaga 2940
caccaaggaa gctttagaca agatagagga agagcaaaac aaaagtaaga ccaccgcaca 3000
gcaagcggcc gctgatcttc agacctggag gaggagatat gagggacaat tggagaagtg 3060
aattatataa atataaagta gtaaaaattg aaccattagg agtagcaccc accaaggcaa 3120
agagaagagt ggtgcagaga gaaaaaagag cagtgggaat aggagctttg ttccttgggt 3180
tcttgggagc agcaggaagc actatgggcg cagcgtcaat gacgctgacg gtacaggcca 3240
gacaattatt gtctggtata gtgcagcagc agaacaattt gctgagggct attgaggcgc 3300
aacagcatct gttgcaactc acagtctggg gcatcaagca gctccaggca agaatcctgg 3360
ctgtggaaag atacctaaag gatcaacagc tcctggggat ttggggttgc tctggaaaac 3420
tcatttgcac cactgctgtg ccttggaatg ctagttggag taataaatct ctggaacaga 3480
tttggaatca cacgacctgg atggagtggg acagagaaat taacaattac acaagcttaa 3540
tacactcctt aattgaagaa tcgcaaaacc agcaagaaaa gaatgaacaa gaattattgg 3600
aattagataa atgggcaagt ttgtggaatt ggtttaacat aacaaattgg ctgtggtata 3660
taaaattatt cataatgata gtaggaggct tggtaggttt aagaatagtt tttgctgtac 3720
tttctatagt gaatagagtt aggcagggat attcaccatt atcgtttcag acccacctcc 3780
caaccccgag gggacccgac aggcccgaag gaatagaaga agaaggtgga gagagagaca 3840
gagacagatc cattcgatta gtgaacggat ctcgacggta tcggttaact tttaaaagaa 3900
aaggggggat tggggggtac agtgcagggg aaagaatagt agacataata gcaacagaca 3960
tacaaactaa agaattacaa aaacaaatta caaaaattca aaattttatc gatcacgaga 4020
ctagcctcga gaagcttgat atccctaggt attgaataag aaaaatgaag ttaaggtggt 4080
tgatggtaac actatgctaa taactgcaga gccagaagca ccataaggga catgataagg 4140
gagccagcag acctctgatc tcttcctgaa tgctaatctt aaacatcctg aggaagaatg 4200
ggacttccat ttggggtggg cctatgacag ggtaataaga cagtagtgaa tatcaagcta 4260
caaaaagccg cctttcaaat tcttctcagt cctaactttt catactaagc ccagtccttc 4320
caaagcagac tgtgaaagag tgatagttcc gggagactag cactgcagat tccgggtcac 4380
tgtgagtggg ggaggcaggg aagaagggct cacaggacag tcaaaccatg acccctgttt 4440
ttccttcttc aagtagacct ctataagaca acagagacaa ctaaggctga gtggccaggc 4500
gaggagaaac catctcgccg taaaacatgg aaggaacact tcaggggaaa ggtggtatct 4560
ctaagcaaga gaactgagtg gagtcaaggc tgagagatgc aggataagca aatgggtagt 4620
gaaaagacat tcatgaggac agctaaaaca ataagtaatg taaaatacag catagcaaaa 4680
ctttaacctc caaatcaagc ctctacttga atccttttct gagggatgaa taaggcatat 4740
gcatcagggg ctgttgccaa tgtgcattag ctgtttgcag cctcaccttc tttcatggag 4800
tttaagatat agtgtatttt cccaaggttt gaactagctc ttcatttctt tatgttttaa 4860
atgcactgac ctcccacatt ccctttttag taaaatattc agaaataatt taaatacatc 4920
attgcaatga aaataaatgt tttttattag gcagaatcca gatgctcaag gcccttcata 4980
atatccccca gtttagtagt tggacttagg gaacaaagga acctttaata gaaattggac 5040
agcaagaaag cgagctcagt gatacttgtg ggccagtgca tttgccactc ctgccaccac 5100
cttctggtat gctgcctgca cagggggtgt aaactccttg ccgaagtggt gggccagcac 5160
gcacaccagc acgttgccca ggagctgtgg gaggaagata agaggtatga acatgattag 5220
caaaagggcc tagcttggac tcagaataat ccagccttat cccaaccata aaataaaagc 5280
agaatggtag ctggattgta gctgctatta gcaatatgaa acctcttaca tcagttacaa 5340
tttatatgca gaaatattta tatgcagaga tattgctatt gccttaaccc agaaattatc 5400
actgttattc tttagaatgg tgcaaagagg catgatacat tgtatcatta ttgccctgaa 5460
agaaagagat tagggaaagt attagaaata agataaacaa aaaagtatat taaaagaaga 5520
aagcattttt taaaattaca aatgcaaaat taccctgatt tggtcaatat gtgtaccctg 5580
ttacttatcc ccttcctatg acatgaactt aaccatagaa aagaagggga aagaaaacat 5640
caagcgtccc atagactcac cctgaagttc tcgggatcca cgtgcagctt gtcgcagtgc 5700
agctcgctca gctgggcaaa ggtgcccttc agattatcca ggtgggccag tccgtcagag 5760
aaggctccca gcaccttctt gccgtgggcc ttcaccttgg ggttgcccat cacggcatct 5820
ggtgtggaca ggtcgccgaa gctctcaaag aatctctggg tccatgggta caccaccagc 5880
agcctaaggg tgggaaaata gaccaatagg cagagagagt cagtgcctat cagaaaccca 5940
agagtcttct ctgtctccac atgcccagtt tctattggtc tccttaaacc tgtcttgtaa 6000
ccttgatacc aacctgccca gggcctctcc tcccacttcg tccacgttca ccttgcccca 6060
cagggctgtc acggcgctct tctcctcagg ggtcaggtgc accatggtgt ctgtttgagg 6120
ttgctagtga acacagttgt gtcagaagca aatgtaagca atagatggct ctgccctgac 6180
ttttatgccc agccctggct cctgccctcc ctgctcctgg gagtagattg gccaacccta 6240
gggtgtggct ccacagggtg aggtctaagt gatgacagcc gtacctgtcc ttggctcttc 6300
tggcactggc ttaggagttg gacttcaaac cctcagccct ccctctaaga tatatctctt 6360
ggccccatac catcagtaca aattgctact aaaaacatcc tcctttgcaa gtgtatttac 6420
gtatctagaa tatgtcacat tctgtctcag gcatccattt tctttatgat gccgtttgag 6480
gtggagtttt agtcaggtgg tcagcttctc cttttttttg ccatctgccc tgtaagcatc 6540
ctgctgggga cccagatagg agtcatcact ctaggctgag aacatctggg cacacaccct 6600
aagcctcagc atgactcatc atgactcagc attgctgtgc ttgagccaga aggtttgctt 6660
agaaggttac acagaaccag aaggcggggg tggggcactg accccgacag gggcctggcc 6720
agaactgctc atgcttggac tatgggaggt cactaatgga gacacacaga aatgtaacag 6780
gaactaagga aaaactgaag cttatttaat cagagatgag gatgctggaa gggatagagg 6840
gagctgagct tgtaaaaagt atagtaatca ttcagcaaat ggttttgaag cacctgctgg 6900
atgctaaaca ctattttcag tgcttgaatc ataaataaga ataaaacatg tatcttattc 6960
cccacaagag tccaagtaaa aaataacagt taattataat gtgctctgtc ccccaggctg 7020
gagtgcagtg gcacgatctc agctcactgc aacctccgcc tcccgggctg gttagaaggt 7080
tctactggag gagggtccca gcccattgct aaattaacat caggctctga gactggcagt 7140
atatctctaa cagtggttga tgctatcttc tggaacttgc ctgctacatt gagaccactg 7200
acccatacat aggaagccca tagctctgtc ctgaactgtt aggccactgg tccagagagt 7260
gtgcatctcc tttgatcctc ataataaccc tatgagatag acacaattat tactcttact 7320
ttatagatga tgatcctgaa aacataggag tcaaggcact tgcccctagc tgggggtata 7380
ggggagcagt cccatgtagt agtagaatga aaaatgctgc tatgctgtgc ctcccccacc 7440
tttcccatgt ctgccctcta ctcatggtct atctctcctg gctcctggga gtcatggact 7500
ccacccagca ccaccaacct gacctaacca cctatctgag cctgccagcc tataacccat 7560
ctgggccctg atagctggtg gccagccctg accccacccc accctccctg gaacctctga 7620
tagacacatc tggcacacca gctcgcaaag tcaccgtgag ggtcttgtgt ttgctgagtc 7680
aaaattcctt gaaatccaag tccttagaga ctcctgctcc caaatttaca gtcatagact 7740
tcttcatggc tgtctccttt atccacagaa tgattccttt gcttcattgc cccatccatc 7800
tgatcctcct catcagtgca gcacagggcc catgagcagt agctgcagag tctcacatag 7860
gtctggcact gcctctgaca tgtccgacct taggcaaatg cttgactctt ctctagtgca 7920
tgcaaatctg acactcagtg ggcctgggtg aaggtgagaa ttttattgct gaatgagagc 7980
ctctggggac atcttgccag tcaatgagtc tcaggttcaa tttccttctc agtcttggag 8040
taacagaagc tcatgcattt aataaacgga aattttgtat tgaaatgaga gccattggaa 8100
atcatttact ccagactcct acttataaaa agagaaactg aggctcagag aagggtgggg 8160
actttctcag tatgacatgg aaatgatcag gcttggattc aaagctcctg actttctgtc 8220
tagtgtatgt gcagtgagcc ccttttcctc taactgaaag aaggaaaaaa aaatggaacc 8280
caaaatattc tacatagttt ccatgtcaca gccagggctg ggcagtctcc tgttatttct 8340
tttaaaataa atatatcatt taaatgcata aataagcaaa ccctgctcgg gaatgggagg 8400
gagagtctct ggagtccacc ccttctcggc cctggctctg cagatagtgc tatcaaagcc 8460
ctgacagagc cctgcccatt gctgggcctt ggagtgagtc agcctagtag agaggcaggg 8520
caagccatct catagctgct gagtgggaga gagaaaaggg ctcattgtct ataaactcag 8580
gtcatggcta ttcttattct cacactaaga aaaagaatga gatgtctaca tataccctgc 8640
gtcccctctt gtgtactggg gtccccaaga gctctctaaa agtgatggca aagtcattgc 8700
gctagatgcc atcccatcta ttataaacct gcatttgtct ccacacacca gtcatggaca 8760
ataaccctcc tcccaggtcc acgtgcttgt ctttgtataa tactcaagta atttcggaaa 8820
atgtattctt tcaatcttgt tctgttattc ctgtttcaat ggcttagtag aaaaagtaca 8880
tacttgtttt cccataaatt gacaatagac aatttcacat caatgtctat atgggtcgtt 8940
gtgtttgctg tgtttgcaaa aactcacaat aactttatat tgttactact ctaagaaagt 9000
tacaacatgg tgaatacaag agaaagctat tacaagtcca gaaaataaaa gttatcatct 9060
tgagggtcga caatcaacct ctggattaca aaatttgtga aagattgact ggtattctta 9120
actatgttgc tccttttacg ctatgtggat acgctgcttt aatgcctttg tatcatgcta 9180
ttgcttcccg tatggctttc attttctcct ccttgtataa atcctggttg ctgtctcttt 9240
atgaggagtt gtggcccgtt gtcaggcaac gtggcgtggt gtgcactgtg tttgctgacg 9300
caacccccac tggttggggc attgccacca cctgtcagct cctttccggg actttcgctt 9360
tccccctccc tattgccacg gcggaactca tcgccgcctg ccttgcccgc tgctggacag 9420
gggctcggct gttgggcact gacaattccg tggtgttgtc ggggaagctg acgtcctttc 9480
catggctgct cgcctgtgtt gccacctgga ttctgcgcgg gacgtccttc tgctacgtcc 9540
cttcggccct caatccagcg gaccttcctt cccgcggcct gctgccggct ctgcggcctc 9600
ttccgcgtct tcgccttcgc cctcagacga gtcggatctc cctttgggcc gcctccccgc 9660
ctggaattcg agctcggtac ctttaagacc aatgacttac aaggcagctg tagatcttag 9720
ccacttttta aaagaaaagg ggggactgga agggctaatt cactcccaac gaagacaaga 9780
tctgcttttt gcttgtactg ggtctctctg gttagaccag atctgagcct gggagctctc 9840
tggctaacta gggaacccac tgcttaagcc tcaataaagc ttgccttgag tgcttcaagt 9900
agtgtgtgcc cgtctgttgt gtgactctgg taactagaga tccctcagac ccttttagtc 9960
agtgtggaaa atctctagca gtagtagttc atgtcatctt attattcagt atttataact 10020
tgcaaagaaa tgaatatcag agagtgagag gaacttgttt attgcagctt ataatggtta 10080
caaataaagc aatagcatca caaatttcac aaataaagca tttttttcac tgcattctag 10140
ttgtggtttg tccaaactca tcaatgtatc ttatcatgtc tggctctagc tatcccgccc 10200
ctaactccgc ccatcccgcc cctaactccg cccagttccg cccattctcc gccccatggc 10260
tgactaattt tttttattta tgcagaggcc gaggccgcct cggcctctga gctattccag 10320
aagtagtgag gaggcttttt tggaggccgc tagcgtcgac cattacttat tgttttagct 10380
gtcctcatga atgtcttttc actacccatt tgcttatcct gcatctctca gccttgactc 10440
cactcagttc tcttgcttag agataccacc tttcccctga agtgttcctt ccatgtttta 10500
cggcgagatg gtttctcctc gcctggccac tcagccttag ttgtctctgt tgtcttatag 10560
aggtctactt gaagaaggaa aaacaggggg catggtttga ctgtcctgtg agcccttctt 10620
ccctgcctcc cccactcaca gtgacccgga atccctcgac atggcagtct agcactagtg 10680
cggccgcaga tctgcttcct cgctcactga ctcgctgcgc tcggtcgttc ggctgcggcg 10740
agcggtatca gctcactcaa aggcggtaat acggttatcc acagaatcag gggataacgc 10800
aggaaagaac atgtgagcaa aaggccagca aaaggccagg aaccgtaaaa a 10851
SEQ ID NO: 16
acatttgctt ctgacacaac tgtgttcact agcaacctca aacagacacc atggtgcacc 60
taaccccgga ggagaaaagc gccgtgacgg ccctctgggg caaggtgaac gtcgacgagg 120
tcggcgggga ggccctgggc aggttggtat caaggttaca agacaggttt aaggagacca 180
atagaaactg ggcatgtgga gacagagaag actcttgggt ttctgatagg cactgactct 240
ctctgcctat tggtctattt tcccaccctt aggctgctgg tggtctaccc ctggacgcag 300
cgcttttttg agagcttcgg ggatctgtcc acccccgacg cggtgatggg caaccctaag 360
gtcaaggccc acggcaagaa ggtgctcggc gccttctccg acggcctggc tcacctggac 420
aacctcaagg gcaccttcgc ccagctgtcg gagctgcact gcgacaagct gcacgtcgac 480
cccgagaact tcagggtgag tctatgggac gcttgatgtt ttctttcccc ttcttttcta 540
tggttaagtt catgtcatag gaaggggata agtaacaggg tacacatatt gaccaaatca 600
gggtaatttt gcatttgtaa ttttaaaaaa tgctttcttc ttttaatata cttttttgtt 660
tatcttattt ctaatacttt ccctaatctc tttctttcag ggcaataatg atacaatgta 720
tcatgcctct ttgcaccatt ctaaagaata acagtgataa tttctgggtt aaggcaatag 780
caatatctct gcatataaat atttctgcat ataaattgta actgatgtaa gaggtttcat 840
attgctaata gcagctacaa tccagctacc attctgcttt tattttatgg ttgggataag 900
gctggattat tctgagtcca agctaggccc ttttgctaat catgttcata cctcttatct 960
tcctcccaca gctcctgggc aacgtgctgg tgtgcgtgct ggcccaccac ttcgggaagg 1020
aattcacgcc cccagtgcag gccgcgtacc agaaggtggt ggccggggtg gccaacgccc 1080
tggcccacaa gtaccactga gctcgctttc ttgctgtcca atttctatta aaggttcctt 1140
tgttccctaa gtccaactac taaactgggg gatattatga agggccttga gcatctggat 1200
tctgcctaat aaaaaacatt tattttcatt gcaa 1234
SEQ ID NO: 17
ggccgcgttg ctggcgtttt tccataggct ccgcccccct gacgagcatc acaaaaatcg 60
acgctcaagt cagaggtggc gaaacccgac aggactataa agataccagg cgtttccccc 120
tggaagctcc ctcgtgcgct ctcctgttcc gaccctgccg cttaccggat acctgtccgc 180
ctttctccct tcgggaagcg tggcgctttc tcatagctca cgctgtaggt atctcagttc 240
ggtgtaggtc gttcgctcca agctgggctg tgtgcacgaa ccccccgttc agcccgaccg 300
ctgcgcctta tccggtaact atcgtcttga gtccaacccg gtaagacacg acttatcgcc 360
actggcagca gccactggta acaggattag cagagcgagg tatgtaggcg gtgctacaga 420
gttcttgaag tggtggccta actacggcta cactagaaga acagtatttg gtatctgcgc 480
tctgctgaag ccagttacct tcggaaaaag agttggtagc tcttgatccg gcaaacaaac 540
caccgctggt agcggtggtt tttttgtttg caagcagcag attacgcgca gaaaaaaagg 600
atctcaagaa gatcctttga tcttttctac ggggtctgac gctcagtgga acgaaaactc 660
acgttaaggg attttggtca tgaagcgctt ttgaagctcg gatccgaaca aacgacccaa 720
cacccgtgcg ttttattctg tctttttatt gccgatcccc tcagaagaac tcgtcaagaa 780
ggcgatagaa ggcgatgcgc tgcgaatcgg gagcggcgat accgtaaagc acgaggaagc 840
ggtcagccca ttcgccgcca agctcttcag caatatcacg ggtagccaac gctatgtcct 900
gatagcggtc cgccacaccc agccggccac agtcgatgaa tccagaaaag cggccatttt 960
ccaccatgat attcggcaag caggcatcgc catgggtcac gacgagatcc tcgccgtcgg 1020
gcatgctcgc cttgagcctg gcgaacagtt cggctggcgc gagcccctga tgctcttcgt 1080
ccagatcatc ctgatcgaca agaccggctt ccatccgagt acgtgctcgc tcgatgcgat 1140
gtttcgcttg gtggtcgaat gggcaggtag ccggatcaag cgtatgcagc cgccgcattg 1200
catcagccat gatggatact ttctcggcag gagcaaggtg agatgacagg agatcctgcc 1260
ccggcacttc gcccaatagc agccagtccc ttcccgcttc agtgacaacg tcgagcacag 1320
ctgcgcaagg aacgcccgtc gtggccagcc acgatagccg cgctgcctcg tcttgcagtt 1380
cattcagggc accggacagg tcggtcttga caaaaagaac cgggcgcccc tgcgctgaca 1440
gccggaacac ggcggcatca gagcagccga ttgtctgttg tgcccagtca tagccgaata 1500
gcctctccac ccaagcggcc ggagaacctg cgtgcaatcc atcttgttca atcatgcgaa 1560
acgatcctca tcctgtctct tgatcagagc ttgatcccct gcgccatcag atccttggcg 1620
gcaagaaagc catccagttt actttgcagg gcttcccaac cttaccagag gcctgcgccg 1680
cggccagctg gctagcaatt cccgggttaa ctctagagac attgattatt gactagttat 1740
taatagtaat caattacggg gtcattagtt catagcccat atatggagtt ccgcgttaca 1800
taacttacgg taaatggccc gcctggctga ccgcccaacg acccccgccc attgacgtca 1860
ataatgacgt atgttcccat agtaacgcca atagggactt tccattgacg tcaatgggtg 1920
gagtatttac ggtaaactgc ccacttggca gtacatcaag tgtatcatat gccaagtacg 1980
ccccctattg acgtcaatga cggtaaatgg cccgcctggc attatgccca gtacatgacc 2040
ttatgggact ttcctacttg gcagtacatc tacgtattag tcatcgctat taccatggtg 2100
atgcggtttt ggcagtacat caatgggcgt ggatagcggt ttgactcacg gggatttcca 2160
agtctccacc ccattgacgt caatgggagt ttgttttggc accaaaatca acgggacttt 2220
ccaaaatgtc gtaacaactc cgccccattg acgcaaatgg gcggtaggcg tgtacggtgg 2280
gaggtctata taagcagagc tcgtttagtg aaccggggtc tctctggtta gaccagatct 2340
gagcctggga gctctctggc taactaggga acccactgct taagcctcaa taaagcttgc 2400
cttgagtgct tcaagtagtg tgtgcccgtc tgttgtgtga ctctggtaac tagagatccc 2460
tcagaccctt ttagtcagtg tggaaaatct ctagcagtgg cgcccgaaca gggacttgaa 2520
agcgaaaggg aaaccagagg agctctctcg acgcaggact cggcttgctg aagcgcgcac 2580
ggcaagaggc gaggggcggc gactggtgag tacgccaaaa attttgacta gcggaggcta 2640
gaaggagaga gatgggtgcg agagcgtcag tattaagcgg gggagaatta gatcgcgatg 2700
ggaaaaaatt cggttaaggc cagggggaaa gaaaaaatat aaattaaaac atatagtatg 2760
ggcaagcagg gagctagaac gattcgcagt taatcctggc ctgttagaaa catcagaagg 2820
ctgtagacaa atactgggac agctacaacc atcccttcag acaggatcag aagaacttag 2880
atcattatat aatacagtag caaccctcta ttgtgtgcat caaaggatag agataaaaga 2940
caccaaggaa gctttagaca agatagagga agagcaaaac aaaagtaaga ccaccgcaca 3000
gcaagcggcc gctgatcttc agacctggag gaggagatat gagggacaat tggagaagtg 3060
aattatataa atataaagta gtaaaaattg aaccattagg agtagcaccc accaaggcaa 3120
agagaagagt ggtgcagaga gaaaaaagag cagtgggaat aggagctttg ttccttgggt 3180
tcttgggagc agcaggaagc actatgggcg cagcgtcaat gacgctgacg gtacaggcca 3240
gacaattatt gtctggtata gtgcagcagc agaacaattt gctgagggct attgaggcgc 3300
aacagcatct gttgcaactc acagtctggg gcatcaagca gctccaggca agaatcctgg 3360
ctgtggaaag atacctaaag gatcaacagc tcctggggat ttggggttgc tctggaaaac 3420
tcatttgcac cactgctgtg ccttggaatg ctagttggag taataaatct ctggaacaga 3480
tttggaatca cacgacctgg atggagtggg acagagaaat taacaattac acaagcttaa 3540
tacactcctt aattgaagaa tcgcaaaacc agcaagaaaa gaatgaacaa gaattattgg 3600
aattagataa atgggcaagt ttgtggaatt ggtttaacat aacaaattgg ctgtggtata 3660
taaaattatt cataatgata gtaggaggct tggtaggttt aagaatagtt tttgctgtac 3720
tttctatagt gaatagagtt aggcagggat attcaccatt atcgtttcag acccacctcc 3780
caaccccgag gggacccgac aggcccgaag gaatagaaga agaaggtgga gagagagaca 3840
gagacagatc cattcgatta gtgaacggat ctcgacggta tcggttaact tttaaaagaa 3900
aaggggggat tggggggtac agtgcagggg aaagaatagt agacataata gcaacagaca 3960
tacaaactaa agaattacaa aaacaaatta caaaaattca aaattttatc gatcacgaga 4020
ctagcctcga gaagcttgat atccctaggt attgaataag aaaaatgaag ttaaggtggt 4080
tgatggtaac actatgctaa taactgcaga gccagaagca ccataaggga catgataagg 4140
gagccagcag acctctgatc tcttcctgaa tgctaatctt aaacatcctg aggaagaatg 4200
ggacttccat ttggggtggg cctatgacag ggtaataaga cagtagtgaa tatcaagcta 4260
caaaaagccg cctttcaaat tcttctcagt cctaactttt catactaagc ccagtccttc 4320
caaagcagac tgtgaaagag tgatagttcc gggagactag cactgcagat tccgggtcac 4380
tgtgagtggg ggaggcaggg aagaagggct cacaggacag tcaaaccatg acccctgttt 4440
ttccttcttc aagtagacct ctataagaca acagagacaa ctaaggctga gtggccaggc 4500
gaggagaaac catctcgccg taaaacatgg aaggaacact tcaggggaaa ggtggtatct 4560
ctaagcaaga gaactgagtg gagtcaaggc tgagagatgc aggataagca aatgggtagt 4620
gaaaagacat tcatgaggac agctaaaaca ataagtaatg taaaatacag catagcaaaa 4680
ctttaacctc caaatcaagc ctctacttga atccttttct gagggatgaa taaggcatat 4740
gcatcagggg ctgttgccaa tgtgcattag ctgtttgcag cctcaccttc tttcatggag 4800
tttaagatat agtgtatttt cccaaggttt gaactagctc ttcatttctt tatgttttaa 4860
atgcactgac ctcccacatt ccctttttag taaaatattc agaaataatt taaatacatc 4920
attgcaatga aaataaatgt tttttattag gcagaatcca gatgctcaag gcccttcata 4980
atatccccca gtttagtagt tggacttagg gaacaaagga acctttaata gaaattggac 5040
agcaagaaag cgagctcagt ggtacttgtg ggccagggcg ttggccaccc cggccaccac 5100
cttctggtac gcggcctgca ctgggggcgt gaattccttc ccgaagtggt gggccagcac 5160
gcacaccagc acgttgccca ggagctgtgg gaggaagata agaggtatga acatgattag 5220
caaaagggcc tagcttggac tcagaataat ccagccttat cccaaccata aaataaaagc 5280
agaatggtag ctggattgta gctgctatta gcaatatgaa acctcttaca tcagttacaa 5340
tttatatgca gaaatattta tatgcagaga tattgctatt gccttaaccc agaaattatc 5400
actgttattc tttagaatgg tgcaaagagg catgatacat tgtatcatta ttgccctgaa 5460
agaaagagat tagggaaagt attagaaata agataaacaa aaaagtatat taaaagaaga 5520
aagcattttt taaaattaca aatgcaaaat taccctgatt tggtcaatat gtgtaccctg 5580
ttacttatcc ccttcctatg acatgaactt aaccatagaa aagaagggga aagaaaacat 5640
caagcgtccc atagactcac cctgaagttc tcggggtcga cgtgcagctt gtcgcagtgc 5700
agctccgaca gctgggcgaa ggtgcccttg aggttgtcca ggtgagccag gccgtcggag 5760
aaggcgccga gcaccttctt gccgtgggcc ttgaccttag ggttgcccat caccgcgtcg 5820
ggggtggaca gatccccgaa gctctcaaaa aagcgctgcg tccaggggta gaccaccagc 5880
agcctaaggg tgggaaaata gaccaatagg cagagagagt cagtgcctat cagaaaccca 5940
agagtcttct ctgtctccac atgcccagtt tctattggtc tccttaaacc tgtcttgtaa 6000
ccttgatacc aacctgccca gggcctcccc gccgacctcg tcgacgttca ccttgcccca 6060
gagggccgtc acggcgcttt tctcctccgg ggttaggtgc accatggtgt ctgtttgagg 6120
ttgctagtga acacagttgt gtcagaagca aatgtaagca atagatggct ctgccctgac 6180
ttttatgccc agccctggct cctgccctcc ctgctcctgg gagtagattg gccaacccta 6240
gggtgtggct ccacagggtg aggtctaagt gatgacagcc gtacctgtcc ttggctcttc 6300
tggcactggc ttaggagttg gacttcaaac cctcagccct ccctctaaga tatatctctt 6360
ggccccatac catcagtaca aattgctact aaaaacatcc tcctttgcaa gtgtatttac 6420
gtatctagaa tatgtcacat tctgtctcag gcatccattt tctttatgat gccgtttgag 6480
gtggagtttt agtcaggtgg tcagcttctc cttttttttg ccatctgccc tgtaagcatc 6540
ctgctgggga cccagatagg agtcatcact ctaggctgag aacatctggg cacacaccct 6600
aagcctcagc atgactcatc atgactcagc attgctgtgc ttgagccaga aggtttgctt 6660
agaaggttac acagaaccag aaggcggggg tggggcactg accccgacag gggcctggcc 6720
agaactgctc atgcttggac tatgggaggt cactaatgga gacacacaga aatgtaacag 6780
gaactaagga aaaactgaag cttatttaat cagagatgag gatgctggaa gggatagagg 6840
gagctgagct tgtaaaaagt atagtaatca ttcagcaaat ggttttgaag cacctgctgg 6900
atgctaaaca ctattttcag tgcttgaatc ataaataaga ataaaacatg tatcttattc 6960
cccacaagag tccaagtaaa aaataacagt taattataat gtgctctgtc ccccaggctg 7020
gagtgcagtg gcacgatctc agctcactgc aacctccgcc tcccgggctg gttagaaggt 7080
tctactggag gagggtccca gcccattgct aaattaacat caggctctga gactggcagt 7140
atatctctaa cagtggttga tgctatcttc tggaacttgc ctgctacatt gagaccactg 7200
acccatacat aggaagccca tagctctgtc ctgaactgtt aggccactgg tccagagagt 7260
gtgcatctcc tttgatcctc ataataaccc tatgagatag acacaattat tactcttact 7320
ttatagatga tgatcctgaa aacataggag tcaaggcact tgcccctagc tgggggtata 7380
ggggagcagt cccatgtagt agtagaatga aaaatgctgc tatgctgtgc ctcccccacc 7440
tttcccatgt ctgccctcta ctcatggtct atctctcctg gctcctggga gtcatggact 7500
ccacccagca ccaccaacct gacctaacca cctatctgag cctgccagcc tataacccat 7560
ctgggccctg atagctggtg gccagccctg accccacccc accctccctg gaacctctga 7620
tagacacatc tggcacacca gctcgcaaag tcaccgtgag ggtcttgtgt ttgctgagtc 7680
aaaattcctt gaaatccaag tccttagaga ctcctgctcc caaatttaca gtcatagact 7740
tcttcatggc tgtctccttt atccacagaa tgattccttt gcttcattgc cccatccatc 7800
tgatcctcct catcagtgca gcacagggcc catgagcagt agctgcagag tctcacatag 7860
gtctggcact gcctctgaca tgtccgacct taggcaaatg cttgactctt ctctagtgca 7920
tgcaaatctg acactcagtg ggcctgggtg aaggtgagaa ttttattgct gaatgagagc 7980
ctctggggac atcttgccag tcaatgagtc tcaggttcaa tttccttctc agtcttggag 8040
taacagaagc tcatgcattt aataaacgga aattttgtat tgaaatgaga gccattggaa 8100
atcatttact ccagactcct acttataaaa agagaaactg aggctcagag aagggtgggg 8160
actttctcag tatgacatgg aaatgatcag gcttggattc aaagctcctg actttctgtc 8220
tagtgtatgt gcagtgagcc ccttttcctc taactgaaag aaggaaaaaa aaatggaacc 8280
caaaatattc tacatagttt ccatgtcaca gccagggctg ggcagtctcc tgttatttct 8340
tttaaaataa atatatcatt taaatgcata aataagcaaa ccctgctcgg gaatgggagg 8400
gagagtctct ggagtccacc ccttctcggc cctggctctg cagatagtgc tatcaaagcc 8460
ctgacagagc cctgcccatt gctgggcctt ggagtgagtc agcctagtag agaggcaggg 8520
caagccatct catagctgct gagtgggaga gagaaaaggg ctcattgtct ataaactcag 8580
gtcatggcta ttcttattct cacactaaga aaaagaatga gatgtctaca tataccctgc 8640
gtcccctctt gtgtactggg gtccccaaga gctctctaaa agtgatggca aagtcattgc 8700
gctagatgcc atcccatcta ttataaacct gcatttgtct ccacacacca gtcatggaca 8760
ataaccctcc tcccaggtcc acgtgcttgt ctttgtataa tactcaagta atttcggaaa 8820
atgtattctt tcaatcttgt tctgttattc ctgtttcaat ggcttagtag aaaaagtaca 8880
tacttgtttt cccataaatt gacaatagac aatttcacat caatgtctat atgggtcgtt 8940
gtgtttgctg tgtttgcaaa aactcacaat aactttatat tgttactact ctaagaaagt 9000
tacaacatgg tgaatacaag agaaagctat tacaagtcca gaaaataaaa gttatcatct 9060
tgagggtcga caatcaacct ctggattaca aaatttgtga aagattgact ggtattctta 9120
actatgttgc tccttttacg ctatgtggat acgctgcttt aatgcctttg tatcatgcta 9180
ttgcttcccg tatggctttc attttctcct ccttgtataa atcctggttg ctgtctcttt 9240
atgaggagtt gtggcccgtt gtcaggcaac gtggcgtggt gtgcactgtg tttgctgacg 9300
caacccccac tggttggggc attgccacca cctgtcagct cctttccggg actttcgctt 9360
tccccctccc tattgccacg gcggaactca tcgccgcctg ccttgcccgc tgctggacag 9420
gggctcggct gttgggcact gacaattccg tggtgttgtc ggggaagctg acgtcctttc 9480
catggctgct cgcctgtgtt gccacctgga ttctgcgcgg gacgtccttc tgctacgtcc 9540
cttcggccct caatccagcg gaccttcctt cccgcggcct gctgccggct ctgcggcctc 9600
ttccgcgtct tcgccttcgc cctcagacga gtcggatctc cctttgggcc gcctccccgc 9660
ctggaattcg agctcggtac ctttaagacc aatgacttac aaggcagctg tagatcttag 9720
ccacttttta aaagaaaagg ggggactgga agggctaatt cactcccaac gaagacaaga 9780
tctgcttttt gcttgtactg ggtctctctg gttagaccag atctgagcct gggagctctc 9840
tggctaacta gggaacccac tgcttaagcc tcaataaagc ttgccttgag tgcttcaagt 9900
agtgtgtgcc cgtctgttgt gtgactctgg taactagaga tccctcagac ccttttagtc 9960
agtgtggaaa atctctagca gtagtagttc atgtcatctt attattcagt atttataact 10020
tgcaaagaaa tgaatatcag agagtgagag gaacttgttt attgcagctt ataatggtta 10080
caaataaagc aatagcatca caaatttcac aaataaagca tttttttcac tgcattctag 10140
ttgtggtttg tccaaactca tcaatgtatc ttatcatgtc tggctctagc tatcccgccc 10200
ctaactccgc ccatcccgcc cctaactccg cccagttccg cccattctcc gccccatggc 10260
tgactaattt tttttattta tgcagaggcc gaggccgcct cggcctctga gctattccag 10320
aagtagtgag gaggcttttt tggaggccgc tagcgtcgac cattacttat tgttttagct 10380
gtcctcatga atgtcttttc actacccatt tgcttatcct gcatctctca gccttgactc 10440
cactcagttc tcttgcttag agataccacc tttcccctga agtgttcctt ccatgtttta 10500
cggcgagatg gtttctcctc gcctggccac tcagccttag ttgtctctgt tgtcttatag 10560
aggtctactt gaagaaggaa aaacaggggg catggtttga ctgtcctgtg agcccttctt 10620
ccctgcctcc cccactcaca gtgacccgga atccctcgac atggcagtct agcactagtg 10680
cggccgcaga tctgcttcct cgctcactga ctcgctgcgc tcggtcgttc ggctgcggcg 10740
agcggtatca gctcactcaa aggcggtaat acggttatcc acagaatcag gggataacgc 10800
aggaaagaac atgtgagcaa aaggccagca aaaggccagg aaccgtaaaa a 10851
SEQ ID NO: 18
ggccgcgttg ctggcgtttt tccataggct ccgcccccct gacgagcatc acaaaaatcg 60
acgctcaagt cagaggtggc gaaacccgac aggactataa agataccagg cgtttccccc 120
tggaagctcc ctcgtgcgct ctcctgttcc gaccctgccg cttaccggat acctgtccgc 180
ctttctccct tcgggaagcg tggcgctttc tcatagctca cgctgtaggt atctcagttc 240
ggtgtaggtc gttcgctcca agctgggctg tgtgcacgaa ccccccgttc agcccgaccg 300
ctgcgcctta tccggtaact atcgtcttga gtccaacccg gtaagacacg acttatcgcc 360
actggcagca gccactggta acaggattag cagagcgagg tatgtaggcg gtgctacaga 420
gttcttgaag tggtggccta actacggcta cactagaaga acagtatttg gtatctgcgc 480
tctgctgaag ccagttacct tcggaaaaag agttggtagc tcttgatccg gcaaacaaac 540
caccgctggt agcggtggtt tttttgtttg caagcagcag attacgcgca gaaaaaaagg 600
atctcaagaa gatcctttga tcttttctac ggggtctgac gctcagtgga acgaaaactc 660
acgttaaggg attttggtca tgaagcgctt ttgaagctcg gatccgaaca aacgacccaa 720
cacccgtgcg ttttattctg tctttttatt gccgatcccc tcagaagaac tcgtcaagaa 780
ggcgatagaa ggcgatgcgc tgcgaatcgg gagcggcgat accgtaaagc acgaggaagc 840
ggtcagccca ttcgccgcca agctcttcag caatatcacg ggtagccaac gctatgtcct 900
gatagcggtc cgccacaccc agccggccac agtcgatgaa tccagaaaag cggccatttt 960
ccaccatgat attcggcaag caggcatcgc catgggtcac gacgagatcc tcgccgtcgg 1020
gcatgctcgc cttgagcctg gcgaacagtt cggctggcgc gagcccctga tgctcttcgt 1080
ccagatcatc ctgatcgaca agaccggctt ccatccgagt acgtgctcgc tcgatgcgat 1140
gtttcgcttg gtggtcgaat gggcaggtag ccggatcaag cgtatgcagc cgccgcattg 1200
catcagccat gatggatact ttctcggcag gagcaaggtg agatgacagg agatcctgcc 1260
ccggcacttc gcccaatagc agccagtccc ttcccgcttc agtgacaacg tcgagcacag 1320
ctgcgcaagg aacgcccgtc gtggccagcc acgatagccg cgctgcctcg tcttgcagtt 1380
cattcagggc accggacagg tcggtcttga caaaaagaac cgggcgcccc tgcgctgaca 1440
gccggaacac ggcggcatca gagcagccga ttgtctgttg tgcccagtca tagccgaata 1500
gcctctccac ccaagcggcc ggagaacctg cgtgcaatcc atcttgttca atcatgcgaa 1560
acgatcctca tcctgtctct tgatcagagc ttgatcccct gcgccatcag atccttggcg 1620
gcaagaaagc catccagttt actttgcagg gcttcccaac cttaccagag gcctgcgccg 1680
cggccagctg gctagcaatt cccgggttaa ctctagagac attgattatt gactagttat 1740
taatagtaat caattacggg gtcattagtt catagcccat atatggagtt ccgcgttaca 1800
taacttacgg taaatggccc gcctggctga ccgcccaacg acccccgccc attgacgtca 1860
ataatgacgt atgttcccat agtaacgcca atagggactt tccattgacg tcaatgggtg 1920
gagtatttac ggtaaactgc ccacttggca gtacatcaag tgtatcatat gccaagtacg 1980
ccccctattg acgtcaatga cggtaaatgg cccgcctggc attatgccca gtacatgacc 2040
ttatgggact ttcctacttg gcagtacatc tacgtattag tcatcgctat taccatggtg 2100
atgcggtttt ggcagtacat caatgggcgt ggatagcggt ttgactcacg gggatttcca 2160
agtctccacc ccattgacgt caatgggagt ttgttttggc accaaaatca acgggacttt 2220
ccaaaatgtc gtaacaactc cgccccattg acgcaaatgg gcggtaggcg tgtacggtgg 2280
gaggtctata taagcagagc tcgtttagtg aaccggggtc tctctggtta gaccagatct 2340
gagcctggga gctctctggc taactaggga acccactgct taagcctcaa taaagcttgc 2400
cttgagtgct tcaagtagtg tgtgcccgtc tgttgtgtga ctctggtaac tagagatccc 2460
tcagaccctt ttagtcagtg tggaaaatct ctagcagtgg cgcccgaaca gggacttgaa 2520
agcgaaaggg aaaccagagg agctctctcg acgcaggact cggcttgctg aagcgcgcac 2580
ggcaagaggc gaggggcggc gactggtgag tacgccaaaa attttgacta gcggaggcta 2640
gaaggagaga gatgggtgcg agagcgtcag tattaagcgg gggagaatta gatcgcgatg 2700
ggaaaaaatt cggttaaggc cagggggaaa gaaaaaatat aaattaaaac atatagtatg 2760
ggcaagcagg gagctagaac gattcgcagt taatcctggc ctgttagaaa catcagaagg 2820
ctgtagacaa atactgggac agctacaacc atcccttcag acaggatcag aagaacttag 2880
atcattatat aatacagtag caaccctcta ttgtgtgcat caaaggatag agataaaaga 2940
caccaaggaa gctttagaca agatagagga agagcaaaac aaaagtaaga ccaccgcaca 3000
gcaagcggcc gctgatcttc agacctggag gaggagatat gagggacaat tggagaagtg 3060
aattatataa atataaagta gtaaaaattg aaccattagg agtagcaccc accaaggcaa 3120
agagaagagt ggtgcagaga gaaaaaagag cagtgggaat aggagctttg ttccttgggt 3180
tcttgggagc agcaggaagc actatgggcg cagcgtcaat gacgctgacg gtacaggcca 3240
gacaattatt gtctggtata gtgcagcagc agaacaattt gctgagggct attgaggcgc 3300
aacagcatct gttgcaactc acagtctggg gcatcaagca gctccaggca agaatcctgg 3360
ctgtggaaag atacctaaag gatcaacagc tcctggggat ttggggttgc tctggaaaac 3420
tcatttgcac cactgctgtg ccttggaatg ctagttggag taataaatct ctggaacaga 3480
tttggaatca cacgacctgg atggagtggg acagagaaat taacaattac acaagcttaa 3540
tacactcctt aattgaagaa tcgcaaaacc agcaagaaaa gaatgaacaa gaattattgg 3600
aattagataa atgggcaagt ttgtggaatt ggtttaacat aacaaattgg ctgtggtata 3660
taaaattatt cataatgata gtaggaggct tggtaggttt aagaatagtt tttgctgtac 3720
tttctatagt gaatagagtt aggcagggat attcaccatt atcgtttcag acccacctcc 3780
caaccccgag gggacccgac aggcccgaag gaatagaaga agaaggtgga gagagagaca 3840
gagacagatc cattcgatta gtgaacggat ctcgacggta tcggttaact tttaaaagaa 3900
aaggggggat tggggggtac agtgcagggg aaagaatagt agacataata gcaacagaca 3960
tacaaactaa agaattacaa aaacaaatta caaaaattca aaattttatc gatcacgaga 4020
ctagcctcga gaagcttgat atccctaggt attgaataag aaaaatgaag ttaaggtggt 4080
tgatggtaac actatgctaa taactgcaga gccagaagca ccataaggga catgataagg 4140
gagccagcag acctctgatc tcttcctgaa tgctaatctt aaacatcctg aggaagaatg 4200
ggacttccat ttggggtggg cctatgacag ggtaataaga cagtagtgaa tatcaagcta 4260
caaaaagccg cctttcaaat tcttctcagt cctaactttt catactaagc ccagtccttc 4320
caaagcagac tgtgaaagag tgatagttcc gggagactag cactgcagat tccgggtcac 4380
tgtgagtggg ggaggcaggg aagaagggct cacaggacag tcaaaccatg acccctgttt 4440
ttccttcttc aagtagacct ctataagaca acagagacaa ctaaggctga gtggccaggc 4500
gaggagaaac catctcgccg taaaacatgg aaggaacact tcaggggaaa ggtggtatct 4560
ctaagcaaga gaactgagtg gagtcaaggc tgagagatgc aggataagca aatgggtagt 4620
gaaaagacat tcatgaggac agctaaaaca ataagtaatg taaaatacag catagcaaaa 4680
ctttaacctc caaatcaagc ctctacttga atccttttct gagggatgaa taaggcatat 4740
gcatcagggg ctgttgccaa tgtgcattag ctgtttgcag cctcaccttc tttcatggag 4800
tttaagatat agtgtatttt cccaaggttt gaactagctc ttcatttctt tatgttttaa 4860
atgcactgac ctcccacatt ccctttttag taaaatattc agaaataatt taaatacatc 4920
attgcaatga aaataaatgt tttttattag gcagaatcca gatgctcaag gcccttcata 4980
atatccccca gtttagtagt tggacttagg gaacaaagga acctttaata gaaattggac 5040
agcaagaaag cgagctcagt ggtacttgtg ggccagggcg ttggccaccc cggccaccac 5100
cttctggtac gcggcctgca ctgggggcgt gaattccttc ccgaagtggt gggccagcac 5160
gcacaccagc acgttgccca ggagctgtgg gaggaagata agaggtatga acatgattag 5220
caaaagggcc tagcttggac tcagaataat ccagccttat cccaaccata aaataaaagc 5280
agaatggtag ctggattgta gctgctatta gcaatatgaa acctcttaca tcagttacaa 5340
tttatatgca gaaatattta tatgcagaga tattgctatt gccttaaccc agaaattatc 5400
actgttattc tttagaatgg tgcaaagagg catgatacat tgtatcatta ttgccctgaa 5460
agaaagagat tagggaaagt attagaaata agataaacaa aaaagtatat taaaagaaga 5520
aagcattttt taaaattaca aatgcaaaat taccctgatt tggtcaatat gtgtacacat 5580
attaaaacat tacactttaa cccataaata tgtataatga ttatgtatca attaaaaata 5640
aaagaaaata aagtagggag attatgaata tgcaaataag cacacatata ttccaaatag 5700
taatgtacta ggcagactgt gtaaagtttt tttttaagtt acttaatgta tctcagagat 5760
atttcctttt gttatacaca atgttaaggc attaagtata atagtaaaaa ttgcggagaa 5820
gaaaaaaaaa gaaagcaaga attaaacaaa agaaaacaat tgttatgaac agcaaataaa 5880
agaaactaaa acgatcctga gacttccaca ctgatgcaat cattcgtctg tttcccattc 5940
taaactgtac cctgttactt atccccttcc tatgacatga acttaaccat agaaaagaag 6000
gggaaagaaa acatcaagcg tcccatagac tcaccctgaa gttctcgggg tcgacgtgca 6060
gcttgtcgca gtgcagctcc gacagctggg cgaaggtgcc cttgaggttg tccaggtgag 6120
ccaggccgtc ggagaaggcg ccgagcacct tcttgccgtg ggccttgacc ttagggttgc 6180
ccatcaccgc gtcgggggtg gacagatccc cgaagctctc aaaaaagcgc tgcgtccagg 6240
ggtagaccac cagcagccta agggtgggaa aatagaccaa taggcagaga gagtcagtgc 6300
ctatcagaaa cccaagagtc ttctctgtct ccacatgccc agtttctatt ggtctcctta 6360
aacctgtctt gtaaccttga taccaacctg cccagggcct ccccgccgac ctcgtcgacg 6420
ttcaccttgc cccagagggc cgtcacggcg cttttctcct ccggggttag gtgcaccatg 6480
gtgtctgttt gaggttgcta gtgaacacag ttgtgtcaga agcaaatgta agcaatagat 6540
ggctctgccc tgacttttat gcccagccct ggctcctgcc ctccctgctc ctgggagtag 6600
attggccaac cctagggtgt ggctccacag ggtgaggtct aagtgatgac agccgtacct 6660
gtccttggct cttctggcac tggcttagga gttggacttc aaaccctcag ccctccctct 6720
aagatatatc tcttggcccc ataccatcag tacaaattgc tactaaaaac atcctccttt 6780
gcaagtgtat ttacgtatct agaatatgtc acattctgtc tcaggcatcc attttcttta 6840
tgatgccgtt tgaggtggag ttttagtcag gtggtcagct tctccttttt tttgccatct 6900
gccctgtaag catcctgctg gggacccaga taggagtcat cactctaggc tgagaacatc 6960
tgggcacaca ccctaagcct cagcatgact catcatgact cagcattgct gtgcttgagc 7020
cagaaggttt gcttagaagg ttacacagaa ccagaaggcg ggggtggggc actgaccccg 7080
acaggggcct ggccagaact gctcatgctt ggactatggg aggtcactaa tggagacaca 7140
cagaaatgta acaggaacta aggaaaaact gaagcttatt taatcagaga tgaggatgct 7200
ggaagggata gagggagctg agcttgtaaa aagtatagta atcattcagc aaatggtttt 7260
gaagcacctg ctggatgcta aacactattt tcagtgcttg aatcataaat aagaataaaa 7320
catgtatctt attccccaca agagtccaag taaaaaataa cagttaatta taatgtgctc 7380
tgtcccccag gctggagtgc agtggcacga tctcagctca ctgcaacctc cgcctcccgg 7440
gctggttaga aggttctact ggaggagggt cccagcccat tgctaaatta acatcaggct 7500
ctgagactgg cagtatatct ctaacagtgg ttgatgctat cttctggaac ttgcctgcta 7560
cattgagacc actgacccat acataggaag cccatagctc tgtcctgaac tgttaggcca 7620
ctggtccaga gagtgtgcat ctcctttgat cctcataata accctatgag atagacacaa 7680
ttattactct tactttatag atgatgatcc tgaaaacata ggagtcaagg cacttgcccc 7740
tagctggggg tataggggag cagtcccatg tagtagtaga atgaaaaatg ctgctatgct 7800
gtgcctcccc cacctttccc atgtctgccc tctactcatg gtctatctct cctggctcct 7860
gggagtcatg gactccaccc agcaccacca acctgaccta accacctatc tgagcctgcc 7920
agcctataac ccatctgggc cctgatagct ggtggccagc cctgacccca ccccaccctc 7980
cctggaacct ctgatagaca catctggcac accagctcgc aaagtcaccg tgagggtctt 8040
gtgtttgctg agtcaaaatt ccttgaaatc caagtcctta gagactcctg ctcccaaatt 8100
tacagtcata gacttcttca tggctgtctc ctttatccac agaatgattc ctttgcttca 8160
ttgccccatc catctgatcc tcctcatcag tgcagcacag ggcccatgag cagtagctgc 8220
agagtctcac ataggtctgg cactgcctct gacatgtccg accttaggca aatgcttgac 8280
tcttctctag tgcatgcaaa tctgacactc agtgggcctg ggtgaaggtg agaattttat 8340
tgctgaatga gagcctctgg ggacatcttg ccagtcaatg agtctcaggt tcaatttcct 8400
tctcagtctt ggagtaacag aagctcatgc atttaataaa cggaaatttt gtattgaaat 8460
gagagccatt ggaaatcatt tactccagac tcctacttat aaaaagagaa actgaggctc 8520
agagaagggt ggggactttc tcagtatgac atggaaatga tcaggcttgg attcaaagct 8580
cctgactttc tgtctagtgt atgtgcagtg agcccctttt cctctaactg aaagaaggaa 8640
aaaaaaatgg aacccaaaat attctacata gtttccatgt cacagccagg gctgggcagt 8700
ctcctgttat ttcttttaaa ataaatatat catttaaatg cataaataag caaaccctgc 8760
tcgggaatgg gagggagagt ctctggagtc caccccttct cggccctggc tctgcagata 8820
gtgctatcaa agccctgaca gagccctgcc cattgctggg ccttggagtg agtcagccta 8880
gtagagaggc agggcaagcc atctcatagc tgctgagtgg gagagagaaa agggctcatt 8940
gtctataaac tcaggtcatg gctattctta ttctcacact aagaaaaaga atgagatgtc 9000
tacatatacc ctgcgtcccc tcttgtgtac tggggtcccc aagagctctc taaaagtgat 9060
ggcaaagtca ttgcgctaga tgccatccca tctattataa acctgcattt gtctccacac 9120
accagtcatg gacaataacc ctcctcccag gtccacgtgc ttgtctttgt ataatactca 9180
agtaatttcg gaaaatgtat tctttcaatc ttgttctgtt attcctgttt caatggctta 9240
gtagaaaaag tacatacttg ttttcccata aattgacaat agacaatttc acatcaatgt 9300
ctatatgggt cgttgtgttt gctgtgtttg caaaaactca caataacttt atattgttac 9360
tactctaaga aagttacaac atggtgaata caagagaaag ctattacaag tccagaaaat 9420
aaaagttatc atcttgaggg tcgacaatca acctctggat tacaaaattt gtgaaagatt 9480
gactggtatt cttaactatg ttgctccttt tacgctatgt ggatacgctg ctttaatgcc 9540
tttgtatcat gctattgctt cccgtatggc tttcattttc tcctccttgt ataaatcctg 9600
gttgctgtct ctttatgagg agttgtggcc cgttgtcagg caacgtggcg tggtgtgcac 9660
tgtgtttgct gacgcaaccc ccactggttg gggcattgcc accacctgtc agctcctttc 9720
cgggactttc gctttccccc tccctattgc cacggcggaa ctcatcgccg cctgccttgc 9780
ccgctgctgg acaggggctc ggctgttggg cactgacaat tccgtggtgt tgtcggggaa 9840
gctgacgtcc tttccatggc tgctcgcctg tgttgccacc tggattctgc gcgggacgtc 9900
cttctgctac gtcccttcgg ccctcaatcc agcggacctt ccttcccgcg gcctgctgcc 9960
ggctctgcgg cctcttccgc gtcttcgcct tcgccctcag acgagtcgga tctccctttg 10020
ggccgcctcc ccgcctggaa ttcgagctcg gtacctttaa gaccaatgac ttacaaggca 10080
gctgtagatc ttagccactt tttaaaagaa aaggggggac tggaagggct aattcactcc 10140
caacgaagac aagatctgct ttttgcttgt actgggtctc tctggttaga ccagatctga 10200
gcctgggagc tctctggcta actagggaac ccactgctta agcctcaata aagcttgcct 10260
tgagtgcttc aagtagtgtg tgcccgtctg ttgtgtgact ctggtaacta gagatccctc 10320
agaccctttt agtcagtgtg gaaaatctct agcagtagta gttcatgtca tcttattatt 10380
cagtatttat aacttgcaaa gaaatgaata tcagagagtg agaggaactt gtttattgca 10440
gcttataatg gttacaaata aagcaatagc atcacaaatt tcacaaataa agcatttttt 10500
tcactgcatt ctagttgtgg tttgtccaaa ctcatcaatg tatcttatca tgtctggctc 10560
tagctatccc gcccctaact ccgcccatcc cgcccctaac tccgcccagt tccgcccatt 10620
ctccgcccca tggctgacta atttttttta tttatgcaga ggccgaggcc gcctcggcct 10680
ctgagctatt ccagaagtag tgaggaggct tttttggagg ccgctagcgt cgaccattac 10740
ttattgtttt agctgtcctc atgaatgtct tttcactacc catttgctta tcctgcatct 10800
ctcagccttg actccactca gttctcttgc ttagagatac cacctttccc ctgaagtgtt 10860
ccttccatgt tttacggcga gatggtttct cctcgcctgg ccactcagcc ttagttgtct 10920
ctgttgtctt atagaggtct acttgaagaa ggaaaaacag ggggcatggt ttgactgtcc 10980
tgtgagccct tcttccctgc ctcccccact cacagtgacc cggaatccct cgacatggca 11040
gtctagcact agtgcggccg cagatctgct tcctcgctca ctgactcgct gcgctcggtc 11100
gttcggctgc ggcgagcggt atcagctcac tcaaaggcgg taatacggtt atccacagaa 11160
tcaggggata acgcaggaaa gaacatgtga gcaaaaggcc agcaaaaggc caggaaccgt 11220
aaaaa 11225
SEQ ID NO: 19
ggccgcgttg ctggcgtttt tccataggct ccgcccccct gacgagcatc acaaaaatcg 60
acgctcaagt cagaggtggc gaaacccgac aggactataa agataccagg cgtttccccc 120
tggaagctcc ctcgtgcgct ctcctgttcc gaccctgccg cttaccggat acctgtccgc 180
ctttctccct tcgggaagcg tggcgctttc tcatagctca cgctgtaggt atctcagttc 240
ggtgtaggtc gttcgctcca agctgggctg tgtgcacgaa ccccccgttc agcccgaccg 300
ctgcgcctta tccggtaact atcgtcttga gtccaacccg gtaagacacg acttatcgcc 360
actggcagca gccactggta acaggattag cagagcgagg tatgtaggcg gtgctacaga 420
gttcttgaag tggtggccta actacggcta cactagaaga acagtatttg gtatctgcgc 480
tctgctgaag ccagttacct tcggaaaaag agttggtagc tcttgatccg gcaaacaaac 540
caccgctggt agcggtggtt tttttgtttg caagcagcag attacgcgca gaaaaaaagg 600
atctcaagaa gatcctttga tcttttctac ggggtctgac gctcagtgga acgaaaactc 660
acgttaaggg attttggtca tgaagcgctt ttgaagctcg gatccgaaca aacgacccaa 720
cacccgtgcg ttttattctg tctttttatt gccgatcccc tcagaagaac tcgtcaagaa 780
ggcgatagaa ggcgatgcgc tgcgaatcgg gagcggcgat accgtaaagc acgaggaagc 840
ggtcagccca ttcgccgcca agctcttcag caatatcacg ggtagccaac gctatgtcct 900
gatagcggtc cgccacaccc agccggccac agtcgatgaa tccagaaaag cggccatttt 960
ccaccatgat attcggcaag caggcatcgc catgggtcac gacgagatcc tcgccgtcgg 1020
gcatgctcgc cttgagcctg gcgaacagtt cggctggcgc gagcccctga tgctcttcgt 1080
ccagatcatc ctgatcgaca agaccggctt ccatccgagt acgtgctcgc tcgatgcgat 1140
gtttcgcttg gtggtcgaat gggcaggtag ccggatcaag cgtatgcagc cgccgcattg 1200
catcagccat gatggatact ttctcggcag gagcaaggtg agatgacagg agatcctgcc 1260
ccggcacttc gcccaatagc agccagtccc ttcccgcttc agtgacaacg tcgagcacag 1320
ctgcgcaagg aacgcccgtc gtggccagcc acgatagccg cgctgcctcg tcttgcagtt 1380
cattcagggc accggacagg tcggtcttga caaaaagaac cgggcgcccc tgcgctgaca 1440
gccggaacac ggcggcatca gagcagccga ttgtctgttg tgcccagtca tagccgaata 1500
gcctctccac ccaagcggcc ggagaacctg cgtgcaatcc atcttgttca atcatgcgaa 1560
acgatcctca tcctgtctct tgatcagagc ttgatcccct gcgccatcag atccttggcg 1620
gcaagaaagc catccagttt actttgcagg gcttcccaac cttaccagag gcctgcgccg 1680
cggccagctg gctagcaatt cccgggttaa ctctagagac attgattatt gactagttat 1740
taatagtaat caattacggg gtcattagtt catagcccat atatggagtt ccgcgttaca 1800
taacttacgg taaatggccc gcctggctga ccgcccaacg acccccgccc attgacgtca 1860
ataatgacgt atgttcccat agtaacgcca atagggactt tccattgacg tcaatgggtg 1920
gagtatttac ggtaaactgc ccacttggca gtacatcaag tgtatcatat gccaagtacg 1980
ccccctattg acgtcaatga cggtaaatgg cccgcctggc attatgccca gtacatgacc 2040
ttatgggact ttcctacttg gcagtacatc tacgtattag tcatcgctat taccatggtg 2100
atgcggtttt ggcagtacat caatgggcgt ggatagcggt ttgactcacg gggatttcca 2160
agtctccacc ccattgacgt caatgggagt ttgttttggc accaaaatca acgggacttt 2220
ccaaaatgtc gtaacaactc cgccccattg acgcaaatgg gcggtaggcg tgtacggtgg 2280
gaggtctata taagcagagc tcgtttagtg aaccggggtc tctctggtta gaccagatct 2340
gagcctggga gctctctggc taactaggga acccactgct taagcctcaa taaagcttgc 2400
cttgagtgct tcaagtagtg tgtgcccgtc tgttgtgtga ctctggtaac tagagatccc 2460
tcagaccctt ttagtcagtg tggaaaatct ctagcagtgg cgcccgaaca gggacttgaa 2520
agcgaaaggg aaaccagagg agctctctcg acgcaggact cggcttgctg aagcgcgcac 2580
ggcaagaggc gaggggcggc gactggtgag tacgccaaaa attttgacta gcggaggcta 2640
gaaggagaga gatgggtgcg agagcgtcag tattaagcgg gggagaatta gatcgcgatg 2700
ggaaaaaatt cggttaaggc cagggggaaa gaaaaaatat aaattaaaac atatagtatg 2760
ggcaagcagg gagctagaac gattcgcagt taatcctggc ctgttagaaa catcagaagg 2820
ctgtagacaa atactgggac agctacaacc atcccttcag acaggatcag aagaacttag 2880
atcattatat aatacagtag caaccctcta ttgtgtgcat caaaggatag agataaaaga 2940
caccaaggaa gctttagaca agatagagga agagcaaaac aaaagtaaga ccaccgcaca 3000
gcaagcggcc gctgatcttc agacctggag gaggagatat gagggacaat tggagaagtg 3060
aattatataa atataaagta gtaaaaattg aaccattagg agtagcaccc accaaggcaa 3120
agagaagagt ggtgcagaga gaaaaaagag cagtgggaat aggagctttg ttccttgggt 3180
tcttgggagc agcaggaagc actatgggcg cagcgtcaat gacgctgacg gtacaggcca 3240
gacaattatt gtctggtata gtgcagcagc agaacaattt gctgagggct attgaggcgc 3300
aacagcatct gttgcaactc acagtctggg gcatcaagca gctccaggca agaatcctgg 3360
ctgtggaaag atacctaaag gatcaacagc tcctggggat ttggggttgc tctggaaaac 3420
tcatttgcac cactgctgtg ccttggaatg ctagttggag taataaatct ctggaacaga 3480
tttggaatca cacgacctgg atggagtggg acagagaaat taacaattac acaagcttaa 3540
tacactcctt aattgaagaa tcgcaaaacc agcaagaaaa gaatgaacaa gaattattgg 3600
aattagataa atgggcaagt ttgtggaatt ggtttaacat aacaaattgg ctgtggtata 3660
taaaattatt cataatgata gtaggaggct tggtaggttt aagaatagtt tttgctgtac 3720
tttctatagt gaatagagtt aggcagggat attcaccatt atcgtttcag acccacctcc 3780
caaccccgag gggacccgac aggcccgaag gaatagaaga agaaggtgga gagagagaca 3840
gagacagatc cattcgatta gtgaacggat ctcgacggta tcggttaact tttaaaagaa 3900
aaggggggat tggggggtac agtgcagggg aaagaatagt agacataata gcaacagaca 3960
tacaaactaa agaattacaa aaacaaatta caaaaattca aaattttatc gatcacgaga 4020
ctagcctcga gaagcttgat atccctaggt attgaataag aaaaatgaag ttaaggtggt 4080
tgatggtaac actatgctaa taactgcaga gccagaagca ccataaggga catgataagg 4140
gagccagcag acctctgatc tcttcctgaa tgctaatctt aaacatcctg aggaagaatg 4200
ggacttccat ttggggtggg cctatgacag ggtaataaga cagtagtgaa tatcaagcta 4260
caaaaagccg cctttcaaat tcttctcagt cctaactttt catactaagc ccagtccttc 4320
caaagcagac tgtgaaagag tgatagttcc gggagactag cactgcagat tccgggtcac 4380
tgtgagtggg ggaggcaggg aagaagggct cacaggacag tcaaaccatg acccctgttt 4440
ttccttcttc aagtagacct ctataagaca acagagacaa ctaaggctga gtggccaggc 4500
gaggagaaac catctcgccg taaaacatgg aaggaacact tcaggggaaa ggtggtatct 4560
ctaagcaaga gaactgagtg gagtcaaggc tgagagatgc aggataagca aatgggtagt 4620
gaaaagacat tcatgaggac agctaaaaca ataagtaatg taaaatacag catagcaaaa 4680
ctttaacctc caaatcaagc ctctacttga atccttttct gagggatgaa taaggcatat 4740
gcatcagggg ctgttgccaa tgtgcattag ctgtttgcag cctcaccttc tttcatggag 4800
tttaagatat agtgtatttt cccaaggttt gaactagctc ttcatttctt tatgttttaa 4860
atgcactgac ctcccacatt ccctttttag taaaatattc agaaataatt taaatacatc 4920
attgcaatga aaataaatgt tttttattag gcagaatcca gatgctcaag gcccttcata 4980
atatccccca gtttagtagt tggacttagg gaacaaagga acctttaata gaaattggac 5040
agcaagaaag cgagctcagt ggtacttgtg ggccagggcg ttggccaccc cggccaccac 5100
cttctggtac gcggcctgca ctgggggcgt gaattccttc ccgaagtggt gggccagcac 5160
gcacaccagc acgttgccca ggagctgtgg gaggaagata agaggtatga acatgattag 5220
caaaagggcc tagcttggac tcagaataat ccagccttat cccaaccata aaataaaagc 5280
agaatggtag ctggattgta gctgctatta gcaatatgaa acctcttaca tcagttacaa 5340
tttatatgca gaaatattta tatgcagaga tattgctatt gccttaaccc agaaattatc 5400
actgttattc tttagaatgg tgcaaagagg catgatacat tgtatcatta ttgccctgaa 5460
agaaagagat tagggaaagt attagaaata agataaacaa aaaagtatat taaaagaaga 5520
aagcattttt taaaattaca aatgcaaaat taccctgatt tggtcaatat gtgtacacat 5580
attaaaacat tacactttaa cccataaata tgtataatga ttatgtatca attaaaaata 5640
aaagaaaata aagtagggag attatgaata tgcaaataag cacacatata ttccaaatag 5700
taatgtacta ggcagactgt gtaaagtttt tttttaagtt acttaatgta tctcagagat 5760
atttcctttt gttatacaca atgttaaggc attaagtata atagtaaaaa ttgcggagaa 5820
gaaaaaaaaa gaaagcaaga attaaacaaa agaaaacaat tgttatgaac agcaaataaa 5880
agaaactaaa acgatcctga gacttccaca ctgatgcaat cattcgtctg tttcccattc 5940
taaactgtac cctgttactt atccccttcc tatgacatga acttaaccat agaaaagaag 6000
gggaaagaaa acatcaagcg tcccatagac tcaccctgaa gttctcgggg tcgacgtgca 6060
gcttgtcgca gtgcagctcc gacagtgtgg cgaaggtgcc cttgaggttg tccaggtgag 6120
ccaggccgtc ggagaaggcg ccgagcacct tcttgccgtg ggccttgacc ttagggttgc 6180
ccatcaccgc gtcgggggtg gacagatccc cgaagctctc aaaaaagcgc tgcgtccagg 6240
ggtagaccac cagcagccta agggtgggaa aatagaccaa taggcagaga gagtcagtgc 6300
ctatcagaaa cccaagagtc ttctctgtct ccacatgccc agtttctatt ggtctcctta 6360
aacctgtctt gtaaccttga taccaacctg cccagggcct ccccgccgac ctcgtcgacg 6420
ttcaccttgc cccagagggc cgtcacggcg cttttctcct ccggggttag gtgcaccatg 6480
gtgtctgttt gaggttgcta gtgaacacag ttgtgtcaga agcaaatgta agcaatagat 6540
ggctctgccc tgacttttat gcccagccct ggctcctgcc ctccctgctc ctgggagtag 6600
attggccaac cctagggtgt ggctccacag ggtgaggtct aagtgatgac agccgtacct 6660
gtccttggct cttctggcac tggcttagga gttggacttc aaaccctcag ccctccctct 6720
aagatatatc tcttggcccc ataccatcag tacaaattgc tactaaaaac atcctccttt 6780
gcaagtgtat ttacgtatct agaatatgtc acattctgtc tcaggcatcc attttcttta 6840
tgatgccgtt tgaggtggag ttttagtcag gtggtcagct tctccttttt tttgccatct 6900
gccctgtaag catcctgctg gggacccaga taggagtcat cactctaggc tgagaacatc 6960
tgggcacaca ccctaagcct cagcatgact catcatgact cagcattgct gtgcttgagc 7020
cagaaggttt gcttagaagg ttacacagaa ccagaaggcg ggggtggggc actgaccccg 7080
acaggggcct ggccagaact gctcatgctt ggactatggg aggtcactaa tggagacaca 7140
cagaaatgta acaggaacta aggaaaaact gaagcttatt taatcagaga tgaggatgct 7200
ggaagggata gagggagctg agcttgtaaa aagtatagta atcattcagc aaatggtttt 7260
gaagcacctg ctggatgcta aacactattt tcagtgcttg aatcataaat aagaataaaa 7320
catgtatctt attccccaca agagtccaag taaaaaataa cagttaatta taatgtgctc 7380
tgtcccccag gctggagtgc agtggcacga tctcagctca ctgcaacctc cgcctcccgg 7440
gctggttaga aggttctact ggaggagggt cccagcccat tgctaaatta acatcaggct 7500
ctgagactgg cagtatatct ctaacagtgg ttgatgctat cttctggaac ttgcctgcta 7560
cattgagacc actgacccat acataggaag cccatagctc tgtcctgaac tgttaggcca 7620
ctggtccaga gagtgtgcat ctcctttgat cctcataata accctatgag atagacacaa 7680
ttattactct tactttatag atgatgatcc tgaaaacata ggagtcaagg cacttgcccc 7740
tagctggggg tataggggag cagtcccatg tagtagtaga atgaaaaatg ctgctatgct 7800
gtgcctcccc cacctttccc atgtctgccc tctactcatg gtctatctct cctggctcct 7860
gggagtcatg gactccaccc agcaccacca acctgaccta accacctatc tgagcctgcc 7920
agcctataac ccatctgggc cctgatagct ggtggccagc cctgacccca ccccaccctc 7980
cctggaacct ctgatagaca catctggcac accagctcgc aaagtcaccg tgagggtctt 8040
gtgtttgctg agtcaaaatt ccttgaaatc caagtcctta gagactcctg ctcccaaatt 8100
tacagtcata gacttcttca tggctgtctc ctttatccac agaatgattc ctttgcttca 8160
ttgccccatc catctgatcc tcctcatcag tgcagcacag ggcccatgag cagtagctgc 8220
agagtctcac ataggtctgg cactgcctct gacatgtccg accttaggca aatgcttgac 8280
tcttctctag tgcatgcaaa tctgacactc agtgggcctg ggtgaaggtg agaattttat 8340
tgctgaatga gagcctctgg ggacatcttg ccagtcaatg agtctcaggt tcaatttcct 8400
tctcagtctt ggagtaacag aagctcatgc atttaataaa cggaaatttt gtattgaaat 8460
gagagccatt ggaaatcatt tactccagac tcctacttat aaaaagagaa actgaggctc 8520
agagaagggt ggggactttc tcagtatgac atggaaatga tcaggcttgg attcaaagct 8580
cctgactttc tgtctagtgt atgtgcagtg agcccctttt cctctaactg aaagaaggaa 8640
aaaaaaatgg aacccaaaat attctacata gtttccatgt cacagccagg gctgggcagt 8700
ctcctgttat ttcttttaaa ataaatatat catttaaatg cataaataag caaaccctgc 8760
tcgggaatgg gagggagagt ctctggagtc caccccttct cggccctggc tctgcagata 8820
gtgctatcaa agccctgaca gagccctgcc cattgctggg ccttggagtg agtcagccta 8880
gtagagaggc agggcaagcc atctcatagc tgctgagtgg gagagagaaa agggctcatt 8940
gtctataaac tcaggtcatg gctattctta ttctcacact aagaaaaaga atgagatgtc 9000
tacatatacc ctgcgtcccc tcttgtgtac tggggtcccc aagagctctc taaaagtgat 9060
ggcaaagtca ttgcgctaga tgccatccca tctattataa acctgcattt gtctccacac 9120
accagtcatg gacaataacc ctcctcccag gtccacgtgc ttgtctttgt ataatactca 9180
agtaatttcg gaaaatgtat tctttcaatc ttgttctgtt attcctgttt caatggctta 9240
gtagaaaaag tacatacttg ttttcccata aattgacaat agacaatttc acatcaatgt 9300
ctatatgggt cgttgtgttt gctgtgtttg caaaaactca caataacttt atattgttac 9360
tactctaaga aagttacaac atggtgaata caagagaaag ctattacaag tccagaaaat 9420
aaaagttatc atcttgaggg tcgacaatca acctctggat tacaaaattt gtgaaagatt 9480
gactggtatt cttaactatg ttgctccttt tacgctatgt ggatacgctg ctttaatgcc 9540
tttgtatcat gctattgctt cccgtatggc tttcattttc tcctccttgt ataaatcctg 9600
gttgctgtct ctttatgagg agttgtggcc cgttgtcagg caacgtggcg tggtgtgcac 9660
tgtgtttgct gacgcaaccc ccactggttg gggcattgcc accacctgtc agctcctttc 9720
cgggactttc gctttccccc tccctattgc cacggcggaa ctcatcgccg cctgccttgc 9780
ccgctgctgg acaggggctc ggctgttggg cactgacaat tccgtggtgt tgtcggggaa 9840
gctgacgtcc tttccatggc tgctcgcctg tgttgccacc tggattctgc gcgggacgtc 9900
cttctgctac gtcccttcgg ccctcaatcc agcggacctt ccttcccgcg gcctgctgcc 9960
ggctctgcgg cctcttccgc gtcttcgcct tcgccctcag acgagtcgga tctccctttg 10020
ggccgcctcc ccgcctggaa ttcgagctcg gtacctttaa gaccaatgac ttacaaggca 10080
gctgtagatc ttagccactt tttaaaagaa aaggggggac tggaagggct aattcactcc 10140
caacgaagac aagatctgct ttttgcttgt actgggtctc tctggttaga ccagatctga 10200
gcctgggagc tctctggcta actagggaac ccactgctta agcctcaata aagcttgcct 10260
tgagtgcttc aagtagtgtg tgcccgtctg ttgtgtgact ctggtaacta gagatccctc 10320
agaccctttt agtcagtgtg gaaaatctct agcagtagta gttcatgtca tcttattatt 10380
cagtatttat aacttgcaaa gaaatgaata tcagagagtg agaggaactt gtttattgca 10440
gcttataatg gttacaaata aagcaatagc atcacaaatt tcacaaataa agcatttttt 10500
tcactgcatt ctagttgtgg tttgtccaaa ctcatcaatg tatcttatca tgtctggctc 10560
tagctatccc gcccctaact ccgcccatcc cgcccctaac tccgcccagt tccgcccatt 10620
ctccgcccca tggctgacta atttttttta tttatgcaga ggccgaggcc gcctcggcct 10680
ctgagctatt ccagaagtag tgaggaggct tttttggagg ccgctagcgt cgaccattac 10740
ttattgtttt agctgtcctc atgaatgtct tttcactacc catttgctta tcctgcatct 10800
ctcagccttg actccactca gttctcttgc ttagagatac cacctttccc ctgaagtgtt 10860
ccttccatgt tttacggcga gatggtttct cctcgcctgg ccactcagcc ttagttgtct 10920
ctgttgtctt atagaggtct acttgaagaa ggaaaaacag ggggcatggt ttgactgtcc 10980
tgtgagccct tcttccctgc ctcccccact cacagtgacc cggaatccct cgacatggca 11040
gtctagcact agtgcggccg cagatctgct tcctcgctca ctgactcgct gcgctcggtc 11100
gttcggctgc ggcgagcggt atcagctcac tcaaaggcgg taatacggtt atccacagaa 11160
tcaggggata acgcaggaaa gaacatgtga gcaaaaggcc agcaaaaggc caggaaccgt 11220
aaaaa 11225
SEQ ID NO: 20
ggccgcgttg ctggcgtttt tccataggct ccgcccccct gacgagcatc acaaaaatcg 60
acgctcaagt cagaggtggc gaaacccgac aggactataa agataccagg cgtttccccc 120
tggaagctcc ctcgtgcgct ctcctgttcc gaccctgccg cttaccggat acctgtccgc 180
ctttctccct tcgggaagcg tggcgctttc tcatagctca cgctgtaggt atctcagttc 240
ggtgtaggtc gttcgctcca agctgggctg tgtgcacgaa ccccccgttc agcccgaccg 300
ctgcgcctta tccggtaact atcgtcttga gtccaacccg gtaagacacg acttatcgcc 360
actggcagca gccactggta acaggattag cagagcgagg tatgtaggcg gtgctacaga 420
gttcttgaag tggtggccta actacggcta cactagaaga acagtatttg gtatctgcgc 480
tctgctgaag ccagttacct tcggaaaaag agttggtagc tcttgatccg gcaaacaaac 540
caccgctggt agcggtggtt tttttgtttg caagcagcag attacgcgca gaaaaaaagg 600
atctcaagaa gatcctttga tcttttctac ggggtctgac gctcagtgga acgaaaactc 660
acgttaaggg attttggtca tgaagcgctt ttgaagctcg gatccgaaca aacgacccaa 720
cacccgtgcg ttttattctg tctttttatt gccgatcccc tcagaagaac tcgtcaagaa 780
ggcgatagaa ggcgatgcgc tgcgaatcgg gagcggcgat accgtaaagc acgaggaagc 840
ggtcagccca ttcgccgcca agctcttcag caatatcacg ggtagccaac gctatgtcct 900
gatagcggtc cgccacaccc agccggccac agtcgatgaa tccagaaaag cggccatttt 960
ccaccatgat attcggcaag caggcatcgc catgggtcac gacgagatcc tcgccgtcgg 1020
gcatgctcgc cttgagcctg gcgaacagtt cggctggcgc gagcccctga tgctcttcgt 1080
ccagatcatc ctgatcgaca agaccggctt ccatccgagt acgtgctcgc tcgatgcgat 1140
gtttcgcttg gtggtcgaat gggcaggtag ccggatcaag cgtatgcagc cgccgcattg 1200
catcagccat gatggatact ttctcggcag gagcaaggtg agatgacagg agatcctgcc 1260
ccggcacttc gcccaatagc agccagtccc ttcccgcttc agtgacaacg tcgagcacag 1320
ctgcgcaagg aacgcccgtc gtggccagcc acgatagccg cgctgcctcg tcttgcagtt 1380
cattcagggc accggacagg tcggtcttga caaaaagaac cgggcgcccc tgcgctgaca 1440
gccggaacac ggcggcatca gagcagccga ttgtctgttg tgcccagtca tagccgaata 1500
gcctctccac ccaagcggcc ggagaacctg cgtgcaatcc atcttgttca atcatgcgaa 1560
acgatcctca tcctgtctct tgatcagagc ttgatcccct gcgccatcag atccttggcg 1620
gcaagaaagc catccagttt actttgcagg gcttcccaac cttaccagag gcctgcgccg 1680
cggccagctg gctagcaatt cccgggttaa ctctagagac attgattatt gactagttat 1740
taatagtaat caattacggg gtcattagtt catagcccat atatggagtt ccgcgttaca 1800
taacttacgg taaatggccc gcctggctga ccgcccaacg acccccgccc attgacgtca 1860
ataatgacgt atgttcccat agtaacgcca atagggactt tccattgacg tcaatgggtg 1920
gagtatttac ggtaaactgc ccacttggca gtacatcaag tgtatcatat gccaagtacg 1980
ccccctattg acgtcaatga cggtaaatgg cccgcctggc attatgccca gtacatgacc 2040
ttatgggact ttcctacttg gcagtacatc tacgtattag tcatcgctat taccatggtg 2100
atgcggtttt ggcagtacat caatgggcgt ggatagcggt ttgactcacg gggatttcca 2160
agtctccacc ccattgacgt caatgggagt ttgttttggc accaaaatca acgggacttt 2220
ccaaaatgtc gtaacaactc cgccccattg acgcaaatgg gcggtaggcg tgtacggtgg 2280
gaggtctata taagcagagc tcgtttagtg aaccggggtc tctctggtta gaccagatct 2340
gagcctggga gctctctggc taactaggga acccactgct taagcctcaa taaagcttgc 2400
cttgagtgct tcaagtagtg tgtgcccgtc tgttgtgtga ctctggtaac tagagatccc 2460
tcagaccctt ttagtcagtg tggaaaatct ctagcagtgg cgcccgaaca gggacttgaa 2520
agcgaaaggg aaaccagagg agctctctcg acgcaggact cggcttgctg aagcgcgcac 2580
ggcaagaggc gaggggcggc gactggtgag tacgccaaaa attttgacta gcggaggcta 2640
gaaggagaga gatgggtgcg agagcgtcag tattaagcgg gggagaatta gatcgcgatg 2700
ggaaaaaatt cggttaaggc cagggggaaa gaaaaaatat aaattaaaac atatagtatg 2760
ggcaagcagg gagctagaac gattcgcagt taatcctggc ctgttagaaa catcagaagg 2820
ctgtagacaa atactgggac agctacaacc atcccttcag acaggatcag aagaacttag 2880
atcattatat aatacagtag caaccctcta ttgtgtgcat caaaggatag agataaaaga 2940
caccaaggaa gctttagaca agatagagga agagcaaaac aaaagtaaga ccaccgcaca 3000
gcaagcggcc gctgatcttc agacctggag gaggagatat gagggacaat tggagaagtg 3060
aattatataa atataaagta gtaaaaattg aaccattagg agtagcaccc accaaggcaa 3120
agagaagagt ggtgcagaga gaaaaaagag cagtgggaat aggagctttg ttccttgggt 3180
tcttgggagc agcaggaagc actatgggcg cagcgtcaat gacgctgacg gtacaggcca 3240
gacaattatt gtctggtata gtgcagcagc agaacaattt gctgagggct attgaggcgc 3300
aacagcatct gttgcaactc acagtctggg gcatcaagca gctccaggca agaatcctgg 3360
ctgtggaaag atacctaaag gatcaacagc tcctggggat ttggggttgc tctggaaaac 3420
tcatttgcac cactgctgtg ccttggaatg ctagttggag taataaatct ctggaacaga 3480
tttggaatca cacgacctgg atggagtggg acagagaaat taacaattac acaagcttaa 3540
tacactcctt aattgaagaa tcgcaaaacc agcaagaaaa gaatgaacaa gaattattgg 3600
aattagataa atgggcaagt ttgtggaatt ggtttaacat aacaaattgg ctgtggtata 3660
taaaattatt cataatgata gtaggaggct tggtaggttt aagaatagtt tttgctgtac 3720
tttctatagt gaatagagtt aggcagggat attcaccatt atcgtttcag acccacctcc 3780
caaccccgag gggacccgac aggcccgaag gaatagaaga agaaggtgga gagagagaca 3840
gagacagatc cattcgatta gtgaacggat ctcgacggta tcggttaact tttaaaagaa 3900
aaggggggat tggggggtac agtgcagggg aaagaatagt agacataata gcaacagaca 3960
tacaaactaa agaattacaa aaacaaatta caaaaattca aaattttatc gatcacgaga 4020
ctagcctcga gaagcttgat atccctaggt attgaataag aaaaatgaag ttaaggtggt 4080
tgatggtaac actatgctaa taactgcaga gccagaagca ccataaggga catgataagg 4140
gagccagcag acctctgatc tcttcctgaa tgctaatctt aaacatcctg aggaagaatg 4200
ggacttccat ttggggtggg cctatgacag ggtaataaga cagtagtgaa tatcaagcta 4260
caaaaagccg cctttcaaat tcttctcagt cctaactttt catactaagc ccagtccttc 4320
caaagcagac tgtgaaagag tgatagttcc gggagactag cactgcagat tccgggtcac 4380
tgtgagtggg ggaggcaggg aagaagggct cacaggacag tcaaaccatg acccctgttt 4440
ttccttcttc aagtagacct ctataagaca acagagacaa ctaaggctga gtggccaggc 4500
gaggagaaac catctcgccg taaaacatgg aaggaacact tcaggggaaa ggtggtatct 4560
ctaagcaaga gaactgagtg gagtcaaggc tgagagatgc aggataagca aatgggtagt 4620
gaaaagacat tcatgaggac agctaaaaca ataagtaatg taaaatacag catagcaaaa 4680
ctttaacctc caaatcaagc ctctacttga atccttttct gagggatgaa taaggcatat 4740
gcatcagggg ctgttgccaa tgtgcattag ctgtttgcag cctcaccttc tttcatggag 4800
tttaagatat agtgtatttt cccaaggttt gaactagctc ttcatttctt tatgttttaa 4860
atgcactgac ctcccacatt ccctttttag taaaatattc agaaataatt taaatacatc 4920
attgcaatga aaataaatgt tttttattag gcagaatcca gatgctcaag gcccttcata 4980
atatccccca gtttagtagt tggacttagg gaacaaagga acctttaata gaaattggac 5040
agcaagaaag cgagctcagt ggtacttgtg ggccagggcg ttggccaccc cggccaccac 5100
cttctggtac gcggcctgca ctgggggcgt gaattccttc ccgaagtggt gggccagcac 5160
gcacaccagc acgttgccca ggagctgtgg gaggaagata agaggtatga acatgattag 5220
caaaagggcc tagcttggac tcagaataat ccagccttat cccaaccata aaataaaagc 5280
agaatggtag ctggattgta gctgctatta gcaatatgaa acctcttaca tcagttacaa 5340
tttatatgca gaaatattta tatgcagaga tattgctatt gccttaaccc agaaattatc 5400
actgttattc tttagaatgg tgcaaagagg catgatacat tgtatcatta ttgccctgaa 5460
agaaagagat tagggaaagt attagaaata agataaacaa aaaagtatat taaaagaaga 5520
aagcattttt taaaattaca aatgcaaaat taccctgatt tggtcaatat gtgtacacat 5580
attaaaacat tacactttaa cccataaata tgtataatga ttatgtatca attaaaaata 5640
aaagaaaata aagtagggag attatgaata tgcaaataag cacacatata ttccaaatag 5700
taatgtacta ggcagactgt gtaaagtttt tttttaagtt acttaatgta tctcagagat 5760
atttcctttt gttatacaca atgttaaggc attaagtata atagtaaaaa ttgcggagaa 5820
gaaaaaaaaa gaaagcaaga attaaacaaa agaaaacaat tgttatgaac agcaaataaa 5880
agaaactaaa acgatcctga gacttccaca ctgatgcaat cattcgtctg tttcccattc 5940
taaactgtac cctgttactt atccccttcc tatgacatga acttaaccat agaaaagaag 6000
gggaaagaaa acatcaagcg tcccatagac tcaccctgaa gttctcgggg tcgacgtgca 6060
gcttgtcgca gtgcagctcc gacagctggg cgaaggtgcc cttgaggttg tccaggtgag 6120
ccaggccgtc ggagaaggcg ccgagcacct tcttgccgtg ggccttgacc ttagggttgc 6180
ccatcaccgc gtcgggggtg gacagatccc cgaagctctc aaaaaagcgc tgcgtccagg 6240
ggtagaccac cagcagccta agggtgggaa aatagaccaa taggcagaga gagtcagtgc 6300
ctatcagaaa cccaagagtc ttctctgtct ccacatgccc agtttctatt ggtctcctta 6360
aacctgtctt gtaaccttga taccaacctg cccagggcct ccccgccgac ctcgtcgacg 6420
ttcaccttgc cccagagggc cgtcacggcg cttttctcct ccggggttag gtgcaccatg 6480
gtgtctgttt gaggttgcta gtgaacacag ttgtgtcaga agcaaatgta agcaatagat 6540
ggctctgccc tgacttttat gcccagccct ggctcctgcc ctccctgctc ctgggagtag 6600
attggccaac cctagggtgt ggctccacag ggtgaggtct aagtgatgac agccgtacct 6660
gtccttggct cttctggcac tggcttagga gttggacttc aaaccctcag ccctccctct 6720
aagatatatc tcttggcccc ataccatcag tacaaattgc tactaaaaac atcctccttt 6780
gcaagtgtat ttacgtatct agaatatgtc acattctgtc tcaggcatcc attttcttta 6840
tgatgccgtt tgaggtggag ttttagtcag gtggtcagct tctccttttt tttgccatct 6900
gccctgtaag catcctgctg gggacccaga taggagtcat cactctaggc tgagaacatc 6960
tgggcacaca ccctaagcct cagcatgact catcatgact cagcattgct gtgcttgagc 7020
cagaaggttt gcttagaagg ttacacagaa ccagaaggcg ggggtggggc actgaccccg 7080
acaggggcct ggccagaact gctcatgctt ggactatggg aggtcactaa tggagacaca 7140
cagaaatgta acaggaacta aggaaaaact gaagcttatt taatcagaga tgaggatgct 7200
ggaagggata gagggagctg agcttgtaaa aagtatagta atcattcagc aaatggtttt 7260
gaagcacctg ctggatgcta aacactattt tcagtgcttg aatcataaat aagaataaaa 7320
catgtatctt attccccaca agagtccaag taaaaaataa cagttaatta taatgtgctc 7380
tgtcccccag gctggagtgc agtggcacga tctcagctca ctgcaacctc cgcctcccgg 7440
gctggttaga aggttctact ggaggagggt cccagcccat tgctaaatta acatcaggct 7500
ctgagactgg cagtatatct ctaacagtgg ttgatgctat cttctggaac ttgcctgcta 7560
cattgagacc actgacccat acataggaag cccatagctc tgtcctgaac tgttaggcca 7620
ctggtccaga gagtgtgcat ctcctttgat cctcataata accctatgag atagacacaa 7680
ttattactct tactttatag atgatgatcc tgaaaacata ggagtcaagg cacttgcccc 7740
tagctggggg tataggggag cagtcccatg tagtagtaga atgaaaaatg ctgctatgct 7800
gtgcctcccc cacctttccc atgtctgccc tctactcatg gtctatctct cctggctcct 7860
gggagtcatg gactccaccc agcaccacca acctgaccta accacctatc tgagcctgcc 7920
agcctataac ccatctgggc cctgatagct ggtggccagc cctgacccca ccccaccctc 7980
cctggaacct ctgatagaca catctggcac accagctcgc aaagtcaccg tgagggtctt 8040
gtgtttgctg agtcaaaatt ccttgaaatc caagtcctta gagactcctg ctcccaaatt 8100
tacagtcata gacttcttca tggctgtctc ctttatccac agaatgattc ctttgcttca 8160
ttgccccatc catctgatcc tcctcatcag tgcagcacag ggcccatgag cagtagctgc 8220
agagtctcac ataggtctgg cactgcctct gacatgtccg accttaggca aatgcttgac 8280
tcttctctag tgcatgcaaa tctgacactc agtgggcctg ggtgaaggtg agaattttat 8340
tgctgaatga gagcctctgg ggacatcttg ccagtcaatg agtctcaggt tcaatttcct 8400
tctcagtctt ggagtaacag aagctcatgc atttaataaa cggaaatttt gtattgaaat 8460
gagagccatt ggaaatcatt tactccagac tcctacttat aaaaagagaa actgaggctc 8520
agagaagggt ggggactttc tcagtatgac atggaaatga tcaggcttgg attcaaagct 8580
cctgactttc tgtctagtgt atgtgcagtg agcccctttt cctctaactg aaagaaggaa 8640
aaaaaaatgg aacccaaaat attctacata gtttccatgt cacagccagg gctgggcagt 8700
ctcctgttat ttcttttaaa ataaatatat catttaaatg cataaataag caaaccctgc 8760
tcgggaatgg gagggagagt ctctggagtc caccccttct cggccctggc tctgcagata 8820
gtgctatcaa agccctgaca gagccctgcc cattgctggg ccttggagtg agtcagccta 8880
gtagagaggc agggcaagcc atctcatagc tgctgagtgg gagagagaaa agggctcatt 8940
gtctataaac tcaggtcatg gctattctta ttctcacact aagaaaaaga atgagatgtc 9000
tacatatacc ctgcgtcccc tcttgtgtac tggggtcccc aagagctctc taaaagtgat 9060
ggcaaagtca ttgcgctaga tgccatccca tctattataa acctgcattt gtctccacac 9120
accagtcatg gacaataacc ctcctcccag gtccacgtgc ttgtctttgt ataatactca 9180
agtaatttcg gaaaatgtat tctttcaatc ttgttctgtt attcctgttt caatggctta 9240
gtagaaaaag tacatacttg ttttcccata aattgacaat agacaatttc acatcaatgt 9300
ctatatgggt cgttgtgttt gctgtgtttg caaaaactca caataacttt atattgttac 9360
tactctaaga aagttacaac atggtgaata caagagaaag ctattacaag tccagaaaat 9420
aaaagttatc atcttgaggg tcgacctgga attcgagctc ggtaccttta agaccaatga 9480
cttacaaggc agctgtagat cttagccact ttttaaaaga aaagggggga ctggaagggc 9540
taattcactc ccaacgaaga caagatctgc tttttgcttg tactgggtct ctctggttag 9600
accagatctg agcctgggag ctctctggct aactagggaa cccactgctt aagcctcaat 9660
aaagcttgcc ttgagtgctt caagtagtgt gtgcccgtct gttgtgtgac tctggtaact 9720
agagatccct cagacccttt tagtcagtgt ggaaaatctc tagcagtagt agttcatgtc 9780
atcttattat tcagtattta taacttgcaa agaaatgaat atcagagagt gagaggaact 9840
tgtttattgc agcttataat ggttacaaat aaagcaatag catcacaaat ttcacaaata 9900
aagcattttt ttcactgcat tctagttgtg gtttgtccaa actcatcaat gtatcttatc 9960
atgtctggct ctagctatcc cgcccctaac tccgcccatc ccgcccctaa ctccgcccag 10020
ttccgcccat tctccgcccc atggctgact aatttttttt atttatgcag aggccgaggc 10080
cgcctcggcc tctgagctat tccagaagta gtgaggaggc ttttttggag gccgctagcg 10140
tcgaccatta cttattgttt tagctgtcct catgaatgtc ttttcactac ccatttgctt 10200
atcctgcatc tctcagcctt gactccactc agttctcttg cttagagata ccacctttcc 10260
cctgaagtgt tccttccatg ttttacggcg agatggtttc tcctcgcctg gccactcagc 10320
cttagttgtc tctgttgtct tatagaggtc tacttgaaga aggaaaaaca gggggcatgg 10380
tttgactgtc ctgtgagccc ttcttccctg cctcccccac tcacagtgac ccggaatccc 10440
tcgacatggc agtctagcac tagtgcggcc gcagatctgc ttcctcgctc actgactcgc 10500
tgcgctcggt cgttcggctg cggcgagcgg tatcagctca ctcaaaggcg gtaatacggt 10560
tatccacaga atcaggggat aacgcaggaa agaacatgtg agcaaaaggc cagcaaaagg 10620
ccaggaaccg taaaaa 10636
SEQ ID NO: 21
gtgagtctat gggacgcttg atgttttctt tccccttctt ttctatggtt aagttcatgt 60
cataggaagg ggataagtaa cagggtacac atattgacca aatcagggta attttgcatt 120
tgtaatttta aaaaatgctt tcttctttta atatactttt ttgtttatct tatttctaat 180
actttcccta atctctttct ttcagggcaa taatgataca atgtatcatg cctctttgca 240
ccattctaaa gaataacagt gataatttct gggttaaggc aatagcaata tctctgcata 300
taaatatttc tgcatataaa ttgtaactga tgtaagaggt ttcatattgc taatagcagc 360
tacaatccag ctaccattct gcttttattt tatggttggg ataaggctgg attattctga 420
gtccaagcta ggcccttttg ctaatcatgt tcatacctct tatcttcctc ccacag 476
SEQ ID NO: 22
cctcaagatg ataactttta ttttctggac ttgtaatagc tttctcttgt attcaccatg 60
ttgtaacttt cttagagtag taacaatata aagttattgt gagtttttgc aaacacagca 120
aacacaacga cccatataga cattgatgtg aaattgtcta ttgtcaattt atgggaaaac 180
aagtatgtac tttttctact aagccattga aacaggaata acagaacaag attgaaagaa 240
tacattttcc gaaattactt gagtattata caaagacaag cacgtggacc tgggaggagg 300
gttattgtcc atgactggtg tgtggagaca aatgcaggtt tataatagat gggatggcat 360
ctagcgcaat gactttgcca tcacttttag agagctcttg gggaccccag tacacaagag 420
gggacgcagg gtatatgtag acatctcatt ctttttctta gtgtgagaat aagaatagcc 480
atgacctgag tttatagaca atgagccctt ttctctctcc cactcagcag ctatgagatg 540
gcttgccctg cctctctact aggctgactc actccaaggc ccagcaatgg gcagggctct 600
gtcagggctt tgatagcact atctgcagag ccagggccga gaaggggtgg actccagaga 660
ctctccctcc cattcccgag cagggtttgc ttatttatgc atttaaatga tatatttatt 720
ttaaaagaaa taacaggaga ctgcccagcc ctggctgtga catggaaact atgtagaata 780
ttttgggttc catttttttt tccttctttc agttagagga aaaggggctc actgcacata 840
cactagacag aaagtcagga gctttgaatc caagcctgat catttccatg tcatactgag 900
aaagtcccca cccttctctg agcctcagtt tctcttttta taagtaggag tctggagtaa 960
atgatttcca atggctctca tttcaataca aaatttccgt ttattaaatg catgagcttc 1020
tgttactcca agactgagaa ggaaattgaa cctgagactc attgactggc aagatgtccc 1080
cagaggctct cattcagcaa taaaattctc accttcaccc aggcccactg agtgtcagat 1140
ttgcatgcac tagagaagag tcaagcattt gcctaaggtc ggacatgtca gaggcagtgc 1200
cagacctatg tgagactctg cagctactgc tcatgggccc tgtgctgcac tgatgaggag 1260
gatcagatgg atggggcaat gaagcaaagg aatcattctg tggataaagg agacagccat 1320
gaagaagtct atgactgtaa atttgggagc aggagtctct aaggacttgg atttcaagga 1380
attttgactc agcaaacaca agaccctcac ggtgactttg cgagctggtg tgccagatgt 1440
gtctatcaga ggttccaggg agggtggggt ggggtcaggg ctggccacca gctatcaggg 1500
cccagatggg ttataggctg gcaggctcag ataggtggtt aggtcaggtt ggtggtgctg 1560
ggtggagtcc atgactccca ggagccagga gagatagacc atgagtagag ggcagacatg 1620
ggaaaggtgg gggaggcaca gcatagcagc atttttcatt ctactactac atgggactgc 1680
tcccctatac ccccagctag gggcaagtgc cttgactcct atgttttcag gatcatcatc 1740
tataaagtaa gagtaataat tgtgtctatc tcatagggtt attatgagga tcaaaggaga 1800
tgcacactct ctggaccagt ggcctaacag ttcaggacag agctatgggc ttcctatgta 1860
tgggtcagtg gtctcaatgt agcaggcaag ttccagaaga tagcatcaac cactgttaga 1920
gatatactgc cagtctcaga gcctgatgtt aatttagcaa tgggctggga ccctcctcca 1980
gtagaacctt ctaaccagcc cgggaggcgg aggttgcagt gagctgagat cgtgccactg 2040
cactccagcc tgggggacag agcacattat aattaactgt tattttttac ttggactctt 2100
gtggggaata agatacatgt tttattctta tttatgattc aagcactgaa aatagtgttt 2160
agcatccagc aggtgcttca aaaccatttg ctgaatgatt actatacttt ttacaagctc 2220
agctccctct atcccttcca gcatcctcat ctctgattaa ataagcttca gtttttcctt 2280
agttcctgtt acatttctgt gtgtctccat tagtgacctc ccatagtcca agcatgagca 2340
gttctggcca ggcccctgtc ggggtcagtg ccccaccccc gccttctggt tctgtgtaac 2400
cttctaagca aaccttctgg ctcaagcaca gcaatgctga gtcatgatga gtcatgctga 2460
ggcttagggt gtgtgcccag atgttctcag cctagagtga tgactcctat ctgggtcccc 2520
agcaggatgc ttacagggca gatggcaaaa aaaaggagaa gctgaccacc tgactaaaac 2580
tccacctcaa acggcatcat aaagaaaatg gatgcctgag acagaatgtg acatattcta 2640
ga 2642
SEQ ID NO: 23
acatttgctt ctgacacaac tgtgttcact agcaacctca aacagacacc atggtgcacc 60
tgacccctga ggagaagagc gccgtgacag ccctgtgggg caaggtgaac gtggacgaag 120
tgggaggaga ggccctgggc aggctgctgg tggtgtaccc atggacccag agattctttg 180
agagcttcgg cgacctgtcc acaccagatg ccgtgatggg caaccccaag gtgaaggccc 240
acggcaagaa ggtgctggga gccttctctg acggactggc ccacctggat aatctgaagg 300
gcacctttgc ccagctgagc gagctgcact gcgacaagct gcacgtggat cccgagaact 360
tcaggctcct gggcaacgtg ctggtgtgcg tgctggccca ccacttcggc aaggagttta 420
caccccctgt gcaggcagca taccagaagg tggtggcagg agtggcaaat gcactggccc 480
acaagtatca ctgagctcgc tttcttgctg tccaatttct attaaaggtt cctttgttcc 540
ctaagtccaa ctactaaact gggggatatt atgaagggcc ttgagcatct ggattctgcc 600
taataaaaaa catttatttt cattgcaa 628
SEQ ID NO: 24
acatttgctt ctgacacaac tgtgttcact agcaacctca aacagacacc atggtgcacc 60
taaccccgga ggagaaaagc gccgtgacgg ccctctgggg caaggtgaac gtcgacgagg 120
tcggcgggga ggccctgggc aggctgctgg tggtctaccc ctggacgcag cgcttttttg 180
agagcttcgg ggatctgtcc acccccgacg cggtgatggg caaccctaag gtcaaggccc 240
acggcaagaa ggtgctcggc gccttctccg acggcctggc tcacctggac aacctcaagg 300
gcaccttcgc ccagctgtcg gagctgcact gcgacaagct gcacgtcgac cccgagaact 360
tcaggctcct gggcaacgtg ctggtgtgcg tgctggccca ccacttcggg aaggaattca 420
cgcccccagt gcaggccgcg taccagaagg tggtggccgg ggtggccaac gccctggccc 480
acaagtacca ctgagctcgc tttcttgctg tccaatttct attaaaggtt cctttgttcc 540
ctaagtccaa ctactaaact gggggatatt atgaagggcc ttgagcatct ggattctgcc 600
taataaaaaa catttatttt cattgcaa 628
SEQ ID NO: 25
acatttgctt ctgacacaac tgtgttcact agcaacctca aacagacacc atggtgcacc 60
taaccccgga ggagaaaagc gccgtgacgg ccctctgggg caaggtgaac gtcgacgagg 120
tcggcgggga ggccctgggc aggctgctgg tggtctaccc ctggacgcag cgcttttttg 180
agagcttcgg ggatctgtcc acccccgacg cggtgatggg caaccctaag gtcaaggccc 240
acggcaagaa ggtgctcggc gccttctccg acggcctggc tcacctggac aacctcaagg 300
gcaccttcgc cacactgtcg gagctgcact gcgacaagct gcacgtcgac cccgagaact 360
tcaggctcct gggcaacgtg ctggtgtgcg tgctggccca ccacttcggg aaggaattca 420
cgcccccagt gcaggccgcg taccagaagg tggtggccgg ggtggccaac gccctggccc 480
acaagtacca ctgagctcgc tttcttgctg tccaatttct attaaaggtt cctttgttcc 540
ctaagtccaa ctactaaact gggggatatt atgaagggcc ttgagcatct ggattctgcc 600
taataaaaaa catttatttt cattgcaa 628

1. A vector comprising:
a) a left (5′) retroviral LTR;
b) a human β-globin gene;
c) a human β-globin gene upstream locus control region (LCR);
d) a cis-acting posttranscriptional regulatory element;
e) a right (3′) retroviral LTR; and
f) a cis-acting element SV40 polyadenylation signal and/or SV40 origin.

2. The vector of claim 1, wherein the human β-globin gene comprises a human β-globin promoter, exon 1, intron 1, exon 2, intron 2, exon 3, and a human β-globin 3′-enhancer.

3. The vector of claim 1, wherein the human β-globin gene comprises a wild-type exon 2 or an exon 2 encoding a threonine to glutamine mutation at codon 87 (T87Q).

4. The vector of claim 1, wherein the human β-globin gene upstream LCR comprises one or more truncated DNase I hypersensitive sites selected from the group consisting of HS2, HS3, and HS4.

5. The vector of claim 1, wherein the posttranscriptional regulatory element is a woodchuck hepatitis virus post-transcriptional regulatory element (WPRE), optionally wherein the WPRE is a mutated WPRE comprising the nucleotide sequence of SEQ ID NO: 33.

6. (canceled)

7. The vector of claim 1, wherein the SV40 polyadenylation signal and/or SV40 origin is located 3′ downstream of the right (3′) retroviral LTR.

8. The vector of claim 1, wherein the human β-globin gene comprises one, two, or all of the nucleotide sequences of SEQ ID NO: 23, SEQ ID NO: 24, or SEQ ID NO: 25, or a nucleotide sequence having at least 85%, 90%, 95%, or 99% identity thereto.

9. The vector of claim 1, wherein the left (5′) retroviral LTR, the right (3′) retroviral LTR, or both, is a lentiviral LTR, wherein the left (5′) LTR comprises a promoter that is replaced with a heterologous promoter, and wherein the right (3′) LTR is a self-inactivating (SIN) LTR.

10. The vector of claim 1, further comprising one or more of a Psi packaging sequence (Ψ+), a central polypurine tract/DNA flap (cPPT/FLAP), or a retroviral export element-rev response element (RRE).

11. The vector of claim 1, which comprises the nucleotide sequence of any of SEQ ID NO: 5, SEQ ID NO: 7, SEQ ID NO: 8, SEQ ID NO: 13, SEQ ID NO: 15, SEQ ID NO: 17, SEQ ID NO: 18, SEQ ID NO: 19, or SEQ ID NO: 20, or a nucleotide sequence having at least 85%, 90%, 95%, or 99% identity thereto.

12. A composition comprising the vector of claim 1 and a pharmaceutically acceptable carrier.

13. A human cell transduced with the vector of claim 1.

14. The cell of claim 13, which is an embryonic stem cell, an adult stem cell, an adult progenitor cell, a differentiated adult cell, a hematopoietic stem cell, or a hematopoietic progenitor cell, optionally wherein the hematopoietic stem cell or hematopoietic progenitor cell is obtained from bone marrow, cord blood, placental blood, or peripheral blood.

15. (canceled)

16. A composition comprising a cell transduced with a vector comprising:
a) a left (5′) retroviral LTR;
b) a human β-globin gene;
c) a human β-globin gene upstream locus control region (LCR);
d) a cis-acting posttranscriptional regulatory element;
e) a right (3′) retroviral LTR; and
f) a cis-acting element SV40 polyadenylation signal and/or SV40 origin, and a pharmaceutically acceptable carrier.

17. A method of treating β-thalassemia, comprising administering to a subject in need thereof an effective amount of a cell transduced with the vector of claim 1.

18. The method of claim 17, further comprising obtaining a cell from the subject and transducing the cell with the vector, optionally wherein the cell is a hematopoietic stem cell or a hematopoietic progenitor cell.

19. (canceled)

20. The method of claim 17, further comprising administering to the subject an effective amount of busulfan and cyclophosphamide prior to administering the cell transduced with the vector to the subject, optionally wherein:
(a) busulfan is administered at a dose of 2.4 to 4.8 mg/kg/day intravenously;
(b) cyclophosphamide is administered at a dose of 45 to 65 mg/kg/day intravenously;
(c) cyclophosphamide is administered 24 hours after busulfan is administered;
(d) busulfan is administered for 2-4 days and cyclophosphamide is administered for 1-5 days; and
(e) the administration of the cell transduced the vector is initiated 24-72 hours after the administration of cyclophshamide is completed.

21. (canceled)

22. A formulation comprising the vector of claim 1, a buffer, a stabilizer, and sodium chloride.

23. The formulation of claim 22, wherein:
(a) the vector is present at a concentration of 5×10⁸ TU/mL to 5×10⁹ TU/mL;
(b) the buffer comprises a phosphate buffer, sodium citrate, or PIPES;
(c) the buffer is present at a concentration of 10 mM to 50 mM;
(d) the stabilizer comprises sucrose, trehalose, sorbitol, inositol, or glucose;
(e) the stabilizer is present at a concentration of 1% to 5%; or
(f) sodium chloride is present at a concentration of 50 mM to 200 mM.

24.-28. (canceled)

29. The formulation of claim 22, wherein the buffer comprises sodium citrate and the stabilizer comprises sucrose and/or the vector is present at a concentration of 5×10⁸ TU/mL to 5×10⁹ TU/mL, sodium citrate is present at a concentration of 20 mM to 40 mM, sucrose is present at a concentration of 1% to 2%, and sodium chloride is present at a concentration of 100 mM to 150 mM.

30. (canceled)

31. A method of treating sickle cell anemia, comprising administering to a subject in need thereof an effective amount of a cell transduced with the vector of claim 1.


SEQUENCE LISTING (33 sequences)

SEQ ID NO: 1 - DNA, 4928 bp, synthetic construct
  source 1..4928: mol_type = other DNA
ggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaagcgcttttgaagctcggatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcaagaaagccatccagtttactttgcagggcttcccaaccttaccagaggcctgcgccgcggccagctggctagcaattcccgggttaactctagagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgaggcatgcctgcaggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagagggtcgaccattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaa

SEQ ID NO: 2 - DNA, 5022 bp, synthetic construct
  source 1..5022: mol_type = other DNA
cctcaagatgataacttttattttctggacttgtaatagctttctcttgtattcaccatgttgtaactttcttagagtagtaacaatataaagttattgtgagtttttgcaaacacagcaaacacaacgacccatatagacattgatgtgaaattgtctattgtcaatttatgggaaaacaagtatgtactttttctactaagccattgaaacaggaataacagaacaagattgaaagaatacattttccgaaattacttgagtattatacaaagacaagcacgtggacctgggaggagggttattgtccatgactggtgtgtggagacaaatgcaggtttataatagatgggatggcatctagcgcaatgactttgccatcacttttagagagctcttggggaccccagtacacaagaggggacgcagggtatatgtagacatctcattctttttcttagtgtgagaataagaatagccatgacctgagtttatagacaatgagcccttttctctctcccactcagcagctatgagatggcttgccctgcctctctactaggctgactcactccaaggcccagcaatgggcagggctctgtcagggctttgatagcactatctgcagagccagggccgagaaggggtggactccagagactctccctcccattcccgagcagggtttgcttatttatgcatttaaatgatatatttattttaaaagaaataacaggagactgcccagccctggctgtgacatggaaactatgtagaatattttgggttccatttttttttccttctttcagttagaggaaaaggggctcactgcacatacactagacagaaagtcaggagctttgaatccaagcctgatcatttccatgtcatactgagaaagtccccacccttctctgagcctcagtttctctttttataagtaggagtctggagtaaatgatttccaatggctctcatttcaatacaaaatttccgtttattaaatgcatgagcttctgttactccaagactgagaaggaaattgaacctgagactcattgactggcaagatgtccccagaggctctcattcagcaataaaattctcaccttcacccaggcccactgagtgtcagatttgcatgcactagagaagagtcaagcatttgcctaaggtcggacatgtcagaggcagtgccagacctatgtgagactctgcagctactgctcatgggccctgtgctgcactgatgaggaggatcagatggatggggcaatgaagcaaaggaatcattctgtggataaaggagacagccatgaagaagtctatgactgtaaatttgggagcaggagtctctaaggacttggatttcaaggaattttgactcagcaaacacaagaccctcacggtgactttgcgagctggtgtgccagatgtgtctatcagaggttccagggagggtggggtggggtcagggctggccaccagctatcagggcccagatgggttataggctggcaggctcagataggtggttaggtcaggttggtggtgctgggtggagtccatgactcccaggagccaggagagatagaccatgagtagagggcagacatgggaaaggtgggggaggcacagcatagcagcatttttcattctactactacatgggactgctcccctatacccccagctaggggcaagtgccttgactcctatgttttcaggatcatcatctataaagtaagagtaataattgtgtctatctcatagggttattatgaggatcaaaggagatgcacactctctggaccagtggcctaacagttcaggacagagctatgggcttcctatgtatgggtcagtggtctcaatgtagcaggcaagttccagaagatagcatcaaccactgttagagatatactgccagtctcagagcctgatgttaatttagcaatgggctgggaccctcctccagtagaaccttctaaccagcccgggaggcggaggttgcagtgagctgagatcgtgccactgcactccagcctgggggacagagcacattataattaactgttattttttacttggactcttgtggggaataagatacatgttttattcttatttatgattcaagcactgaaaatagtgtttagcatccagcaggtgcttcaaaaccatttgctgaatgattactatactttttacaagctcagctccctctatcccttccagcatcctcatctctgattaaataagcttcagtttttccttagttcctgttacatttctgtgtgtctccattagtgacctcccatagtccaagcatgagcagttctggccaggcccctgtcggggtcagtgccccacccccgccttctggttctgtgtaaccttctaagcaaaccttctggctcaagcacagcaatgctgagtcatgatgagtcatgctgaggcttagggtgtgtgcccagatgttctcagcctagagtgatgactcctatctgggtccccagcaggatgcttacagggcagatggcaaaaaaaaggagaagctgaccacctgactaaaactccacctcaaacggcatcataaagaaaatggatgcctgagacagaatgtgacatattctagatacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgcccagctgagtgagctgcactgtgacaagctgcacgtggatcctgagaacttcagggtgagtctatgggacgcttgatgttttctttccccttcttttctatggttaagttcatgtcataggaaggggataagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcatatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacaggggtcatggtttgactgtcctgtgagcccttcttccctgcctcccccactcacagtgacccggaatctgcagtgctagtctcccggaactatcactctttcacagtctgctttggaaggactgggcttagtatgaaaagttaggactgagaagaatttgaaaggcggctttttgtagcttgatattcactactgtcttattaccctgtcataggcccaccccaaatggaagtcccattcttcctcaggatgtttaagattagcattcaggaagagatcagaggtctgctggctcccttatcatgtcccttatggtgcttctggctctgcagttattagcatagtgttaccatcaaccaccttaacttcatttttcttattcaatacctagg

SEQ ID NO: 3 - DNA, 9959 bp, synthetic construct
  source 1..9959: mol_type = other DNA
ggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaagcgcttttgaagctcggatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcaagaaagccatccagtttactttgcagggcttcccaaccttaccagaggcctgcgccgcggccagctggctagcaattcccgggttaactctagagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatccctaggtattgaataagaaaaatgaagttaaggtggttgatggtaacactatgctaataactgcagagccagaagcaccataagggacatgataagggagccagcagacctctgatctcttcctgaatgctaatcttaaacatcctgaggaagaatgggacttccatttggggtgggcctatgacagggtaataagacagtagtgaatatcaagctacaaaaagccgcctttcaaattcttctcagtcctaacttttcatactaagcccagtccttccaaagcagactgtgaaagagtgatagttccgggagactagcactgcagattccgggtcactgtgagtgggggaggcagggaagaagggctcacaggacagtcaaaccatgacccctgtttttccttcttcaagtagacctctataagacaacagagacaactaaggctgagtggccaggcgaggagaaaccatctcgccgtaaaacatggaaggaacacttcaggggaaaggtggtatctctaagcaagagaactgagtggagtcaaggctgagagatgcaggataagcaaatgggtagtgaaaagacattcatgaggacagctaaaacaataagtaatgtaaaatacagcatagcaaaactttaacctccaaatcaagcctctacttgaatccttttctgagggatgaataaggcatatgcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagagatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttatccccttcctatgacatgaacttaaccatagaaaagaaggggaaagaaaacatcaagcgtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacttcatccacgttcaccttgccccacagggcagtaacggcagacttctcctcaggagtcagatgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacgtatctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctctagtgcatgcaaatctgacactcagtgggcctgggtgaaggtgagaattttattgctgaatgagagcctctggggacatcttgccagtcaatgagtctcaggttcaatttccttctcagtcttggagtaacagaagctcatgcatttaataaacggaaattttgtattgaaatgagagccattggaaatcatttactccagactcctacttataaaaagagaaactgaggctcagagaagggtggggactttctcagtatgacatggaaatgatcaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattctcacactaagaaaaagaatgagatgtctacatataccctgcgtcccctcttgtgtactggggtccccaagagctctctaaaagtgatggcaaagtcattgcgctagatgccatcccatctattataaacctgcatttgtctccacacaccagtcatggacaataaccctcctcccaggtccacgtgcttgtctttgtataatactcaagtaatttcggaaaatgtattctttcaatcttgttctgttattcctgtttcaatggcttagtagaaaaagtacatacttgttttcccataaattgacaatagacaatttcacatcaatgtctatatgggtcgttgtgtttgctgtgtttgcaaaaactcacaataactttatattgttactactctaagaaagttacaacatggtgaatacaagagaaagctattacaagtccagaaaataaaagttatcatcttgagggtcgacctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagagggtcgaccattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaa

SEQ ID NO: 4 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 5 - DNA, 10548 bp, synthetic construct
  source 1..10548: mol_type = other DNA
ggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaagcgcttttgaagctcggatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcaagaaagccatccagtttactttgcagggcttcccaaccttaccagaggcctgcgccgcggccagctggctagcaattcccgggttaactctagagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatccctaggtattgaataagaaaaatgaagttaaggtggttgatggtaacactatgctaataactgcagagccagaagcaccataagggacatgataagggagccagcagacctctgatctcttcctgaatgctaatcttaaacatcctgaggaagaatgggacttccatttggggtgggcctatgacagggtaataagacagtagtgaatatcaagctacaaaaagccgcctttcaaattcttctcagtcctaacttttcatactaagcccagtccttccaaagcagactgtgaaagagtgatagttccgggagactagcactgcagattccgggtcactgtgagtgggggaggcagggaagaagggctcacaggacagtcaaaccatgacccctgtttttccttcttcaagtagacctctataagacaacagagacaactaaggctgagtggccaggcgaggagaaaccatctcgccgtaaaacatggaaggaacacttcaggggaaaggtggtatctctaagcaagagaactgagtggagtcaaggctgagagatgcaggataagcaaatgggtagtgaaaagacattcatgaggacagctaaaacaataagtaatgtaaaatacagcatagcaaaactttaacctccaaatcaagcctctacttgaatccttttctgagggatgaataaggcatatgcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagagatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttatccccttcctatgacatgaacttaaccatagaaaagaaggggaaagaaaacatcaagcgtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacttcatccacgttcaccttgccccacagggcagtaacggcagacttctcctcaggagtcagatgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacgtatctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctctagtgcatgcaaatctgacactcagtgggcctgggtgaaggtgagaattttattgctgaatgagagcctctggggacatcttgccagtcaatgagtctcaggttcaatttccttctcagtcttggagtaacagaagctcatgcatttaataaacggaaattttgtattgaaatgagagccattggaaatcatttactccagactcctacttataaaaagagaaactgaggctcagagaagggtggggactttctcagtatgacatggaaatgatcaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattctcacactaagaaaaagaatgagatgtctacatataccctgcgtcccctcttgtgtactggggtccccaagagctctctaaaagtgatggcaaagtcattgcgctagatgccatcccatctattataaacctgcatttgtctccacacaccagtcatggacaataaccctcctcccaggtccacgtgcttgtctttgtataatactcaagtaatttcggaaaatgtattctttcaatcttgttctgttattcctgtttcaatggcttagtagaaaaagtacatacttgttttcccataaattgacaatagacaatttcacatcaatgtctatatgggtcgttgtgtttgctgtgtttgcaaaaactcacaataactttatattgttactactctaagaaagttacaacatggtgaatacaagagaaagctattacaagtccagaaaataaaagttatcatcttgagggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagagggtcgaccattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaa

SEQ ID NO: 6 - DNA, 297 bp, synthetic construct
  source 1..297: mol_type = other DNA
aacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcc

SEQ ID NO: 7 - DNA, 10262 bp, synthetic construct
  source 1..10262: mol_type = other DNA
ggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaagcgcttttgaagctcggatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcaagaaagccatccagtttactttgcagggcttcccaaccttaccagaggcctgcgccgcggccagctggctagcaattcccgggttaactctagagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatccctaggtattgaataagaaaaatgaagttaaggtggttgatggtaacactatgctaataactgcagagccagaagcaccataagggacatgataagggagccagcagacctctgatctcttcctgaatgctaatcttaaacatcctgaggaagaatgggacttccatttggggtgggcctatgacagggtaataagacagtagtgaatatcaagctacaaaaagccgcctttcaaattcttctcagtcctaacttttcatactaagcccagtccttccaaagcagactgtgaaagagtgatagttccgggagactagcactgcagattccgggtcactgtgagtgggggaggcagggaagaagggctcacaggacagtcaaaccatgacccctgtttttccttcttcaagtagacctctataagacaacagagacaactaaggctgagtggccaggcgaggagaaaccatctcgccgtaaaacatggaaggaacacttcaggggaaaggtggtatctctaagcaagagaactgagtggagtcaaggctgagagatgcaggataagcaaatgggtagtgaaaagacattcatgaggacagctaaaacaataagtaatgtaaaatacagcatagcaaaactttaacctccaaatcaagcctctacttgaatccttttctgagggatgaataaggcatatgcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagagatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttatccccttcctatgacatgaacttaaccatagaaaagaaggggaaagaaaacatcaagcgtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacttcatccacgttcaccttgccccacagggcagtaacggcagacttctcctcaggagtcagatgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacgtatctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctctagtgcatgcaaatctgacactcagtgggcctgggtgaaggtgagaattttattgctgaatgagagcctctggggacatcttgccagtcaatgagtctcaggttcaatttccttctcagtcttggagtaacagaagctcatgcatttaataaacggaaattttgtattgaaatgagagccattggaaatcatttactccagactcctacttataaaaagagaaactgaggctcagagaagggtggggactttctcagtatgacatggaaatgatcaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattctcacactaagaaaaagaatgagatgtctacatataccctgcgtcccctcttgtgtactggggtccccaagagctctctaaaagtgatggcaaagtcattgcgctagatgccatcccatctattataaacctgcatttgtctccacacaccagtcatggacaataaccctcctcccaggtccacgtgcttgtctttgtataatactcaagtaatttcggaaaatgtattctttcaatcttgttctgttattcctgtttcaatggcttagtagaaaaagtacatacttgttttcccataaattgacaatagacaatttcacatcaatgtctatatgggtcgttgtgtttgctgtgtttgcaaaaactcacaataactttatattgttactactctaagaaagttacaacatggtgaatacaagagaaagctattacaagtccagaaaataaaagttatcatcttgagggtcgacctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggccgctagcgtcgaccattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaa

SEQ ID NO: 8 - DNA, 10851 bp, synthetic construct
  source 1..10851: mol_type = other DNA
ggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaagcgcttttgaagctcggatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcaagaaagccatccagtttactttgcagggcttcccaaccttaccagaggcctgcgccgcggccagctggctagcaattcccgggttaactctagagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatccctaggtattgaataagaaaaatgaagttaaggtggttgatggtaacactatgctaataactgcagagccagaagcaccataagggacatgataagggagccagcagacctctgatctcttcctgaatgctaatcttaaacatcctgaggaagaatgggacttccatttggggtgggcctatgacagggtaataagacagtagtgaatatcaagctacaaaaagccgcctttcaaattcttctcagtcctaacttttcatactaagcccagtccttccaaagcagactgtgaaagagtgatagttccgggagactagcactgcagattccgggtcactgtgagtgggggaggcagggaagaagggctcacaggacagtcaaaccatgacccctgtttttccttcttcaagtagacctctataagacaacagagacaactaaggctgagtggccaggcgaggagaaaccatctcgccgtaaaacatggaaggaacacttcaggggaaaggtggtatctctaagcaagagaactgagtggagtcaaggctgagagatgcaggataagcaaatgggtagtgaaaagacattcatgaggacagctaaaacaataagtaatgtaaaatacagcatagcaaaactttaacctccaaatcaagcctctacttgaatccttttctgagggatgaataaggcatatgcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagagatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttatccccttcctatgacatgaacttaaccatagaaaagaaggggaaagaaaacatcaagcgtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacttcatccacgttcaccttgccccacagggcagtaacggcagacttctcctcaggagtcagatgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacgtatctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctctagtgcatgcaaatctgacactcagtgggcctgggtgaaggtgagaattttattgctgaatgagagcctctggggacatcttgccagtcaatgagtctcaggttcaatttccttctcagtcttggagtaacagaagctcatgcatttaataaacggaaattttgtattgaaatgagagccattggaaatcatttactccagactcctacttataaaaagagaaactgaggctcagagaagggtggggactttctcagtatgacatggaaatgatcaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattctcacactaagaaaaagaatgagatgtctacatataccctgcgtcccctcttgtgtactggggtccccaagagctctctaaaagtgatggcaaagtcattgcgctagatgccatcccatctattataaacctgcatttgtctccacacaccagtcatggacaataaccctcctcccaggtccacgtgcttgtctttgtataatactcaagtaatttcggaaaatgtattctttcaatcttgttctgttattcctgtttcaatggcttagtagaaaaagtacatacttgttttcccataaattgacaatagacaatttcacatcaatgtctatatgggtcgttgtgtttgctgtgtttgcaaaaactcacaataactttatattgttactactctaagaaagttacaacatggtgaatacaagagaaagctattacaagtccagaaaataaaagttatcatcttgagggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggccgctagcgtcgaccattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaa

SEQ ID NO: 9 - DNA, 5612 bp, synthetic construct
  source 1..5612: mol_type = other DNA
ggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaagcgcttttgaagctcggatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcaagaaagccatccagtttactttgcagggcttcccaaccttaccagaggcctgcgccgcggccagctggctagcaattcccgggttaactctagagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattctgacactatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcaaatcctgcacaacagattcttcatgtttggaccaaatcaacttgtgataccatgctcaaagaggcctcaattatatttgagtttttaatttttatgaaaaaaaaaaaaaaaaacggaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttagtcgaccattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaa

SEQ ID NO: 10 - DNA, 3387 bp, synthetic construct
  source 1..3387: mol_type = other DNA
ggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaagcgcttttgaagctcggatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcaagaaagccatccagtttactttgcagggcttcccaaccttaccagaggcctgcgccgcggccagctggctagcaattcccgggttaactctagagaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaagctcgagctcgtttagtgaaccgtcagatcgcctggagacgccatccacgctgttttgacctccatagaagacaccgggaccgatccagcctcccctcgaagctagtcgattaggcatctcctatggcaggaagaagcggagacagcgacgaagacctcctcaaggcagtcagactcatcaagtttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtgctgttagcttgctcaatgccacagctatagcagtagctgaggggacagatagggttatagaagtagtacaagaagcttggcactggccgtcgttttacaacgtcgtgatctgagcctgggagatctctggctaactagggaacccactgcttaagcctcaataaagcttgccttgagtgcttcaagtagtgtgtgcccgtctgttgtgtgactctggtaactagagatcaggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtgtcgaccattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaa

SEQ ID NO: 11 - DNA, 9171 bp, synthetic construct
  source 1..9171: mol_type = other DNA
ggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaagcgcttttgaagctcggatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcaagaaagccatccagtttactttgcagggcttcccaaccttaccagaggcctgcgccgcggccagctggctagcaattcccgggttaactctagagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattcgagatctgccgccgccatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggatcagacatcgctggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatactggtgctgacgacacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccgcagtcacaaggagtaatagaatctatgaataaagaattaaagaaaattataggacaggtaagagatcaggctgaacatcttaaaacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaattccggagcggccgcaggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttccgcggaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaagtcgaccattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaa

SEQ ID NO: 12 - DNA, 2754 bp, synthetic construct
  source 1..2754: mol_type = other DNA
tacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcatatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacaggggtcatggtttgactgtcctgtgagcccttcttccctgcctcccccactcacagtgacccggaatctgcagtgctagtctcccggaactatcactctttcacagtctgctttggaaggactgggcttagtatgaaaagttaggactgagaagaatttgaaaggcggctttttgtagcttgatattcactactgtcttattaccctgtcataggcccaccccaaatggaagtcccattcttcctcaggatgtttaagattagcattcaggaagagatcagaggtctgctggctcccttatcatgtcccttatggtgcttctggctctgcagttattagcatagtgttaccatcaaccaccttaacttcatttttcttattcaatacctagg

SEQ ID NO: 13 - DNA, 11225 bp, synthetic construct
  source 1..11225: mol_type = other DNA
ggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaagcgcttttgaagctcggatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcaagaaagccatccagtttactttgcagggcttcccaaccttaccagaggcctgcgccgcggccagctggctagcaattcccgggttaactctagagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatccctaggtattgaataagaaaaatgaagttaaggtggttgatggtaacactatgctaataactgcagagccagaagcaccataagggacatgataagggagccagcagacctctgatctcttcctgaatgctaatcttaaacatcctgaggaagaatgggacttccatttggggtgggcctatgacagggtaataagacagtagtgaatatcaagctacaaaaagccgcctttcaaattcttctcagtcctaacttttcatactaagcccagtccttccaaagcagactgtgaaagagtgatagttccgggagactagcactgcagattccgggtcactgtgagtgggggaggcagggaagaagggctcacaggacagtcaaaccatgacccctgtttttccttcttcaagtagacctctataagacaacagagacaactaaggctgagtggccaggcgaggagaaaccatctcgccgtaaaacatggaaggaacacttcaggggaaaggtggtatctctaagcaagagaactgagtggagtcaaggctgagagatgcaggataagcaaatgggtagtgaaaagacattcatgaggacagctaaaacaataagtaatgtaaaatacagcatagcaaaactttaacctccaaatcaagcctctacttgaatccttttctgagggatgaataaggcatatgcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagagatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtacacatattaaaacattacactttaacccataaatatgtataatgattatgtatcaattaaaaataaaagaaaataaagtagggagattatgaatatgcaaataagcacacatatattccaaatagtaatgtactaggcagactgtgtaaagtttttttttaagttacttaatgtatctcagagatatttccttttgttatacacaatgttaaggcattaagtataatagtaaaaattgcggagaagaaaaaaaaagaaagcaagaattaaacaaaagaaaacaattgttatgaacagcaaataaaagaaactaaaacgatcctgagacttccacactgatgcaatcattcgtctgtttcccattctaaactgtaccctgttacttatccccttcctatgacatgaacttaaccatagaaaagaaggggaaagaaaacatcaagcgtcccatagactcaccctgaagttctcaggatccacgtgcagcttgtcacagtgcagctcactcagtgtggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacttcatccacgttcaccttgccccacagggcagtaacggcagacttctcctcaggagtcagatgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacgtatctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctctagtgcatgcaaatctgacactcagtgggcctgggtgaaggtgagaattttattgctgaatgagagcctctggggacatcttgccagtcaatgagtctcaggttcaatttccttctcagtcttggagtaacagaagctcatgcatttaataaacggaaattttgtattgaaatgagagccattggaaatcatttactccagactcctacttataaaaagagaaactgaggctcagagaagggtggggactttctcagtatgacatggaaatgatcaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattctcacactaagaaaaagaatgagatgtctacatataccctgcgtcccctcttgtgtactggggtccccaagagctctctaaaagtgatggcaaagtcattgcgctagatgccatcccatctattataaacctgcatttgtctccacacaccagtcatggacaataaccctcctcccaggtccacgtgcttgtctttgtataatactcaagtaatttcggaaaatgtattctttcaatcttgttctgttattcctgtttcaatggcttagtagaaaaagtacatacttgttttcccataaattgacaatagacaatttcacatcaatgtctatatgggtcgttgtgtttgctgtgtttgcaaaaactcacaataactttatattgttactactctaagaaagttacaacatggtgaatacaagagaaagctattacaagtccagaaaataaaagttatcatcttgagggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggccgctagcgtcgaccattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaa

SEQ ID NO: 14 - DNA, 1234 bp, synthetic construct
  source 1..1234: mol_type = other DNA
acatttgcttctgacacaactgtgttcactagcaacctcaaacagacaccatggtgcacctgacccctgaggagaagagcgccgtgacagccctgtggggcaaggtgaacgtggacgaagtgggaggagaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtgtacccatggacccagagattctttgagagcttcggcgacctgtccacaccagatgccgtgatgggcaaccccaaggtgaaggcccacggcaagaaggtgctgggagccttctctgacggactggcccacctggataatctgaagggcacctttgcccagctgagcgagctgcactgcgacaagctgcacgtggatcccgagaacttcagggtgagtctatgggacgcttgatgttttctttccccttcttttctatggttaagttcatgtcataggaaggggataagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtgtgcgtgctggcccaccacttcggcaaggagtttacaccccctgtgcaggcagcataccagaaggtggtggcaggagtggcaaatgcactggcccacaagtatcactgagctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 15 - DNA, 10851 bp, synthetic construct
  source 1..10851: mol_type = other DNA
ggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaagcgcttttgaagctcggatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcaagaaagccatccagtttactttgcagggcttcccaaccttaccagaggcctgcgccgcggccagctggctagcaattcccgggttaactctagagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatccctaggtattgaataagaaaaatgaagttaaggtggttgatggtaacactatgctaataactgcagagccagaagcaccataagggacatgataagggagccagcagacctctgatctcttcctgaatgctaatcttaaacatcctgaggaagaatgggacttccatttggggtgggcctatgacagggtaataagacagtagtgaatatcaagctacaaaaagccgcctttcaaattcttctcagtcctaacttttcatactaagcccagtccttccaaagcagactgtgaaagagtgatagttccgggagactagcactgcagattccgggtcactgtgagtgggggaggcagggaagaagggctcacaggacagtcaaaccatgacccctgtttttccttcttcaagtagacctctataagacaacagagacaactaaggctgagtggccaggcgaggagaaaccatctcgccgtaaaacatggaaggaacacttcaggggaaaggtggtatctctaagcaagagaactgagtggagtcaaggctgagagatgcaggataagcaaatgggtagtgaaaagacattcatgaggacagctaaaacaataagtaatgtaaaatacagcatagcaaaactttaacctccaaatcaagcctctacttgaatccttttctgagggatgaataaggcatatgcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagctcagtgatacttgtgggccagtgcatttgccactcctgccaccaccttctggtatgctgcctgcacagggggtgtaaactccttgccgaagtggtgggccagcacgcacaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagagatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttatccccttcctatgacatgaacttaaccatagaaaagaaggggaaagaaaacatcaagcgtcccatagactcaccctgaagttctcgggatccacgtgcagcttgtcgcagtgcagctcgctcagctgggcaaaggtgcccttcagattatccaggtgggccagtccgtcagagaaggctcccagcaccttcttgccgtgggccttcaccttggggttgcccatcacggcatctggtgtggacaggtcgccgaagctctcaaagaatctctgggtccatgggtacaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctctcctcccacttcgtccacgttcaccttgccccacagggctgtcacggcgctcttctcctcaggggtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacgtatctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctctagtgcatgcaaatctgacactcagtgggcctgggtgaaggtgagaattttattgctgaatgagagcctctggggacatcttgccagtcaatgagtctcaggttcaatttccttctcagtcttggagtaacagaagctcatgcatttaataaacggaaattttgtattgaaatgagagccattggaaatcatttactccagactcctacttataaaaagagaaactgaggctcagagaagggtggggactttctcagtatgacatggaaatgatcaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattctcacactaagaaaaagaatgagatgtctacatataccctgcgtcccctcttgtgtactggggtccccaagagctctctaaaagtgatggcaaagtcattgcgctagatgccatcccatctattataaacctgcatttgtctccacacaccagtcatggacaataaccctcctcccaggtccacgtgcttgtctttgtataatactcaagtaatttcggaaaatgtattctttcaatcttgttctgttattcctgtttcaatggcttagtagaaaaagtacatacttgttttcccataaattgacaatagacaatttcacatcaatgtctatatgggtcgttgtgtttgctgtgtttgcaaaaactcacaataactttatattgttactactctaagaaagttacaacatggtgaatacaagagaaagctattacaagtccagaaaataaaagttatcatcttgagggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggccgctagcgtcgaccattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaa

SEQ ID NO: 16 - DNA, 1234 bp, synthetic construct
  source 1..1234: mol_type = other DNA
acatttgcttctgacacaactgtgttcactagcaacctcaaacagacaccatggtgcacctaaccccggaggagaaaagcgccgtgacggccctctggggcaaggtgaacgtcgacgaggtcggcggggaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccctggacgcagcgcttttttgagagcttcggggatctgtccacccccgacgcggtgatgggcaaccctaaggtcaaggcccacggcaagaaggtgctcggcgccttctccgacggcctggctcacctggacaacctcaagggcaccttcgcccagctgtcggagctgcactgcgacaagctgcacgtcgaccccgagaacttcagggtgagtctatgggacgcttgatgttttctttccccttcttttctatggttaagttcatgtcataggaaggggataagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtgtgcgtgctggcccaccacttcgggaaggaattcacgcccccagtgcaggccgcgtaccagaaggtggtggccggggtggccaacgccctggcccacaagtaccactgagctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 17 - DNA, 10851 bp, synthetic construct
  source 1..10851: mol_type = other DNA
ggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaagcgcttttgaagctcggatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcaagaaagccatccagtttactttgcagggcttcccaaccttaccagaggcctgcgccgcggccagctggctagcaattcccgggttaactctagagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatccctaggtattgaataagaaaaatgaagttaaggtggttgatggtaacactatgctaataactgcagagccagaagcaccataagggacatgataagggagccagcagacctctgatctcttcctgaatgctaatcttaaacatcctgaggaagaatgggacttccatttggggtgggcctatgacagggtaataagacagtagtgaatatcaagctacaaaaagccgcctttcaaattcttctcagtcctaacttttcatactaagcccagtccttccaaagcagactgtgaaagagtgatagttccgggagactagcactgcagattccgggtcactgtgagtgggggaggcagggaagaagggctcacaggacagtcaaaccatgacccctgtttttccttcttcaagtagacctctataagacaacagagacaactaaggctgagtggccaggcgaggagaaaccatctcgccgtaaaacatggaaggaacacttcaggggaaaggtggtatctctaagcaagagaactgagtggagtcaaggctgagagatgcaggataagcaaatgggtagtgaaaagacattcatgaggacagctaaaacaataagtaatgtaaaatacagcatagcaaaactttaacctccaaatcaagcctctacttgaatccttttctgagggatgaataaggcatatgcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagctcagtggtacttgtgggccagggcgttggccaccccggccaccaccttctggtacgcggcctgcactgggggcgtgaattccttcccgaagtggtgggccagcacgcacaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagagatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttatccccttcctatgacatgaacttaaccatagaaaagaaggggaaagaaaacatcaagcgtcccatagactcaccctgaagttctcggggtcgacgtgcagcttgtcgcagtgcagctccgacagctgggcgaaggtgcccttgaggttgtccaggtgagccaggccgtcggagaaggcgccgagcaccttcttgccgtgggccttgaccttagggttgcccatcaccgcgtcgggggtggacagatccccgaagctctcaaaaaagcgctgcgtccaggggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctccccgccgacctcgtcgacgttcaccttgccccagagggccgtcacggcgcttttctcctccggggttaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacgtatctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctctagtgcatgcaaatctgacactcagtgggcctgggtgaaggtgagaattttattgctgaatgagagcctctggggacatcttgccagtcaatgagtctcaggttcaatttccttctcagtcttggagtaacagaagctcatgcatttaataaacggaaattttgtattgaaatgagagccattggaaatcatttactccagactcctacttataaaaagagaaactgaggctcagagaagggtggggactttctcagtatgacatggaaatgatcaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattctcacactaagaaaaagaatgagatgtctacatataccctgcgtcccctcttgtgtactggggtccccaagagctctctaaaagtgatggcaaagtcattgcgctagatgccatcccatctattataaacctgcatttgtctccacacaccagtcatggacaataaccctcctcccaggtccacgtgcttgtctttgtataatactcaagtaatttcggaaaatgtattctttcaatcttgttctgttattcctgtttcaatggcttagtagaaaaagtacatacttgttttcccataaattgacaatagacaatttcacatcaatgtctatatgggtcgttgtgtttgctgtgtttgcaaaaactcacaataactttatattgttactactctaagaaagttacaacatggtgaatacaagagaaagctattacaagtccagaaaataaaagttatcatcttgagggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggccgctagcgtcgaccattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaa

SEQ ID NO: 18 - DNA, 11225 bp, synthetic construct
  source 1..11225: mol_type = other DNA
ggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaagcgcttttgaagctcggatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcaagaaagccatccagtttactttgcagggcttcccaaccttaccagaggcctgcgccgcggccagctggctagcaattcccgggttaactctagagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatccctaggtattgaataagaaaaatgaagttaaggtggttgatggtaacactatgctaataactgcagagccagaagcaccataagggacatgataagggagccagcagacctctgatctcttcctgaatgctaatcttaaacatcctgaggaagaatgggacttccatttggggtgggcctatgacagggtaataagacagtagtgaatatcaagctacaaaaagccgcctttcaaattcttctcagtcctaacttttcatactaagcccagtccttccaaagcagactgtgaaagagtgatagttccgggagactagcactgcagattccgggtcactgtgagtgggggaggcagggaagaagggctcacaggacagtcaaaccatgacccctgtttttccttcttcaagtagacctctataagacaacagagacaactaaggctgagtggccaggcgaggagaaaccatctcgccgtaaaacatggaaggaacacttcaggggaaaggtggtatctctaagcaagagaactgagtggagtcaaggctgagagatgcaggataagcaaatgggtagtgaaaagacattcatgaggacagctaaaacaataagtaatgtaaaatacagcatagcaaaactttaacctccaaatcaagcctctacttgaatccttttctgagggatgaataaggcatatgcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagctcagtggtacttgtgggccagggcgttggccaccccggccaccaccttctggtacgcggcctgcactgggggcgtgaattccttcccgaagtggtgggccagcacgcacaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagagatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtacacatattaaaacattacactttaacccataaatatgtataatgattatgtatcaattaaaaataaaagaaaataaagtagggagattatgaatatgcaaataagcacacatatattccaaatagtaatgtactaggcagactgtgtaaagtttttttttaagttacttaatgtatctcagagatatttccttttgttatacacaatgttaaggcattaagtataatagtaaaaattgcggagaagaaaaaaaaagaaagcaagaattaaacaaaagaaaacaattgttatgaacagcaaataaaagaaactaaaacgatcctgagacttccacactgatgcaatcattcgtctgtttcccattctaaactgtaccctgttacttatccccttcctatgacatgaacttaaccatagaaaagaaggggaaagaaaacatcaagcgtcccatagactcaccctgaagttctcggggtcgacgtgcagcttgtcgcagtgcagctccgacagctgggcgaaggtgcccttgaggttgtccaggtgagccaggccgtcggagaaggcgccgagcaccttcttgccgtgggccttgaccttagggttgcccatcaccgcgtcgggggtggacagatccccgaagctctcaaaaaagcgctgcgtccaggggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctccccgccgacctcgtcgacgttcaccttgccccagagggccgtcacggcgcttttctcctccggggttaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacgtatctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctctagtgcatgcaaatctgacactcagtgggcctgggtgaaggtgagaattttattgctgaatgagagcctctggggacatcttgccagtcaatgagtctcaggttcaatttccttctcagtcttggagtaacagaagctcatgcatttaataaacggaaattttgtattgaaatgagagccattggaaatcatttactccagactcctacttataaaaagagaaactgaggctcagagaagggtggggactttctcagtatgacatggaaatgatcaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattctcacactaagaaaaagaatgagatgtctacatataccctgcgtcccctcttgtgtactggggtccccaagagctctctaaaagtgatggcaaagtcattgcgctagatgccatcccatctattataaacctgcatttgtctccacacaccagtcatggacaataaccctcctcccaggtccacgtgcttgtctttgtataatactcaagtaatttcggaaaatgtattctttcaatcttgttctgttattcctgtttcaatggcttagtagaaaaagtacatacttgttttcccataaattgacaatagacaatttcacatcaatgtctatatgggtcgttgtgtttgctgtgtttgcaaaaactcacaataactttatattgttactactctaagaaagttacaacatggtgaatacaagagaaagctattacaagtccagaaaataaaagttatcatcttgagggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggccgctagcgtcgaccattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaa

SEQ ID NO: 19 - DNA, 11225 bp, synthetic construct
  source 1..11225: mol_type = other DNA
ggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaagcgcttttgaagctcggatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcaagaaagccatccagtttactttgcagggcttcccaaccttaccagaggcctgcgccgcggccagctggctagcaattcccgggttaactctagagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatccctaggtattgaataagaaaaatgaagttaaggtggttgatggtaacactatgctaataactgcagagccagaagcaccataagggacatgataagggagccagcagacctctgatctcttcctgaatgctaatcttaaacatcctgaggaagaatgggacttccatttggggtgggcctatgacagggtaataagacagtagtgaatatcaagctacaaaaagccgcctttcaaattcttctcagtcctaacttttcatactaagcccagtccttccaaagcagactgtgaaagagtgatagttccgggagactagcactgcagattccgggtcactgtgagtgggggaggcagggaagaagggctcacaggacagtcaaaccatgacccctgtttttccttcttcaagtagacctctataagacaacagagacaactaaggctgagtggccaggcgaggagaaaccatctcgccgtaaaacatggaaggaacacttcaggggaaaggtggtatctctaagcaagagaactgagtggagtcaaggctgagagatgcaggataagcaaatgggtagtgaaaagacattcatgaggacagctaaaacaataagtaatgtaaaatacagcatagcaaaactttaacctccaaatcaagcctctacttgaatccttttctgagggatgaataaggcatatgcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagctcagtggtacttgtgggccagggcgttggccaccccggccaccaccttctggtacgcggcctgcactgggggcgtgaattccttcccgaagtggtgggccagcacgcacaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagagatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtacacatattaaaacattacactttaacccataaatatgtataatgattatgtatcaattaaaaataaaagaaaataaagtagggagattatgaatatgcaaataagcacacatatattccaaatagtaatgtactaggcagactgtgtaaagtttttttttaagttacttaatgtatctcagagatatttccttttgttatacacaatgttaaggcattaagtataatagtaaaaattgcggagaagaaaaaaaaagaaagcaagaattaaacaaaagaaaacaattgttatgaacagcaaataaaagaaactaaaacgatcctgagacttccacactgatgcaatcattcgtctgtttcccattctaaactgtaccctgttacttatccccttcctatgacatgaacttaaccatagaaaagaaggggaaagaaaacatcaagcgtcccatagactcaccctgaagttctcggggtcgacgtgcagcttgtcgcagtgcagctccgacagtgtggcgaaggtgcccttgaggttgtccaggtgagccaggccgtcggagaaggcgccgagcaccttcttgccgtgggccttgaccttagggttgcccatcaccgcgtcgggggtggacagatccccgaagctctcaaaaaagcgctgcgtccaggggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctccccgccgacctcgtcgacgttcaccttgccccagagggccgtcacggcgcttttctcctccggggttaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacgtatctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctctagtgcatgcaaatctgacactcagtgggcctgggtgaaggtgagaattttattgctgaatgagagcctctggggacatcttgccagtcaatgagtctcaggttcaatttccttctcagtcttggagtaacagaagctcatgcatttaataaacggaaattttgtattgaaatgagagccattggaaatcatttactccagactcctacttataaaaagagaaactgaggctcagagaagggtggggactttctcagtatgacatggaaatgatcaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattctcacactaagaaaaagaatgagatgtctacatataccctgcgtcccctcttgtgtactggggtccccaagagctctctaaaagtgatggcaaagtcattgcgctagatgccatcccatctattataaacctgcatttgtctccacacaccagtcatggacaataaccctcctcccaggtccacgtgcttgtctttgtataatactcaagtaatttcggaaaatgtattctttcaatcttgttctgttattcctgtttcaatggcttagtagaaaaagtacatacttgttttcccataaattgacaatagacaatttcacatcaatgtctatatgggtcgttgtgtttgctgtgtttgcaaaaactcacaataactttatattgttactactctaagaaagttacaacatggtgaatacaagagaaagctattacaagtccagaaaataaaagttatcatcttgagggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggccgctagcgtcgaccattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaa

SEQ ID NO: 20 - DNA, 10636 bp, synthetic construct
  source 1..10636: mol_type = other DNA
ggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaagcgcttttgaagctcggatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcaagaaagccatccagtttactttgcagggcttcccaaccttaccagaggcctgcgccgcggccagctggctagcaattcccgggttaactctagagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatccctaggtattgaataagaaaaatgaagttaaggtggttgatggtaacactatgctaataactgcagagccagaagcaccataagggacatgataagggagccagcagacctctgatctcttcctgaatgctaatcttaaacatcctgaggaagaatgggacttccatttggggtgggcctatgacagggtaataagacagtagtgaatatcaagctacaaaaagccgcctttcaaattcttctcagtcctaacttttcatactaagcccagtccttccaaagcagactgtgaaagagtgatagttccgggagactagcactgcagattccgggtcactgtgagtgggggaggcagggaagaagggctcacaggacagtcaaaccatgacccctgtttttccttcttcaagtagacctctataagacaacagagacaactaaggctgagtggccaggcgaggagaaaccatctcgccgtaaaacatggaaggaacacttcaggggaaaggtggtatctctaagcaagagaactgagtggagtcaaggctgagagatgcaggataagcaaatgggtagtgaaaagacattcatgaggacagctaaaacaataagtaatgtaaaatacagcatagcaaaactttaacctccaaatcaagcctctacttgaatccttttctgagggatgaataaggcatatgcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagctcagtggtacttgtgggccagggcgttggccaccccggccaccaccttctggtacgcggcctgcactgggggcgtgaattccttcccgaagtggtgggccagcacgcacaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagagatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtacacatattaaaacattacactttaacccataaatatgtataatgattatgtatcaattaaaaataaaagaaaataaagtagggagattatgaatatgcaaataagcacacatatattccaaatagtaatgtactaggcagactgtgtaaagtttttttttaagttacttaatgtatctcagagatatttccttttgttatacacaatgttaaggcattaagtataatagtaaaaattgcggagaagaaaaaaaaagaaagcaagaattaaacaaaagaaaacaattgttatgaacagcaaataaaagaaactaaaacgatcctgagacttccacactgatgcaatcattcgtctgtttcccattctaaactgtaccctgttacttatccccttcctatgacatgaacttaaccatagaaaagaaggggaaagaaaacatcaagcgtcccatagactcaccctgaagttctcggggtcgacgtgcagcttgtcgcagtgcagctccgacagctgggcgaaggtgcccttgaggttgtccaggtgagccaggccgtcggagaaggcgccgagcaccttcttgccgtgggccttgaccttagggttgcccatcaccgcgtcgggggtggacagatccccgaagctctcaaaaaagcgctgcgtccaggggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctccccgccgacctcgtcgacgttcaccttgccccagagggccgtcacggcgcttttctcctccggggttaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacgtatctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctctagtgcatgcaaatctgacactcagtgggcctgggtgaaggtgagaattttattgctgaatgagagcctctggggacatcttgccagtcaatgagtctcaggttcaatttccttctcagtcttggagtaacagaagctcatgcatttaataaacggaaattttgtattgaaatgagagccattggaaatcatttactccagactcctacttataaaaagagaaactgaggctcagagaagggtggggactttctcagtatgacatggaaatgatcaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattctcacactaagaaaaagaatgagatgtctacatataccctgcgtcccctcttgtgtactggggtccccaagagctctctaaaagtgatggcaaagtcattgcgctagatgccatcccatctattataaacctgcatttgtctccacacaccagtcatggacaataaccctcctcccaggtccacgtgcttgtctttgtataatactcaagtaatttcggaaaatgtattctttcaatcttgttctgttattcctgtttcaatggcttagtagaaaaagtacatacttgttttcccataaattgacaatagacaatttcacatcaatgtctatatgggtcgttgtgtttgctgtgtttgcaaaaactcacaataactttatattgttactactctaagaaagttacaacatggtgaatacaagagaaagctattacaagtccagaaaataaaagttatcatcttgagggtcgacctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggccgctagcgtcgaccattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaa

SEQ ID NO: 21 - DNA, 476 bp, synthetic construct
  source 1..476: mol_type = other DNA
gtgagtctatgggacgcttgatgttttctttccccttcttttctatggttaagttcatgtcataggaaggggataagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 22 - DNA, 2642 bp, synthetic construct
  source 1..2642: mol_type = other DNA
cctcaagatgataacttttattttctggacttgtaatagctttctcttgtattcaccatgttgtaactttcttagagtagtaacaatataaagttattgtgagtttttgcaaacacagcaaacacaacgacccatatagacattgatgtgaaattgtctattgtcaatttatgggaaaacaagtatgtactttttctactaagccattgaaacaggaataacagaacaagattgaaagaatacattttccgaaattacttgagtattatacaaagacaagcacgtggacctgggaggagggttattgtccatgactggtgtgtggagacaaatgcaggtttataatagatgggatggcatctagcgcaatgactttgccatcacttttagagagctcttggggaccccagtacacaagaggggacgcagggtatatgtagacatctcattctttttcttagtgtgagaataagaatagccatgacctgagtttatagacaatgagcccttttctctctcccactcagcagctatgagatggcttgccctgcctctctactaggctgactcactccaaggcccagcaatgggcagggctctgtcagggctttgatagcactatctgcagagccagggccgagaaggggtggactccagagactctccctcccattcccgagcagggtttgcttatttatgcatttaaatgatatatttattttaaaagaaataacaggagactgcccagccctggctgtgacatggaaactatgtagaatattttgggttccatttttttttccttctttcagttagaggaaaaggggctcactgcacatacactagacagaaagtcaggagctttgaatccaagcctgatcatttccatgtcatactgagaaagtccccacccttctctgagcctcagtttctctttttataagtaggagtctggagtaaatgatttccaatggctctcatttcaatacaaaatttccgtttattaaatgcatgagcttctgttactccaagactgagaaggaaattgaacctgagactcattgactggcaagatgtccccagaggctctcattcagcaataaaattctcaccttcacccaggcccactgagtgtcagatttgcatgcactagagaagagtcaagcatttgcctaaggtcggacatgtcagaggcagtgccagacctatgtgagactctgcagctactgctcatgggccctgtgctgcactgatgaggaggatcagatggatggggcaatgaagcaaaggaatcattctgtggataaaggagacagccatgaagaagtctatgactgtaaatttgggagcaggagtctctaaggacttggatttcaaggaattttgactcagcaaacacaagaccctcacggtgactttgcgagctggtgtgccagatgtgtctatcagaggttccagggagggtggggtggggtcagggctggccaccagctatcagggcccagatgggttataggctggcaggctcagataggtggttaggtcaggttggtggtgctgggtggagtccatgactcccaggagccaggagagatagaccatgagtagagggcagacatgggaaaggtgggggaggcacagcatagcagcatttttcattctactactacatgggactgctcccctatacccccagctaggggcaagtgccttgactcctatgttttcaggatcatcatctataaagtaagagtaataattgtgtctatctcatagggttattatgaggatcaaaggagatgcacactctctggaccagtggcctaacagttcaggacagagctatgggcttcctatgtatgggtcagtggtctcaatgtagcaggcaagttccagaagatagcatcaaccactgttagagatatactgccagtctcagagcctgatgttaatttagcaatgggctgggaccctcctccagtagaaccttctaaccagcccgggaggcggaggttgcagtgagctgagatcgtgccactgcactccagcctgggggacagagcacattataattaactgttattttttacttggactcttgtggggaataagatacatgttttattcttatttatgattcaagcactgaaaatagtgtttagcatccagcaggtgcttcaaaaccatttgctgaatgattactatactttttacaagctcagctccctctatcccttccagcatcctcatctctgattaaataagcttcagtttttccttagttcctgttacatttctgtgtgtctccattagtgacctcccatagtccaagcatgagcagttctggccaggcccctgtcggggtcagtgccccacccccgccttctggttctgtgtaaccttctaagcaaaccttctggctcaagcacagcaatgctgagtcatgatgagtcatgctgaggcttagggtgtgtgcccagatgttctcagcctagagtgatgactcctatctgggtccccagcaggatgcttacagggcagatggcaaaaaaaaggagaagctgaccacctgactaaaactccacctcaaacggcatcataaagaaaatggatgcctgagacagaatgtgacatattctaga

SEQ ID NO: 23 - DNA, 628 bp, synthetic construct
  source 1..628: mol_type = other DNA
acatttgcttctgacacaactgtgttcactagcaacctcaaacagacaccatggtgcacctgacccctgaggagaagagcgccgtgacagccctgtggggcaaggtgaacgtggacgaagtgggaggagaggccctgggcaggctgctggtggtgtacccatggacccagagattctttgagagcttcggcgacctgtccacaccagatgccgtgatgggcaaccccaaggtgaaggcccacggcaagaaggtgctgggagccttctctgacggactggcccacctggataatctgaagggcacctttgcccagctgagcgagctgcactgcgacaagctgcacgtggatcccgagaacttcaggctcctgggcaacgtgctggtgtgcgtgctggcccaccacttcggcaaggagtttacaccccctgtgcaggcagcataccagaaggtggtggcaggagtggcaaatgcactggcccacaagtatcactgagctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 24 - DNA, 628 bp, synthetic construct
  source 1..628: mol_type = other DNA
acatttgcttctgacacaactgtgttcactagcaacctcaaacagacaccatggtgcacctaaccccggaggagaaaagcgccgtgacggccctctggggcaaggtgaacgtcgacgaggtcggcggggaggccctgggcaggctgctggtggtctacccctggacgcagcgcttttttgagagcttcggggatctgtccacccccgacgcggtgatgggcaaccctaaggtcaaggcccacggcaagaaggtgctcggcgccttctccgacggcctggctcacctggacaacctcaagggcaccttcgcccagctgtcggagctgcactgcgacaagctgcacgtcgaccccgagaacttcaggctcctgggcaacgtgctggtgtgcgtgctggcccaccacttcgggaaggaattcacgcccccagtgcaggccgcgtaccagaaggtggtggccggggtggccaacgccctggcccacaagtaccactgagctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 25 - DNA, 628 bp, synthetic construct
  source 1..628: mol_type = other DNA
acatttgcttctgacacaactgtgttcactagcaacctcaaacagacaccatggtgcacctaaccccggaggagaaaagcgccgtgacggccctctggggcaaggtgaacgtcgacgaggtcggcggggaggccctgggcaggctgctggtggtctacccctggacgcagcgcttttttgagagcttcggggatctgtccacccccgacgcggtgatgggcaaccctaaggtcaaggcccacggcaagaaggtgctcggcgccttctccgacggcctggctcacctggacaacctcaagggcaccttcgccacactgtcggagctgcactgcgacaagctgcacgtcgaccccgagaacttcaggctcctgggcaacgtgctggtgtgcgtgctggcccaccacttcgggaaggaattcacgcccccagtgcaggccgcgtaccagaaggtggtggccggggtggccaacgccctggcccacaagtaccactgagctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 26 - DNA, 20 bp, synthetic construct
  source 1..20: mol_type = other DNA
agtaagaccaccgcacagca

SEQ ID NO: 27 - DNA, 20 bp, synthetic construct
  source 1..20: mol_type = other DNA
ccttggtgggtgctactcct

SEQ ID NO: 28 - DNA, 27 bp, synthetic construct
  source 1..27: mol_type = other DNA
cctccaggtctgaagatcagcggccgc

SEQ ID NO: 29 - DNA, 22 bp, synthetic construct
  source 1..22: mol_type = other DNA
gctgtcatctcttgtgggctgt

SEQ ID NO: 30 - DNA, 26 bp, synthetic construct
  source 1..26: mol_type = other DNA
cctgtcatgcccacacaaatctctcc

SEQ ID NO: 31 - DNA, 21 bp, synthetic construct
  source 1..21: mol_type = other DNA
actcatgggagctgctggttc

SEQ ID NO: 32 - DNA, 589 bp, synthetic construct; identical to SEQ ID NO: 4
  source 1..589: mol_type = other DNA

SEQ ID NO: 33 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc